(19) United States
(12) Patent Application Publication
(10) Pub. No.: US 2025/0170137 A1
(43) Pub. Date: May 29, 2025

(54) Compositions for preventing or treating heart failure (HF)

(71) Applicant: CHONG KUN DANG PHARMACEUTICAL CORP., Seoul (KR)

(72) Inventors: Ju Hee LEE, Yongin-si, Gyeonggi-do (KR); Beom Seob LEE, Yongin-si, Gyeonggi-do (KR); Jiyeon KANG, Yongin-si, Gyeonggi-do (KR)

(21) Appl. No.: 18/833,728

(22) PCT Filed: Jan. 26, 2023
(86) PCT No.: PCT/IB2023/050659

§ 371 Date: Jul. 26, 2024

(30) Foreign Application Priority Data
Jan. 28, 2022 (KR) 10-2022-0013636

(51) Int. Cl. A61K 31/5377; A61K 31/4245; A61K 31/438; A61K 31/454; A61K 31/496; A61K 31/541; A61K 31/551; A61K 31/553; A61P 9/04

(52) U.S. Cl. CPC A61K 31/5377; A61K 31/4245; A61K 31/438; A61K 31/454; A61K 31/496; A61K 31/541; A61K 31/551; A61K 31/553; A61P 9/04

(57) ABSTRACT

The present invention relates to a pharmaceutical composition for preventing or treating heart failure, comprising a compound represented by formula I, optical isomers thereof or pharmaceutically acceptable salts thereof as an active ingredient, a method for preventing or treating heart failure using the compound, a use of the compound for preventing or treating heart failure, and a use of the compound in preparing a medicament for preventing or treating heart failure.

### TECHNICAL FIELD

The present disclosure relates to a pharmaceutical composition for preventing or treating heart failure, comprising a compound represented by formula I, optical isomers thereof or pharmaceutically acceptable salts thereof as an active ingredient, a method for preventing or treating heart failure using the compound, optical isomers thereof or pharmaceutically acceptable salts thereof, a use of the compound, optical isomers thereof or pharmaceutically acceptable salts thereof for preventing or treating heart failure, and a use of the compound, optical isomers thereof or pharmaceutically acceptable salts thereof in preparing a medicament for preventing or treating heart failure.

### BACKGROUND

“Heart failure (HF)” refers to a disease in which the function of the heart is impaired due to cardiovascular and coronary artery diseases, hypertension, and various genetic factors, thus preventing the heart from supplying oxygen and nutrient-filled blood to other parts of the body.

The HF is divided into Heart Failure with Preserved Ejection Fraction (HFpEF), Heart Failure with Midrange Ejection Fraction (HFmrEF), and Heart Failure with Reduced Ejection Fraction (HFrEF) (Theresa A M et al,. Eur Heart J. 2021; 42(36):3599-3726). Until now, the HF appears as a final complication of coronary artery disease, hypertension, cardiomyopathy, etc., and is a severe chronic disease with a long hospitalization period and a high rehospitalization rate due to frequent dysfunction of other organs such as the kidneys, etc. The prognosis of the HF is poorest in cardiovascular disease, which thus has a higher mortality than most solid tumors, with a one-year mortality rate of 37% and a five-year mortality rate of 78% (Braunwald E et al,. Lancet. 2015; 385(9970):812-24).

As a method for treating the HF, drug treatment and surgical treatment have been considered so far. However, most of the methods are to relieve heart functions and pains rather than to treat the underlying cause to ameliorate myocardial cell damages. Even if the heart functions are restored by treating the HF, four out of ten patients are shown to experience the recurrence of the HF within six months if the drug treatment is stopped (Brian P H et al,. Lancet. 2019; 393(10166):61-73).

Accordingly, there is an urgent need for developing a drug capable of effectively treating the HF.

### RELATED ART REFERENCE

### Patent Documents

(Patent Document 1) Korean Unexamined Patent Application Publication No. 2017-0017792

### Non-Patent Documents

(non-Patent Document 1) Theresa A M et al., Eur Heart J 2021; 42(36):3599-3726
(non-Patent Document 1) Braunwald E. et al., Lancet 2015; 385(9970):812-24
(non-Patent Document 1) Yao T P et al., Mol. Cell 2005; 18, 601-607
(non-Patent Document 1) Brundel B et al., Nat Rev Cardiol 2017; 14(11):637-653
(non-Patent Document 1) Pareyson et al., (2011) 10(4):3205
(non-Patent Document 1) Brian P H et al,. Lancet. 2019; 393(10166):61-73)

### DETAILED DESCRIPTION OF THE INVENTION

### Technical Problem

The present disclosure may provide a pharmaceutical composition for preventing or treating heart failure, containing a compound represented by formula I, optical isomers thereof or pharmaceutically acceptable salts thereof as an active ingredient.

The present disclosure may provide a method for preventing or treating heart failure, including administering the compound represented by above formula I, optical isomers thereof or pharmaceutically acceptable salts thereof into an individual.

The present disclosure may provide a use of the compound represented by above formula I, optical isomers thereof or pharmaceutically acceptable salts thereof for preventing or treating heart failure.

The present disclosure may provide a use of the compound represented by above formula I, optical isomers thereof or pharmaceutically acceptable salts thereof in preparing a medicament for preventing or treating heart failure.

### Technical Solution

This is described in detail as follows. Meanwhile, each description and embodiment disclosed in the present invention may be also applied to other descriptions and embodiments thereof, respectively. In other words, all the combinations of various elements disclosed in the present invention fall within the scope of the present invention. Also, it cannot be seen that the scope of the present invention is limited to the specific description described below.

The present disclosure provides a pharmaceutical composition for preventing or treating heart failure, including a compound represented by formula I below, optical isomers thereof or pharmaceutically acceptable salts thereof as an active ingredient.

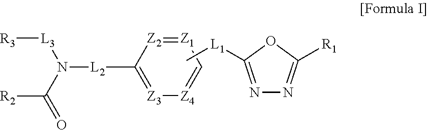

In Formula I,
wherein L₁, L₂ or L₃ are each independently a bond or —(C₁-C₂ alkylene)-;
R₁ is —CX₂H or —CX₃;
R₂ is —NR^{A}R^{B}, —OR^{C},

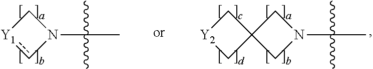

{wherein at least one H

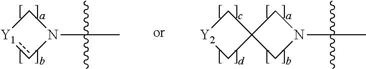

may be substituted with —X, —OH, —O(C₁-C₄ alkyl), —NR^{D}R^{E}—(C₁-C₄ alkyl), —CF₃, —CF₂H, —CN, -aryl, -heteroaryl, —(C₁-C₄ alkyl)-aryl or —(C₁-C₄ alkyl)-heteroaryl, [wherein at least one H of the -aryl, -heteroaryl, —(C₁-C₄ alkyl)-aryl or —(C₁-C₄ alkyl)-heteroaryl may be substituted with —X, —OH, —CF₃ or —CF₂H]};
R₃ is —H, —(C₁-C₄ alkyl), —(C₁-C₄ alkyl)-O(C₁-C₄ alkyl), —(C₁-C₄ alkyl)-C(═O)—O(C₁-C₄ alkyl), —(C₃-C₇ cycloalkyl), —(C₂-C₆ cycloheteroalkyl), -aryl, -heteroaryl, -adamantyl,

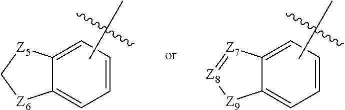

{wherein, at least one H of —(C₁-C₄ alkyl) may be substituted with —X or —OH,
at least one H of -aryl or -heteroaryl each independently may be substituted with —X, —OH, —O(C₁-C₄ alkyl), —OCF₃, —O-aryl, —NR^{D}R^{E}, —(C₁-C₄ alkyl), —CF₃, —CF₂H, —C(═O)—(C₁-C₄ alkyl), —C(═O)—O(C₁-C₄ alkyl), —C(═O)—NR^{D}R^{E}, —S(═O)₂—(C₁-C₄ alkyl), aryl, heteroaryl,

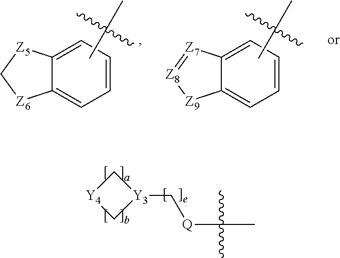

wherein, at least one H of

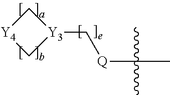

may be substituted with —X, —(C₁-C₄ alkyl), —NR^{D}R^{E}, —CF₃ or —CF₂H], at least one H of —(C₃-C₇ cycloalkyl), —(C₂-C₆ cycloheteroalkyl), adamantyl,

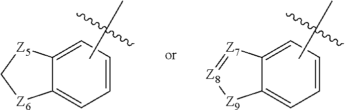

may be each independently substituted with —X, —OH or —(C₁-C₄ alkyl)};
Y₁, Y₂ and Y₄ are each independently —CH₂—, —NR—, —O—, —C(═O)— or —S(═O)₂—;
Y₃ is —CH— or —N—;
Z₁ to Z₄ are each independently N or CR^{Z}, {wherein at least three of Z₁ to Z₄ may not be simultaneously N, and R^{Z} is —H, —X or —O(C₁-C₄ alkyl)};
Z₅ and Z₆ are each independently —CH₂— or —O—;
Z₇ and Z₈ are each independently ═CH— or ═N—;
Z₉ is —NR^{G}— or —S—;
R^{A} and R^{B} are each independently —H, —(C₁-C₄ alkyl), —(C₁-C₄ alkyl)-OH, —(C₁-C₄ alkyl)-NR^{D}R^{E}, -aryl, —(C₁-C₄ alkyl)-aryl, -heteroaryl, —(C₁-C₄ aryl)-heteroaryl, —(C₃-C₇ cycloalkyl), —(C₂-C₆ heterocycloalkyl) or

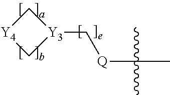

{wherein, at least one H of the —(C₁-C₄ alkyl), —(C₁-C₄ alkyl)-OH or —(C₁-C₄ alkyl)-NR^{D}R^{E} may be substituted with —X,
at least one H of the -aryl, —(C₁-C₄ alkyl)-aryl, -heteroaryl, —(C₁-C₄ alkyl)-heteroaryl, —(C₃-C₇ cycloalkyl) or —(C₂-C₆ heterocycloalkyl) may be substituted with —X, —OH, —O(C₁-C₄ alkyl), —(C₁-C₄ alkyl), —CF₃, —CF₂H or —CN,
at least one H of

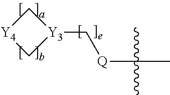

may be substituted with —X, —OH, —O(C₁-C₄ alkyl), —(C₁-C₄ alkyl), —CF₃, —CF₂H, —CN, —(C₂-C₆ heterocycloalkyl), -aryl, —(C₁-C₄ alkyl)-aryl, -heteroaryl or -heteroaryl-(C₁-C₄ alkyl)};
R^{C} is —(C₁-C₄ alkyl), -aryl, —(C₁-C₄ alkyl)-aryl, -heteroaryl or —(C₁-C₄ alkyl)-heteroaryl {wherein, at least one H of —(C₁-C₄ alkyl) may be substituted with —X or —OH, at least one H of -aryl, —(C₁-C₄ alkyl)-aryl, -heteroaryl or —(C₁-C₄ alkyl)-heteroaryl may be substituted with —X, —OH, —CF₃ or —CF₂H}; R^{D} and R^{E} are each independently —H, —(C₁-C₄ alkyl), -aryl or —(C₁-C₄ alkyl)-aryl {wherein, at least one H of —(C₁-C₄ alkyl) may be substituted with —X or —OH, at least one H of -aryl or —(C₁-C₄ alkyl)-aryl may be substituted with —X, —OH, —CF₃ or —CF₂H};
R^{F} is —H, —(C₁-C₆ alkyl), —(C₁-C₄ alkyl)-OH, —(C₁-C₄ alkyl)-O—(C₁-C₄ alkyl), —C(═O)—(C₁-C₄ alkyl), —C(═O)—O(C₁-C₄ alkyl), —(C₁-C₄ alkyl)-C(═O)—O(C₁-C₄ alkyl), —(C₁-C₄ alkyl)-NR^{D}R^{E}, —S(═O)₂—(C₁-C₄ alkyl), -aryl, —(C₁-C₄ alkyl)-aryl, —(C₂-C₄ alkenyl)-aryl, -heteroaryl, —(C₁-C₄ alkyl)-heteroaryl, —C(═O)—(C₃-C₇ cycloalkyl), —(C₂-C₆ heterocycloalkyl) or —(C₁-C₄ alkyl)-C(═O)—(C₂-C₆ heterocycloalkyl)
{wherein, at least one H of —(C₁-C₄ alkyl), —(C₁-C₄ alkyl)-OH, —(C₁-C₄ alkyl)-O—(C₁-C₄ alkyl), —C(═O)—(C₁-C₄ alkyl), —C(═O)—O(C₁-C₄ alkyl), —(C₁-C₄ alkyl)-C(═O)—O(C₁-C₄ alkyl), —(C₁-C₄ alkyl)-NR^{D}R^{E} or —S(═O)₂—(C₁-C₄ alkyl) may be substituted with —X,
at least one H of -aryl, —(C₁-C₄alkyl)-aryl, —(C₂-C₄ alkenyl)-aryl, -heteroaryl, —(C₁-C₄ alkyl)-heteroaryl, —C(═O)—(C₃-C₇ cycloalkyl), —C₂-C₆ heterocycloalkyl or —(C₁-C₄alkyl)-C(═O)—(C₂-C₆heterocycloalkyl) may be substituted with —X, —OH, —CF₃ or —CF₂H};
R^{G} is —H or —(C₁-C₄ alkyl);
Q is —O— or a bond;
[symbol] is a single bond or double bond {provided that, [symbol] is a double bond, Y₁ is ═CH—};
a to e are each independently an integer of 0, 1, 2, 3 or 4 {provided that, a and b may not be simultaneously 0, and c and d may not be simultaneously 0};
X is each independently F, Cl, Br or I.

In the pharmaceutical composition according to the present disclosure, the compound represented by formula I may be below:
L₁, L₂ or L₃ are each independently a bond or —(C₁-C₂alkylene)-;
R₁ is —CX₂H or —CX₃;
R₂ is —NR^{A}R^{B}, —OR^{C},

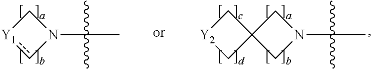

{wherein at least one of H of

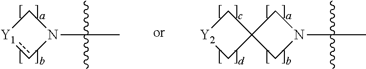

may be substituted with —X, —OH, —NR^{D}R^{E}, —(C₁-C₄ alkyl}
R₃ is —(C₁-C₄ alkyl), —(C₃-C₇ cycloalkyl), -aryl, -heteroaryl, -adamantyl,

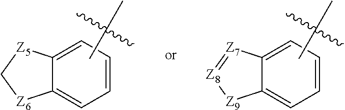

{wherein at least one H of -aryl or -heteroaryl may be each independently substituted with —X, —O(C₁-C₄alkyl), —OCF₃, —O-aryl, —NR^{D}R^{E}, —(C₁-C₄ alkyl), —CF₃, —S(═O)₂—(C₁-C₄alkyl), -aryl, -heteroaryl,

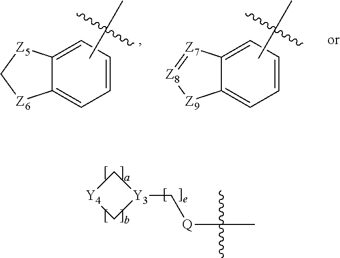

wherein
   at last one H of

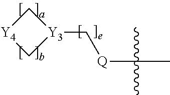

may be substituted with —NR^{D}R^{E} or —(C₁-C₄ alkyl)],
at least one H of

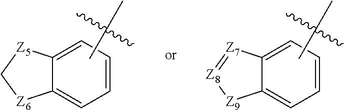

maybe each independently substituted with —(C₁-C₄ alkyl)};
Y₁, Y₂ and Y₄ are each independently —CH₂—, —NR^{F}—, —O—, —C(═O)— or —S(═)₂—;
Y₃ is —CH— or —N—;
Z₁ to Z₄ is each independently N or CR^{Z} {wherein at least three of Z₁ to Z₄ may not be simultaneously N, and R^{Z} is —H, —X or —O(C₁-C₄ alkyl)};
Z₅ and Z₆ are each independently —CH₂— or —O—;
Z₇ and Z₈ are each independently ═CH— or ═N—;
Z₉ is —NR^{G}— or —S—;
R^{A} and R^{B} are each independently —H, —(C₁-C₄ alkyl), —(C₁-C₄ alkyl)-OH, —(C₁-C₄ alkyl)-NR^{D}R^{E}, -aryl, —(C₁-C₄ alkyl)-aryl, —(C₃-C₇ cycloalkyl) or

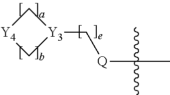

{wherein at least one H of

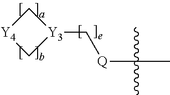

may be substituted with —X, —(C₁-C₄alkyl), —CF₃, —(C₂-C₆ heterocycloalkyl), —(C₁-C₄ alkyl)-aryl, -heteroaryl or heteroaryl-(C₁-C₄ alkyl)};
R^{C} is —(C₁-C₄ alkyl) or -aryl;
R^{D} and R^{E} are each independently —H, —(C₁-C₄alkyl) or —(C₁-C₄ alkyl)-aryl;
R^{F} is —H, —(C₁-C₆alkyl), —(C₁-C₄alkyl)-OH, —(C₁-C₄ alkyl)-O—(C₁-C₄ alkyl), —C(═O)—(C₁-C₄alkyl), —C(═O)—O(C₁-C₄ alkyl), —(C₁-C₄ alkyl)-C(═O)—O(C₁-C₄alkyl), —(C₁-C₄alkyl)-NR^{D}R^{E}, —S(═O)₂—(C₁-C₄ alkyl), -aryl, —(C₁-C₄ alkyl)-aryl, —(C₂-C₄ alkenyl)-aryl, -heteroaryl, —(C₁-C₄ alkyl)-heteroaryl, —C(═O)—(C₃-C₇ cycloalkyl), —(C₂-C₆ heterocycloalkyl) or —(C₁-C₄ alkyl)-C(═O)—(C₂-C₆ heterocycloalkyl)
{wherein at least one H of —(C₁-C₄alkyl) or —C(═O)—O(C₁-C₄alkyl) may be substituted with —X,
at least one H of -aryl may be substituted with —X};
R^{G} is —(C₁-C₄ alkyl);
Q is —O— or a bond;
[symbol] is a single bond or a double bond {provided that [symbol] is a double bond, Y₁ is —CH—};
a to e are each independently an integer of 0, 1, 2, 3 or 4 {provided that a and b may not be simultaneously 0, and c and d may not be simultaneously 0};
X is each independently F, Cl, Br or I.

In the pharmaceutical composition according to the present disclosure, the compound represented by the formula I may be the compound represented by formula Ia:
[Formula Ia]

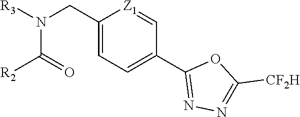

in Formula Ia,
R₂ is

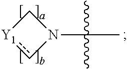

R₃ is -aryl {wherein at least one H of -aryl may be each independently substituted with —X};
Y₁ is —O— or —S(═O)₂—;
Z₁ is N or CR^{Z} {wherein R^{Z} is —X};
a and b are each independently an integer of 0, 1, 2, 3 or 4 {wherein a and b may not be simultaneously 0};
X is each independently F, Cl, Br or I.

In the pharmaceutical composition according to the present disclosure, the compound represented by formula Ia may be below:
R₂ is

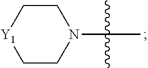

R₃ is -phenyl {wherein at least one H of -phenyl each independently may be substituted with —F or —Cl};
Y₁ is —O— or —S(═O)₂—;
Z₁ is N or CF.

In the pharmaceutical composition according to the present disclosure, the compounds represented by formula I may be shown in Table A below:

TABLE A

| Compound | Structure |
|---|---|
| 1 | 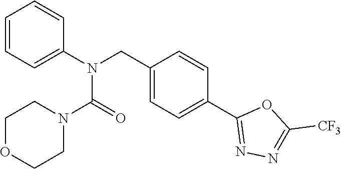 [SMILES: O=C(N1CCOCC1)N(Cc1ccc(-c2nnc(C(F)(F)F)o2)cc1)c1ccccc1] |
| 2 | 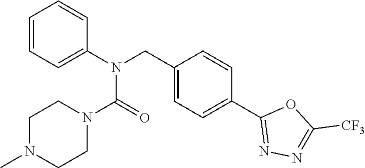 [SMILES: CN1CCN(C(=O)N(Cc2ccc(-c3nnc(C(F)(F)F)o3)cc2)c2ccccc2)CC1] |
| 3 | 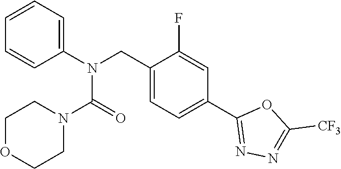 [SMILES: O=C(N1CCOCC1)N(Cc1ccc(-c2nnc(C(F)(F)F)o2)cc1F)c1ccccc1] |
| 4 | 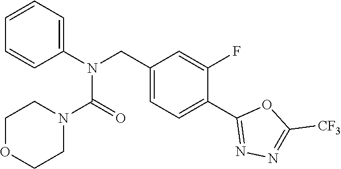 [SMILES: O=C(N1CCOCC1)N(Cc1ccc(-c2nnc(C(F)(F)F)o2)c(F)c1)c1ccccc1] |
| 5 | 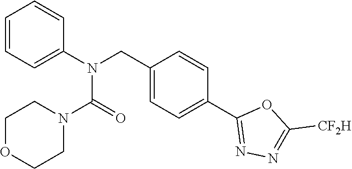 [SMILES: O=C(N1CCOCC1)N(Cc1ccc(-c2nnc(C(F)F)o2)cc1)c1ccccc1] |
| 6 | 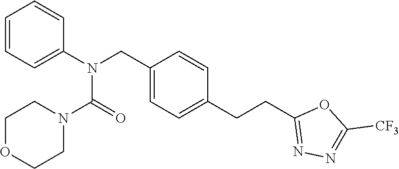 [SMILES: O=C(N1CCOCC1)N(Cc1ccc(CCc2nnc(C(F)(F)F)o2)cc1)c1ccccc1] |
| 7 | 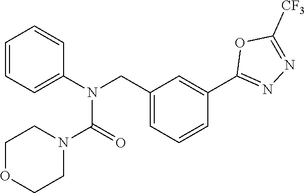 [SMILES: O=C(N1CCOCC1)N(Cc1cccc(-c2nnc(C(F)(F)F)o2)c1)c1ccccc1] |
| 8 | 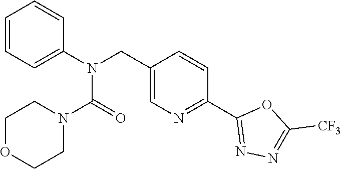 [SMILES: O=C(N1CCOCC1)N(Cc1ccc(-c2nnc(C(F)(F)F)o2)nc1)c1ccccc1] |
| 9 | 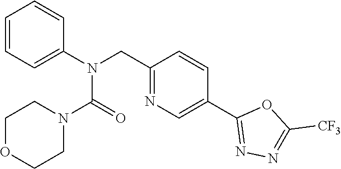 [SMILES: O=C(N1CCOCC1)N(Cc1ccc(-c2nnc(C(F)(F)F)o2)cn1)c1ccccc1] |
| 10 | 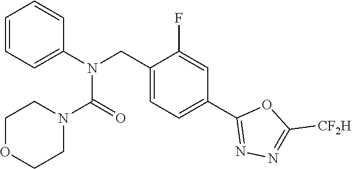 [SMILES: O=C(N1CCOCC1)N(Cc1ccc(-c2nnc(C(F)F)o2)cc1F)c1ccccc1] |
| 11 | 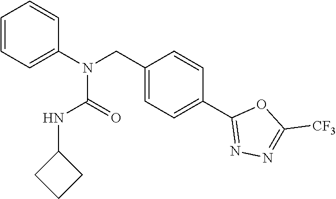 [SMILES: O=C(NC1CCC1)N(Cc1ccc(-c2nnc(C(F)(F)F)o2)cc1)c1ccccc1] |
| 12 | 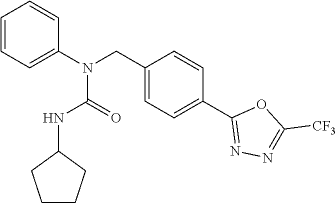 [SMILES: O=C(NC1CCCC1)N(Cc1ccc(-c2nnc(C(F)(F)F)o2)cc1)c1ccccc1] |
| 13 | 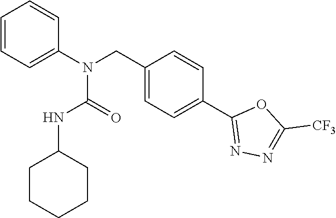 [SMILES: O=C(NC1CCCCC1)N(Cc1ccc(-c2nnc(C(F)(F)F)o2)cc1)c1ccccc1] |
| 14 | 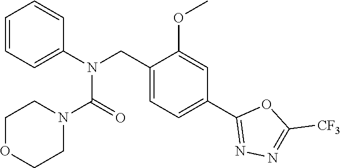 [SMILES: COc1cc(-c2nnc(C(F)(F)F)o2)ccc1CN(C(=O)N1CCOCC1)c1ccccc1] |
| 15 | 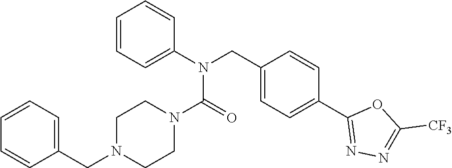 [SMILES: O=C(N1CCN(Cc2ccccc2)CC1)N(Cc1ccc(-c2nnc(C(F)(F)F)o2)cc1)c1ccccc1] |
| 16 | 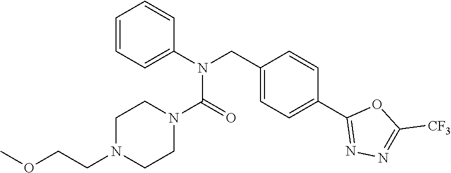 [SMILES: COCCN1CCN(C(=O)N(Cc2ccc(-c3nnc(C(F)(F)F)o3)cc2)c2ccccc2)CC1] |
| 17 | 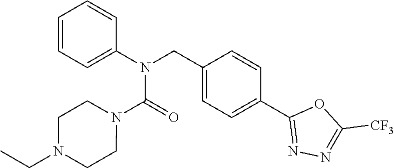 [SMILES: CCN1CCN(C(=O)N(Cc2ccc(-c3nnc(C(F)(F)F)o3)cc2)c2ccccc2)CC1] |
| 18 | 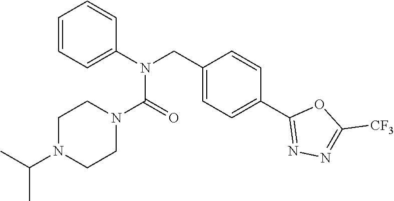 [SMILES: CC(C)N1CCN(C(=O)N(Cc2ccc(-c3nnc(C(F)(F)F)o3)cc2)c2ccccc2)CC1] |
| 19 | 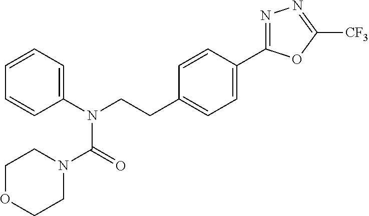 [SMILES: O=C(N1CCOCC1)N(CCc1ccc(-c2nnc(C(F)(F)F)o2)cc1)c1ccccc1] |
| 20 | 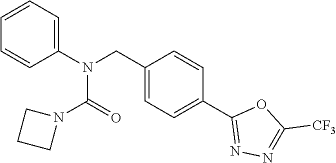 [SMILES: O=C(N1CCC1)N(Cc1ccc(-c2nnc(C(F)(F)F)o2)cc1)c1ccccc1] |
| 21 | 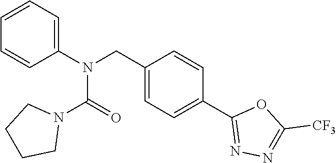 [SMILES: O=C(N1CCCC1)N(Cc1ccc(-c2nnc(C(F)(F)F)o2)cc1)c1ccccc1] |
| 22 | 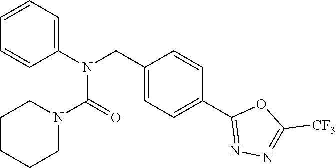 [SMILES: O=C(N1CCCCC1)N(Cc1ccc(-c2nnc(C(F)(F)F)o2)cc1)c1ccccc1] |
| 23 | 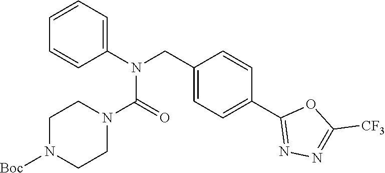 [SMILES: CC(C)(C)OC(=O)N1CCN(C(=O)N(Cc2ccc(-c3nnc(C(F)(F)F)o3)cc2)c2ccccc2)CC1] |
| 24 | 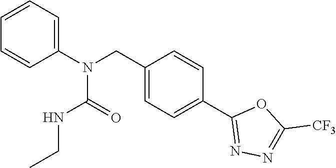 [SMILES: CCNC(=O)N(Cc1ccc(-c2nnc(C(F)(F)F)o2)cc1)c1ccccc1] |
| 25 | 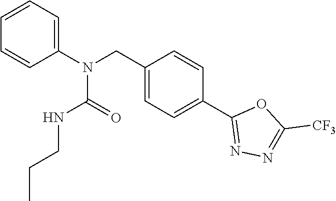 [SMILES: CCCNC(=O)N(Cc1ccc(-c2nnc(C(F)(F)F)o2)cc1)c1ccccc1] |
| 26 | 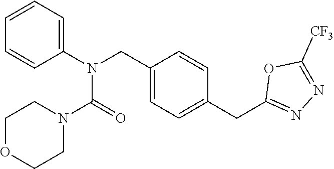 [SMILES: O=C(N1CCOCC1)N(Cc1ccc(Cc2nnc(C(F)(F)F)o2)cc1)c1ccccc1] |
| 27 | 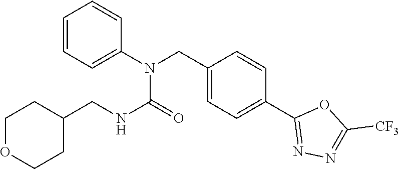 [SMILES: O=C(NCC1CCOCC1)N(Cc1ccc(-c2nnc(C(F)(F)F)o2)cc1)c1ccccc1] |
| 28 | 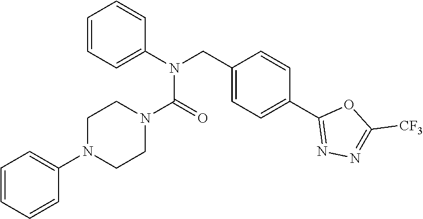 [SMILES: O=C(N1CCN(c2ccccc2)CC1)N(Cc1ccc(-c2nnc(C(F)(F)F)o2)cc1)c1ccccc1] |
| 29 | 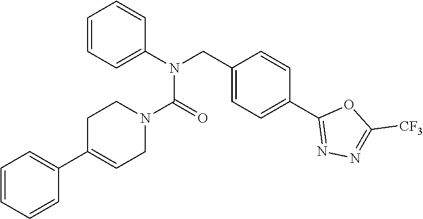 [SMILES: O=C(N1CC=C(c2ccccc2)CC1)N(Cc1ccc(-c2nnc(C(F)(F)F)o2)cc1)c1ccccc1] |
| 30 | 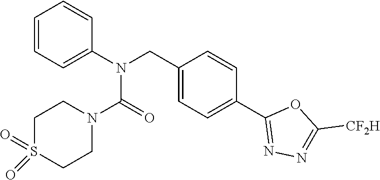 [SMILES: O=C(N1CCS(=O)(=O)CC1)N(Cc1ccc(-c2nnc(C(F)F)o2)cc1)c1ccccc1] |
| 31 | 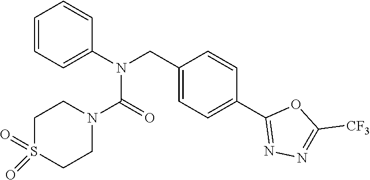 [SMILES: O=C(N1CCS(=O)(=O)CC1)N(Cc1ccc(-c2nnc(C(F)(F)F)o2)cc1)c1ccccc1] |
| 32 | 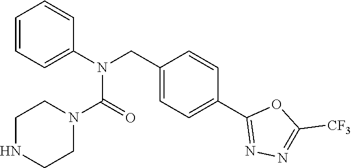 [SMILES: O=C(N1CCNCC1)N(Cc1ccc(-c2nnc(C(F)(F)F)o2)cc1)c1ccccc1] |
| 33 | 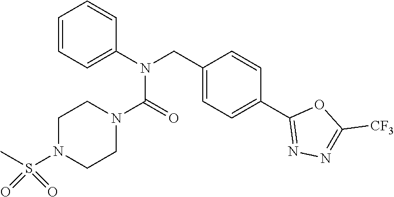 [SMILES: CS(=O)(=O)N1CCN(C(=O)N(Cc2ccc(-c3nnc(C(F)(F)F)o3)cc2)c2ccccc2)CC1] |
| 34 | 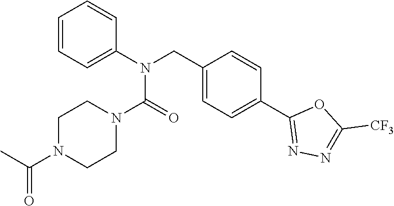 [SMILES: CC(=O)N1CCN(C(=O)N(Cc2ccc(-c3nnc(C(F)(F)F)o3)cc2)c2ccccc2)CC1] |
| 35 | 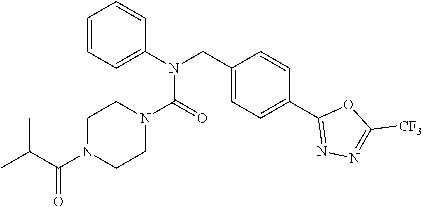 [SMILES: CC(C)C(=O)N1CCN(C(=O)N(Cc2ccc(-c3nnc(C(F)(F)F)o3)cc2)c2ccccc2)CC1] |
| 36 | 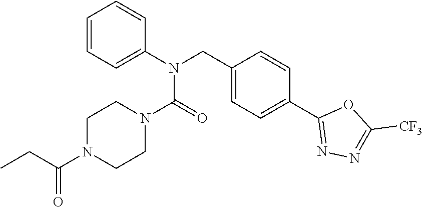 [SMILES: CCC(=O)N1CCN(C(=O)N(Cc2ccc(-c3nnc(C(F)(F)F)o3)cc2)c2ccccc2)CC1] |
| 37 | 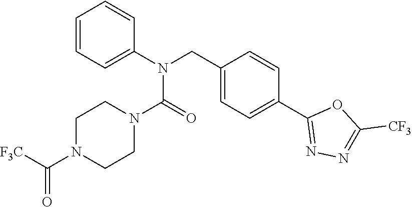 [SMILES: O=C(N1CCN(C(=O)C(F)(F)F)CC1)N(Cc1ccc(-c2nnc(C(F)(F)F)o2)cc1)c1ccccc1] |
| 38 | 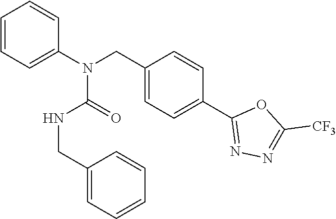 [SMILES: O=C(NCc1ccccc1)N(Cc1ccc(-c2nnc(C(F)(F)F)o2)cc1)c1ccccc1] |
| 39 | 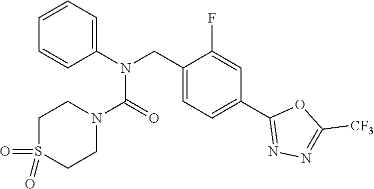 [SMILES: O=C(N1CCS(=O)(=O)CC1)N(Cc1ccc(-c2nnc(C(F)(F)F)o2)cc1F)c1ccccc1] |
| 40 | 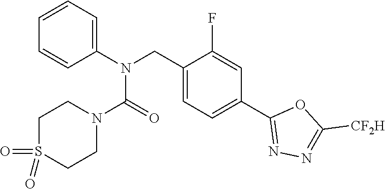 [SMILES: O=C(N1CCS(=O)(=O)CC1)N(Cc1ccc(-c2nnc(C(F)F)o2)cc1F)c1ccccc1] |
| 41 | 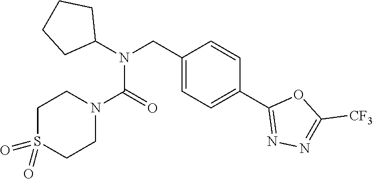 [SMILES: O=C(N1CCS(=O)(=O)CC1)N(Cc1ccc(-c2nnc(C(F)(F)F)o2)cc1)C1CCCC1] |
| 42 | 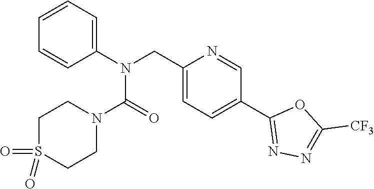 [SMILES: O=C(N1CCS(=O)(=O)CC1)N(Cc1ccc(-c2nnc(C(F)(F)F)o2)cn1)c1ccccc1] |
| 43 | 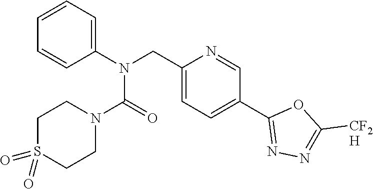 [SMILES: O=C(N1CCS(=O)(=O)CC1)N(Cc1ccc(-c2nnc(C(F)F)o2)cn1)c1ccccc1] |
| 44 | 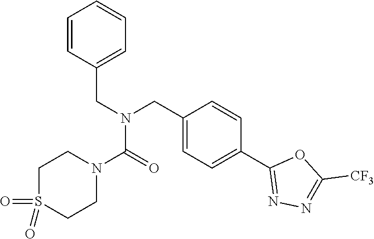 [SMILES: O=C(N1CCS(=O)(=O)CC1)N(Cc1ccccc1)Cc1ccc(-c2nnc(C(F)(F)F)o2)cc1] |
| 45 | 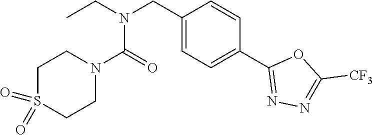 [SMILES: CCN(Cc1ccc(-c2nnc(C(F)(F)F)o2)cc1)C(=O)N1CCS(=O)(=O)CC1] |
| 46 | 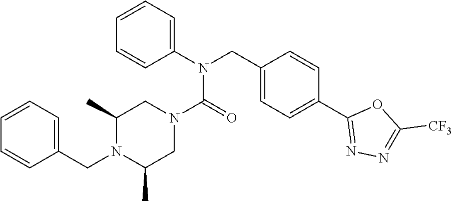 [SMILES: C[C@@H]1CN(C(=O)N(Cc2ccc(-c3nnc(C(F)(F)F)o3)cc2)c2ccccc2)C[C@H](C)N1Cc1ccccc1] |
| 47 | 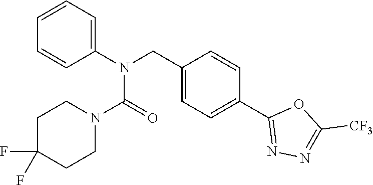 [SMILES: O=C(N1CCC(F)(F)CC1)N(Cc1ccc(-c2nnc(C(F)(F)F)o2)cc1)c1ccccc1] |
| 48 | 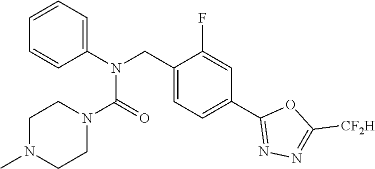 [SMILES: CN1CCN(C(=O)N(Cc2ccc(-c3nnc(C(F)F)o3)cc2F)c2ccccc2)CC1] |
| 49 | 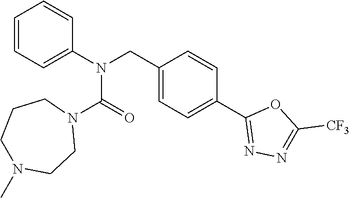 [SMILES: CN1CCCN(C(=O)N(Cc2ccc(-c3nnc(C(F)(F)F)o3)cc2)c2ccccc2)CC1] |
| 50 | 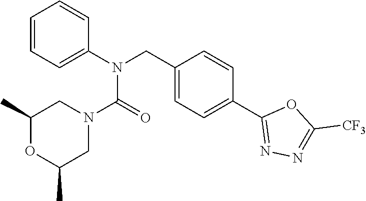 [SMILES: C[C@@H]1CN(C(=O)N(Cc2ccc(-c3nnc(C(F)(F)F)o3)cc2)c2ccccc2)C[C@H](C)O1] |
| 51 | 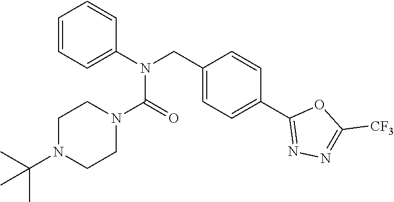 [SMILES: CC(C)(C)N1CCN(C(=O)N(Cc2ccc(-c3nnc(C(F)(F)F)o3)cc2)c2ccccc2)CC1] |
| 52 | 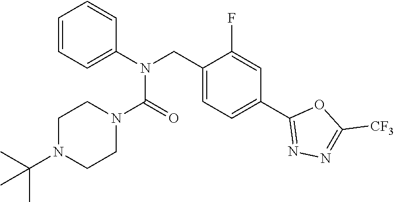 [SMILES: CC(C)(C)N1CCN(C(=O)N(Cc2ccc(-c3nnc(C(F)(F)F)o3)cc2F)c2ccccc2)CC1] |
| 53 | 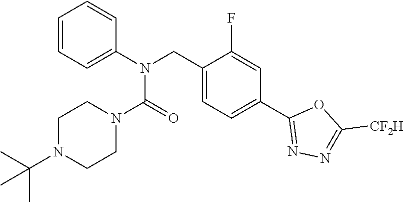 [SMILES: CC(C)(C)N1CCN(C(=O)N(Cc2ccc(-c3nnc(C(F)F)o3)cc2F)c2ccccc2)CC1] |
| 54 | 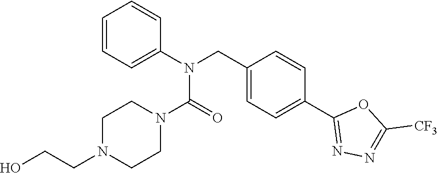 [SMILES: O=C(N1CCN(CCO)CC1)N(Cc1ccc(-c2nnc(C(F)(F)F)o2)cc1)c1ccccc1] |
| 55 | 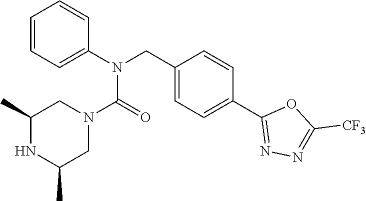 [SMILES: C[C@@H]1CN(C(=O)N(Cc2ccc(-c3nnc(C(F)(F)F)o3)cc2)c2ccccc2)C[C@H](C)N1] |
| 56 | 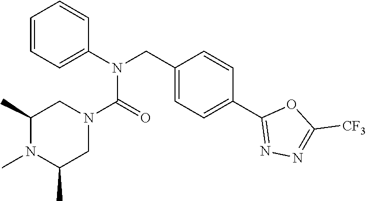 [SMILES: C[C@@H]1CN(C(=O)N(Cc2ccc(-c3nnc(C(F)(F)F)o3)cc2)c2ccccc2)C[C@H](C)N1C] |
| 57 | 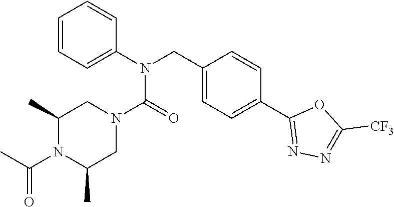 [SMILES: CC(=O)N1[C@H](C)CN(C(=O)N(Cc2ccc(-c3nnc(C(F)(F)F)o3)cc2)c2ccccc2)C[C@@H]1C] |
| 58 | 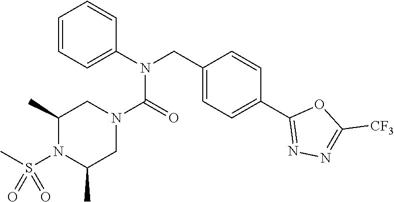 [SMILES: C[C@@H]1CN(C(=O)N(Cc2ccc(-c3nnc(C(F)(F)F)o3)cc2)c2ccccc2)C[C@H](C)N1S(C)(=O)=O] |
| 59 | 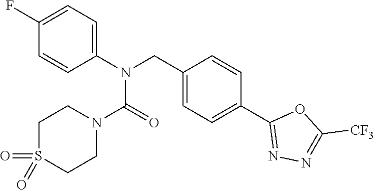 [SMILES: O=C(N1CCS(=O)(=O)CC1)N(Cc1ccc(-c2nnc(C(F)(F)F)o2)cc1)c1ccc(F)cc1] |
| 60 | 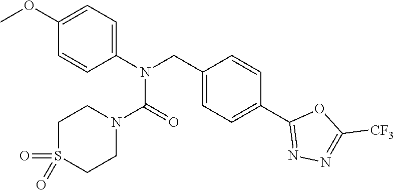 [SMILES: COc1ccc(N(Cc2ccc(-c3nnc(C(F)(F)F)o3)cc2)C(=O)N2CCS(=O)(=O)CC2)cc1] |
| 61 | 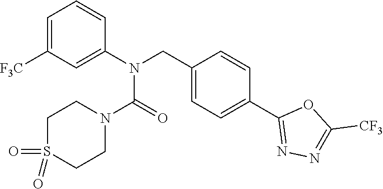 [SMILES: O=C(N1CCS(=O)(=O)CC1)N(Cc1ccc(-c2nnc(C(F)(F)F)o2)cc1)c1cccc(C(F)(F)F)c1] |
| 62 | 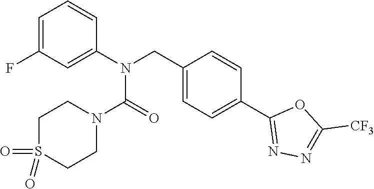 [SMILES: O=C(N1CCS(=O)(=O)CC1)N(Cc1ccc(-c2nnc(C(F)(F)F)o2)cc1)c1cccc(F)c1] |
| 63 | 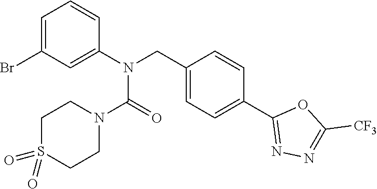 [SMILES: O=C(N1CCS(=O)(=O)CC1)N(Cc1ccc(-c2nnc(C(F)(F)F)o2)cc1)c1cccc(Br)c1] |
| 64 | 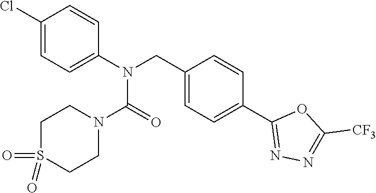 [SMILES: O=C(N1CCS(=O)(=O)CC1)N(Cc1ccc(-c2nnc(C(F)(F)F)o2)cc1)c1ccc(Cl)cc1] |
| 65 | 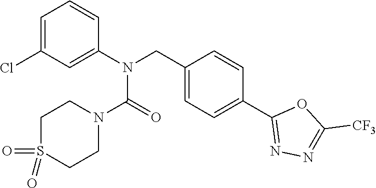 [SMILES: O=C(N1CCS(=O)(=O)CC1)N(Cc1ccc(-c2nnc(C(F)(F)F)o2)cc1)c1cccc(Cl)c1] |
| 66 | 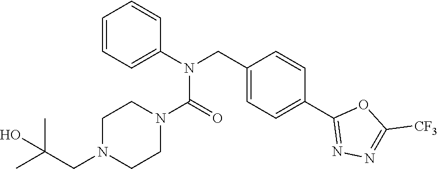 [SMILES: CC(C)(O)CN1CCN(C(=O)N(Cc2ccc(-c3nnc(C(F)(F)F)o3)cc2)c2ccccc2)CC1] |
| 67 | 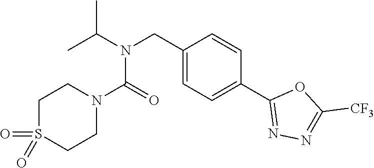 [SMILES: CC(C)N(Cc1ccc(-c2nnc(C(F)(F)F)o2)cc1)C(=O)N1CCS(=O)(=O)CC1] |
| 68 | 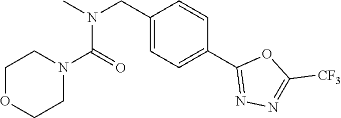 [SMILES: CN(Cc1ccc(-c2nnc(C(F)(F)F)o2)cc1)C(=O)N1CCOCC1] |
| 69 | 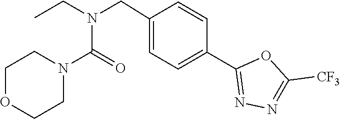 [SMILES: CCN(Cc1ccc(-c2nnc(C(F)(F)F)o2)cc1)C(=O)N1CCOCC1] |
| 70 | 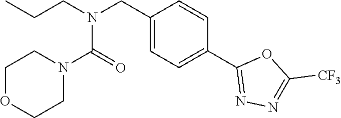 [SMILES: CCCN(Cc1ccc(-c2nnc(C(F)(F)F)o2)cc1)C(=O)N1CCOCC1] |
| 71 | 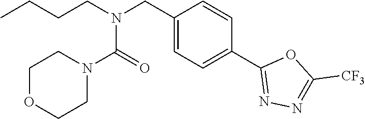 [SMILES: CCCCN(Cc1ccc(-c2nnc(C(F)(F)F)o2)cc1)C(=O)N1CCOCC1] |
| 72 | 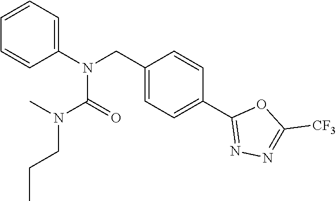 [SMILES: CCCN(C)C(=O)N(Cc1ccc(-c2nnc(C(F)(F)F)o2)cc1)c1ccccc1] |
| 73 | 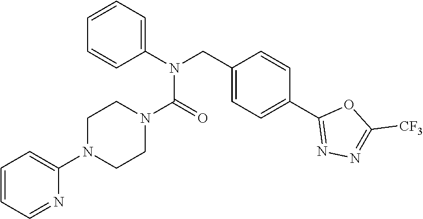 [SMILES: O=C(N1CCN(c2ccccn2)CC1)N(Cc1ccc(-c2nnc(C(F)(F)F)o2)cc1)c1ccccc1] |
| 74 | 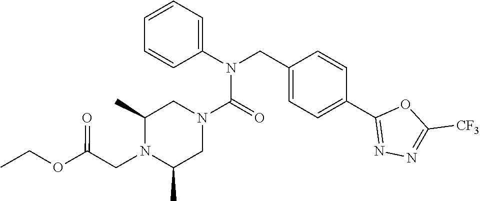 [SMILES: CCOC(=O)CN1[C@H](C)CN(C(=O)N(Cc2ccc(-c3nnc(C(F)(F)F)o3)cc2)c2ccccc2)C[C@@H]1C] |
| 75 | 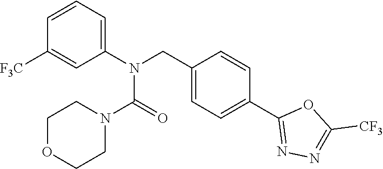 [SMILES: O=C(N1CCOCC1)N(Cc1ccc(-c2nnc(C(F)(F)F)o2)cc1)c1cccc(C(F)(F)F)c1] |
| 76 | 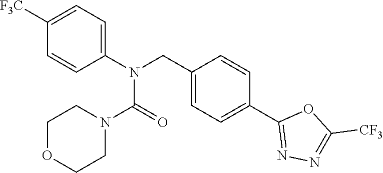 [SMILES: O=C(N1CCOCC1)N(Cc1ccc(-c2nnc(C(F)(F)F)o2)cc1)c1ccc(C(F)(F)F)cc1] |
| 77 | 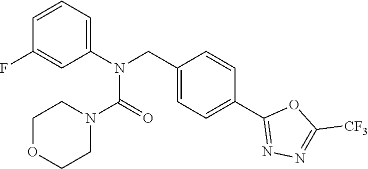 [SMILES: O=C(N1CCOCC1)N(Cc1ccc(-c2nnc(C(F)(F)F)o2)cc1)c1cccc(F)c1] |
| 78 | 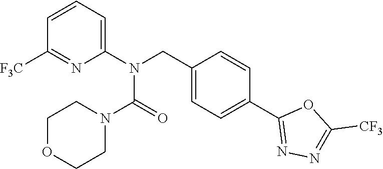 [SMILES: O=C(N1CCOCC1)N(Cc1ccc(-c2nnc(C(F)(F)F)o2)cc1)c1cccc(C(F)(F)F)n1] |
| 79 | 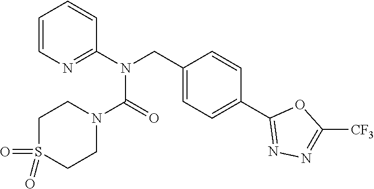 [SMILES: O=C(N1CCS(=O)(=O)CC1)N(Cc1ccc(-c2nnc(C(F)(F)F)o2)cc1)c1ccccn1] |
| 80 | 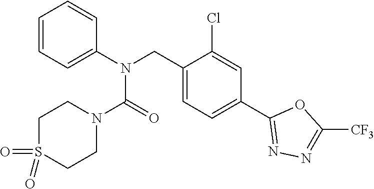 [SMILES: O=C(N1CCS(=O)(=O)CC1)N(Cc1ccc(-c2nnc(C(F)(F)F)o2)cc1Cl)c1ccccc1] |
| 81 | 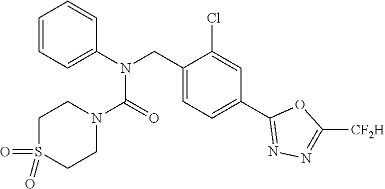 [SMILES: O=C(N1CCS(=O)(=O)CC1)N(Cc1ccc(-c2nnc(C(F)F)o2)cc1Cl)c1ccccc1] |
| 82 | 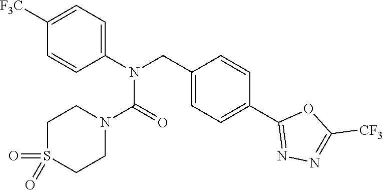 [SMILES: O=C(N1CCS(=O)(=O)CC1)N(Cc1ccc(-c2nnc(C(F)(F)F)o2)cc1)c1ccc(C(F)(F)F)cc1] |
| 83 | 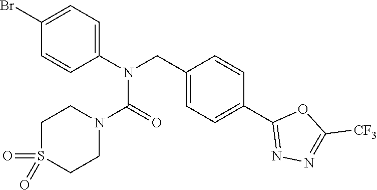 [SMILES: O=C(N1CCS(=O)(=O)CC1)N(Cc1ccc(-c2nnc(C(F)(F)F)o2)cc1)c1ccc(Br)cc1] |
| 84 | 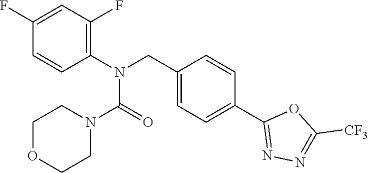 [SMILES: O=C(N1CCOCC1)N(Cc1ccc(-c2nnc(C(F)(F)F)o2)cc1)c1ccc(F)cc1F] |
| 85 | 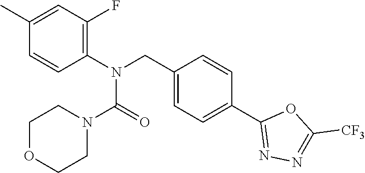 [SMILES: Cc1ccc(N(Cc2ccc(-c3nnc(C(F)(F)F)o3)cc2)C(=O)N2CCOCC2)c(F)c1] |
| 86 | 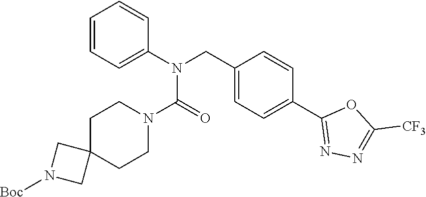 [SMILES: CC(C)(C)OC(=O)N1CC2(CCN(C(=O)N(Cc3ccc(-c4nnc(C(F)(F)F)o4)cc3)c3ccccc3)CC2)C1] |
| 87 | 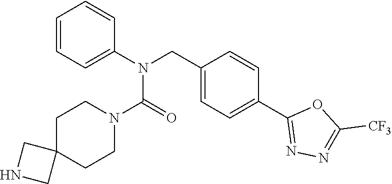 [SMILES: O=C(N1CCC2(CC1)CNC2)N(Cc1ccc(-c2nnc(C(F)(F)F)o2)cc1)c1ccccc1] |
| 88 | 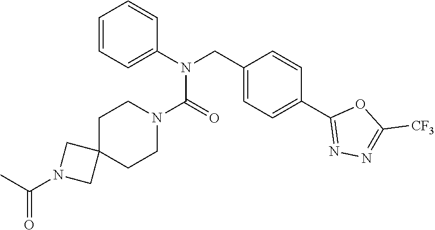 [SMILES: CC(=O)N1CC2(CCN(C(=O)N(Cc3ccc(-c4nnc(C(F)(F)F)o4)cc3)c3ccccc3)CC2)C1] |
| 89 | 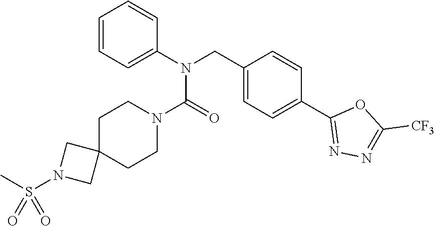 [SMILES: CS(=O)(=O)N1CC2(CCN(C(=O)N(Cc3ccc(-c4nnc(C(F)(F)F)o4)cc3)c3ccccc3)CC2)C1] |
| 90 | 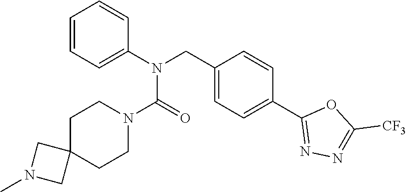 [SMILES: CN1CC2(CCN(C(=O)N(Cc3ccc(-c4nnc(C(F)(F)F)o4)cc3)c3ccccc3)CC2)C1] |
| 91 | 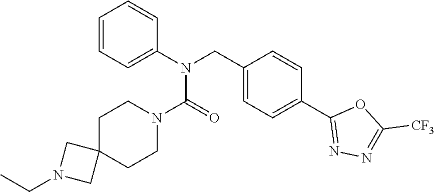 [SMILES: CCN1CC2(CCN(C(=O)N(Cc3ccc(-c4nnc(C(F)(F)F)o4)cc3)c3ccccc3)CC2)C1] |
| 92 | 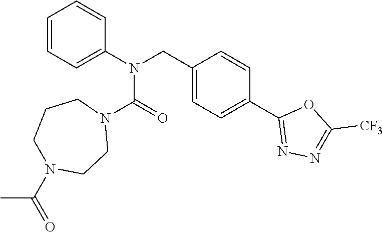 [SMILES: CC(=O)N1CCCN(C(=O)N(Cc2ccc(-c3nnc(C(F)(F)F)o3)cc2)c2ccccc2)CC1] |
| 93 | 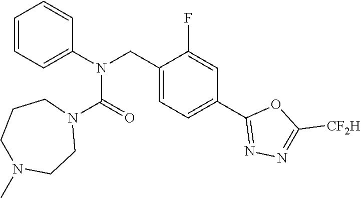 [SMILES: CN1CCCN(C(=O)N(Cc2ccc(-c3nnc(C(F)F)o3)cc2F)c2ccccc2)CC1] |
| 94 | 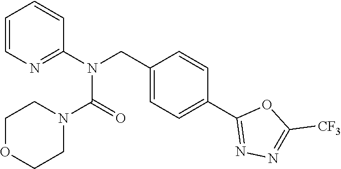 [SMILES: O=C(N1CCOCC1)N(Cc1ccc(-c2nnc(C(F)(F)F)o2)cc1)c1ccccn1] |
| 95 | 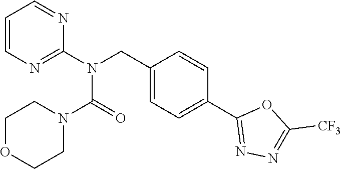 [SMILES: O=C(N1CCOCC1)N(Cc1ccc(-c2nnc(C(F)(F)F)o2)cc1)c1ncccn1] |
| 96 | 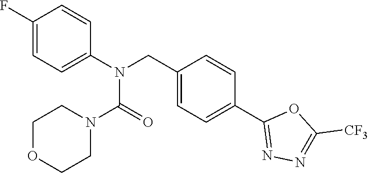 [SMILES: O=C(N1CCOCC1)N(Cc1ccc(-c2nnc(C(F)(F)F)o2)cc1)c1ccc(F)cc1] |
| 97 | 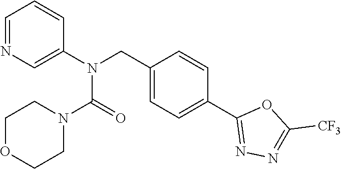 [SMILES: O=C(N1CCOCC1)N(Cc1ccc(-c2nnc(C(F)(F)F)o2)cc1)c1cccnc1] |
| 98 | 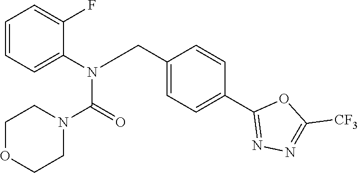 [SMILES: O=C(N1CCOCC1)N(Cc1ccc(-c2nnc(C(F)(F)F)o2)cc1)c1ccccc1F] |
| 99 | 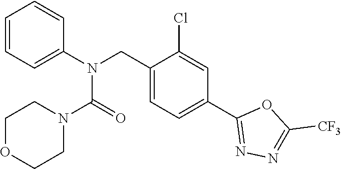 [SMILES: O=C(N1CCOCC1)N(Cc1ccc(-c2nnc(C(F)(F)F)o2)cc1Cl)c1ccccc1] |
| 100 | 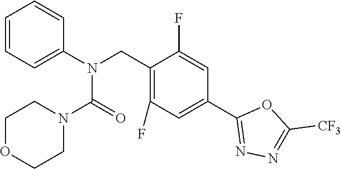 [SMILES: O=C(N1CCOCC1)N(Cc1c(F)cc(-c2nnc(C(F)(F)F)o2)cc1F)c1ccccc1] |
| 101 | 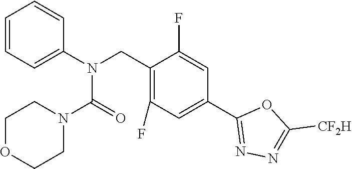 [SMILES: O=C(N1CCOCC1)N(Cc1c(F)cc(-c2nnc(C(F)F)o2)cc1F)c1ccccc1] |
| 102 | 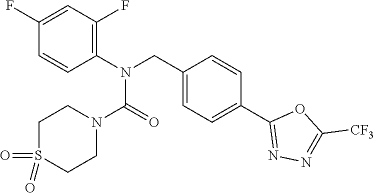 [SMILES: O=C(N1CCS(=O)(=O)CC1)N(Cc1ccc(-c2nnc(C(F)(F)F)o2)cc1)c1ccc(F)cc1F] |
| 103 | 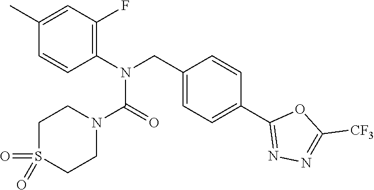 [SMILES: Cc1ccc(N(Cc2ccc(-c3nnc(C(F)(F)F)o3)cc2)C(=O)N2CCS(=O)(=O)CC2)c(F)c1] |
| 104 | 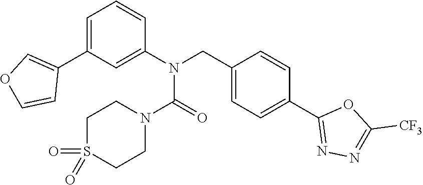 [SMILES: O=C(N1CCS(=O)(=O)CC1)N(Cc1ccc(-c2nnc(C(F)(F)F)o2)cc1)c1cccc(-c2ccoc2)c1] |
| 105 | 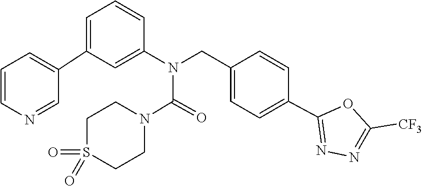 [SMILES: O=C(N1CCS(=O)(=O)CC1)N(Cc1ccc(-c2nnc(C(F)(F)F)o2)cc1)c1cccc(-c2cccnc2)c1] |
| 106 | 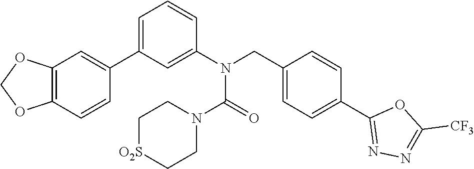 [SMILES: O=C(N1CCS(=O)(=O)CC1)N(Cc1ccc(-c2nnc(C(F)(F)F)o2)cc1)c1cccc(-c2ccc3c(c2)OCO3)c1] |
| 107 | 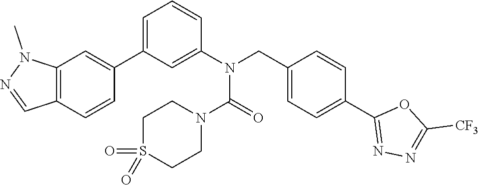 [SMILES: Cn1ncc2ccc(-c3cccc(N(Cc4ccc(-c5nnc(C(F)(F)F)o5)cc4)C(=O)N4CCS(=O)(=O)CC4)c3)cc21] |
| 108 | 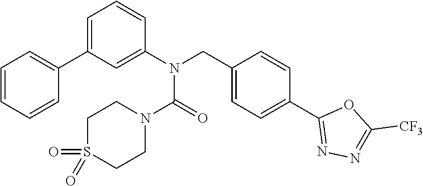 [SMILES: O=C(N1CCS(=O)(=O)CC1)N(Cc1ccc(-c2nnc(C(F)(F)F)o2)cc1)c1cccc(-c2ccccc2)c1] |
| 109 | 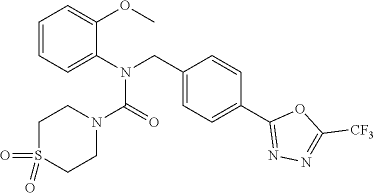 [SMILES: COc1ccccc1N(Cc1ccc(-c2nnc(C(F)(F)F)o2)cc1)C(=O)N1CCS(=O)(=O)CC1] |
| 110 | 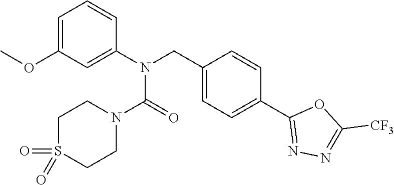 [SMILES: COc1cccc(N(Cc2ccc(-c3nnc(C(F)(F)F)o3)cc2)C(=O)N2CCS(=O)(=O)CC2)c1] |
| 111 | 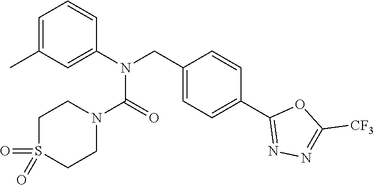 [SMILES: Cc1cccc(N(Cc2ccc(-c3nnc(C(F)(F)F)o3)cc2)C(=O)N2CCS(=O)(=O)CC2)c1] |
| 112 | 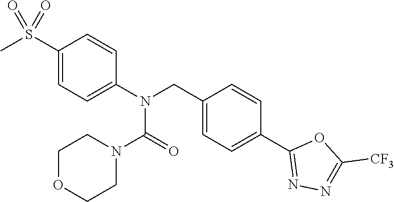 [SMILES: CS(=O)(=O)c1ccc(N(Cc2ccc(-c3nnc(C(F)(F)F)o3)cc2)C(=O)N2CCOCC2)cc1] |
| 113 | 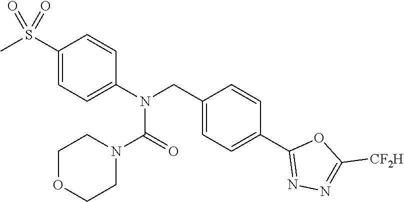 [SMILES: CS(=O)(=O)c1ccc(N(Cc2ccc(-c3nnc(C(F)F)o3)cc2)C(=O)N2CCOCC2)cc1] |
| 114 | 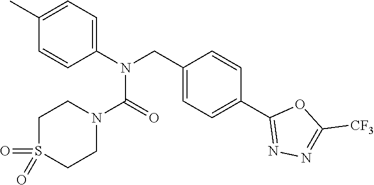 [SMILES: Cc1ccc(N(Cc2ccc(-c3nnc(C(F)(F)F)o3)cc2)C(=O)N2CCS(=O)(=O)CC2)cc1] |
| 115 | 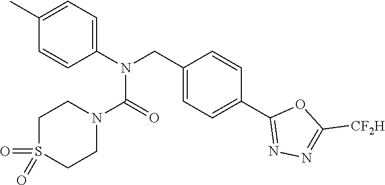 [SMILES: Cc1ccc(N(Cc2ccc(-c3nnc(C(F)F)o3)cc2)C(=O)N2CCS(=O)(=O)CC2)cc1] |
| 116 | 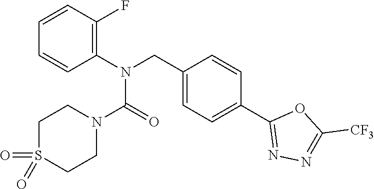 [SMILES: O=C(N1CCS(=O)(=O)CC1)N(Cc1ccc(-c2nnc(C(F)(F)F)o2)cc1)c1ccccc1F] |
| 117 | 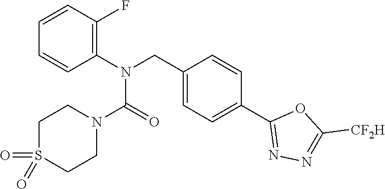 [SMILES: O=C(N1CCS(=O)(=O)CC1)N(Cc1ccc(-c2nnc(C(F)F)o2)cc1)c1ccccc1F] |
| 118 | 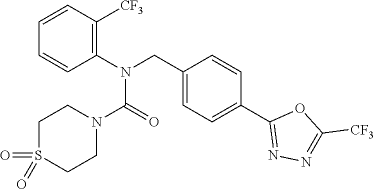 [SMILES: O=C(N1CCS(=O)(=O)CC1)N(Cc1ccc(-c2nnc(C(F)(F)F)o2)cc1)c1ccccc1C(F)(F)F] |
| 119 | 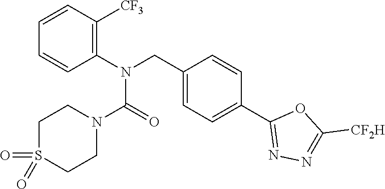 [SMILES: O=C(N1CCS(=O)(=O)CC1)N(Cc1ccc(-c2nnc(C(F)F)o2)cc1)c1ccccc1C(F)(F)F] |
| 120 | 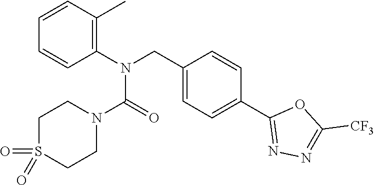 [SMILES: Cc1ccccc1N(Cc1ccc(-c2nnc(C(F)(F)F)o2)cc1)C(=O)N1CCS(=O)(=O)CC1] |
| 121 | 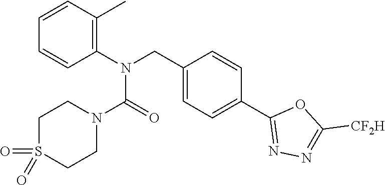 [SMILES: Cc1ccccc1N(Cc1ccc(-c2nnc(C(F)F)o2)cc1)C(=O)N1CCS(=O)(=O)CC1] |
| 122 | 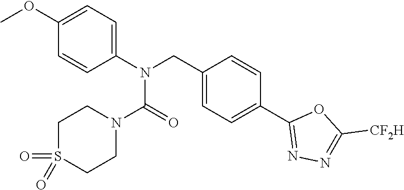 [SMILES: COc1ccc(N(Cc2ccc(-c3nnc(C(F)F)o3)cc2)C(=O)N2CCS(=O)(=O)CC2)cc1] |
| 123 | 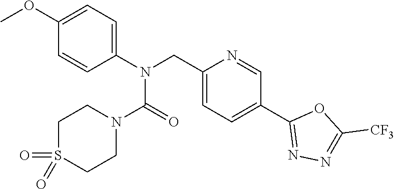 [SMILES: COc1ccc(N(Cc2ccc(-c3nnc(C(F)(F)F)o3)cn2)C(=O)N2CCS(=O)(=O)CC2)cc1] |
| 124 | 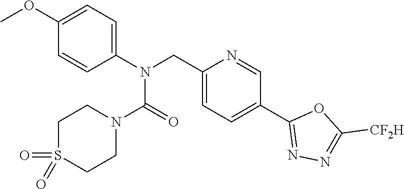 [SMILES: COc1ccc(N(Cc2ccc(-c3nnc(C(F)F)o3)cn2)C(=O)N2CCS(=O)(=O)CC2)cc1] |
| 125 | 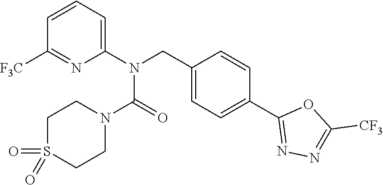 [SMILES: O=C(N1CCS(=O)(=O)CC1)N(Cc1ccc(-c2nnc(C(F)(F)F)o2)cc1)c1cccc(C(F)(F)F)n1] |
| 126 | 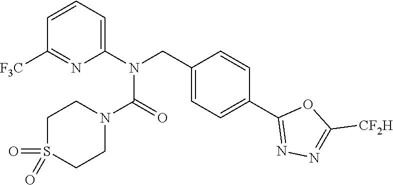 [SMILES: O=C(N1CCS(=O)(=O)CC1)N(Cc1ccc(-c2nnc(C(F)F)o2)cc1)c1cccc(C(F)(F)F)n1] |
| 127 | 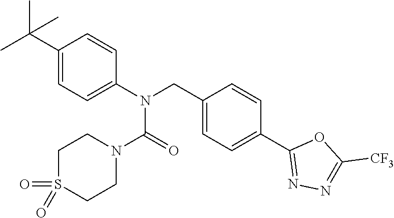 [SMILES: CC(C)(C)c1ccc(N(Cc2ccc(-c3nnc(C(F)(F)F)o3)cc2)C(=O)N2CCS(=O)(=O)CC2)cc1] |
| 128 | 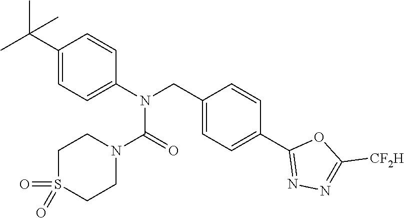 [SMILES: CC(C)(C)c1ccc(N(Cc2ccc(-c3nnc(C(F)F)o3)cc2)C(=O)N2CCS(=O)(=O)CC2)cc1] |
| 129 | 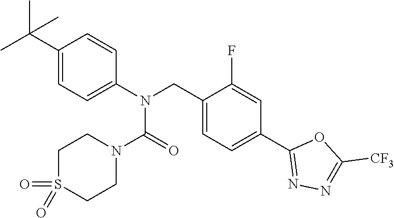 [SMILES: CC(C)(C)c1ccc(N(Cc2ccc(-c3nnc(C(F)(F)F)o3)cc2F)C(=O)N2CCS(=O)(=O)CC2)cc1] |
| 130 | 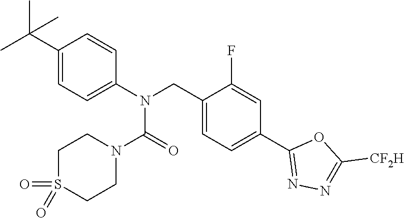 [SMILES: CC(C)(C)c1ccc(N(Cc2ccc(-c3nnc(C(F)F)o3)cc2F)C(=O)N2CCS(=O)(=O)CC2)cc1] |
| 131 | 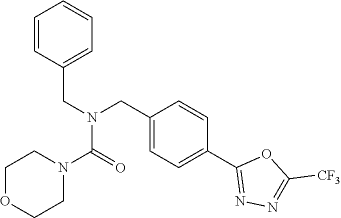 [SMILES: O=C(N1CCOCC1)N(Cc1ccccc1)Cc1ccc(-c2nnc(C(F)(F)F)o2)cc1] |
| 132 | 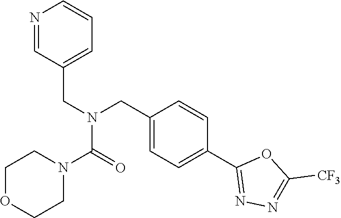 [SMILES: O=C(N1CCOCC1)N(Cc1ccc(-c2nnc(C(F)(F)F)o2)cc1)Cc1cccnc1] |
| 133 | 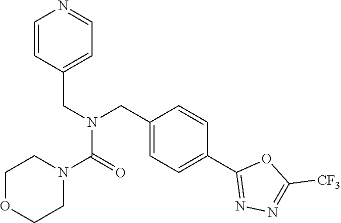 [SMILES: O=C(N1CCOCC1)N(Cc1ccncc1)Cc1ccc(-c2nnc(C(F)(F)F)o2)cc1] |
| 134 | 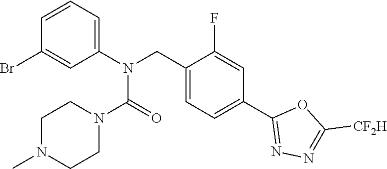 [SMILES: CN1CCN(C(=O)N(Cc2ccc(-c3nnc(C(F)F)o3)cc2F)c2cccc(Br)c2)CC1] |
| 135 | 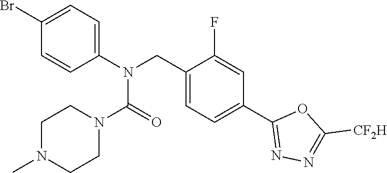 [SMILES: CN1CCN(C(=O)N(Cc2ccc(-c3nnc(C(F)F)o3)cc2F)c2ccc(Br)cc2)CC1] |
| 136 | 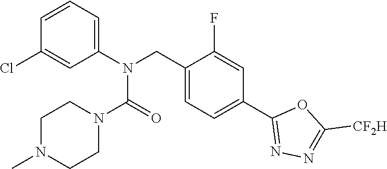 [SMILES: CN1CCN(C(=O)N(Cc2ccc(-c3nnc(C(F)F)o3)cc2F)c2cccc(Cl)c2)CC1] |
| 137 | 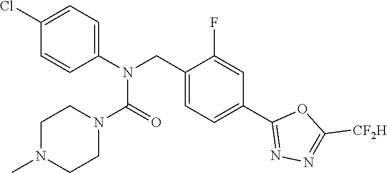 [SMILES: CN1CCN(C(=O)N(Cc2ccc(-c3nnc(C(F)F)o3)cc2F)c2ccc(Cl)cc2)CC1] |
| 138 | 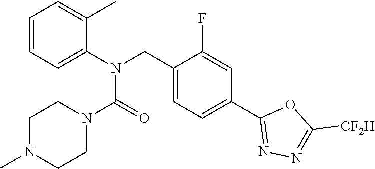 [SMILES: Cc1ccccc1N(Cc1ccc(-c2nnc(C(F)F)o2)cc1F)C(=O)N1CCN(C)CC1] |
| 139 | 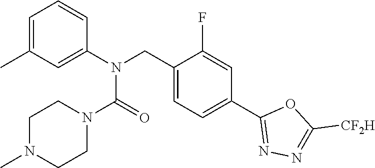 [SMILES: Cc1cccc(N(Cc2ccc(-c3nnc(C(F)F)o3)cc2F)C(=O)N2CCN(C)CC2)c1] |
| 140 | 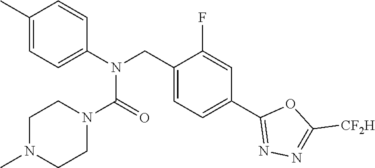 [SMILES: Cc1ccc(N(Cc2ccc(-c3nnc(C(F)F)o3)cc2F)C(=O)N2CCN(C)CC2)cc1] |
| 141 | 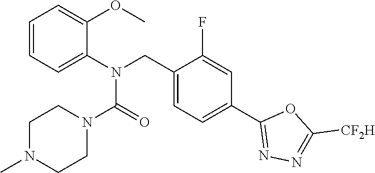 [SMILES: COc1ccccc1N(Cc1ccc(-c2nnc(C(F)F)o2)cc1F)C(=O)N1CCN(C)CC1] |
| 142 | 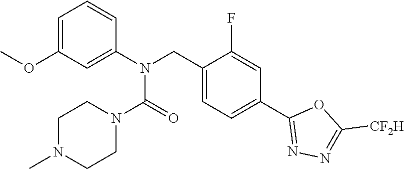 [SMILES: COc1cccc(N(Cc2ccc(-c3nnc(C(F)F)o3)cc2F)C(=O)N2CCN(C)CC2)c1] |
| 143 | 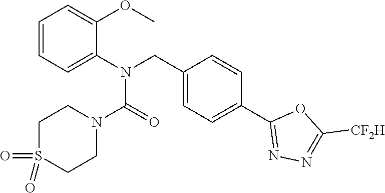 [SMILES: COc1ccccc1N(Cc1ccc(-c2nnc(C(F)F)o2)cc1)C(=O)N1CCS(=O)(=O)CC1] |
| 144 | 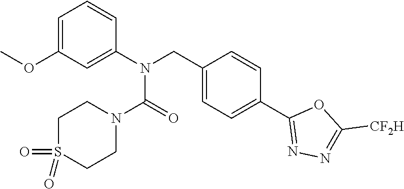 [SMILES: COc1cccc(N(Cc2ccc(-c3nnc(C(F)F)o3)cc2)C(=O)N2CCS(=O)(=O)CC2)c1] |
| 145 | 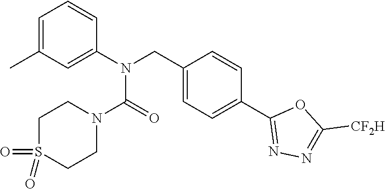 [SMILES: Cc1cccc(N(Cc2ccc(-c3nnc(C(F)F)o3)cc2)C(=O)N2CCS(=O)(=O)CC2)c1] |
| 146 | 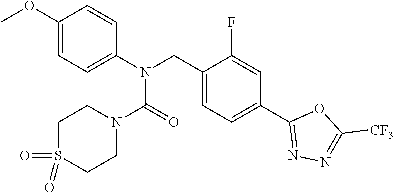 [SMILES: COc1ccc(N(Cc2ccc(-c3nnc(C(F)(F)F)o3)cc2F)C(=O)N2CCS(=O)(=O)CC2)cc1] |
| 147 | 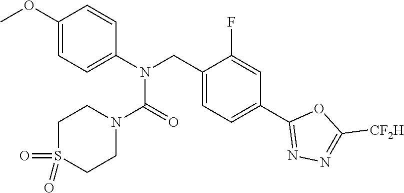 [SMILES: COc1ccc(N(Cc2ccc(-c3nnc(C(F)F)o3)cc2F)C(=O)N2CCS(=O)(=O)CC2)cc1] |
| 148 | 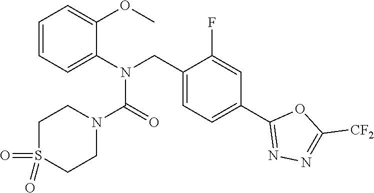 |
| 149 | 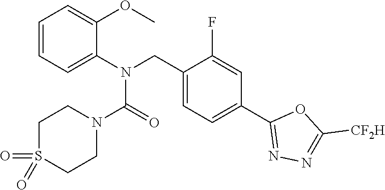 [SMILES: COc1ccccc1N(Cc1ccc(-c2nnc(C(F)F)o2)cc1F)C(=O)N1CCS(=O)(=O)CC1] |
| 150 | 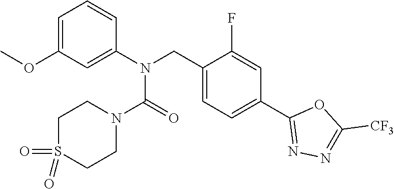 [SMILES: COc1cccc(N(Cc2ccc(-c3nnc(C(F)(F)F)o3)cc2F)C(=O)N2CCS(=O)(=O)CC2)c1] |
| 151 | 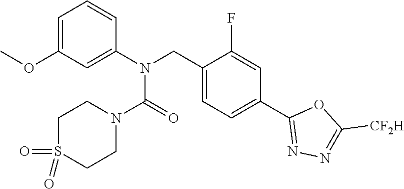 [SMILES: COc1cccc(N(Cc2ccc(-c3nnc(C(F)F)o3)cc2F)C(=O)N2CCS(=O)(=O)CC2)c1] |
| 152 | 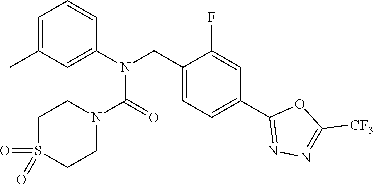 [SMILES: Cc1cccc(N(Cc2ccc(-c3nnc(C(F)(F)F)o3)cc2F)C(=O)N2CCS(=O)(=O)CC2)c1] |
| 153 | 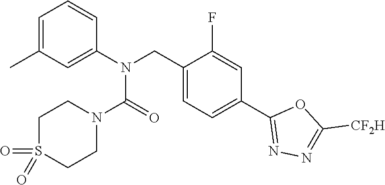 [SMILES: Cc1cccc(N(Cc2ccc(-c3nnc(C(F)F)o3)cc2F)C(=O)N2CCS(=O)(=O)CC2)c1] |
| 154 | 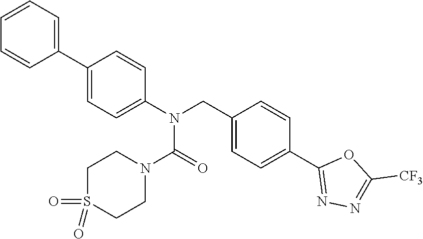 [SMILES: O=C(N1CCS(=O)(=O)CC1)N(Cc1ccc(-c2nnc(C(F)(F)F)o2)cc1)c1ccc(-c2ccccc2)cc1] |
| 155 | 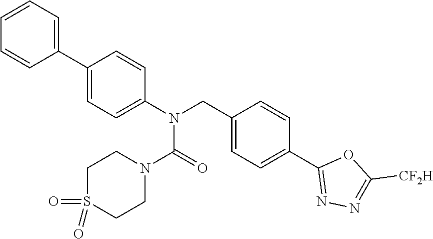 [SMILES: O=C(N1CCS(=O)(=O)CC1)N(Cc1ccc(-c2nnc(C(F)F)o2)cc1)c1ccc(-c2ccccc2)cc1] |
| 156 | 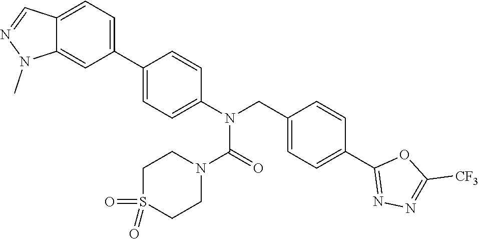 [SMILES: Cn1ncc2ccc(-c3ccc(N(Cc4ccc(-c5nnc(C(F)(F)F)o5)cc4)C(=O)N4CCS(=O)(=O)CC4)cc3)cc21] |
| 157 | 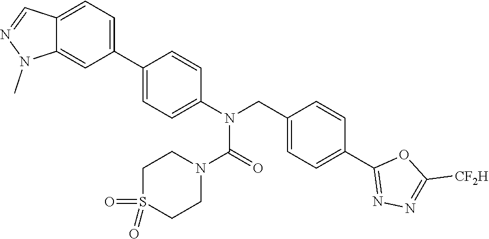 [SMILES: Cn1ncc2ccc(-c3ccc(N(Cc4ccc(-c5nnc(C(F)F)o5)cc4)C(=O)N4CCS(=O)(=O)CC4)cc3)cc21] |
| 158 | 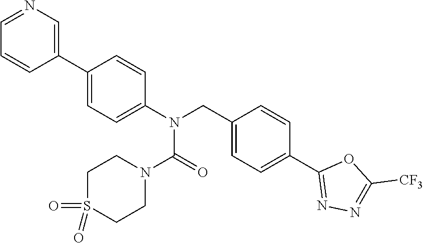 [SMILES: O=C(N1CCS(=O)(=O)CC1)N(Cc1ccc(-c2nnc(C(F)(F)F)o2)cc1)c1ccc(-c2cccnc2)cc1] |
| 159 | 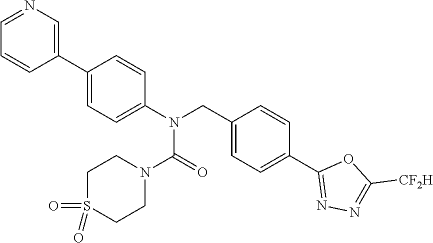 [SMILES: O=C(N1CCS(=O)(=O)CC1)N(Cc1ccc(-c2nnc(C(F)F)o2)cc1)c1ccc(-c2cccnc2)cc1] |
| 160 | 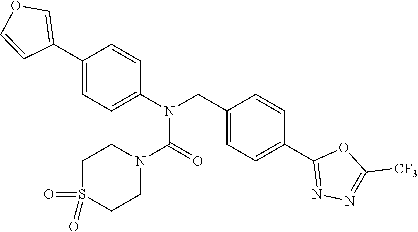 [SMILES: O=C(N1CCS(=O)(=O)CC1)N(Cc1ccc(-c2nnc(C(F)(F)F)o2)cc1)c1ccc(-c2ccoc2)cc1] |
| 161 | 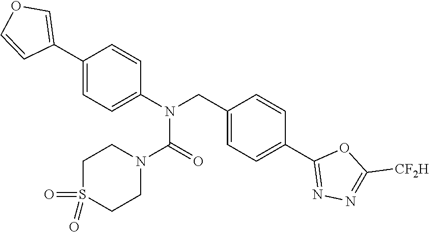 [SMILES: O=C(N1CCS(=O)(=O)CC1)N(Cc1ccc(-c2nnc(C(F)F)o2)cc1)c1ccc(-c2ccoc2)cc1] |
| 162 | 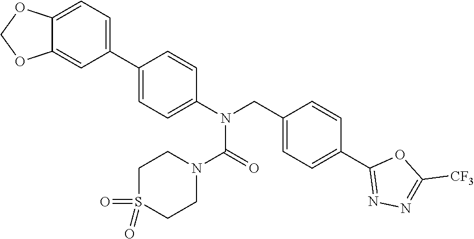 [SMILES: O=C(N1CCS(=O)(=O)CC1)N(Cc1ccc(-c2nnc(C(F)(F)F)o2)cc1)c1ccc(-c2ccc3c(c2)OCO3)cc1] |
| 163 | 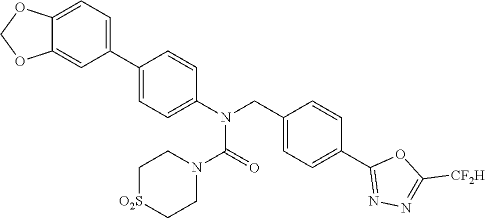 [SMILES: O=C(N1CCS(=O)(=O)CC1)N(Cc1ccc(-c2nnc(C(F)F)o2)cc1)c1ccc(-c2ccc3c(c2)OCO3)cc1] |
| 164 | 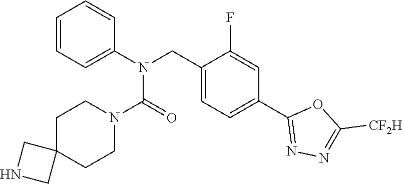 [SMILES: O=C(N1CCC2(CC1)CNC2)N(Cc1ccc(-c2nnc(C(F)F)o2)cc1F)c1ccccc1] |
| 165 | 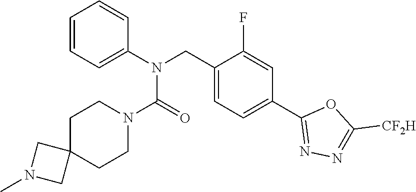 [SMILES: CN1CC2(CCN(C(=O)N(Cc3ccc(-c4nnc(C(F)F)o4)cc3F)c3ccccc3)CC2)C1] |
| 166 | 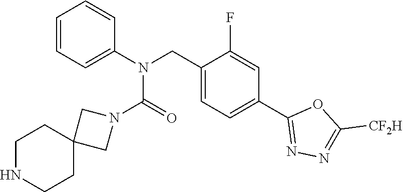 [SMILES: O=C(N1CC2(CCNCC2)C1)N(Cc1ccc(-c2nnc(C(F)F)o2)cc1F)c1ccccc1] |
| 167 | 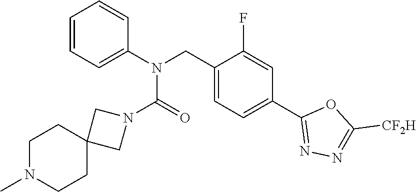 [SMILES: CN1CCC2(CC1)CN(C(=O)N(Cc1ccc(-c3nnc(C(F)F)o3)cc1F)c1ccccc1)C2] |
| 168 | 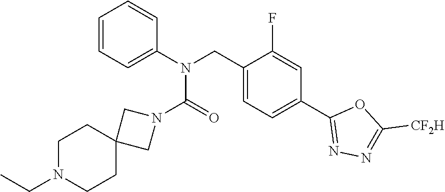 [SMILES: CCN1CCC2(CC1)CN(C(=O)N(Cc1ccc(-c3nnc(C(F)F)o3)cc1F)c1ccccc1)C2] |
| 169 | 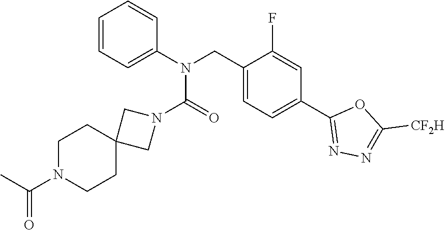 [SMILES: CC(=O)N1CCC2(CC1)CN(C(=O)N(Cc1ccc(-c3nnc(C(F)F)o3)cc1F)c1ccccc1)C2] |
| 170 | 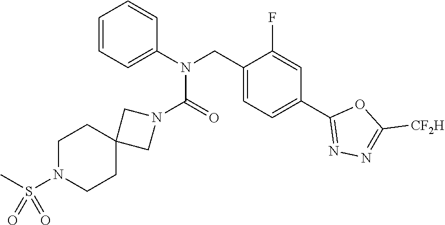 [SMILES: CS(=O)(=O)N1CCC2(CC1)CN(C(=O)N(Cc1ccc(-c3nnc(C(F)F)o3)cc1F)c1ccccc1)C2] |
| 171 | 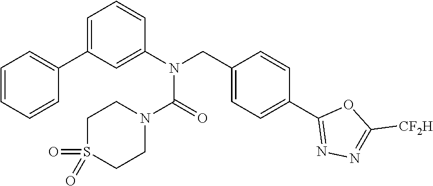 [SMILES: O=C(N1CCS(=O)(=O)CC1)N(Cc1ccc(-c2nnc(C(F)F)o2)cc1)c1cccc(-c2ccccc2)c1] |
| 172 | 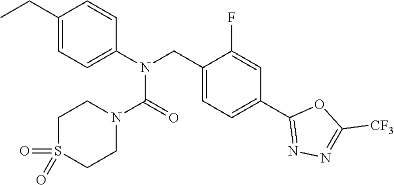 [SMILES: CCc1ccc(N(Cc2ccc(-c3nnc(C(F)(F)F)o3)cc2F)C(=O)N2CCS(=O)(=O)CC2)cc1] |
| 173 | 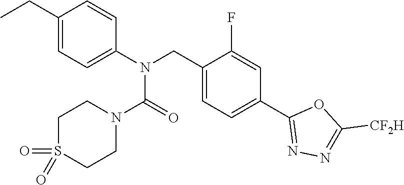 [SMILES: CCc1ccc(N(Cc2ccc(-c3nnc(C(F)F)o3)cc2F)C(=O)N2CCS(=O)(=O)CC2)cc1] |
| 174 | 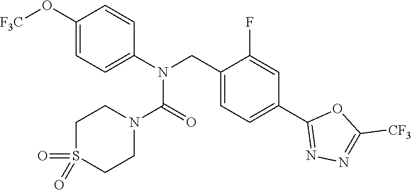 [SMILES: O=C(N1CCS(=O)(=O)CC1)N(Cc1ccc(-c2nnc(C(F)(F)F)o2)cc1F)c1ccc(OC(F)(F)F)cc1] |
| 175 | 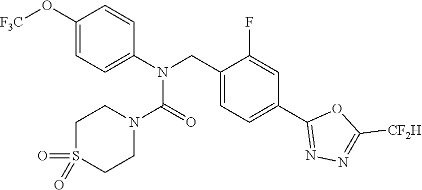 [SMILES: O=C(N1CCS(=O)(=O)CC1)N(Cc1ccc(-c2nnc(C(F)F)o2)cc1F)c1ccc(OC(F)(F)F)cc1] |
| 176 | 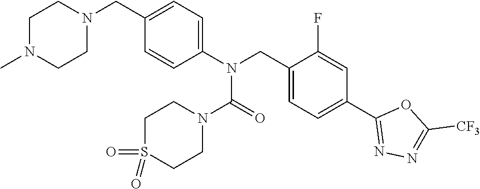 [SMILES: CN1CCN(Cc2ccc(N(Cc3ccc(-c4nnc(C(F)(F)F)o4)cc3F)C(=O)N3CCS(=O)(=O)CC3)cc2)CC1] |
| 177 | 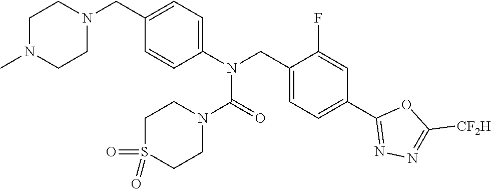 [SMILES: CN1CCN(Cc2ccc(N(Cc3ccc(-c4nnc(C(F)F)o4)cc3F)C(=O)N3CCS(=O)(=O)CC3)cc2)CC1] |
| 178 | 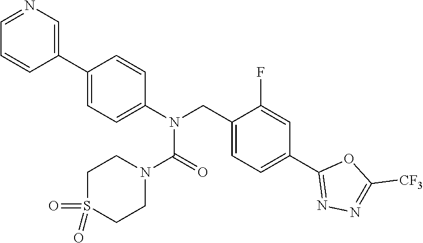 [SMILES: O=C(N1CCS(=O)(=O)CC1)N(Cc1ccc(-c2nnc(C(F)(F)F)o2)cc1F)c1ccc(-c2cccnc2)cc1] |
| 179 | 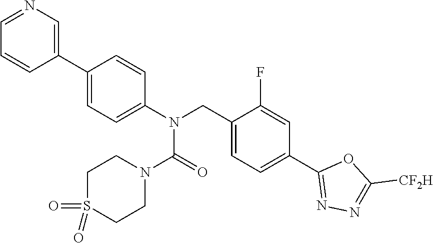 [SMILES: O=C(N1CCS(=O)(=O)CC1)N(Cc1ccc(-c2nnc(C(F)F)o2)cc1F)c1ccc(-c2cccnc2)cc1] |
| 180 | 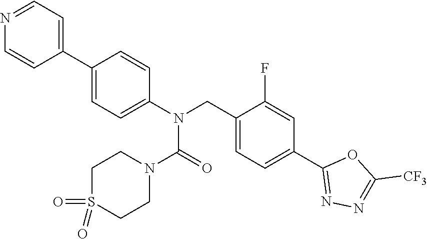 [SMILES: O=C(N1CCS(=O)(=O)CC1)N(Cc1ccc(-c2nnc(C(F)(F)F)o2)cc1F)c1ccc(-c2ccncc2)cc1] |
| 181 | 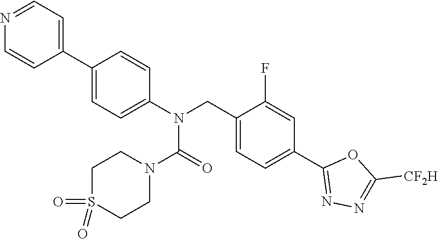 [SMILES: O=C(N1CCS(=O)(=O)CC1)N(Cc1ccc(-c2nnc(C(F)F)o2)cc1F)c1ccc(-c2ccncc2)cc1] |
| 182 | 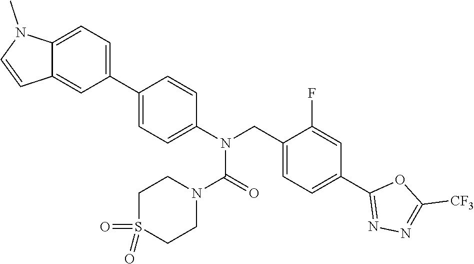 [SMILES: Cn1ccc2cc(-c3ccc(N(Cc4ccc(-c5nnc(C(F)(F)F)o5)cc4F)C(=O)N4CCS(=O)(=O)CC4)cc3)ccc21] |
| 183 | 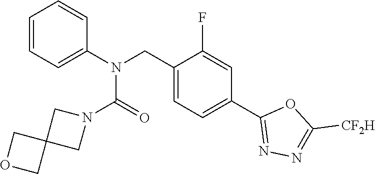 [SMILES: O=C(N1CC2(COC2)C1)N(Cc1ccc(-c2nnc(C(F)F)o2)cc1F)c1ccccc1] |
| 184 | 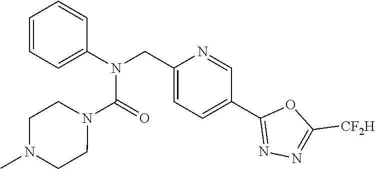 [SMILES: CN1CCN(C(=O)N(Cc2ccc(-c3nnc(C(F)F)o3)cn2)c2ccccc2)CC1] |
| 185 | 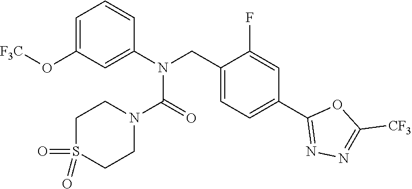 [SMILES: O=C(N1CCS(=O)(=O)CC1)N(Cc1ccc(-c2nnc(C(F)(F)F)o2)cc1F)c1cccc(OC(F)(F)F)c1] |
| 186 | 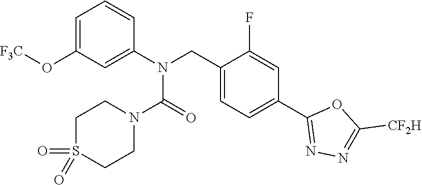 [SMILES: O=C(N1CCS(=O)(=O)CC1)N(Cc1ccc(-c2nnc(C(F)F)o2)cc1F)c1cccc(OC(F)(F)F)c1] |
| 187 | 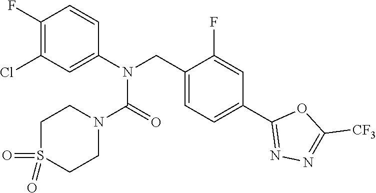 [SMILES: O=C(N1CCS(=O)(=O)CC1)N(Cc1ccc(-c2nnc(C(F)(F)F)o2)cc1F)c1ccc(F)c(Cl)c1] |
| 188 | 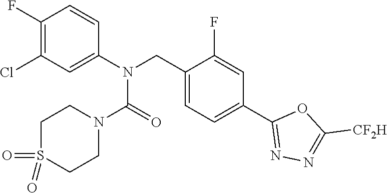 [SMILES: O=C(N1CCS(=O)(=O)CC1)N(Cc1ccc(-c2nnc(C(F)F)o2)cc1F)c1ccc(F)c(Cl)c1] |
| 189 | 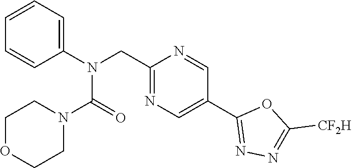 [SMILES: O=C(N1CCOCC1)N(Cc1ncc(-c2nnc(C(F)F)o2)cn1)c1ccccc1] |
| 190 | 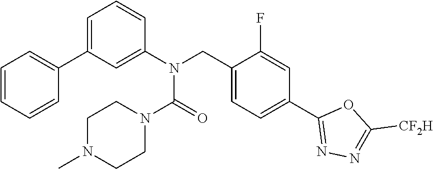 [SMILES: CN1CCN(C(=O)N(Cc2ccc(-c3nnc(C(F)F)o3)cc2F)c2cccc(-c3ccccc3)c2)CC1] |
| 191 | 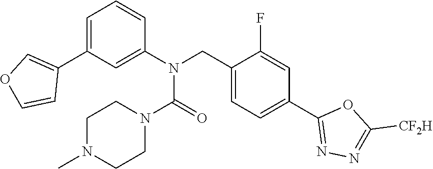 [SMILES: CN1CCN(C(=O)N(Cc2ccc(-c3nnc(C(F)F)o3)cc2F)c2cccc(-c3ccoc3)c2)CC1] |
| 192 | 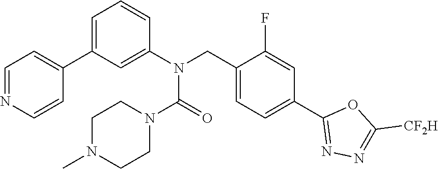 [SMILES: CN1CCN(C(=O)N(Cc2ccc(-c3nnc(C(F)F)o3)cc2F)c2cccc(-c3ccncc3)c2)CC1] |
| 193 | 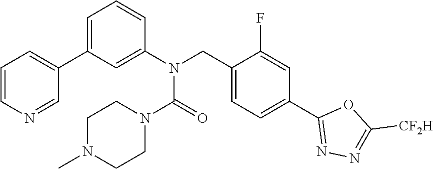 [SMILES: CN1CCN(C(=O)N(Cc2ccc(-c3nnc(C(F)F)o3)cc2F)c2cccc(-c3cccnc3)c2)CC1] |
| 194 | 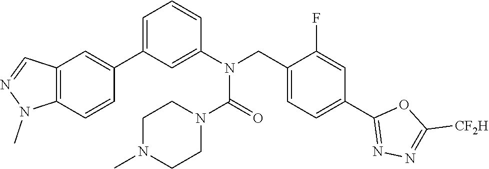 [SMILES: CN1CCN(C(=O)N(Cc2ccc(-c3nnc(C(F)F)o3)cc2F)c2cccc(-c3ccc4c(cnn4C)c3)c2)CC1] |
| 195 | 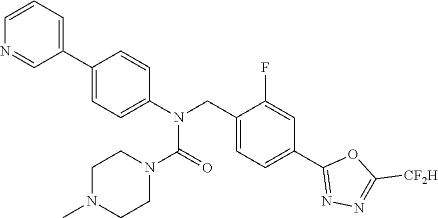 [SMILES: CN1CCN(C(=O)N(Cc2ccc(-c3nnc(C(F)F)o3)cc2F)c2ccc(-c3cccnc3)cc2)CC1] |
| 196 | 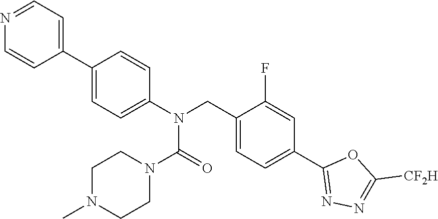 [SMILES: CN1CCN(C(=O)N(Cc2ccc(-c3nnc(C(F)F)o3)cc2F)c2ccc(-c3ccncc3)cc2)CC1] |
| 197 | 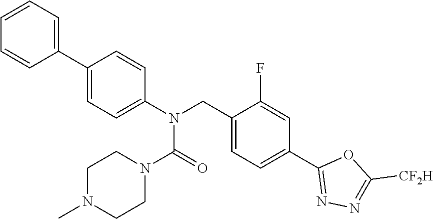 [SMILES: CN1CCN(C(=O)N(Cc2ccc(-c3nnc(C(F)F)o3)cc2F)c2ccc(-c3ccccc3)cc2)CC1] |
| 198 | 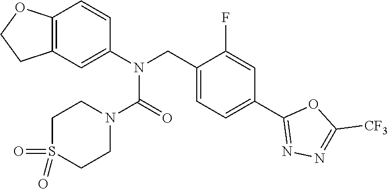 [SMILES: O=C(N1CCS(=O)(=O)CC1)N(Cc1ccc(-c2nnc(C(F)(F)F)o2)cc1F)c1ccc2c(c1)CCO2] |
| 199 | 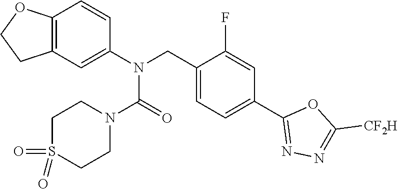 [SMILES: O=C(N1CCS(=O)(=O)CC1)N(Cc1ccc(-c2nnc(C(F)F)o2)cc1F)c1ccc2c(c1)CCO2] |
| 200 | 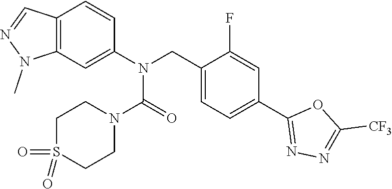 [SMILES: Cn1ncc2ccc(N(Cc3ccc(-c4nnc(C(F)(F)F)o4)cc3F)C(=O)N3CCS(=O)(=O)CC3)cc21] |
| 201 | 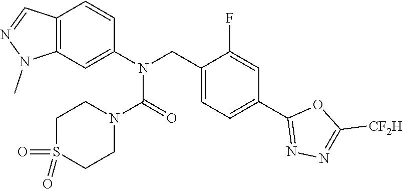 [SMILES: Cn1ncc2ccc(N(Cc3ccc(-c4nnc(C(F)F)o4)cc3F)C(=O)N3CCS(=O)(=O)CC3)cc21] |
| 202 | 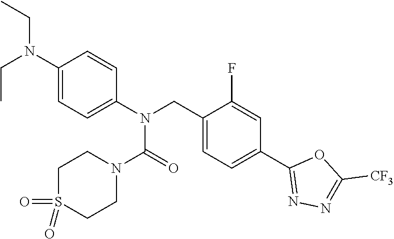 [SMILES: CCN(CC)c1ccc(N(Cc2ccc(-c3nnc(C(F)(F)F)o3)cc2F)C(=O)N2CCS(=O)(=O)CC2)cc1] |
| 203 | 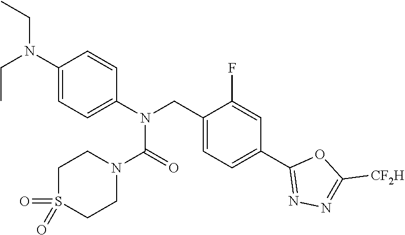 [SMILES: CCN(CC)c1ccc(N(Cc2ccc(-c3nnc(C(F)F)o3)cc2F)C(=O)N2CCS(=O)(=O)CC2)cc1] |
| 204 | 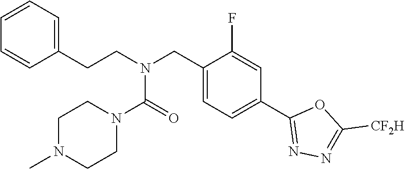 [SMILES: CN1CCN(C(=O)N(CCc2ccccc2)Cc2ccc(-c3nnc(C(F)F)o3)cc2F)CC1] |
| 205 | 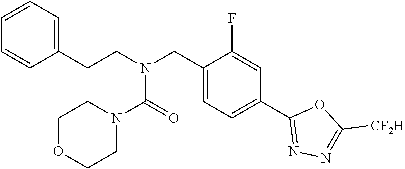 [SMILES: O=C(N1CCOCC1)N(CCc1ccccc1)Cc1ccc(-c2nnc(C(F)F)o2)cc1F] |
| 206 | 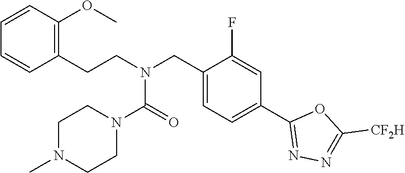 [SMILES: COc1ccccc1CCN(Cc1ccc(-c2nnc(C(F)F)o2)cc1F)C(=O)N1CCN(C)CC1] |
| 207 | 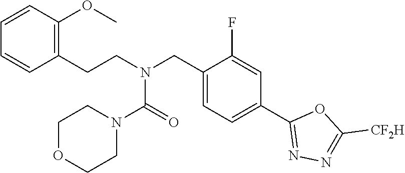 [SMILES: COc1ccccc1CCN(Cc1ccc(-c2nnc(C(F)F)o2)cc1F)C(=O)N1CCOCC1] |
| 208 | 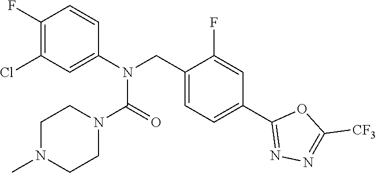 [SMILES: CN1CCN(C(=O)N(Cc2ccc(-c3nnc(C(F)(F)F)o3)cc2F)c2ccc(F)c(Cl)c2)CC1] |
| 209 | 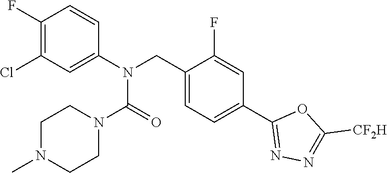 [SMILES: CN1CCN(C(=O)N(Cc2ccc(-c3nnc(C(F)F)o3)cc2F)c2ccc(F)c(Cl)c2)CC1] |
| 210 | 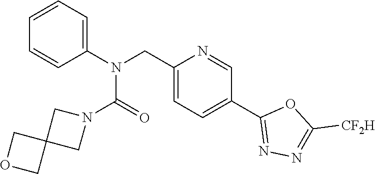 [SMILES: O=C(N1CC2(COC2)C1)N(Cc1ccc(-c2nnc(C(F)F)o2)cn1)c1ccccc1] |
| 211 | 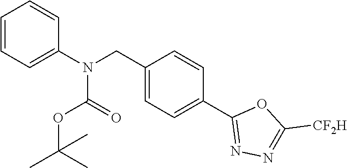 [SMILES: CC(C)(C)OC(=O)N(Cc1ccc(-c2nnc(C(F)F)o2)cc1)c1ccccc1] |
| 212 | 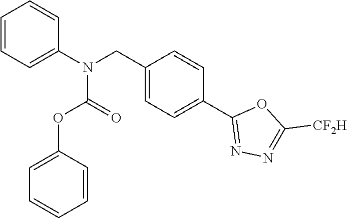 [SMILES: O=C(Oc1ccccc1)N(Cc1ccc(-c2nnc(C(F)F)o2)cc1)c1ccccc1] |
| 213 | 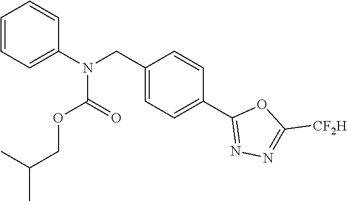 [SMILES: CC(C)COC(=O)N(Cc1ccc(-c2nnc(C(F)F)o2)cc1)c1ccccc1] |
| 214 | 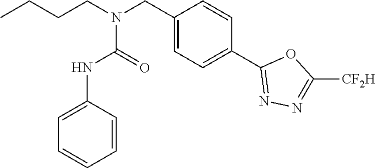 [SMILES: CCCCN(Cc1ccc(-c2nnc(C(F)F)o2)cc1)C(=O)Nc1ccccc1] |
| 215 | 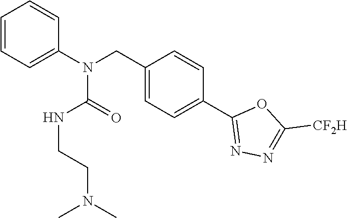 [SMILES: CN(C)CCNC(=O)N(Cc1ccc(-c2nnc(C(F)F)o2)cc1)c1ccccc1] |
| 216 | 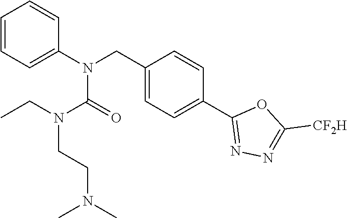 [SMILES: CCN(CCN(C)C)C(=O)N(Cc1ccc(-c2nnc(C(F)F)o2)cc1)c1ccccc1] |
| 217 | 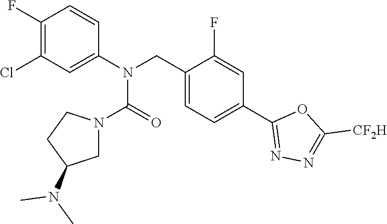 [SMILES: CN(C)[C@H]1CCN(C(=O)N(Cc2ccc(-c3nnc(C(F)F)o3)cc2F)c2ccc(F)c(Cl)c2)C1] |
| 218 | 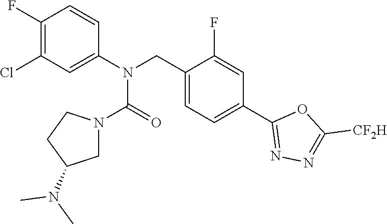 [SMILES: CN(C)[C@@H]1CCN(C(=O)N(Cc2ccc(-c3nnc(C(F)F)o3)cc2F)c2ccc(F)c(Cl)c2)C1] |
| 219 | 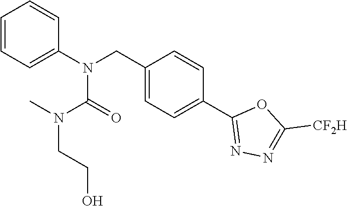 [SMILES: CN(CCO)C(=O)N(Cc1ccc(-c2nnc(C(F)F)o2)cc1)c1ccccc1] |
| 220 | 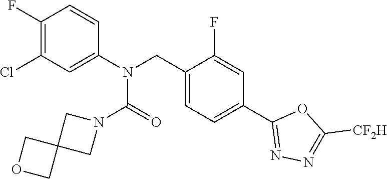 [SMILES: O=C(N1CC2(COC2)C1)N(Cc1ccc(-c2nnc(C(F)F)o2)cc1F)c1ccc(F)c(Cl)c1] |
| 221 | 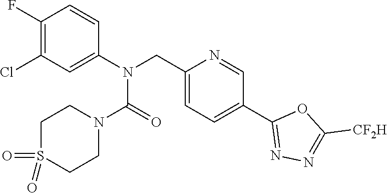 [SMILES: O=C(N1CCS(=O)(=O)CC1)N(Cc1ccc(-c2nnc(C(F)F)o2)cn1)c1ccc(F)c(Cl)c1] |
| 222 | 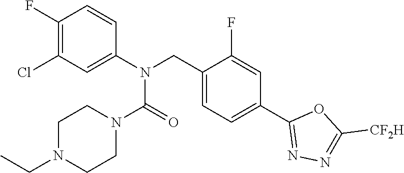 [SMILES: CCN1CCN(C(=O)N(Cc2ccc(-c3nnc(C(F)F)o3)cc2F)c2ccc(F)c(Cl)c2)CC1] |
| 223 | 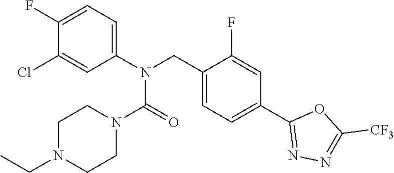 [SMILES: CCN1CCN(C(=O)N(Cc2ccc(-c3nnc(C(F)(F)F)o3)cc2F)c2ccc(F)c(Cl)c2)CC1] |
| 224 | 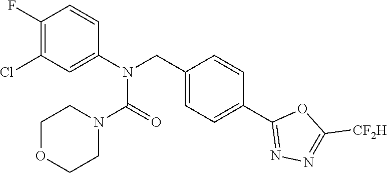 [SMILES: O=C(N1CCOCC1)N(Cc1ccc(-c2nnc(C(F)F)o2)cc1)c1ccc(F)c(Cl)c1] |
| 225 | 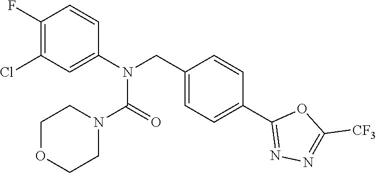 [SMILES: O=C(N1CCOCC1)N(Cc1ccc(-c2nnc(C(F)(F)F)o2)cc1)c1ccc(F)c(Cl)c1] |
| 226 | 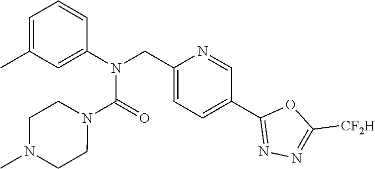 [SMILES: Cc1cccc(N(Cc2ccc(-c3nnc(C(F)F)o3)cn2)C(=O)N2CCN(C)CC2)c1] |
| 227 | 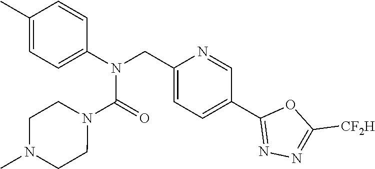 [SMILES: Cc1ccc(N(Cc2ccc(-c3nnc(C(F)F)o3)cn2)C(=O)N2CCN(C)CC2)cc1] |
| 228 | 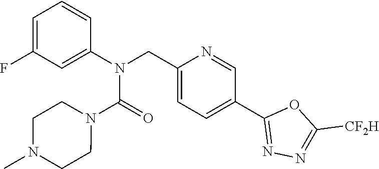 [SMILES: CN1CCN(C(=O)N(Cc2ccc(-c3nnc(C(F)F)o3)cn2)c2cccc(F)c2)CC1] |
| 229 | 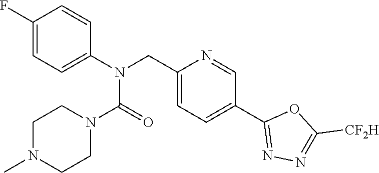 [SMILES: CN1CCN(C(=O)N(Cc2ccc(-c3nnc(C(F)F)o3)cn2)c2ccc(F)cc2)CC1] |
| 230 | 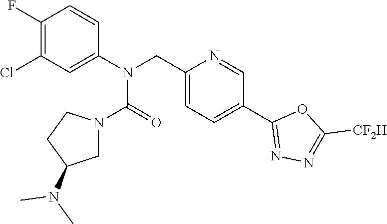 [SMILES: CN(C)[C@H]1CCN(C(=O)N(Cc2ccc(-c3nnc(C(F)F)o3)cn2)c2ccc(F)c(Cl)c2)C1] |
| 231 | 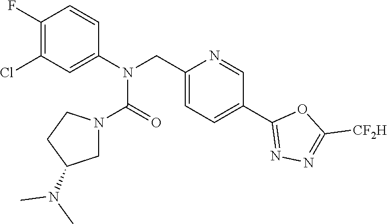 [SMILES: CN(C)[C@@H]1CCN(C(=O)N(Cc2ccc(-c3nnc(C(F)F)o3)cn2)c2ccc(F)c(Cl)c2)C1] |
| 232 | 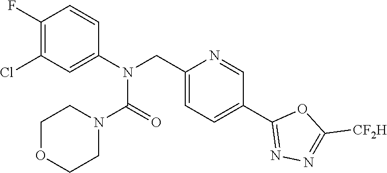 [SMILES: O=C(N1CCOCC1)N(Cc1ccc(-c2nnc(C(F)F)o2)cn1)c1ccc(F)c(Cl)c1] |
| 233 | 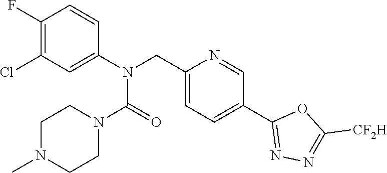 [SMILES: CN1CCN(C(=O)N(Cc2ccc(-c3nnc(C(F)F)o3)cn2)c2ccc(F)c(Cl)c2)CC1] |
| 234 | 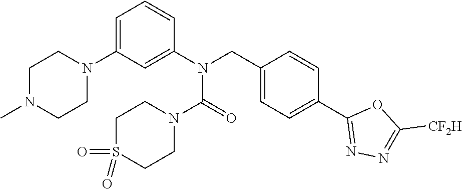 [SMILES: CN1CCN(c2cccc(N(Cc3ccc(-c4nnc(C(F)F)o4)cc3)C(=O)N3CCS(=O)(=O)CC3)c2)CC1] |
| 235 | 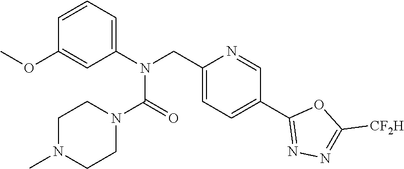 [SMILES: COc1cccc(N(Cc2ccc(-c3nnc(C(F)F)o3)cn2)C(=O)N2CCN(C)CC2)c1] |
| 236 | 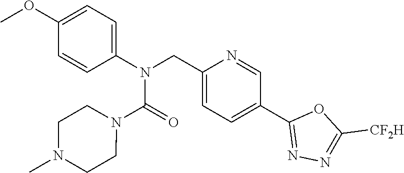 [SMILES: COc1ccc(N(Cc2ccc(-c3nnc(C(F)F)o3)cn2)C(=O)N2CCN(C)CC2)cc1] |
| 237 | 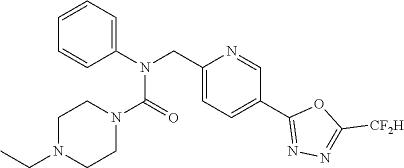 [SMILES: CCN1CCN(C(=O)N(Cc2ccc(-c3nnc(C(F)F)o3)cn2)c2ccccc2)CC1] |
| 238 | 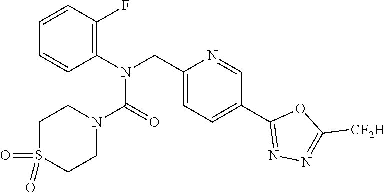 [SMILES: O=C(N1CCS(=O)(=O)CC1)N(Cc1ccc(-c2nnc(C(F)F)o2)cn1)c1ccccc1F] |
| 239 | 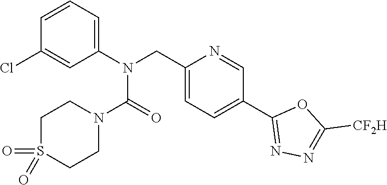 [SMILES: O=C(N1CCS(=O)(=O)CC1)N(Cc1ccc(-c2nnc(C(F)F)o2)cn1)c1cccc(Cl)c1] |
| 240 | 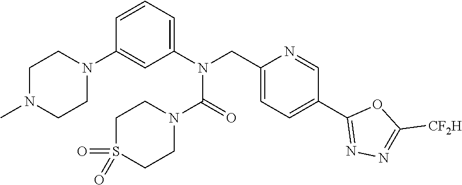 [SMILES: CN1CCN(c2cccc(N(Cc3ccc(-c4nnc(C(F)F)o4)cn3)C(=O)N3CCS(=O)(=O)CC3)c2)CC1] |
| 241 | 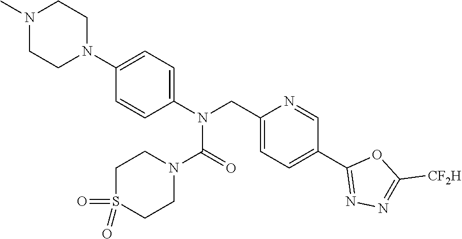 [SMILES: CN1CCN(c2ccc(N(Cc3ccc(-c4nnc(C(F)F)o4)cn3)C(=O)N3CCS(=O)(=O)CC3)cc2)CC1] |
| 242 | 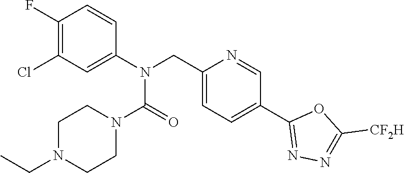 [SMILES: CCN1CCN(C(=O)N(Cc2ccc(-c3nnc(C(F)F)o3)cn2)c2ccc(F)c(Cl)c2)CC1] |
| 243 | 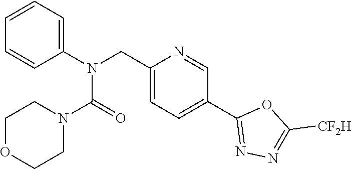 [SMILES: O=C(N1CCOCC1)N(Cc1ccc(-c2nnc(C(F)F)o2)cn1)c1ccccc1] |
| 244 | 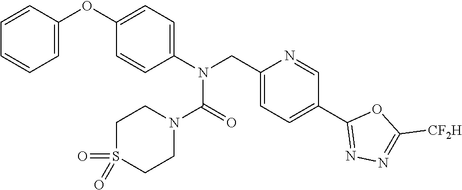 [SMILES: O=C(N1CCS(=O)(=O)CC1)N(Cc1ccc(-c2nnc(C(F)F)o2)cn1)c1ccc(Oc2ccccc2)cc1] |
| 245 | 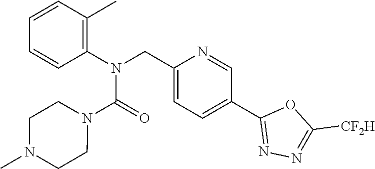 [SMILES: Cc1ccccc1N(Cc1ccc(-c2nnc(C(F)F)o2)cn1)C(=O)N1CCN(C)CC1] |
| 246 | 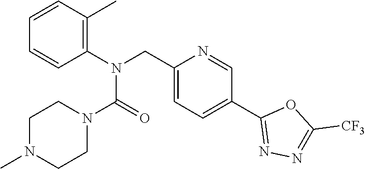 [SMILES: Cc1ccccc1N(Cc1ccc(-c2nnc(C(F)(F)F)o2)cn1)C(=O)N1CCN(C)CC1] |
| 247 | 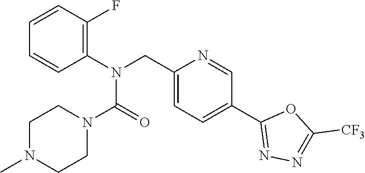 [SMILES: CN1CCN(C(=O)N(Cc2ccc(-c3nnc(C(F)(F)F)o3)cn2)c2ccccc2F)CC1] |
| 248 | 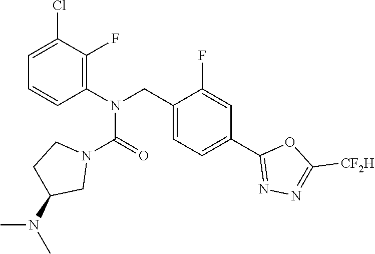 [SMILES: CN(C)[C@H]1CCN(C(=O)N(Cc2ccc(-c3nnc(C(F)F)o3)cc2F)c2cccc(Cl)c2F)C1] |
| 249 | 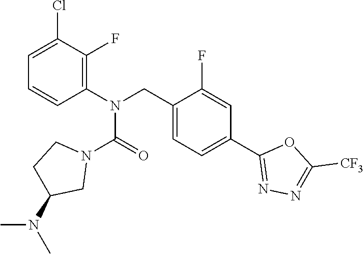 [SMILES: CN(C)[C@H]1CCN(C(=O)N(Cc2ccc(-c3nnc(C(F)(F)F)o3)cc2F)c2cccc(Cl)c2F)C1] |
| 250 | 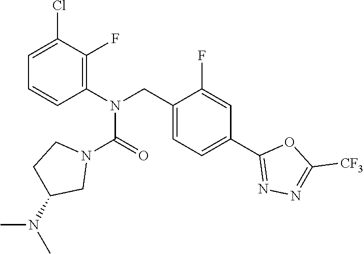 [SMILES: CN(C)[C@@H]1CCN(C(=O)N(Cc2ccc(-c3nnc(C(F)(F)F)o3)cc2F)c2cccc(Cl)c2F)C1] |
| 251 | 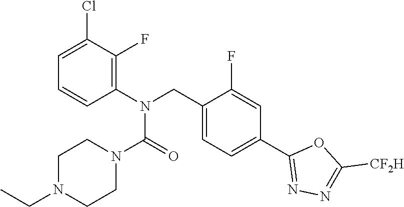 [SMILES: CCN1CCN(C(=O)N(Cc2ccc(-c3nnc(C(F)F)o3)cc2F)c2cccc(Cl)c2F)CC1] |
| 252 | 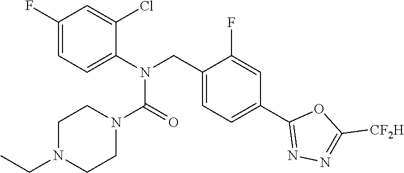 [SMILES: CCN1CCN(C(=O)N(Cc2ccc(-c3nnc(C(F)F)o3)cc2F)c2ccc(F)cc2Cl)CC1] |
| 253 | 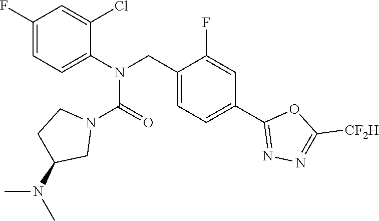 [SMILES: CN(C)[C@H]1CCN(C(=O)N(Cc2ccc(-c3nnc(C(F)F)o3)cc2F)c2ccc(F)cc2Cl)C1] |
| 254 | 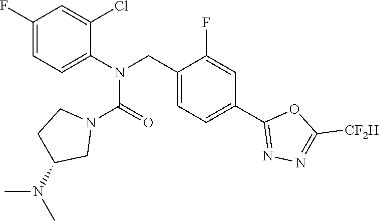 [SMILES: CN(C)[C@@H]1CCN(C(=O)N(Cc2ccc(-c3nnc(C(F)F)o3)cc2F)c2ccc(F)cc2Cl)C1] |
| 255 | 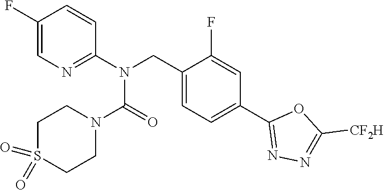 [SMILES: O=C(N1CCS(=O)(=O)CC1)N(Cc1ccc(-c2nnc(C(F)F)o2)cc1F)c1ccc(F)cn1] |
| 256 | 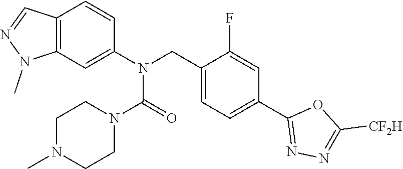 [SMILES: CN1CCN(C(=O)N(Cc2ccc(-c3nnc(C(F)F)o3)cc2F)c2ccc3cnn(C)c3c2)CC1] |
| 257 | 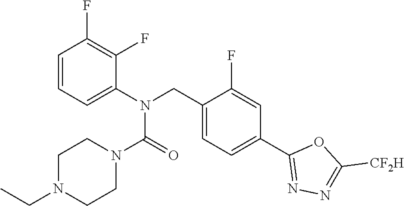 [SMILES: CCN1CCN(C(=O)N(Cc2ccc(-c3nnc(C(F)F)o3)cc2F)c2cccc(F)c2F)CC1] |
| 258 | 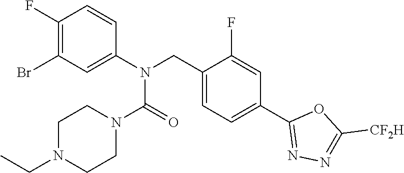 [SMILES: CCN1CCN(C(=O)N(Cc2ccc(-c3nnc(C(F)F)o3)cc2F)c2ccc(F)c(Br)c2)CC1] |
| 259 | 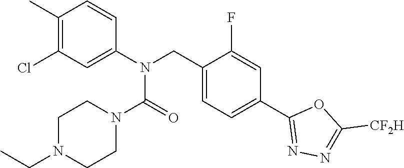 [SMILES: CCN1CCN(C(=O)N(Cc2ccc(-c3nnc(C(F)F)o3)cc2F)c2ccc(C)c(Cl)c2)CC1] |
| 260 | 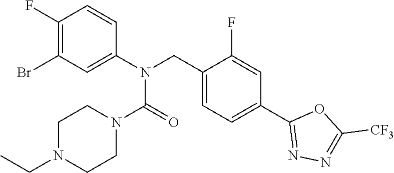 [SMILES: CCN1CCN(C(=O)N(Cc2ccc(-c3nnc(C(F)(F)F)o3)cc2F)c2ccc(F)c(Br)c2)CC1] |
| 261 | 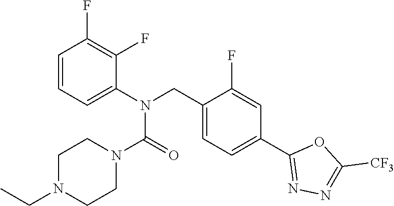 [SMILES: CCN1CCN(C(=O)N(Cc2ccc(-c3nnc(C(F)(F)F)o3)cc2F)c2cccc(F)c2F)CC1] |
| 262 | 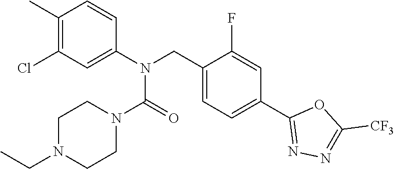 [SMILES: CCN1CCN(C(=O)N(Cc2ccc(-c3nnc(C(F)(F)F)o3)cc2F)c2ccc(C)c(Cl)c2)CC1] |
| 263 | 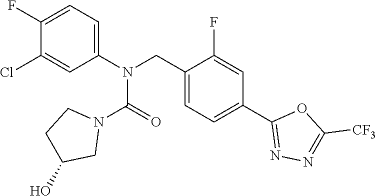 [SMILES: O=C(N1CC[C@@H](O)C1)N(Cc1ccc(-c2nnc(C(F)(F)F)o2)cc1F)c1ccc(F)c(Cl)c1] |
| 264 | 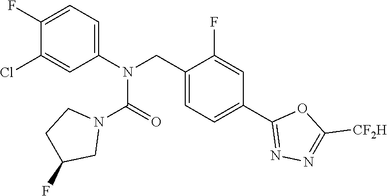 [SMILES: O=C(N1CC[C@H](F)C1)N(Cc1ccc(-c2nnc(C(F)F)o2)cc1F)c1ccc(F)c(Cl)c1] |
| 265 | 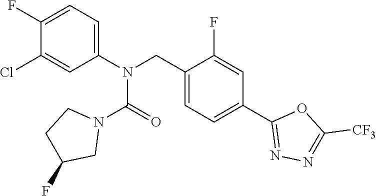 [SMILES: O=C(N1CC[C@H](F)C1)N(Cc1ccc(-c2nnc(C(F)(F)F)o2)cc1F)c1ccc(F)c(Cl)c1] |
| 266 | 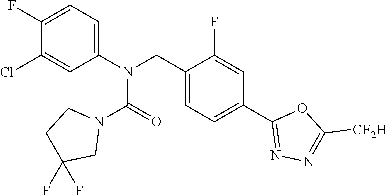 [SMILES: O=C(N1CCC(F)(F)C1)N(Cc1ccc(-c2nnc(C(F)F)o2)cc1F)c1ccc(F)c(Cl)c1] |
| 267 | 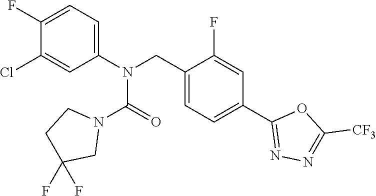 [SMILES: O=C(N1CCC(F)(F)C1)N(Cc1ccc(-c2nnc(C(F)(F)F)o2)cc1F)c1ccc(F)c(Cl)c1] |
| 268 | 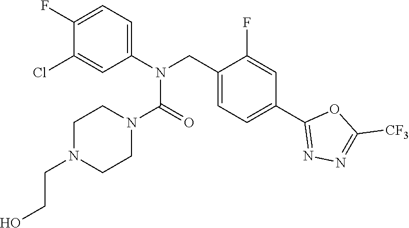 [SMILES: O=C(N1CCN(CCO)CC1)N(Cc1ccc(-c2nnc(C(F)(F)F)o2)cc1F)c1ccc(F)c(Cl)c1] |
| 269 | 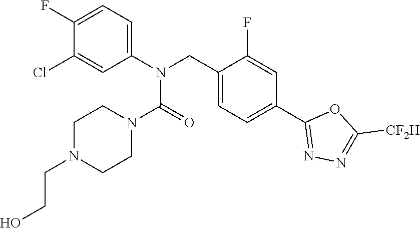 [SMILES: O=C(N1CCN(CCO)CC1)N(Cc1ccc(-c2nnc(C(F)F)o2)cc1F)c1ccc(F)c(Cl)c1] |
| 270 | 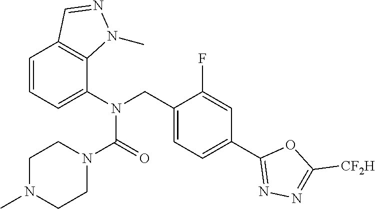 [SMILES: CN1CCN(C(=O)N(Cc2ccc(-c3nnc(C(F)F)o3)cc2F)c2cccc3cnn(C)c23)CC1] |
| 271 | 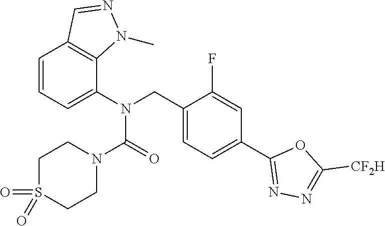 [SMILES: Cn1ncc2cccc(N(Cc3ccc(-c4nnc(C(F)F)o4)cc3F)C(=O)N3CCS(=O)(=O)CC3)c21] |
| 272 | 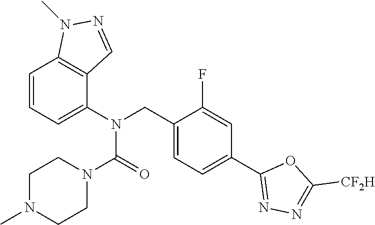 [SMILES: CN1CCN(C(=O)N(Cc2ccc(-c3nnc(C(F)F)o3)cc2F)c2cccc3c2cnn3C)CC1] |
| 273 | 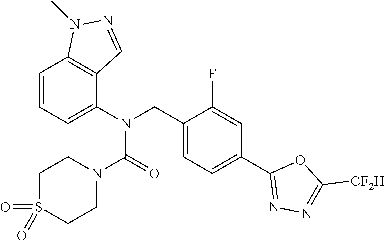 [SMILES: Cn1ncc2c(N(Cc3ccc(-c4nnc(C(F)F)o4)cc3F)C(=O)N3CCS(=O)(=O)CC3)cccc21] |
| 274 | 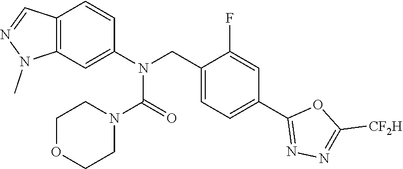 [SMILES: Cn1ncc2ccc(N(Cc3ccc(-c4nnc(C(F)F)o4)cc3F)C(=O)N3CCOCC3)cc21] |
| 275 | 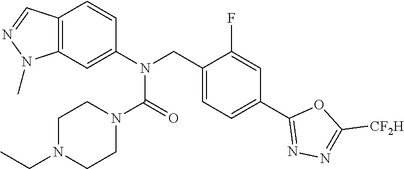 [SMILES: CCN1CCN(C(=O)N(Cc2ccc(-c3nnc(C(F)F)o3)cc2F)c2ccc3cnn(C)c3c2)CC1] |
| 276 | 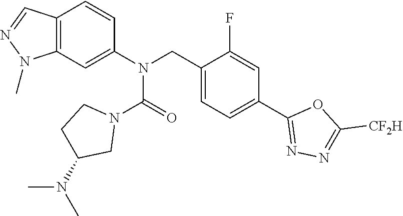 [SMILES: CN(C)[C@@H]1CCN(C(=O)N(Cc2ccc(-c3nnc(C(F)F)o3)cc2F)c2ccc3cnn(C)c3c2)C1] |
| 277 | 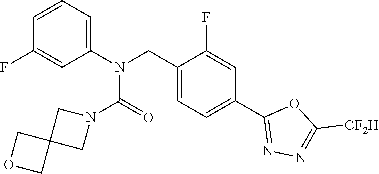 [SMILES: O=C(N1CC2(COC2)C1)N(Cc1ccc(-c2nnc(C(F)F)o2)cc1F)c1cccc(F)c1] |
| 278 | 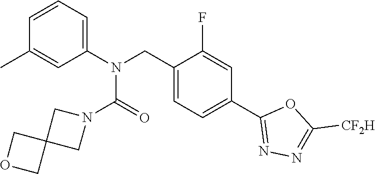 [SMILES: Cc1cccc(N(Cc2ccc(-c3nnc(C(F)F)o3)cc2F)C(=O)N2CC3(COC3)C2)c1] |
| 279 | 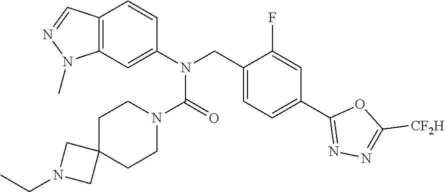 [SMILES: CCN1CC2(CCN(C(=O)N(Cc3ccc(-c4nnc(C(F)F)o4)cc3F)c3ccc4cnn(C)c4c3)CC2)C1] |
| 280 | 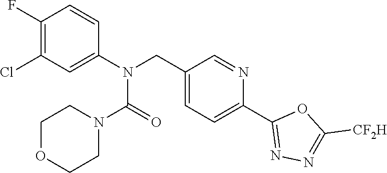 [SMILES: O=C(N1CCOCC1)N(Cc1ccc(-c2nnc(C(F)F)o2)nc1)c1ccc(F)c(Cl)c1] |
| 281 | 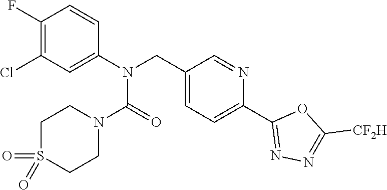 [SMILES: O=C(N1CCS(=O)(=O)CC1)N(Cc1ccc(-c2nnc(C(F)F)o2)nc1)c1ccc(F)c(Cl)c1] |
| 282 | 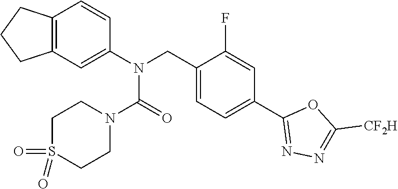 [SMILES: O=C(N1CCS(=O)(=O)CC1)N(Cc1ccc(-c2nnc(C(F)F)o2)cc1F)c1ccc2c(c1)CCC2] |
| 283 | 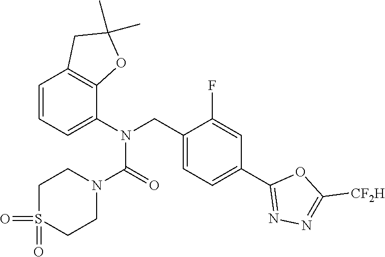 [SMILES: CC1(C)Cc2cccc(N(Cc3ccc(-c4nnc(C(F)F)o4)cc3F)C(=O)N3CCS(=O)(=O)CC3)c2O1] |
| 284 | 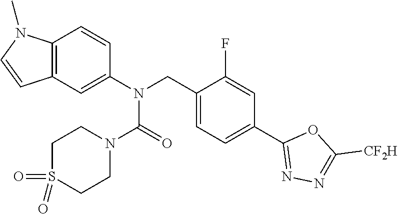 [SMILES: Cn1ccc2cc(N(Cc3ccc(-c4nnc(C(F)F)o4)cc3F)C(=O)N3CCS(=O)(=O)CC3)ccc21] |
| 285 | 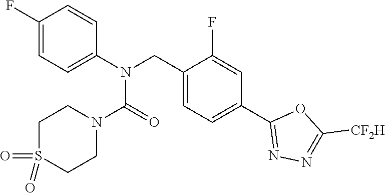 [SMILES: O=C(N1CCS(=O)(=O)CC1)N(Cc1ccc(-c2nnc(C(F)F)o2)cc1F)c1ccc(F)cc1] |
| 286 | 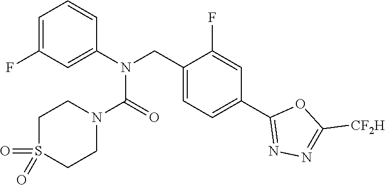 [SMILES: O=C(N1CCS(=O)(=O)CC1)N(Cc1ccc(-c2nnc(C(F)F)o2)cc1F)c1cccc(F)c1] |
| 287 | 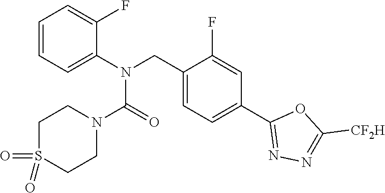 [SMILES: O=C(N1CCS(=O)(=O)CC1)N(Cc1ccc(-c2nnc(C(F)F)o2)cc1F)c1ccccc1F] |
| 288 | 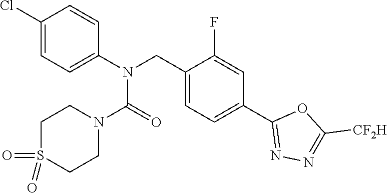 [SMILES: O=C(N1CCS(=O)(=O)CC1)N(Cc1ccc(-c2nnc(C(F)F)o2)cc1F)c1ccc(Cl)cc1] |
| 289 | 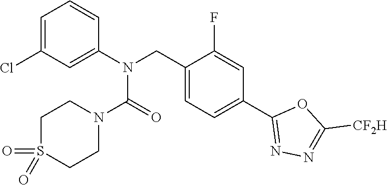 [SMILES: O=C(N1CCS(=O)(=O)CC1)N(Cc1ccc(-c2nnc(C(F)F)o2)cc1F)c1cccc(Cl)c1] |
| 290 | 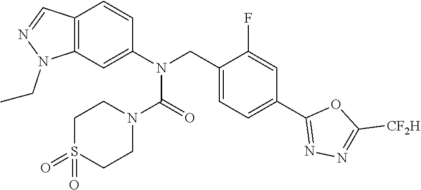 [SMILES: CCn1ncc2ccc(N(Cc3ccc(-c4nnc(C(F)F)o4)cc3F)C(=O)N3CCS(=O)(=O)CC3)cc21] |
| 291 | 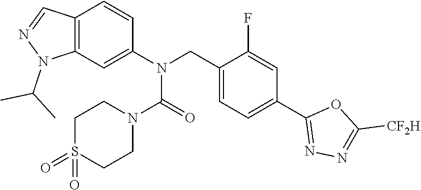 [SMILES: CC(C)n1ncc2ccc(N(Cc3ccc(-c4nnc(C(F)F)o4)cc3F)C(=O)N3CCS(=O)(=O)CC3)cc21] |
| 292 | 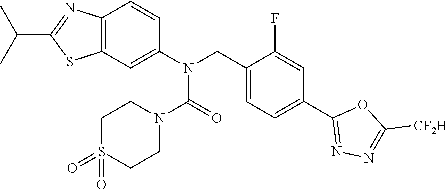 [SMILES: CC(C)c1nc2ccc(N(Cc3ccc(-c4nnc(C(F)F)o4)cc3F)C(=O)N3CCS(=O)(=O)CC3)cc2s1] |
| 293 | 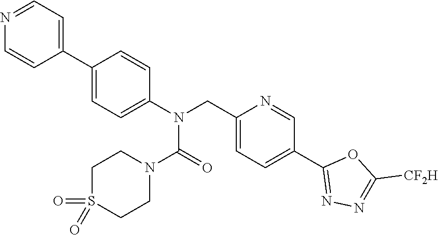 [SMILES: O=C(N1CCS(=O)(=O)CC1)N(Cc1ccc(-c2nnc(C(F)F)o2)cn1)c1ccc(-c2ccncc2)cc1] |
| 294 | 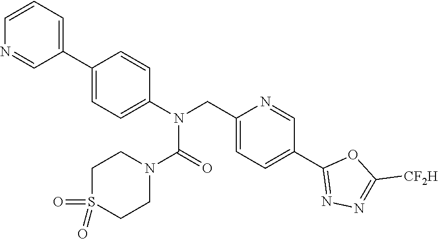 [SMILES: O=C(N1CCS(=O)(=O)CC1)N(Cc1ccc(-c2nnc(C(F)F)o2)cn1)c1ccc(-c2cccnc2)cc1] |
| 295 | 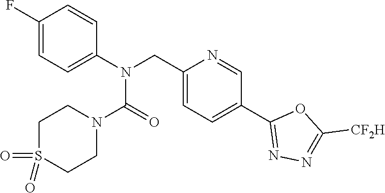 [SMILES: O=C(N1CCS(=O)(=O)CC1)N(Cc1ccc(-c2nnc(C(F)F)o2)cn1)c1ccc(F)cc1] |
| 296 | 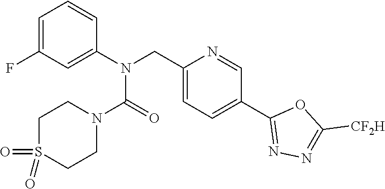 [SMILES: O=C(N1CCS(=O)(=O)CC1)N(Cc1ccc(-c2nnc(C(F)F)o2)cn1)c1cccc(F)c1] |
| 297 | 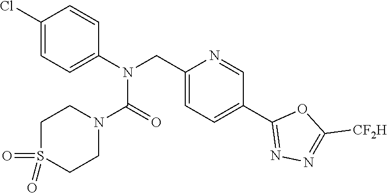 [SMILES: O=C(N1CCS(=O)(=O)CC1)N(Cc1ccc(-c2nnc(C(F)F)o2)cn1)c1ccc(Cl)cc1] |
| 298 | 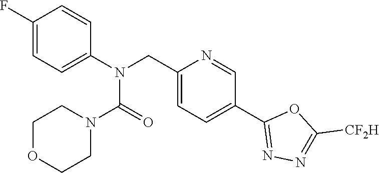 [SMILES: O=C(N1CCOCC1)N(Cc1ccc(-c2nnc(C(F)F)o2)cn1)c1ccc(F)cc1] |
| 299 | 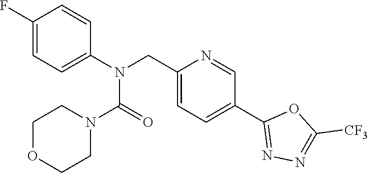 [SMILES: O=C(N1CCOCC1)N(Cc1ccc(-c2nnc(C(F)(F)F)o2)cn1)c1ccc(F)cc1] |
| 300 | 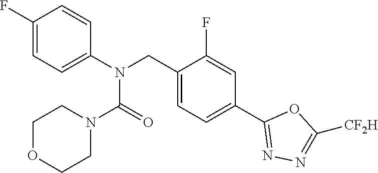 [SMILES: O=C(N1CCOCC1)N(Cc1ccc(-c2nnc(C(F)F)o2)cc1F)c1ccc(F)cc1] |
| 301 | 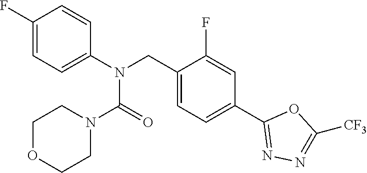 [SMILES: O=C(N1CCOCC1)N(Cc1ccc(-c2nnc(C(F)(F)F)o2)cc1F)c1ccc(F)cc1] |
| 302 | 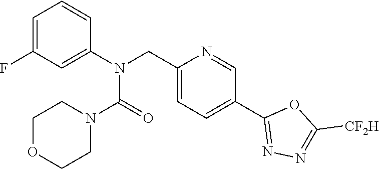 [SMILES: O=C(N1CCOCC1)N(Cc1ccc(-c2nnc(C(F)F)o2)cn1)c1cccc(F)c1] |
| 303 | 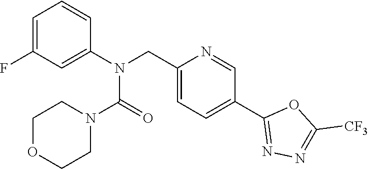 [SMILES: O=C(N1CCOCC1)N(Cc1ccc(-c2nnc(C(F)(F)F)o2)cn1)c1cccc(F)c1] |
| 304 | 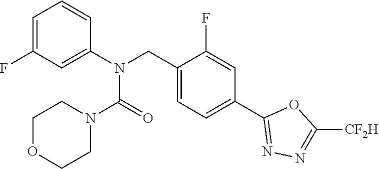 [SMILES: O=C(N1CCOCC1)N(Cc1ccc(-c2nnc(C(F)F)o2)cc1F)c1cccc(F)c1] |
| 305 | 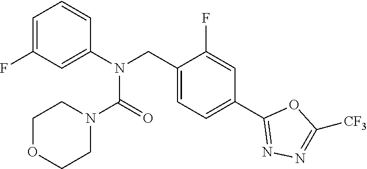 [SMILES: O=C(N1CCOCC1)N(Cc1ccc(-c2nnc(C(F)(F)F)o2)cc1F)c1cccc(F)c1] |
| 306 | 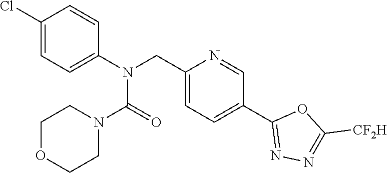 [SMILES: O=C(N1CCOCC1)N(Cc1ccc(-c2nnc(C(F)F)o2)cn1)c1ccc(Cl)cc1] |
| 307 | 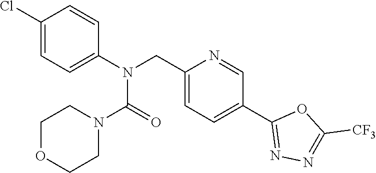 [SMILES: O=C(N1CCOCC1)N(Cc1ccc(-c2nnc(C(F)(F)F)o2)cn1)c1ccc(Cl)cc1] |
| 308 | 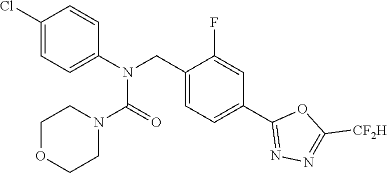 [SMILES: O=C(N1CCOCC1)N(Cc1ccc(-c2nnc(C(F)F)o2)cc1F)c1ccc(Cl)cc1] |
| 309 | 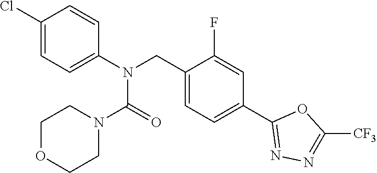 [SMILES: O=C(N1CCOCC1)N(Cc1ccc(-c2nnc(C(F)(F)F)o2)cc1F)c1ccc(Cl)cc1] |
| 310 | 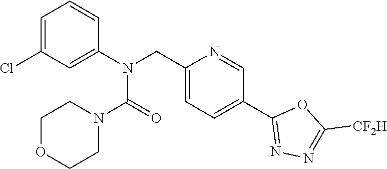 [SMILES: O=C(N1CCOCC1)N(Cc1ccc(-c2nnc(C(F)F)o2)cn1)c1cccc(Cl)c1] |
| 311 | 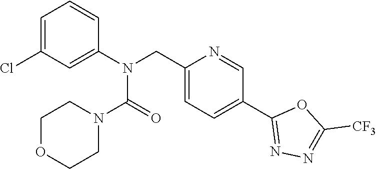 [SMILES: O=C(N1CCOCC1)N(Cc1ccc(-c2nnc(C(F)(F)F)o2)cn1)c1cccc(Cl)c1] |
| 312 | 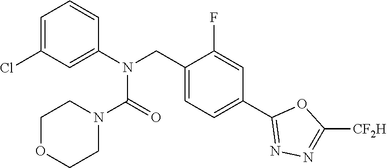 [SMILES: O=C(N1CCOCC1)N(Cc1ccc(-c2nnc(C(F)F)o2)cc1F)c1cccc(Cl)c1] |
| 313 | 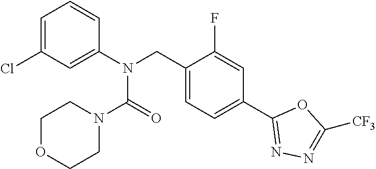 [SMILES: O=C(N1CCOCC1)N(Cc1ccc(-c2nnc(C(F)(F)F)o2)cc1F)c1cccc(Cl)c1] |
| 314 | 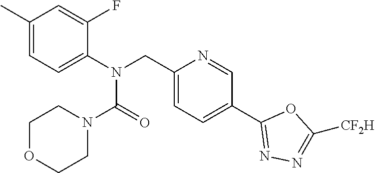 [SMILES: Cc1ccc(N(Cc2ccc(-c3nnc(C(F)F)o3)cn2)C(=O)N2CCOCC2)c(F)c1] |
| 315 | 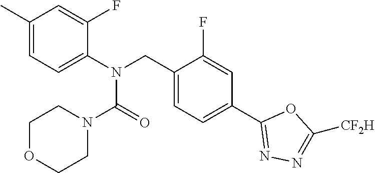 [SMILES: Cc1ccc(N(Cc2ccc(-c3nnc(C(F)F)o3)cc2F)C(=O)N2CCOCC2)c(F)c1] |
| 316 | 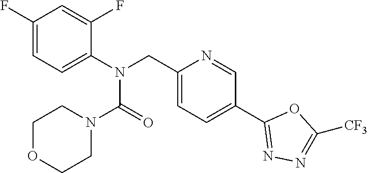 [SMILES: O=C(N1CCOCC1)N(Cc1ccc(-c2nnc(C(F)(F)F)o2)cn1)c1ccc(F)cc1F] |
| 317 | 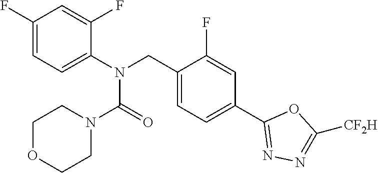 [SMILES: O=C(N1CCOCC1)N(Cc1ccc(-c2nnc(C(F)F)o2)cc1F)c1ccc(F)cc1F] |
| 318 | 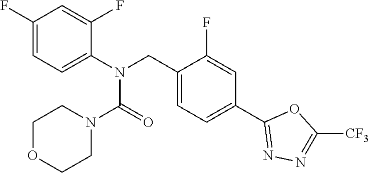 [SMILES: O=C(N1CCOCC1)N(Cc1ccc(-c2nnc(C(F)(F)F)o2)cc1F)c1ccc(F)cc1F] |
| 319 | 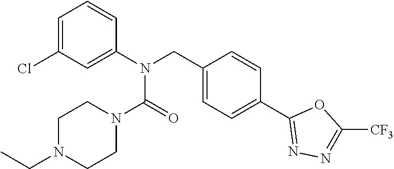 [SMILES: CCN1CCN(C(=O)N(Cc2ccc(-c3nnc(C(F)(F)F)o3)cc2)c2cccc(Cl)c2)CC1] |
| 320 | 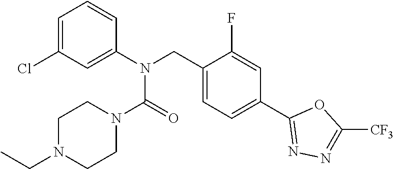 [SMILES: CCN1CCN(C(=O)N(Cc2ccc(-c3nnc(C(F)(F)F)o3)cc2F)c2cccc(Cl)c2)CC1] |
| 321 | 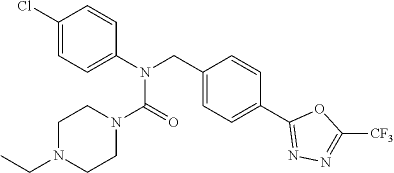 [SMILES: CCN1CCN(C(=O)N(Cc2ccc(-c3nnc(C(F)(F)F)o3)cc2)c2ccc(Cl)cc2)CC1] |
| 322 | 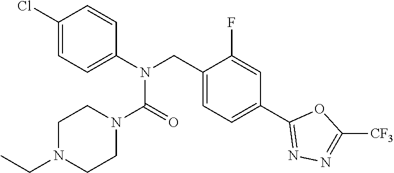 [SMILES: CCN1CCN(C(=O)N(Cc2ccc(-c3nnc(C(F)(F)F)o3)cc2F)c2ccc(Cl)cc2)CC1] |
| 323 | 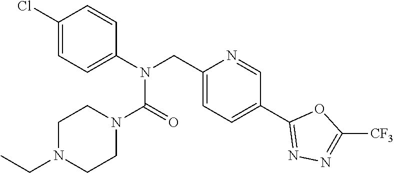 [SMILES: CCN1CCN(C(=O)N(Cc2ccc(-c3nnc(C(F)(F)F)o3)cn2)c2ccc(Cl)cc2)CC1] |
| 324 | 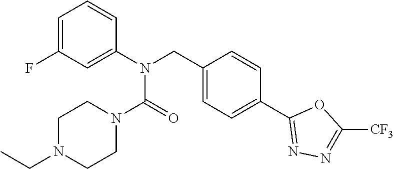 [SMILES: CCN1CCN(C(=O)N(Cc2ccc(-c3nnc(C(F)(F)F)o3)cc2)c2cccc(F)c2)CC1] |
| 325 | 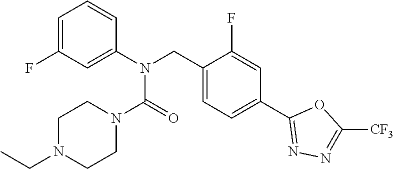 [SMILES: CCN1CCN(C(=O)N(Cc2ccc(-c3nnc(C(F)(F)F)o3)cc2F)c2cccc(F)c2)CC1] |
| 326 | 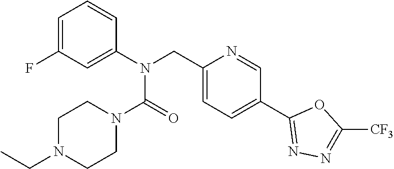 [SMILES: CCN1CCN(C(=O)N(Cc2ccc(-c3nnc(C(F)(F)F)o3)cn2)c2cccc(F)c2)CC1] |
| 327 | 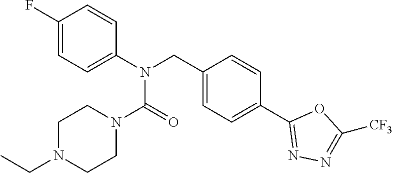 [SMILES: CCN1CCN(C(=O)N(Cc2ccc(-c3nnc(C(F)(F)F)o3)cc2)c2ccc(F)cc2)CC1] |
| 328 | 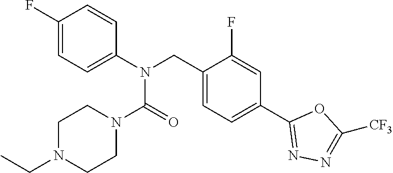 [SMILES: CCN1CCN(C(=O)N(Cc2ccc(-c3nnc(C(F)(F)F)o3)cc2F)c2ccc(F)cc2)CC1] |
| 329 | 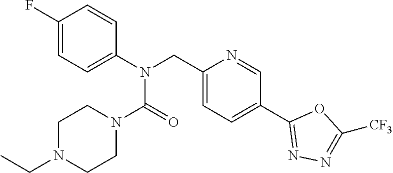 [SMILES: CCN1CCN(C(=O)N(Cc2ccc(-c3nnc(C(F)(F)F)o3)cn2)c2ccc(F)cc2)CC1] |
| 330 | 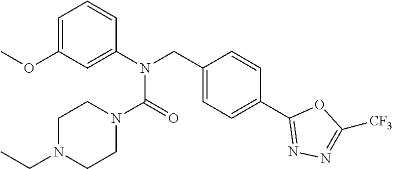 [SMILES: CCN1CCN(C(=O)N(Cc2ccc(-c3nnc(C(F)(F)F)o3)cc2)c2cccc(OC)c2)CC1] |
| 331 | 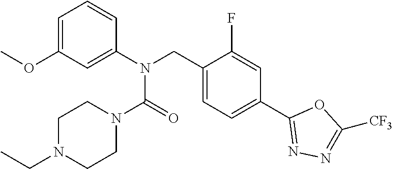 [SMILES: CCN1CCN(C(=O)N(Cc2ccc(-c3nnc(C(F)(F)F)o3)cc2F)c2cccc(OC)c2)CC1] |
| 332 | 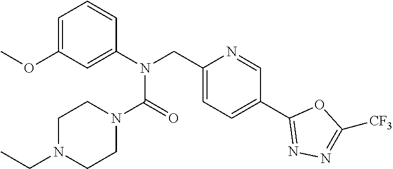 [SMILES: CCN1CCN(C(=O)N(Cc2ccc(-c3nnc(C(F)(F)F)o3)cn2)c2cccc(OC)c2)CC1] |
| 333 | 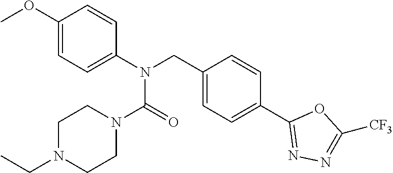 [SMILES: CCN1CCN(C(=O)N(Cc2ccc(-c3nnc(C(F)(F)F)o3)cc2)c2ccc(OC)cc2)CC1] |
| 334 | 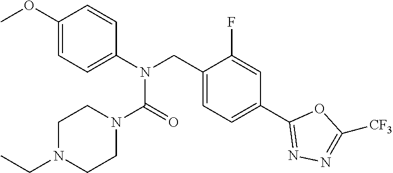 [SMILES: CCN1CCN(C(=O)N(Cc2ccc(-c3nnc(C(F)(F)F)o3)cc2F)c2ccc(OC)cc2)CC1] |
| 335 | 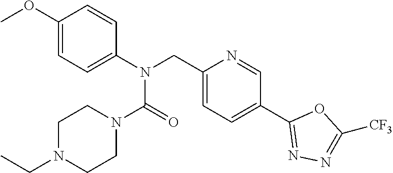 [SMILES: CCN1CCN(C(=O)N(Cc2ccc(-c3nnc(C(F)(F)F)o3)cn2)c2ccc(OC)cc2)CC1] |
| 336 | 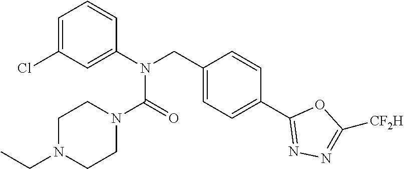 [SMILES: CCN1CCN(C(=O)N(Cc2ccc(-c3nnc(C(F)F)o3)cc2)c2cccc(Cl)c2)CC1] |
| 337 | 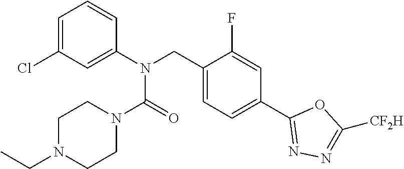 [SMILES: CCN1CCN(C(=O)N(Cc2ccc(-c3nnc(C(F)F)o3)cc2F)c2cccc(Cl)c2)CC1] |
| 338 | 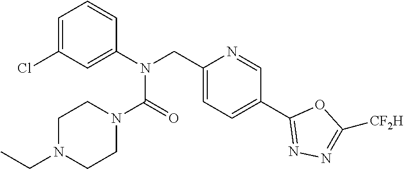 [SMILES: CCN1CCN(C(=O)N(Cc2ccc(-c3nnc(C(F)F)o3)cn2)c2cccc(Cl)c2)CC1] |
| 339 | 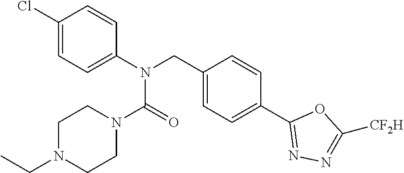 [SMILES: CCN1CCN(C(=O)N(Cc2ccc(-c3nnc(C(F)F)o3)cc2)c2ccc(Cl)cc2)CC1] |
| 340 | 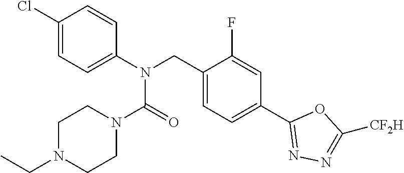 [SMILES: CCN1CCN(C(=O)N(Cc2ccc(-c3nnc(C(F)F)o3)cc2F)c2ccc(Cl)cc2)CC1] |
| 341 | 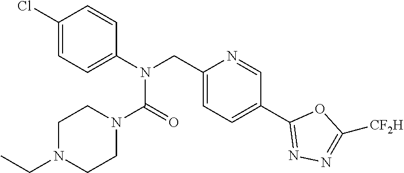 [SMILES: CCN1CCN(C(=O)N(Cc2ccc(-c3nnc(C(F)F)o3)cn2)c2ccc(Cl)cc2)CC1] |
| 342 | 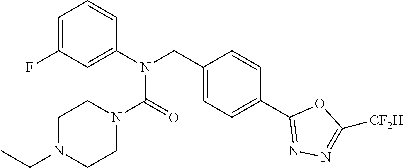 [SMILES: CCN1CCN(C(=O)N(Cc2ccc(-c3nnc(C(F)F)o3)cc2)c2cccc(F)c2)CC1] |
| 343 | 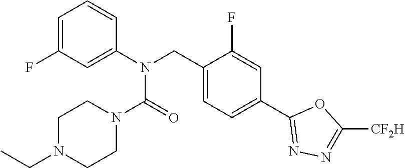 [SMILES: CCN1CCN(C(=O)N(Cc2ccc(-c3nnc(C(F)F)o3)cc2F)c2cccc(F)c2)CC1] |
| 344 | 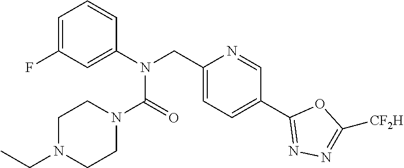 [SMILES: CCN1CCN(C(=O)N(Cc2ccc(-c3nnc(C(F)F)o3)cn2)c2cccc(F)c2)CC1] |
| 345 | 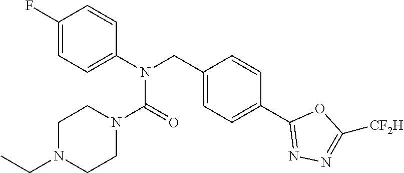 [SMILES: CCN1CCN(C(=O)N(Cc2ccc(-c3nnc(C(F)F)o3)cc2)c2ccc(F)cc2)CC1] |
| 346 | 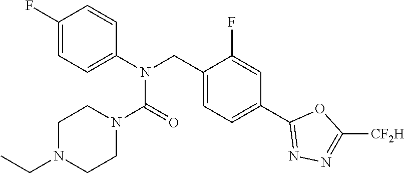 [SMILES: CCN1CCN(C(=O)N(Cc2ccc(-c3nnc(C(F)F)o3)cc2F)c2ccc(F)cc2)CC1] |
| 347 | 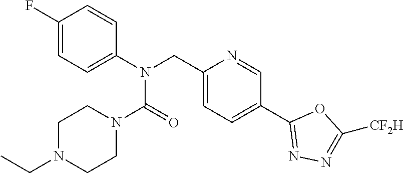 [SMILES: CCN1CCN(C(=O)N(Cc2ccc(-c3nnc(C(F)F)o3)cn2)c2ccc(F)cc2)CC1] |
| 348 | 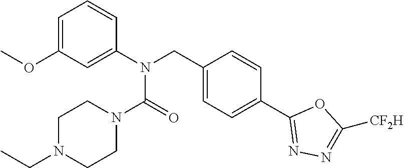 [SMILES: CCN1CCN(C(=O)N(Cc2ccc(-c3nnc(C(F)F)o3)cc2)c2cccc(OC)c2)CC1] |
| 349 | 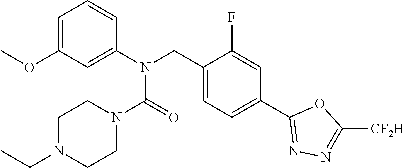 [SMILES: CCN1CCN(C(=O)N(Cc2ccc(-c3nnc(C(F)F)o3)cc2F)c2cccc(OC)c2)CC1] |
| 350 | 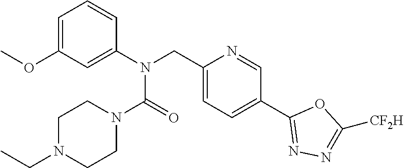 [SMILES: CCN1CCN(C(=O)N(Cc2ccc(-c3nnc(C(F)F)o3)cn2)c2cccc(OC)c2)CC1] |
| 351 | 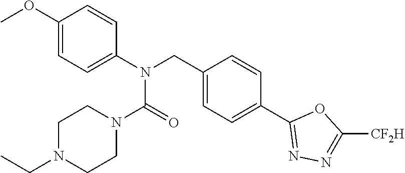 [SMILES: CCN1CCN(C(=O)N(Cc2ccc(-c3nnc(C(F)F)o3)cc2)c2ccc(OC)cc2)CC1] |
| 352 | 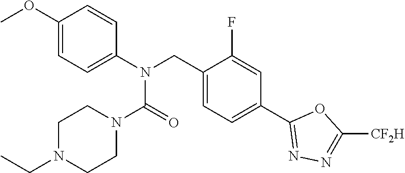 [SMILES: CCN1CCN(C(=O)N(Cc2ccc(-c3nnc(C(F)F)o3)cc2F)c2ccc(OC)cc2)CC1] |
| 353 | 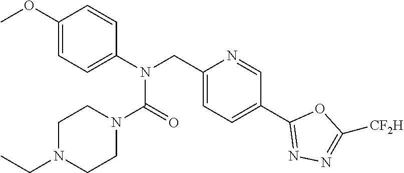 [SMILES: CCN1CCN(C(=O)N(Cc2ccc(-c3nnc(C(F)F)o3)cn2)c2ccc(OC)cc2)CC1] |
| 354 | 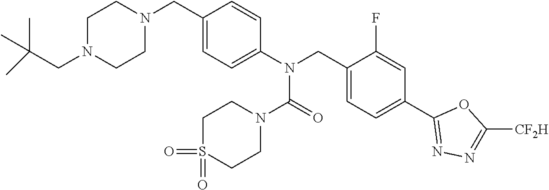 [SMILES: CC(C)(C)CN1CCN(Cc2ccc(N(Cc3ccc(-c4nnc(C(F)F)o4)cc3F)C(=O)N3CCS(=O)(=O)CC3)cc2)CC1] |
| 355 | 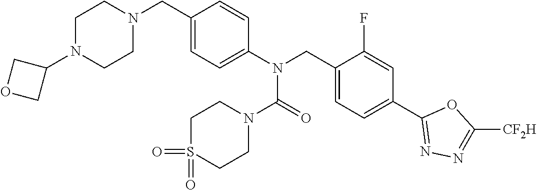 [SMILES: O=C(N1CCS(=O)(=O)CC1)N(Cc1ccc(-c2nnc(C(F)F)o2)cc1F)c1ccc(CN2CCN(C3COC3)CC2)cc1] |
| 356 | 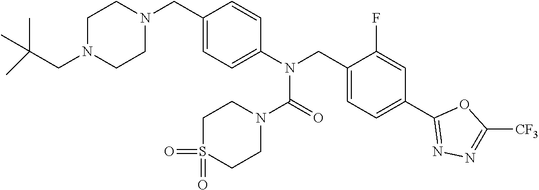 [SMILES: CC(C)(C)CN1CCN(Cc2ccc(N(Cc3ccc(-c4nnc(C(F)(F)F)o4)cc3F)C(=O)N3CCS(=O)(=O)CC3)cc2)CC1] |
| 357 | 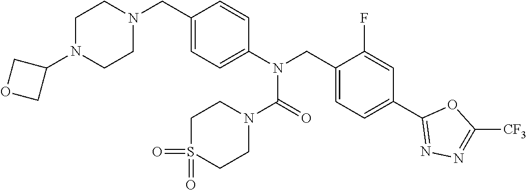 [SMILES: O=C(N1CCS(=O)(=O)CC1)N(Cc1ccc(-c2nnc(C(F)(F)F)o2)cc1F)c1ccc(CN2CCN(C3COC3)CC2)cc1] |
| 358 | 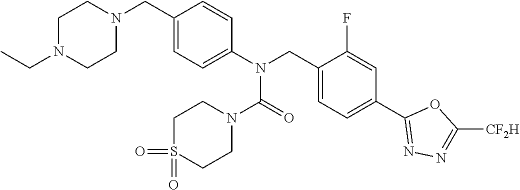 [SMILES: CCN1CCN(Cc2ccc(N(Cc3ccc(-c4nnc(C(F)F)o4)cc3F)C(=O)N3CCS(=O)(=O)CC3)cc2)CC1] |
| 359 | 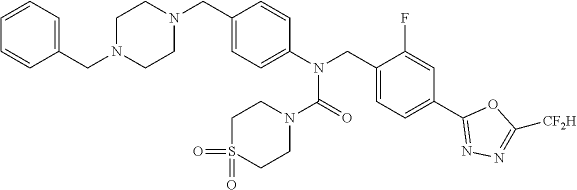 [SMILES: O=C(N1CCS(=O)(=O)CC1)N(Cc1ccc(-c2nnc(C(F)F)o2)cc1F)c1ccc(CN2CCN(Cc3ccccc3)CC2)cc1] |
| 360 | 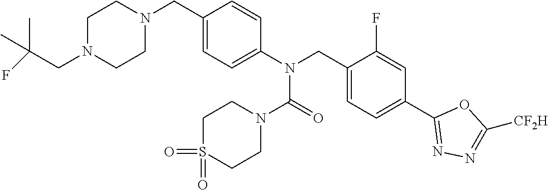 [SMILES: CC(C)(F)CN1CCN(Cc2ccc(N(Cc3ccc(-c4nnc(C(F)F)o4)cc3F)C(=O)N3CCS(=O)(=O)CC3)cc2)CC1] |
| 361 | 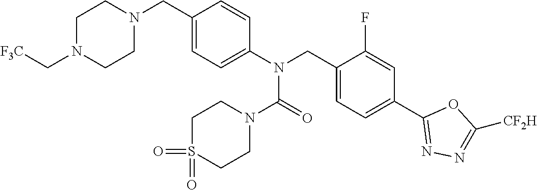 [SMILES: O=C(N1CCS(=O)(=O)CC1)N(Cc1ccc(-c2nnc(C(F)F)o2)cc1F)c1ccc(CN2CCN(CC(F)(F)F)CC2)cc1] |
| 362 | 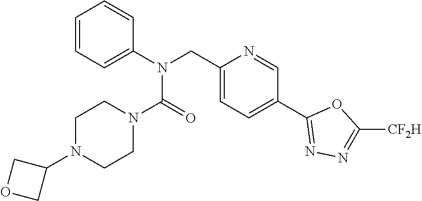 [SMILES: O=C(N1CCN(C2COC2)CC1)N(Cc1ccc(-c2nnc(C(F)F)o2)cn1)c1ccccc1] |
| 363 | 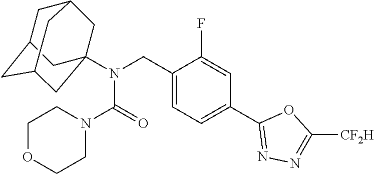 [SMILES: O=C(N1CCOCC1)N(Cc1ccc(-c2nnc(C(F)F)o2)cc1F)C12CC3CC(CC(C3)C1)C2] |
| 364 | 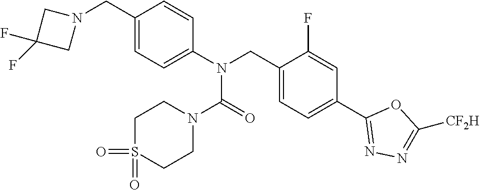 [SMILES: O=C(N1CCS(=O)(=O)CC1)N(Cc1ccc(-c2nnc(C(F)F)o2)cc1F)c1ccc(CN2CC(F)(F)C2)cc1] |
| 365 | 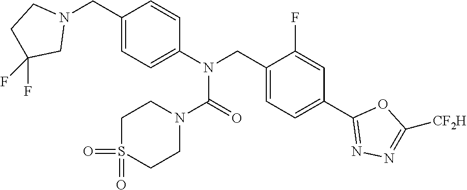 [SMILES: O=C(N1CCS(=O)(=O)CC1)N(Cc1ccc(-c2nnc(C(F)F)o2)cc1F)c1ccc(CN2CCC(F)(F)C2)cc1] |
| 366 | 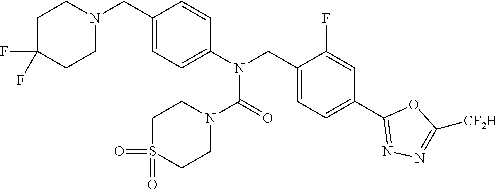 [SMILES: O=C(N1CCS(=O)(=O)CC1)N(Cc1ccc(-c2nnc(C(F)F)o2)cc1F)c1ccc(CN2CCC(F)(F)CC2)cc1] |
| 367 | 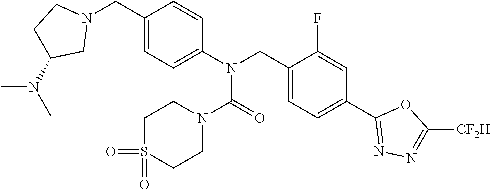 [SMILES: CN(C)[C@@H]1CCN(Cc2ccc(N(Cc3ccc(-c4nnc(C(F)F)o4)cc3F)C(=O)N3CCS(=O)(=O)CC3)cc2)C1] |
| 368 | 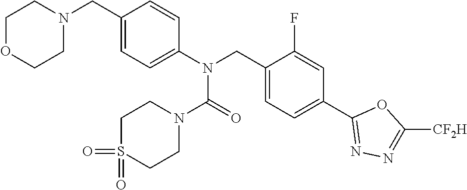 [SMILES: O=C(N1CCS(=O)(=O)CC1)N(Cc1ccc(-c2nnc(C(F)F)o2)cc1F)c1ccc(CN2CCOCC2)cc1] |
| 369 | 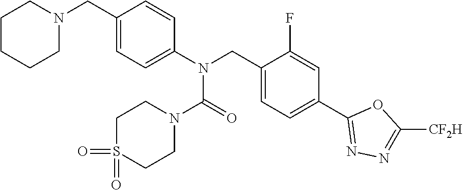 [SMILES: O=C(N1CCS(=O)(=O)CC1)N(Cc1ccc(-c2nnc(C(F)F)o2)cc1F)c1ccc(CN2CCCCC2)cc1] |
| 370 | 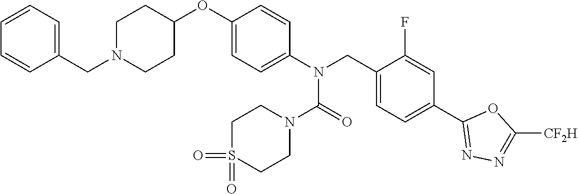 [SMILES: O=C(N1CCS(=O)(=O)CC1)N(Cc1ccc(-c2nnc(C(F)F)o2)cc1F)c1ccc(OC2CCN(Cc3ccccc3)CC2)cc1] |
| 371 | 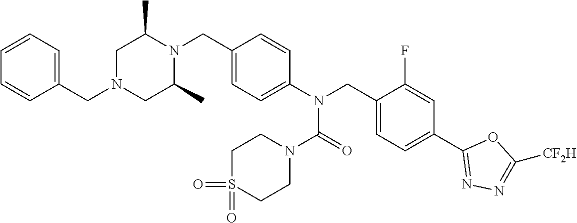 [SMILES: C[C@@H]1CN(Cc2ccccc2)C[C@H](C)N1Cc1ccc(N(Cc2ccc(-c3nnc(C(F)F)o3)cc2F)C(=O)N2CCS(=O)(=O)CC2)cc1] |
| 372 | 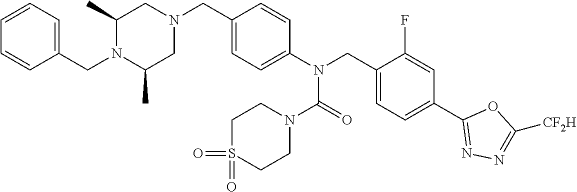 [SMILES: C[C@@H]1CN(Cc2ccc(N(Cc3ccc(-c4nnc(C(F)F)o4)cc3F)C(=O)N3CCS(=O)(=O)CC3)cc2)C[C@H](C)N1Cc1ccccc1] |
| 373 | 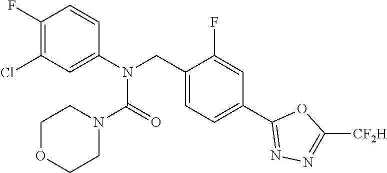 [SMILES: O=C(N1CCOCC1)N(Cc1ccc(-c2nnc(C(F)F)o2)cc1F)c1ccc(F)c(Cl)c1] |
| 374 | 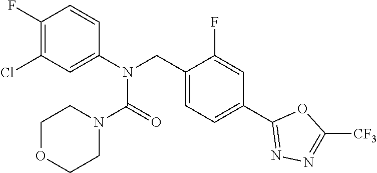 [SMILES: O=C(N1CCOCC1)N(Cc1ccc(-c2nnc(C(F)(F)F)o2)cc1F)c1ccc(F)c(Cl)c1] |
| 375 | 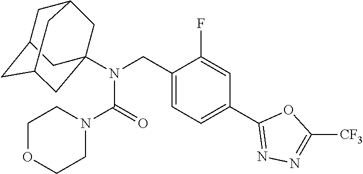 [SMILES: O=C(N1CCOCC1)N(Cc1ccc(-c2nnc(C(F)(F)F)o2)cc1F)C12CC3CC(CC(C3)C1)C2] |
| 376 | 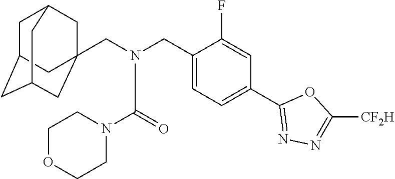 [SMILES: O=C(N1CCOCC1)N(Cc1ccc(-c2nnc(C(F)F)o2)cc1F)CC12CC3CC(CC(C3)C1)C2] |
| 377 | 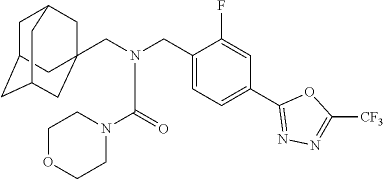 [SMILES: O=C(N1CCOCC1)N(Cc1ccc(-c2nnc(C(F)(F)F)o2)cc1F)CC12CC3CC(CC(C3)C1)C2] |
| 378 | 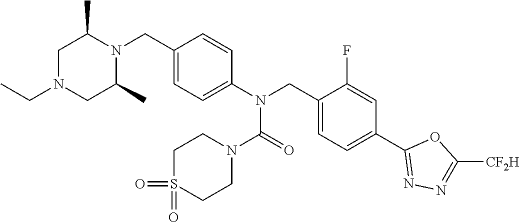 [SMILES: CCN1C[C@@H](C)N(Cc2ccc(N(Cc3ccc(-c4nnc(C(F)F)o4)cc3F)C(=O)N3CCS(=O)(=O)CC3)cc2)[C@@H](C)C1] |
| 379 | 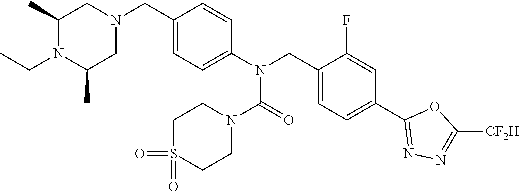 [SMILES: CCN1[C@H](C)CN(Cc2ccc(N(Cc3ccc(-c4nnc(C(F)F)o4)cc3F)C(=O)N3CCS(=O)(=O)CC3)cc2)C[C@@H]1C] |
| 380 | 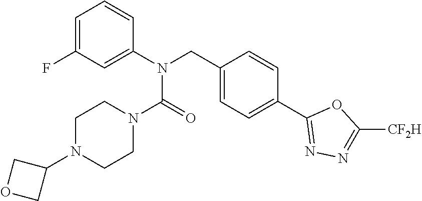 [SMILES: O=C(N1CCN(C2COC2)CC1)N(Cc1ccc(-c2nnc(C(F)F)o2)cc1)c1cccc(F)c1] |
| 381 | 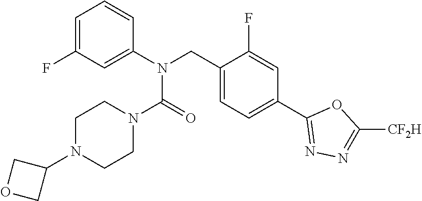 [SMILES: O=C(N1CCN(C2COC2)CC1)N(Cc1ccc(-c2nnc(C(F)F)o2)cc1F)c1cccc(F)c1] |
| 382 | 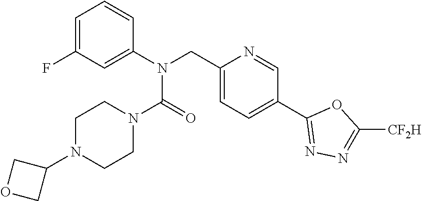 [SMILES: O=C(N1CCN(C2COC2)CC1)N(Cc1ccc(-c2nnc(C(F)F)o2)cn1)c1cccc(F)c1] |
| 383 | 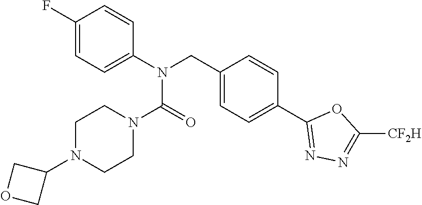 [SMILES: O=C(N1CCN(C2COC2)CC1)N(Cc1ccc(-c2nnc(C(F)F)o2)cc1)c1ccc(F)cc1] |
| 384 | 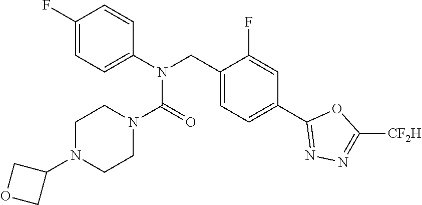 [SMILES: O=C(N1CCN(C2COC2)CC1)N(Cc1ccc(-c2nnc(C(F)F)o2)cc1F)c1ccc(F)cc1] |
| 385 | 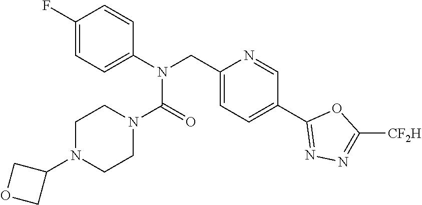 [SMILES: O=C(N1CCN(C2COC2)CC1)N(Cc1ccc(-c2nnc(C(F)F)o2)cn1)c1ccc(F)cc1] |
| 386 | 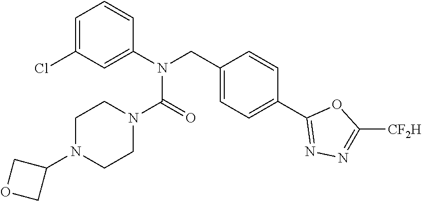 [SMILES: O=C(N1CCN(C2COC2)CC1)N(Cc1ccc(-c2nnc(C(F)F)o2)cc1)c1cccc(Cl)c1] |
| 387 | 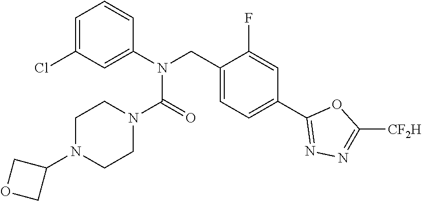 [SMILES: O=C(N1CCN(C2COC2)CC1)N(Cc1ccc(-c2nnc(C(F)F)o2)cc1F)c1cccc(Cl)c1] |
| 388 | 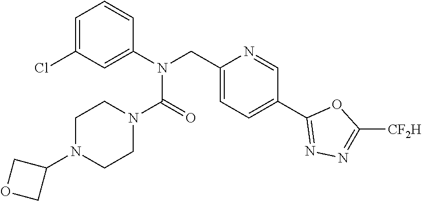 [SMILES: O=C(N1CCN(C2COC2)CC1)N(Cc1ccc(-c2nnc(C(F)F)o2)cn1)c1cccc(Cl)c1] |
| 389 | 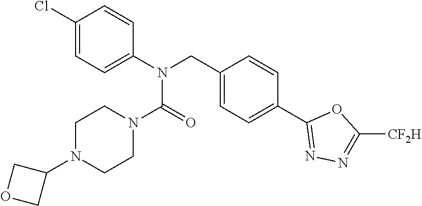 [SMILES: O=C(N1CCN(C2COC2)CC1)N(Cc1ccc(-c2nnc(C(F)F)o2)cc1)c1ccc(Cl)cc1] |
| 390 | 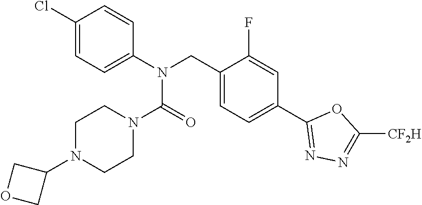 [SMILES: O=C(N1CCN(C2COC2)CC1)N(Cc1ccc(-c2nnc(C(F)F)o2)cc1F)c1ccc(Cl)cc1] |
| 391 | 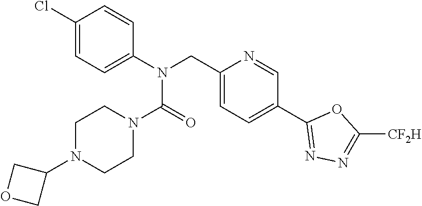 [SMILES: O=C(N1CCN(C2COC2)CC1)N(Cc1ccc(-c2nnc(C(F)F)o2)cn1)c1ccc(Cl)cc1] |
| 392 | 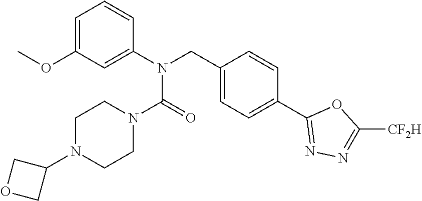 [SMILES: COc1cccc(N(Cc2ccc(-c3nnc(C(F)F)o3)cc2)C(=O)N2CCN(C3COC3)CC2)c1] |
| 393 | 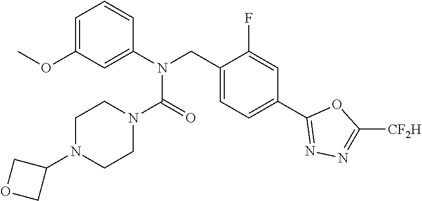 [SMILES: COc1cccc(N(Cc2ccc(-c3nnc(C(F)F)o3)cc2F)C(=O)N2CCN(C3COC3)CC2)c1] |
| 394 | 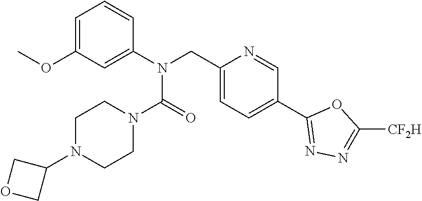 [SMILES: COc1cccc(N(Cc2ccc(-c3nnc(C(F)F)o3)cn2)C(=O)N2CCN(C3COC3)CC2)c1] |
| 395 | 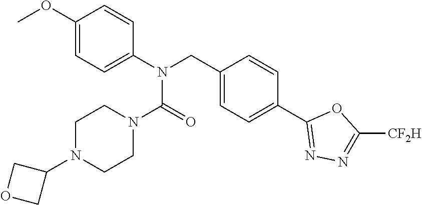 [SMILES: COc1ccc(N(Cc2ccc(-c3nnc(C(F)F)o3)cc2)C(=O)N2CCN(C3COC3)CC2)cc1] |
| 396 | 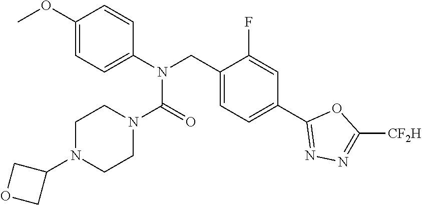 [SMILES: COc1ccc(N(Cc2ccc(-c3nnc(C(F)F)o3)cc2F)C(=O)N2CCN(C3COC3)CC2)cc1] |
| 397 | 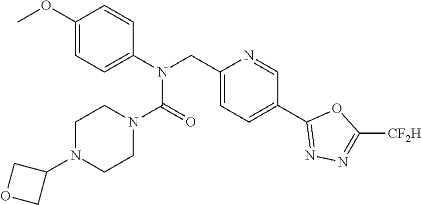 [SMILES: COc1ccc(N(Cc2ccc(-c3nnc(C(F)F)o3)cn2)C(=O)N2CCN(C3COC3)CC2)cc1] |
| 398 | 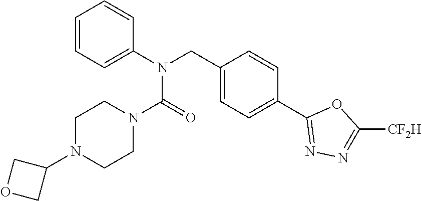 [SMILES: O=C(N1CCN(C2COC2)CC1)N(Cc1ccc(-c2nnc(C(F)F)o2)cc1)c1ccccc1] |
| 399 | 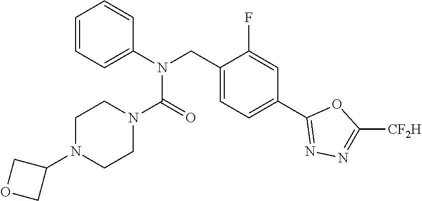 [SMILES: O=C(N1CCN(C2COC2)CC1)N(Cc1ccc(-c2nnc(C(F)F)o2)cc1F)c1ccccc1] |
| 400 | 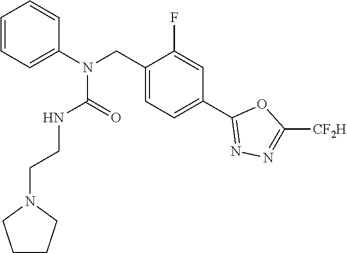 [SMILES: O=C(NCCN1CCCC1)N(Cc1ccc(-c2nnc(C(F)F)o2)cc1F)c1ccccc1] |
| 401 | 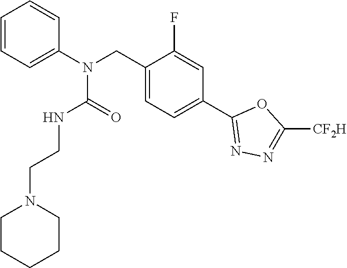 [SMILES: O=C(NCCN1CCCCC1)N(Cc1ccc(-c2nnc(C(F)F)o2)cc1F)c1ccccc1] |
| 402 | 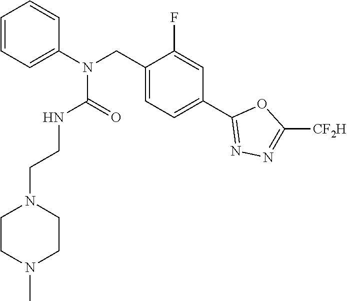 [SMILES: CN1CCN(CCNC(=O)N(Cc2ccc(-c3nnc(C(F)F)o3)cc2F)c2ccccc2)CC1] |
| 403 | 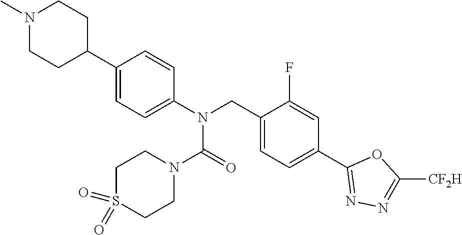 [SMILES: CN1CCC(c2ccc(N(Cc3ccc(-c4nnc(C(F)F)o4)cc3F)C(=O)N3CCS(=O)(=O)CC3)cc2)CC1] |
| 404 | 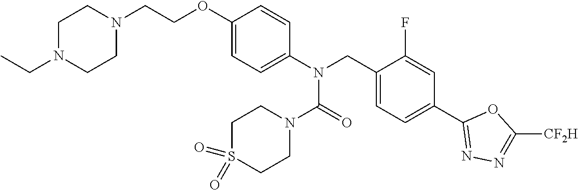 [SMILES: CCN1CCN(CCOc2ccc(N(Cc3ccc(-c4nnc(C(F)F)o4)cc3F)C(=O)N3CCS(=O)(=O)CC3)cc2)CC1] |
| 405 | 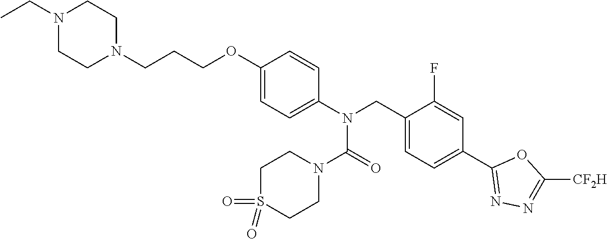 [SMILES: CCN1CCN(CCCOc2ccc(N(Cc3ccc(-c4nnc(C(F)F)o4)cc3F)C(=O)N3CCS(=O)(=O)CC3)cc2)CC1] |
| 406 | 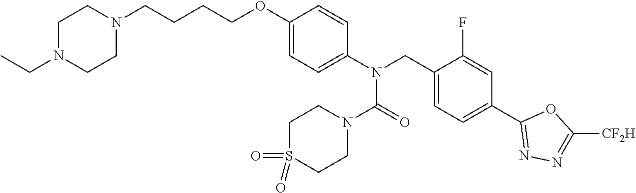 [SMILES: CCN1CCN(CCCCOc2ccc(N(Cc3ccc(-c4nnc(C(F)F)o4)cc3F)C(=O)N3CCS(=O)(=O)CC3)cc2)CC1] |
| 407 | 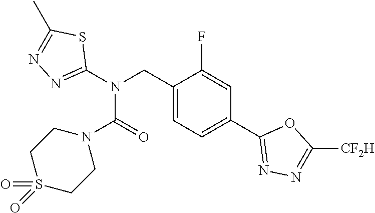 [SMILES: Cc1nnc(N(Cc2ccc(-c3nnc(C(F)F)o3)cc2F)C(=O)N2CCS(=O)(=O)CC2)s1] |
| 408 | 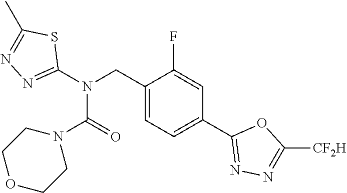 [SMILES: Cc1nnc(N(Cc2ccc(-c3nnc(C(F)F)o3)cc2F)C(=O)N2CCOCC2)s1] |
| 409 | 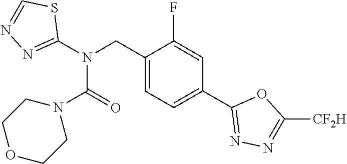 [SMILES: O=C(N1CCOCC1)N(Cc1ccc(-c2nnc(C(F)F)o2)cc1F)c1nncs1] |
| 410 | 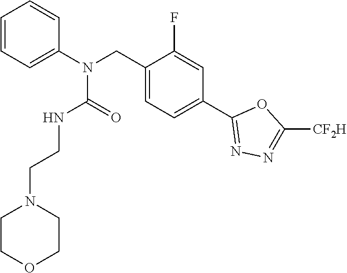 [SMILES: O=C(NCCN1CCOCC1)N(Cc1ccc(-c2nnc(C(F)F)o2)cc1F)c1ccccc1] |
| 411 | 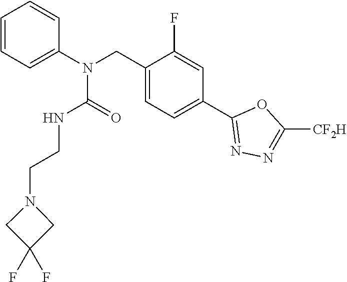 [SMILES: O=C(NCCN1CC(F)(F)C1)N(Cc1ccc(-c2nnc(C(F)F)o2)cc1F)c1ccccc1] |
| 412 | 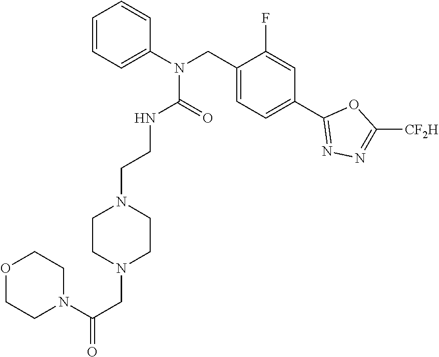 [SMILES: O=C(CN1CCN(CCNC(=O)N(Cc2ccc(-c3nnc(C(F)F)o3)cc2F)c2ccccc2)CC1)N1CCOCC1] |
| 413 | 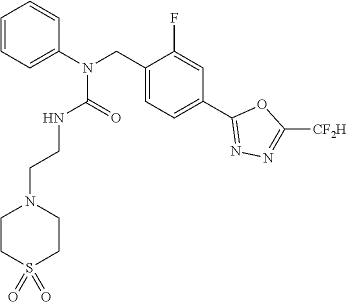 [SMILES: O=C(NCCN1CCS(=O)(=O)CC1)N(Cc1ccc(-c2nnc(C(F)F)o2)cc1F)c1ccccc1] |
| 414 | 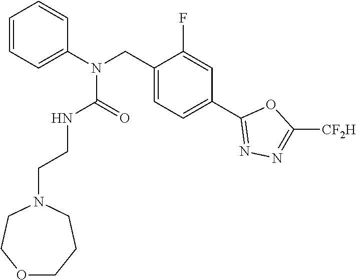 [SMILES: O=C(NCCN1CCCOCC1)N(Cc1ccc(-c2nnc(C(F)F)o2)cc1F)c1ccccc1] |
| 415 | 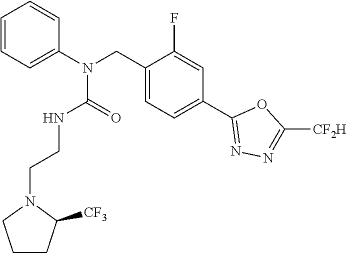 [SMILES: O=C(NCCN1CCC[C@@H]1C(F)(F)F)N(Cc1ccc(-c2nnc(C(F)F)o2)cc1F)c1ccccc1] |
| 416 | 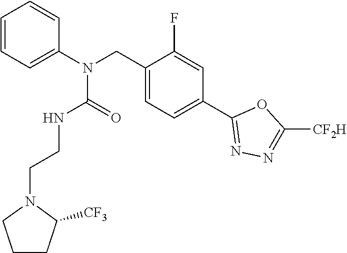 [SMILES: O=C(NCCN1CCC[C@H]1C(F)(F)F)N(Cc1ccc(-c2nnc(C(F)F)o2)cc1F)c1ccccc1] |
| 417 | 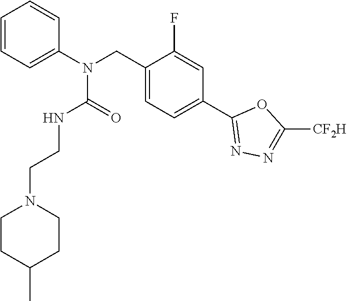 [SMILES: CC1CCN(CCNC(=O)N(Cc2ccc(-c3nnc(C(F)F)o3)cc2F)c2ccccc2)CC1] |
| 418 | 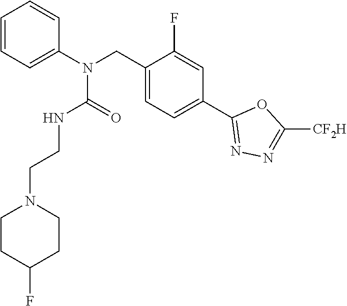 [SMILES: O=C(NCCN1CCC(F)CC1)N(Cc1ccc(-c2nnc(C(F)F)o2)cc1F)c1ccccc1] |
| 419 | 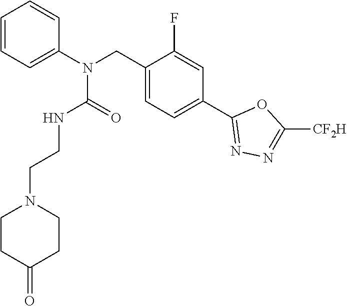 [SMILES: O=C1CCN(CCNC(=O)N(Cc2ccc(-c3nnc(C(F)F)o3)cc2F)c2ccccc2)CC1] |
| 420 | 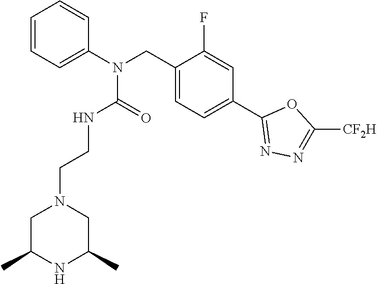 [SMILES: C[C@@H]1CN(CCNC(=O)N(Cc2ccc(-c3nnc(C(F)F)o3)cc2F)c2ccccc2)C[C@H](C)N1] |
| 421 | 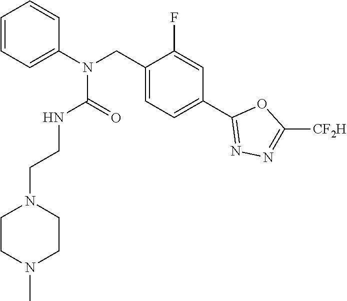 [SMILES: CN1CCN(CCNC(=O)N(Cc2ccc(-c3nnc(C(F)F)o3)cc2F)c2ccccc2)CC1] |
| 422 | 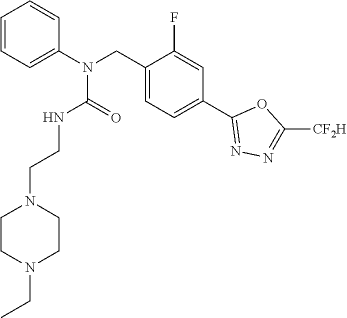 [SMILES: CCN1CCN(CCNC(=O)N(Cc2ccc(-c3nnc(C(F)F)o3)cc2F)c2ccccc2)CC1] |
| 423 | 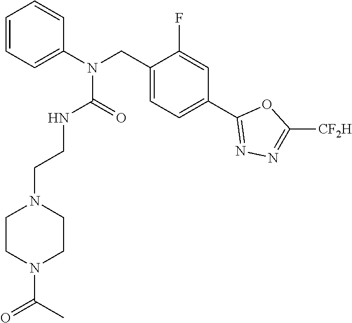 [SMILES: CC(=O)N1CCN(CCNC(=O)N(Cc2ccc(-c3nnc(C(F)F)o3)cc2F)c2ccccc2)CC1] |
| 424 | 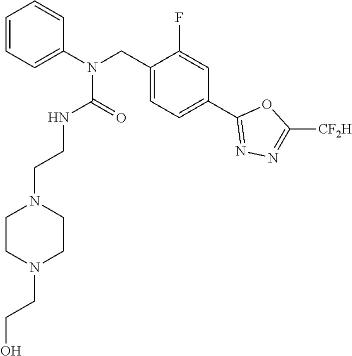 [SMILES: O=C(NCCN1CCN(CCO)CC1)N(Cc1ccc(-c2nnc(C(F)F)o2)cc1F)c1ccccc1] |
| 425 | 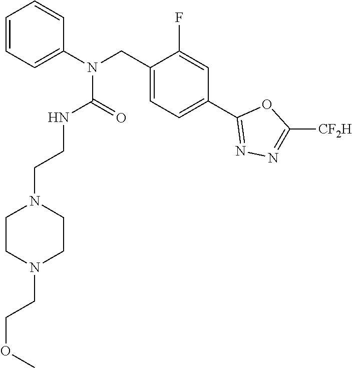 [SMILES: COCCN1CCN(CCNC(=O)N(Cc2ccc(-c3nnc(C(F)F)o3)cc2F)c2ccccc2)CC1] |
| 426 | 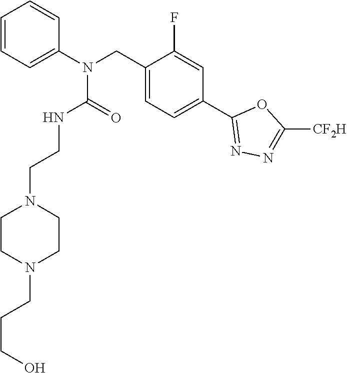 [SMILES: O=C(NCCN1CCN(CCCO)CC1)N(Cc1ccc(-c2nnc(C(F)F)o2)cc1F)c1ccccc1] |
| 427 | 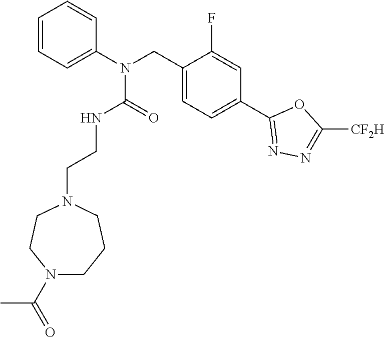 [SMILES: CC(=O)N1CCCN(CCNC(=O)N(Cc2ccc(-c3nnc(C(F)F)o3)cc2F)c2ccccc2)CC1] |
| 428 | 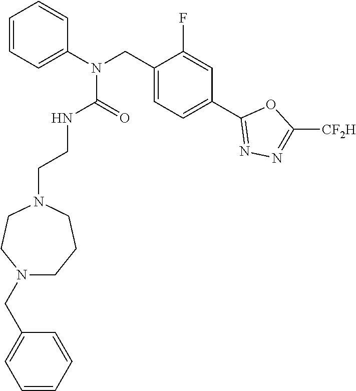 [SMILES: O=C(NCCN1CCCN(Cc2ccccc2)CC1)N(Cc1ccc(-c2nnc(C(F)F)o2)cc1F)c1ccccc1] |
| 429 | 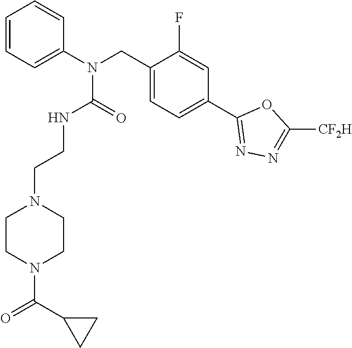 [SMILES: O=C(C1CC1)N1CCN(CCNC(=O)N(Cc2ccc(-c3nnc(C(F)F)o3)cc2F)c2ccccc2)CC1] |
| 430 | 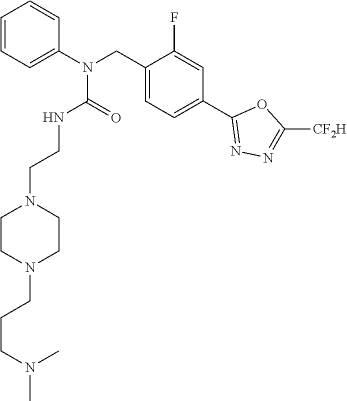 [SMILES: CN(C)CCCN1CCN(CCNC(=O)N(Cc2ccc(-c3nnc(C(F)F)o3)cc2F)c2ccccc2)CC1] |
| 431 | 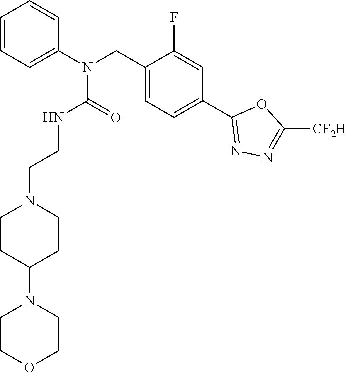 [SMILES: O=C(NCCN1CCC(N2CCOCC2)CC1)N(Cc1ccc(-c2nnc(C(F)F)o2)cc1F)c1ccccc1] |
| 432 | 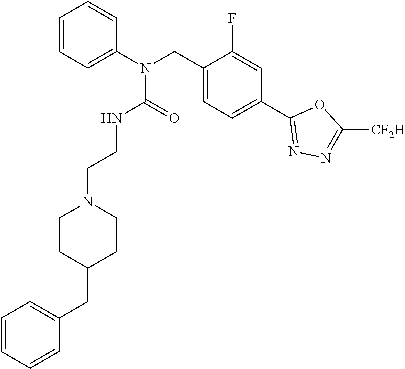 [SMILES: O=C(NCCN1CCC(Cc2ccccc2)CC1)N(Cc1ccc(-c2nnc(C(F)F)o2)cc1F)c1ccccc1] |
| 433 | 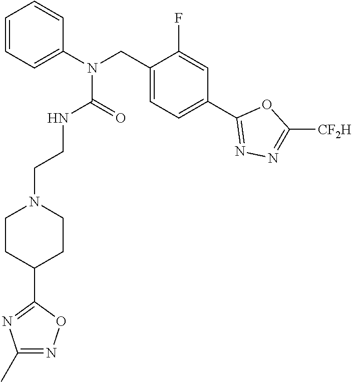 [SMILES: Cc1noc(C2CCN(CCNC(=O)N(Cc3ccc(-c4nnc(C(F)F)o4)cc3F)c3ccccc3)CC2)n1] |
| 434 | 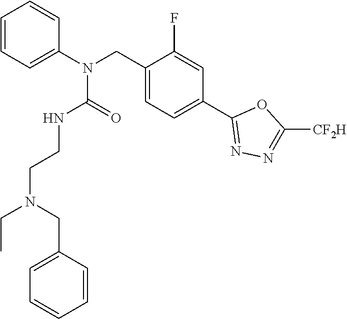 [SMILES: CCN(CCNC(=O)N(Cc1ccc(-c2nnc(C(F)F)o2)cc1F)c1ccccc1)Cc1ccccc1] |
| 435 | 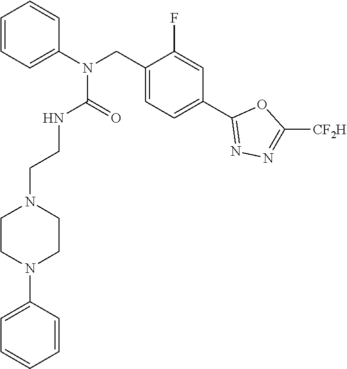 [SMILES: O=C(NCCN1CCN(c2ccccc2)CC1)N(Cc1ccc(-c2nnc(C(F)F)o2)cc1F)c1ccccc1] |
| 436 | 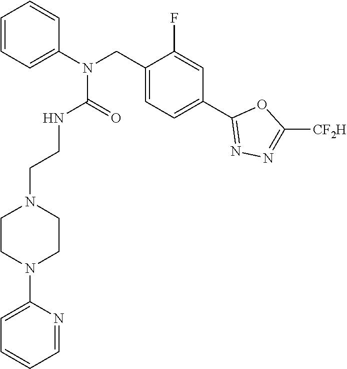 [SMILES: O=C(NCCN1CCN(c2ccccn2)CC1)N(Cc1ccc(-c2nnc(C(F)F)o2)cc1F)c1ccccc1] |
| 437 | 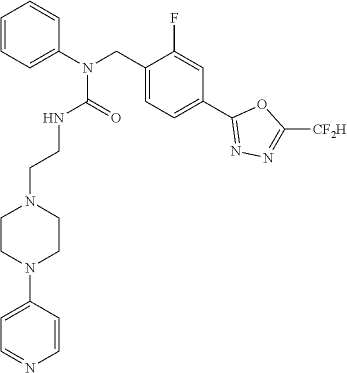 [SMILES: O=C(NCCN1CCN(c2ccncc2)CC1)N(Cc1ccc(-c2nnc(C(F)F)o2)cc1F)c1ccccc1] |
| 438 | 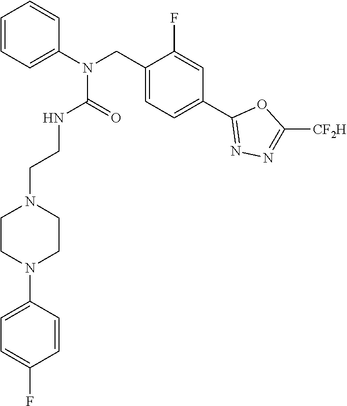 [SMILES: O=C(NCCN1CCN(c2ccc(F)cc2)CC1)N(Cc1ccc(-c2nnc(C(F)F)o2)cc1F)c1ccccc1] |
| 439 | 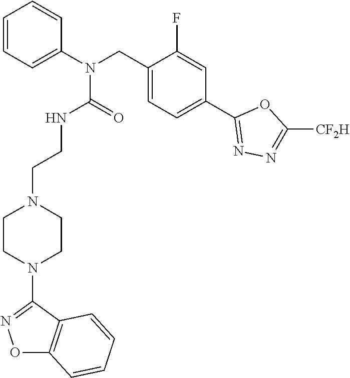 [SMILES: O=C(NCCN1CCN(c2noc3ccccc23)CC1)N(Cc1ccc(-c2nnc(C(F)F)o2)cc1F)c1ccccc1] |
| 440 | 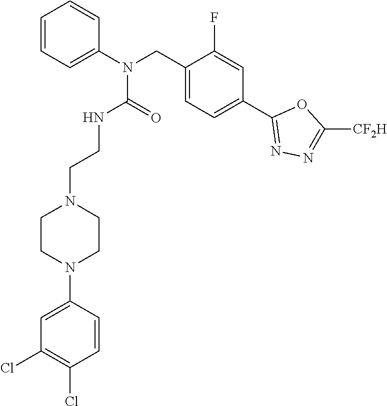 [SMILES: O=C(NCCN1CCN(c2ccc(Cl)c(Cl)c2)CC1)N(Cc1ccc(-c2nnc(C(F)F)o2)cc1F)c1ccccc1] |
| 441 | 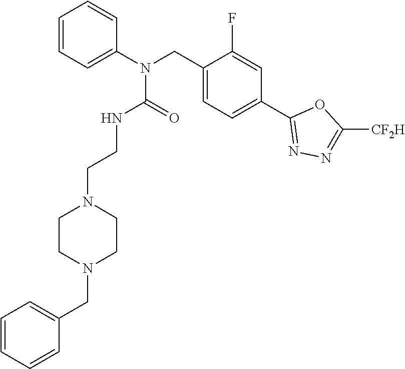 [SMILES: O=C(NCCN1CCN(Cc2ccccc2)CC1)N(Cc1ccc(-c2nnc(C(F)F)o2)cc1F)c1ccccc1] |
| 442 | 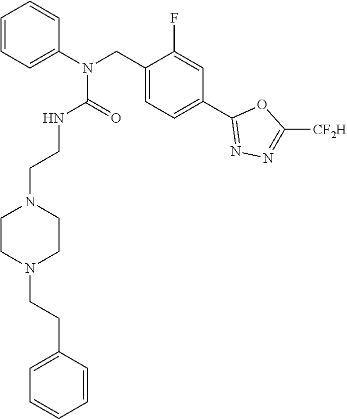 [SMILES: O=C(NCCN1CCN(CCc2ccccc2)CC1)N(Cc1ccc(-c2nnc(C(F)F)o2)cc1F)c1ccccc1] |
| 443 | 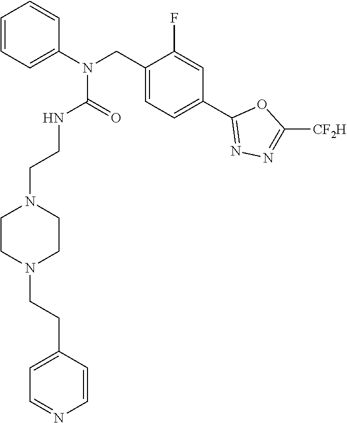 [SMILES: O=C(NCCN1CCN(CCc2ccncc2)CC1)N(Cc1ccc(-c2nnc(C(F)F)o2)cc1F)c1ccccc1] |
| 444 | 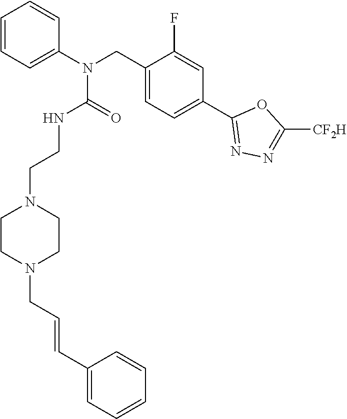 [SMILES: O=C(NCCN1CCN(C/C=C/c2ccccc2)CC1)N(Cc1ccc(-c2nnc(C(F)F)o2)cc1F)c1ccccc1] |
| 445 | 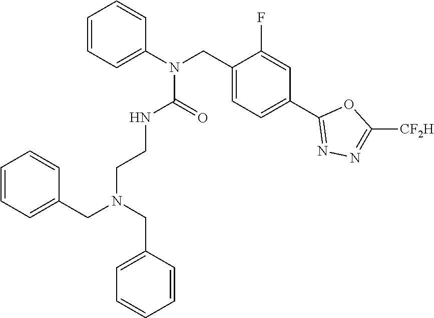 [SMILES: O=C(NCCN(Cc1ccccc1)Cc1ccccc1)N(Cc1ccc(-c2nnc(C(F)F)o2)cc1F)c1ccccc1] |
| 446 | 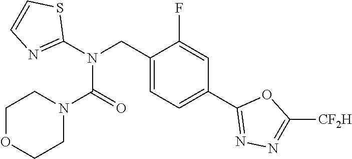 [SMILES: O=C(N1CCOCC1)N(Cc1ccc(-c2nnc(C(F)F)o2)cc1F)c1nccs1] |
| 447 | 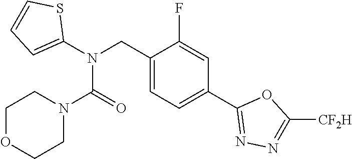 [SMILES: O=C(N1CCOCC1)N(Cc1ccc(-c2nnc(C(F)F)o2)cc1F)c1cccs1] |
| 448 | 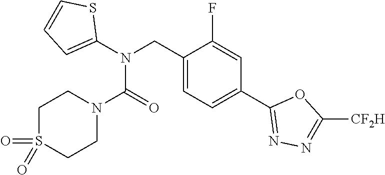 [SMILES: O=C(N1CCS(=O)(=O)CC1)N(Cc1ccc(-c2nnc(C(F)F)o2)cc1F)c1cccs1] |
| 449 | 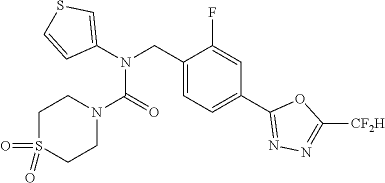 [SMILES: O=C(N1CCS(=O)(=O)CC1)N(Cc1ccc(-c2nnc(C(F)F)o2)cc1F)c1ccsc1] |
| 450 | 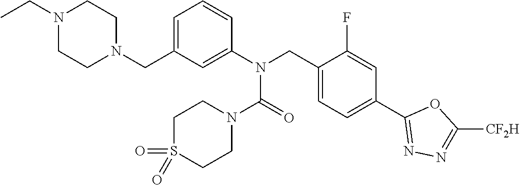 [SMILES: CCN1CCN(Cc2cccc(N(Cc3ccc(-c4nnc(C(F)F)o4)cc3F)C(=O)N3CCS(=O)(=O)CC3)c2)CC1] |

In example embodiments of the present invention, the pharmaceutical composition including a compound of Table A, optical isomers thereof or pharmaceutically acceptable salts thereof as an active ingredient may prevent or treat heart failure.

In the pharmaceutical composition according to the disclosure, the compounds represented by formula I may be shown in Table B below:

TABLE B

| Compound | Structure |
|---|---|
| 40 | 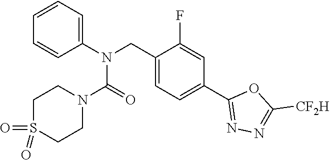 [SMILES: O=C(N1CCS(=O)(=O)CC1)N(Cc1ccc(-c2nnc(C(F)F)o2)cc1F)c1ccccc1] |
| 43 | 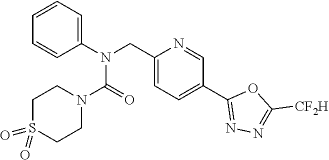 [SMILES: O=C(N1CCS(=O)(=O)CC1)N(Cc1ccc(-c2nnc(C(F)F)o2)cn1)c1ccccc1] |
| 239 | 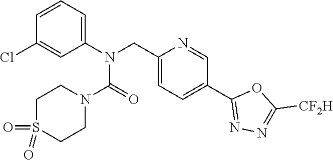 [SMILES: O=C(N1CCS(=O)(=O)CC1)N(Cc1ccc(-c2nnc(C(F)F)o2)cn1)c1cccc(Cl)c1] |
| 285 | 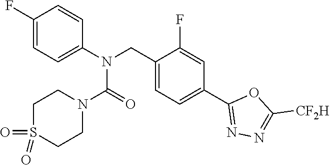 [SMILES: O=C(N1CCS(=O)(=O)CC1)N(Cc1ccc(-c2nnc(C(F)F)o2)cc1F)c1ccc(F)cc1] |
| 295 | 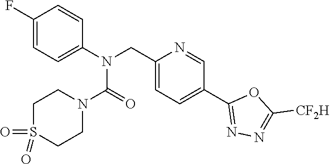 [SMILES: O=C(N1CCS(=O)(=O)CC1)N(Cc1ccc(-c2nnc(C(F)F)o2)cn1)c1ccc(F)cc1] |
| 296 | 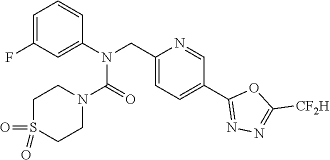 [SMILES: O=C(N1CCS(=O)(=O)CC1)N(Cc1ccc(-c2nnc(C(F)F)o2)cn1)c1cccc(F)c1] |

In example embodiments of the present invention, the pharmaceutical composition including a compound of Table B, optical isomers thereof or pharmaceutically acceptable salts thereof as an active ingredient may prevent or treat heart failure.

In the present disclosure, the compound represented by above formula I may be prepared by a method disclosed in Korean Unexamined Patent Application Publication No. 10-2017-0017792, but is not limited thereto.

In the pharmaceutical composition according to the present disclosure, the compound represented by the above formula I may contain at least one asymmetric carbon, and thus may be present as a racemic mixture, a single enantiomer (optical isomer), a mixture of diastereomers, and a single diastereomer. Such isomer may be separated by being split according to the prior art, for example, column chromatography, HPLC or the like. Alternatively, the isomer may be stereospecifically synthesized with a known array of optically pure starting materials and/or reagents. Particularly, said isomer may be an optical isomer(enantiomer).

In the present disclosure, the term “pharmaceutically acceptable” may refer to the one which is physiologically acceptable and does not conventionally cause gastrointestinal disturbance, an allergic response such as dizziness or other responses similar thereto, when being administered to an individual.

The pharmaceutically acceptable salts according to the embodiments of the present invention may be prepared by a conventional method known to those skilled in the art.

The pharmaceutically acceptable salts according to the embodiment of the present invention may include, for example, inorganic ion salts prepared from calcium, potassium, sodium, magnesium, etc.; inorganic acid salts prepared from hydrochloric acid, nitric acid, phosphoric acid, bromic acid, iodic acid, perchloric acid, sulfuric acid, hydroiodic acid, etc.; organic acid salts prepared from acetic acid, trifluoroacetic acid, citric acid, maleic acid, succinic acid, oxalic acid, benzoic acid, tartaric acid, fumaric acid, mandelic acid, propionic acid, lactic acid, glycolic acid, gluconic acid, galacturonic acid, glutamic acid, glutaric acid, glucuronic acid, aspartic acid, ascorbic acid, carbonic acid, vanillic acid, etc.; sulfonic acid salts prepared from methanesulfonic acid, ethanesulfonic acid, benzenesulfonic acid, p-toluenesulfonic acid, naphthalene sulfonic acid, etc.; amino acid salts prepared from glycine, arginine, lysine, etc.; amine salts prepared from trimethylamine, triethylamine, ammonia, pyridine, picoline, etc.; and the like, but are not limited thereto. In the embodiments of the present invention, salts may include hydrochloric acid, trifluoroacetic acid, citric acid, bromic acid, maleic acid, phosphoric acid, sulfuric acid, tartaric acid or a mixture thereof.

In the present disclosure, the term “heart failure (HF)” may refer to a disease in which the function of the heart is impaired to prevent the heart from supplying oxygen and nutrient-filled blood to other parts of the body, due to myocardial cell damage caused by cardiovascular and coronary artery disease, hypertension, and various genetic factors, etc., cardiomyopathy caused by dysfunction or death of myocardial cells, and/or cardiac fibrosis, etc.

In embodiments of the present invention, the heart failure may include all the heart function damages caused by dysfunction and/or death of myocardial cells, or cardiac fibrosis, etc. due to various causes. For example, the heart failure may be related to cardiovascular disease, metabolic disease or genetic factors, or at least two thereof, but is not limited thereto.

In embodiments of the present invention, the heart failure may be at least one selected from the group consisting of Heart Failure with Preserved Ejection Fraction (HFpEF), Heart Failure with Midrange Ejection Fraction (HFmrEF), and Heart Failure with Reduced Ejection Fraction (HFrEF), but is not limited thereto.

In embodiments of the present invention, the heart failure may include cardiomyopathy. The cardiomyopathy may be at least one selected from the group consisting of hypertrophic cardiomyopathy (HCMP), restrictive cardiomyopathy, and dilated cardiomyopathy (DCMP), but is not limited thereto.

In embodiments of the present invention, the cardiomyopathy may be caused by a genetic abnormality, and in this case, the genetic abnormality cause may be at least one selected from the group consisting of TTN, LMNA, MYH7, MYH6, MYPN, DSP, RBM20, TNNT2, SCN5A, and TPM1 genetic modifications, but is not limited thereto.

As used herein, the term “prevention” may refer to all the acts, which inhibit or delay the occurrence of a disease by administering the compound of formula I of the present invention, optical isomers thereof or pharmaceutically acceptable salts thereof.

In embodiments of the present invention, the “prevention” may include all the acts, which prevent, inhibit or delay the heart function damages caused by myocardial cell damage, cardiomyopathy caused by dysfunction or death of myocardial cells, and/or cardiac fibrosis, etc. Alternatively, in embodiments of the present invention, the prevention may include a case in which heart failure symptoms are slightly expressed according to the heart function damages caused by myocardial cell damage, cardiomyopathy caused by dysfunction or death of myocardial cells, and/or cardiac fibrosis, etc., compared to subjects not administered with the compound of formula I, optical isomers thereof or pharmaceutically acceptable salts thereof.

In embodiments of the present invention, the term “treatment” may refer to all the acts, by which a suspicious symptom of an individual likely to develop a disease or a symptom of an individual suffering from a disease gets better or takes a favorable turn by administering the compound of formula I, optical isomers thereof or pharmaceutically acceptable salts thereof of the present disclosure. In embodiments of the present invention, the “treatment” may include all the acts, which restore heart failure such as the heart function damages caused by myocardial cell damage, cardiomyopathy caused by dysfunction or death of myocardial cells, and/or cardiac fibrosis, etc., alleviate the heart failure, stop the progression of the heart failure, or slow the progression of the heart failure. The pharmaceutical composition including the compound of formula I, optical isomers thereof or pharmaceutically acceptable salts thereof according to the present disclosure may have a remarkably excellent effect on preventing and treating heart failure.

The pharmaceutical composition including the compound of formula I, optical isomers thereof or pharmaceutically acceptable salts thereof according to the present disclosure may exhibit an excellent effect on preventing and treating Heart Failure with Preserved Ejection Fraction (HFpEF), Heart Failure with Midrange Ejection Fraction (HFmrEF), Heart Failure with Reduced Ejection Fraction (HFrEF), or mixtures thereof all.

The pharmaceutical composition including the compound of formula I, optical isomers thereof or pharmaceutically acceptable salts thereof according to the present disclosure may have a remarkably excellent effect on preventing or treating cardiomyopathy.

The pharmaceutical composition including the compound of formula I, optical isomers thereof or pharmaceutically acceptable salts thereof according to the present disclosure may have a remarkably excellent effect on preventing and treating at least one cardiomyopathy selected from the group consisting of hypertrophic cardiomyopathy (HCMP), restrictive cardiomyopathy, and dilated cardiomyopathy (DCMP).

The pharmaceutical composition including the compound of formula I, optical isomers thereof or pharmaceutically acceptable salts thereof according to the present disclosure may have a remarkably excellent effect on preventing and treating cardiomyopathy caused by genetic abnormality.

The pharmaceutical composition including the compound of formula I, optical isomers thereof or pharmaceutically acceptable salts thereof according to the present disclosure may effectively prevent or treat heart damages according to the heart failure. For example, the pharmaceutical composition according to the present disclosure may normally restore an electrocardiogram (ECG) and restore an RR interval in subjects to which heart damage according to the heart failure has occurred.

The pharmaceutical composition including the compound of formula I, optical isomers thereof or pharmaceutically acceptable salts thereof according to the present disclosure may have a remarkably low side effect. For example, the pharmaceutical composition of the present disclosure may exhibit an excellent therapeutic effect without affecting a QT interval of the ECG.

The pharmaceutical composition including the compound of formula I, optical isomers thereof or pharmaceutically acceptable salts thereof according to the present disclosure may effectively inhibit and ameliorate cardiac cell fibrosis in subjects with heart failure, stabilize Ca²⁺ transients in myocardial cells of subjects with heart failure, reduce the expression of a-SMA and TGF-β in subjects with heart damage according to heart failure, and restore the expression of acetylated tubulin to a normal level.

In addition, the pharmaceutical composition including the compound of formula I, optical isomers thereof or pharmaceutically acceptable salts thereof according to the present disclosure may have excellent safety with few or no side effects while exhibiting an excellent therapeutic effect on heart failure. For example, the pharmaceutical composition may have few or no side effects such as induction of ventricular bradycardia.

The pharmaceutical composition including the compound of formula I, optical isomers thereof or pharmaceutically acceptable salts thereof according to the present disclosure may restore a heart function of rabbits in heart damage mimicking conditions (Tachycardia pacing) to a level of a normal group of rabbits in which heart damage mimicking conditions (Tachycardia pacing) are not induced.

The pharmaceutical composition including the compound of formula I, optical isomers thereof or pharmaceutically acceptable salts thereof according to the present disclosure may have significantly fewer or no side effects along with an excellent therapeutic effect compared to conventional drugs. For example, administration of the pharmaceutical composition according to the present disclosure may not result in an increase in the Q-T interval in the electrocardiogram (ECG).

The pharmaceutical composition including the compound of formula I, optical isomers thereof or pharmaceutically acceptable salts thereof according to the present disclosure may effectively treat, inhibit or delay cardiac tissue fibrosis in beagle dogs with induced heart failure and, for example, may remarkably reduce the expression of α-SMA and TGF-β in the beagle dogs with induced heart failure.

The pharmaceutical composition including the compound of formula I, optical isomers thereof or pharmaceutically acceptable salts thereof according to the present disclosure may stabilize Ca²⁺ transients in iPSC-CM myocardial cells derived from patients with heart failure of DCMP (dilated cardiomyopathy) to a level of Ca²⁺ transients in iPSC-CM myocardial cell obtained from normal people.

The pharmaceutical composition including the compound of formula I, optical isomers thereof or pharmaceutically acceptable salts thereof according to the present disclosure may stabilize a degree of expression of acetylated tubulin in tissues obtained from the atrium and ventricle of rabbits in heart damage mimicking conditions (Tachycardia pacing) to a level of a normal group of rabbits in which heart damage mimicking conditions (Tachycardia pacing) are not induced.

The pharmaceutical composition including the compound of formula I, optical isomers thereof or pharmaceutically acceptable salts thereof according to the present disclosure may inhibit decrease of ejection fraction due to the heart failure and increase the weight of lung and heart due to the heart failure in the rat with the included heart failure (TAC model).

The pharmaceutical composition of the present disclosure may further include at least one pharmaceutically acceptable carrier, in addition to the compound represented by above formula I, optical isomers thereof or pharmaceutically acceptable salts thereof. The pharmaceutically acceptable carrier may be the one which is conventionally used in the art, specifically including, but not limited thereto, lactose, dextrose, sucrose, sorbitol, mannitol, starch, acacia rubber, calcium phosphate, alginate, gelatin, calcium silicate, microcrystalline cellulose, polyvinyl pyrrolidine, cellulose, water, syrup, methylcellulose, methyl hydroxybenzoate, propyl hydroxybenzoate, talc, magnesium stearate, mineral, or oil. The pharmaceutical composition of the present invention may further include lubricants, humectants, sweetening agents, flavoring agents, emulsifiers, suspending agents, preservatives, dispersing agents, stabilizing agents, etc., in addition to the above ingredients. In addition, the pharmaceutical composition of the present invention may be formulated into an oral dosage form such as a tablet, powder, granule, pill, capsule, suspension, emulsion, liquid for internal use, oiling agent, syrup, etc., as well as a form of external application, suppository or sterile solution for injection, by using pharmaceutically acceptable carriers and excipients and thus may be prepared in a unit dose form or prepared by being inserted into a multi-dose container. Such preparations may be prepared according to a conventional method used for formulation in the art or a method disclosed in Remington's Pharmaceutical Science (19^{th} ed., 1995), and may be formulated into various preparations depending on each disease or ingredient.

A non-limiting example of preparations for oral administration using the pharmaceutical composition of the present invention may include tablets, troches, lozenges, water-soluble suspensions, oil suspensions, prepared powders, granules, emulsions, hard capsules, soft capsules, syrups, elixirs or the like. To formulate the pharmaceutical composition according to the embodiments of the present invention into preparation for oral administration, the followings may be used: binders such as lactose, saccharose, sorbitol, mannitol, starch, amylopectin, cellulose, gelatin or the like; excipients such as dicalcium phosphate, etc.; disintegrants such as maize starch, sweet potato starch or the like; lubricants such as magnesium stearate, calcium stearate, sodium stearyl fumarate, polyethylene glycol wax, or the like; etc., in which sweetening agents, flavoring agents, syrups, etc. may also be used. Furthermore, in the case of the capsules, liquid carriers such as fatty oil, etc. may be further used in addition to the above-mentioned materials.

A non-limiting example of parenteral preparations using the pharmaceutical composition according to the embodiments of the present invention may include injectable solutions, suppositories, powders for respiratory inhalation, aerosols for spray, ointments, powders for application, oils, creams, etc. To formulate the pharmaceutical composition according to the embodiments of the present invention into preparation for parenteral administration, the following may be used: sterilized aqueous solutions, non-aqueous solvents, suspensions, emulsions, freeze-dried preparations, external preparations, etc. As said non-aqueous solvents and suspensions, the following may be used, but without limitation thereto: propylene glycol, polyethylene glycol, vegetable oils such as olive oil, injectable esters such as ethyl oleate, etc.

The pharmaceutical composition according to the embodiments of the present invention may be subjected to oral administration or parenteral administration according to a targeted method, for example, intravenous, subcutaneous, intraperitoneal or local administration, particularly oral administration, but is not limited thereto.

A daily dosage of the compound represented by formula I, optical isomers thereof or pharmaceutically acceptable salts thereof according to the present disclosure may be particularly about 0.1 to about 10,000 mg/kg, about 1 to about 8,000 mg/kg, about 5 to about 6,000 mg/kg, or about 10 to about 4,000 mg/kg, and more particularly about 50 to about 2,000 mg/kg, but is not limited thereto and may be also administered once a day or several times a day by dividing the daily dosage of the compound.

A pharmaceutically effective dose and an effective dosage of the pharmaceutical composition according to the embodiments of the present invention may vary depending on a method for formulating the pharmaceutical composition, an administration mode, an administration time, an administration route, and/or the like, and may be diversified according to various factors including a type and degree of reaction to be achieved by administration of the pharmaceutical composition, a type of an individual for administration, the individual's age, weight, general health condition, disease symptom or severity, gender, diet and excretion, ingredients of other drug compositions to be used for the corresponding individual at the same time or different times, etc., as well as other similar factors well known in a pharmaceutical field, and those skilled in the art may easily determine and prescribe an effective dosage for the intended treatment.

The pharmaceutical composition according to the embodiments of the present invention may be administered once a day or several times a day by dividing the daily dosage of the composition.

The pharmaceutical composition of the present invention may be administered as an individual therapeutic agent or in combination with other therapeutic agents, and may be administered sequentially or simultaneously with a conventional therapeutic agent. Considering all the above factors, the pharmaceutical composition of the present invention may be administered in such an amount that a maximum effect may be achieved by a minimum amount without a side effect, and such amount may be easily determined by those skilled in the art to which the present invention pertains.

The pharmaceutical composition including the compound of formula I, optical isomers thereof or pharmaceutically acceptable salts thereof according to the embodiments of the present invention may be administered in combination with one or more other therapeutic agents.

The pharmaceutical composition including the compound of formula I, optical isomers thereof or pharmaceutically acceptable salts thereof according to the embodiments of the present invention may show an excellent effect even when solely used, but may be further used in combination with various methods such as hormone therapy, drug treatment, etc. to increase therapeutic efficiency.

The present disclosure may provide a method for preventing or treating heart failure, including administering a compound represented by above formula I, optical isomers thereof or pharmaceutically acceptable salts thereof into an individual.

The present disclosure may provide a method for preventing or treating heart failure, including administering a compound of the above Table A, optical isomers thereof or pharmaceutically acceptable salts thereof into an individual.

The present disclosure may provide a method for preventing or treating heart failure, including administering a compound of the above Table B, optical isomers thereof or pharmaceutically acceptable salts thereof into an individual.

Said terms “heart failure”, “prevention” and “treatment” may be the same as described above.

In the present disclosure, the term “administration” may refer to introducing a predetermined substance into an individual by an appropriate method.

In the present disclosure, the term “individual” may refer to all the animals such as rats, mice, livestock, etc., including humans, who have developed or are likely to develop heart failure, and may be particularly mammals including humans, but is not limited thereto.

The method for preventing or treating the heart failure according to the embodiments of the present invention may include administering a therapeutically effective amount of the compound represented by above formula I, optical isomers thereof or pharmaceutically acceptable salts thereof.

In the present disclosure, the term “therapeutically effective amount” may refer to an amount enough to treat a disease at a reasonable risk/benefit ratio applicable to medical treatment and not to cause a side effect, and may be determined by those skilled in the art according to factors including a patient's gender, age, weight and health condition, a type of disease, severity, the activity of a drug, sensitivity to a drug, an administration method, an administration time, an administration route, an excretion rate, a treatment period, a drug combined or concurrently used, as well as other factors well known in a pharmaceutical field. It is preferable to differently apply a particular therapeutically effective amount for a certain patient depending on various factors including a type and degree of reaction to be achieved therefrom, a particular composition including a presence of other preparations used in some cases, a patient's age, weight, general health condition, gender and diet, an administration time, an administration route, an excretion rate of the composition, a treatment period and a drug used together with the particular composition or simultaneously therewith, as well as other similar factors well known in a pharmaceutical field.

The method for preventing or treating heart failure of the present disclosure may include not only dealing with the disease per se before expression of its symptoms, but also inhibiting or avoiding such symptoms by administering the compound represented by above formula I, isomers thereof or pharmaceutically acceptable salts thereof. In managing the disease, a preventive or therapeutic dose of a certain active ingredient may vary depending on the characteristics and severity of the disease or conditions, and a route in which the active ingredient is administered. A dose and a frequency thereof may vary depending on an individual patient's age, weight and reactions. A suitable dose and usage may be easily selected by those skilled in the art, naturally considering such factors.

In addition, the method for preventing or treating heart failure of the present disclosure may further include administering a therapeutically effective amount of an additional active agent, which helps prevent or treat the disease, along with the compound represented by above formula I, optical isomers thereof or pharmaceutically acceptable salts thereof, and the additional active agent may show a synergy effect or an additive effect together with the compound represented by above formula I, optical isomers thereof or pharmaceutically acceptable salts thereof.

The present disclosure may provide a use of the compound represented by the above formula I, optical isomers thereof or pharmaceutically acceptable salts thereof for preventing or treating heart failure.

The present disclosure may provide a use of the compound of the above Table A, optical isomers thereof or pharmaceutically acceptable salts thereof for preventing or treating heart failure.

The present disclosure may provide a use of the compound of the above Table B, optical isomers thereof or pharmaceutically acceptable salts thereof for preventing or treating heart failure.

The present disclosure may provide a use of the compound represented by above formula I, optical isomers thereof or pharmaceutically acceptable salts thereof in preparing a medicament for preventing or treating heart failure.

The present disclosure may provide a use of the compound of the above Table A, optical isomers thereof or pharmaceutically acceptable salts thereof in preparing a medicament for preventing or treating heart failure.

The present disclosure may provide a use of the compound of the above Table B, optical isomers thereof or pharmaceutically acceptable salts thereof in preparing a medicament for preventing or treating heart failure.

Said terms “heart failure”, “prevention” and “treatment” may be the same as described above.

For the preparation of the medicament, the compound represented by above formula I, optical isomers thereof or pharmaceutically acceptable salts thereof may be mixed with pharmaceutically acceptable adjuvants, diluents, carriers, etc., and may be prepared into a complex preparation together with other active agents, thus providing a synergy action.

Matters mentioned in the pharmaceutical composition, treatment method and use of the present disclosure are applied the same, if not contradictory to each other.

### Advantageous Effects

The compound represented by formula I, optical isomers thereof or pharmaceutically acceptable salts thereof and the pharmaceutical composition including the same as an active ingredient according to the present disclosure may be advantageously used in preventing or treating heart failure.

### BRIEF DESCRIPTION OF THE DRAWINGS

FIGS. 1 and 2 are views of showing the effect of the compound according to the present disclosure on H9c2 cells.

FIG. 3 is a view of electrocardiogram showing the effect of the compound according to the present disclosure on treating and preventing heart failure in heart damage mimicking conditions (Tachycardia pacing).

FIG. 4 is a view of showing the effect of the compound according to the present disclosure on treating and preventing heart failure in heart damage mimicking conditions (Tachycardia pacing).

FIGS. 5 and 6 are views of electrocardiograms showing the safety of the compound according to the present disclosure.

FIG. 7 is a view of showing the effect of the compound according to the present disclosure on treating and preventing heart failure in a heart disease model.

FIGS. 8 and 9 are views of showing the effect of the compound according to the present disclosure on stabilizing Ca²⁺ transients in iPSC-CM myocardial cells derived from normal people and patients with heart failure, respectively.

FIGS. 10 and 11 are views of showing the effect of the compound according to the present disclosure on treating and preventing heart failure in heart damage mimicking conditions (Tachycardia pacing).

FIGS. 12 to 14 are views of showing the effect of the compound according to the present disclosure on treating and preventing heart failure in the animal model of heart failure (TAC model).

### MODE FOR INVENTION

The present disclosure will be described in detail with reference to Examples hereinafter. However, the Examples are only for the purpose of illustrating the present invention and it is obvious to those skilled in the art that the scope of the present invention is not limited to the Examples disclosed hereinafter.

### Synthesis Example 1. Synthesis of compound 43, N-((5-(5-(difluoromethyl)-1,3,4-oxadiazol-2-yl)pyridin-2-yl)methyl)-N-phenylthiomorpholine-4-carboxamide 1,1-dioxide

### [Step 1] N-phenylthiomorpholine-4-carboxamide 1,1-dioxide

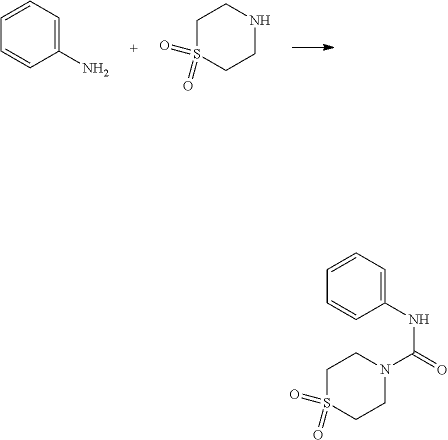 [SMILES: Nc1ccccc1 ; O=S1(=O)CCNCC1 ; O=C(Nc1ccccc1)N1CCS(=O)(=O)CC1]

To a solution of aniline (3.000 g, 32,213 mmol) and N,N-diisopropylethylamine (33.439 mL, 193.278 mmol) in dichloromethane (100 mL) was added at 0° C. triphosgene (4.780 g, 16.107 mmol) and was stirred at the same temperature. Thiomorpholine 1,1-dioxide (4.790 g, 35.434 mmol) was added to the reaction mixture and stirred for an additional 16 hr at room temperature. Then, water was added to the reaction mixture, followed by extraction with ethyl acetate. The organic layer was washed with brine, dried (anhydrous MgSO₄), filtered, and concentrated under the reduced pressure. The concentrate was purified and concentrated by column chromatography (SiO₂, 40 g cartridge; methanol/dichloromethane=2%) to give the title compound as yellow solid (1.325 g, 16.2%).

### [Step 2] Synthesis of Methyl 6-((1,1-dioxido-N-phenylthiomorpholine-4-carboxamido)methyl)nicotinate

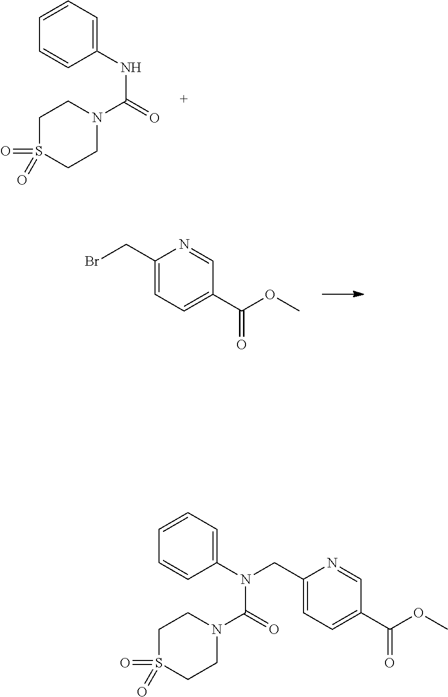 [SMILES: O=C(Nc1ccccc1)N1CCS(=O)(=O)CC1 ; COC(=O)c1ccc(CBr)nc1 ; COC(=O)c1ccc(CN(C(=O)N2CCS(=O)(=O)CC2)c2ccccc2)nc1]

A solution of N-phenylthiomorpholine-4-carboxamide 1,1-dioxide (1.000 g, 3.932 mmol) prepared in Step 1 and sodium hydride (60.00%, 0.157 g, 3.932 mmol) in N,N-dimethylformamide (10 mL) was stirred at 0° C. for 1 hr, and mixed with methyl 4-(bromomethyl)-3-fluorobenzoate (0.905 g, 3.932 mmol). The reaction mixture was stirred at room temperature for an additional 2 hr. The reaction mixture was concentrated under the reduced pressure to remove the solvent, and water was added to the concentrate, followed by extraction with ethyl acetate. The organic layer was washed with brine, dried (anhydrous MgSO₄), filtered, and concentrated under the reduced pressure. The crude product was crystallized at room temperature using methanol (20 mL). The resulting precipitates obtained by filtration were washed by methanol, and dried to give the title compound as brown solid (0.816 g. 51.4%).

### [Step 3] Synthesis of N-((5-(hydrazinecarbonyl)pyridin-2-yl)methyl)-N-phenylthiomorpholine-4-carboxamide 1,1-dioxide

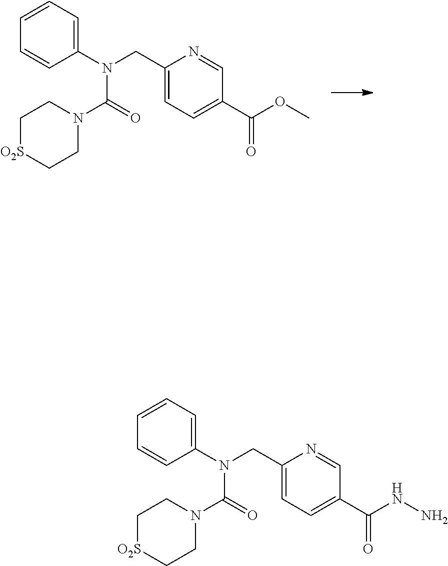 [SMILES: COC(=O)c1ccc(CN(C(=O)N2CCS(=O)(=O)CC2)c2ccccc2)nc1 ; NNC(=O)c1ccc(CN(C(=O)N2CCS(=O)(=O)CC2)c2ccccc2)nc1]

Methyl 6-((1,1-dioxido-N-phenylthiomorpholine-4-carboxamido)methyl)nicotinate (0.816 g, 2.023 mmol) prepared in Step 2 and hydrazine monohydrate (1.910 mL, 40.451 mmol) was mixed in ethanol (10 mL) at the room temperature and then heated at 100° C. under the microwaves for 1 hr, and cooled down to the room temperature to terminate the reaction. The reaction mixture was concentrated under the reduced pressure to remove the solvent. The crude product was crystallized at room temperature using dichloromethane (20 mL). The resulting precipitates obtained by filtration were washed by dichloromethane, and dried to give the title compound as light brown solid (0.560 g, 68.6%).

### [Step 4] Synthesis of N-((5-(2-(2,2-difluoroacetyl)hydrazine-1-carbonyl)pyridin-2-yl)methyl)-N-phenylthiomorpholine-4-carboxamide 1,1-dioxide

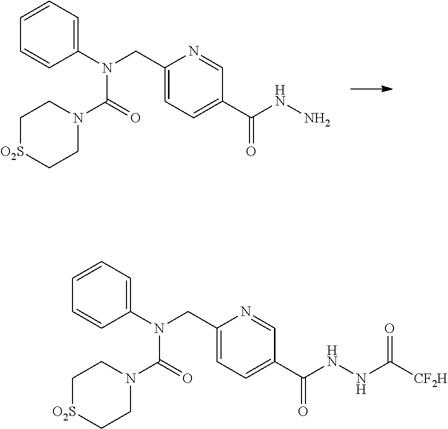 [SMILES: NNC(=O)c1ccc(CN(C(=O)N2CCS(=O)(=O)CC2)c2ccccc2)nc1 ; O=C(NNC(=O)C(F)F)c1ccc(CN(C(=O)N2CCS(=O)(=O)CC2)c2ccccc2)nc1]

A solution of N-((5-(hydrazinecarbonyl)pyridin-2-yl)methyl)-N-phenylthiomorpholine-4-carboxamide 1,1-dioxide (0.260 g, 0.644 mmol) prepared in Step 3 and triethylamine (0.178 mL, 1.289 mmol) in dichloromethane (2 mL) was mixed with Difluoroacetic Anhydride (0.087 mL, 0.580 mmol) at the room temperature. The reaction mixture was stirred at the same temperature for 16 hr. Then, water was added to the reaction mixture, followed by extraction with dichloromethane. The mixture was passed through a plastic frit to remove solid residues and an aqueous layer, and the organic layer collected was concentrated under the reduced pressure. The concentrate was purified and concentrated by column chromatography (SiO₂, 4 g cartridge; methanol/dichloromethane=0% to 5%) to give the title compound as white foam (0.156 g, 50.3%).

### [Step 5] Synthesis of compound 43

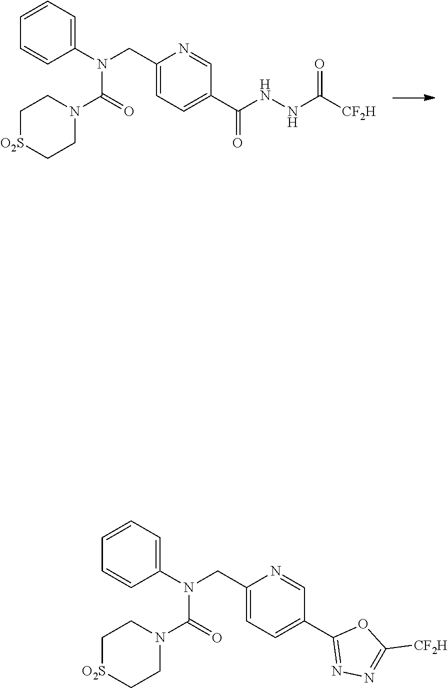 [SMILES: O=C(NNC(=O)C(F)F)c1ccc(CN(C(=O)N2CCS(=O)(=O)CC2)c2ccccc2)nc1 ; O=C(N1CCS(=O)(=O)CC1)N(Cc1ccc(-c2nnc(C(F)F)o2)cn1)c1ccccc1]

A mixture of N-((5-(2-(2,2-difluoroacetyl)hydrazine-1-carbonyl)pyridin-2-yl)methyl)-N-phenylthiomorpholine-4-carboxamide 1,1-dioxide (0.156 g, 0.324 mmol) prepared in Step 4 and 1-methoxy-N-triethylammoniosulfonyl-methanimidate (Burgess reagent, 0.116 g, 0.486 mmol) in tetrahydrofuran (2 mL) was heated at 150° C. for 30 min under the microwaves, and cooled down to the room temperature to terminate the reaction. Then, water was added to the reaction mixture, followed by extraction with dichloromethane. The biphasic mixture was passed through a plastic frit to remove solid residues and an aqueous layer, and the organic layer collected was concentrated under the reduced pressure. The concentrate was purified and concentrated by column chromatography (SiO₂, 4 g cartridge; methanol/dichloromethane=3%) to give the title compound as colorless oil (0.078 g, 51.9%).

¹H NMR (400 MHz, CDCl₃) δ 9.23 (d, 1H, J=2.2 Hz), 8.38 (dd, 1H, J=8.2, 2.2 Hz), 7.54 (d, 1H, J=8.2 Hz), 7.41-7.31 (m, 2H), 7.19 (ddd, 3H, J=6.4, 3.0, 1.6 Hz), 6.94 (m, 1H), 5.10 (s, 2H), 3.72 (dd, 4H, J=6.9, 3.7 Hz), 2.97-2.90 (m, 4H); LRMS (ES) m/z 464.2 (M⁺+1).

### Synthesis Example 2. Synthesis of compound 40, N-(4-(5-(difluoromethyl)-1,3,4-oxadiazol-2-yl)-2-fluorobenzyl)-N-phenylthiomorpholine-4-carboxamide 1,1-dioxide

### [Step 1] Methyl 4-((1,1-dioxido-N-phenylthiomorpholine-4-carboxamido)methyl)-3-fluorobenzoate

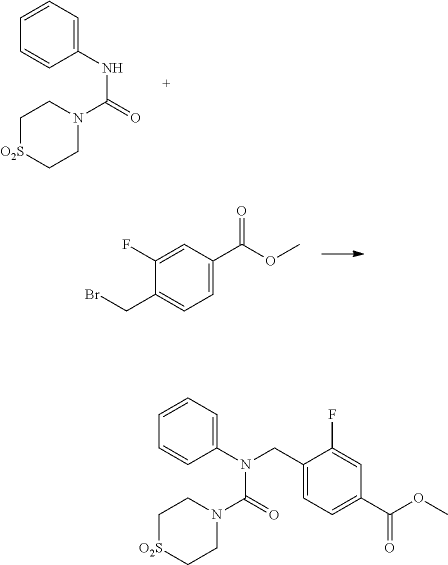 [SMILES: O=C(Nc1ccccc1)N1CCS(=O)(=O)CC1 ; COC(=O)c1ccc(CBr)c(F)c1 ; COC(=O)c1ccc(CN(C(=O)N2CCS(=O)(=O)CC2)c2ccccc2)c(F)c1]

A solution of N-phenylthiomorpholine-4-carboxamide 1,1-dioxide (1.000 g, 3.932 mmol) and sodium hydride (60.00%, 0.189 g, 4.719 mmol) in N,N-dimethylformamide (30 mL) was mixed at 0° C. with methyl 4-(bromomethyl)-3-fluorobenzoate (1.020 g, 4.129 mmol), and stirred at the room temperature for 18 hr. Then, saturated aqueous sodium bicarbonate solution was added to the reaction mixture, followed by extraction with ethyl acetate. The organic layer was washed with brine, dried (anhydrous MgSO₄), filtered, and concentrated in vacuo. The concentrate was purified and concentrated by column chromatography (SiO₂, 40 g cartridge; ethyl acetate/hexane=0% to 50%) to give the title compound methyl 4-((1,1-dioxido-N-phenylthiomorpholine-4-carboxamido)methyl)-3-fluorobenzoate as white solid (1.240 g, 75.0%).

### [Step 2] N-(2-fluoro-4-(hydrazinecarbonyl)benzyl)-N-phenylthiomorpholine-4-carboxamide 1,1-dioxide

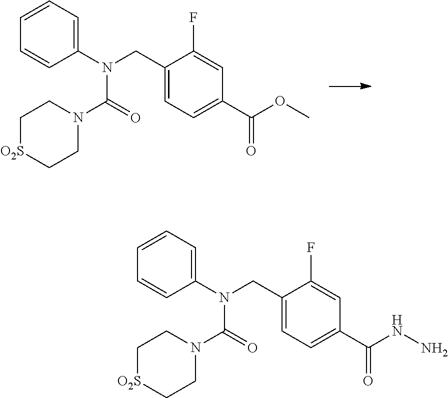 [SMILES: COC(=O)c1ccc(CN(C(=O)N2CCS(=O)(=O)CC2)c2ccccc2)c(F)c1 ; NNC(=O)c1ccc(CN(C(=O)N2CCS(=O)(=O)CC2)c2ccccc2)c(F)c1]

A solution of methyl 4-((1,1-dioxido-N-phenylthiomorpholine-4-carboxamido)methyl)-3-fluorobenzoate (1.240 g, 2.949 mmol) prepared in Step 1 and hydrazine monohydrate (2.786 mL, 58.983 mmol) in ethanol (15 mL) was stirred at 120° C. for 1 hr, and cooled down to the room temperature to terminate the reaction. The reaction mixture was concentrated under the reduced pressure to remove the solvent, and saturated aqueous sodium bicarbonate solution was added to the concentrate, followed by extraction with dichloromethane. The bi-phasic mixture was passed through a plastic frit to remove solid residues and aqueous layer, and the organic layer collected was concentrated in vacuo. The crude title compound N-(2-fluoro-4-(hydrazinecarbonyl)benzyl)-N-phenylthiomorpholine-4-carboxamide 1,1-dioxide was used without further purification (1.240 g, 100.0%, white solid).

### [Step 3] N-(4-(2-(2,2-difluoroacetyl)hydrazine-1-carbonyl)-2-fluorobenzyl)-N-phenylthiomorpholine-4-carboxamide 1,1-dioxide

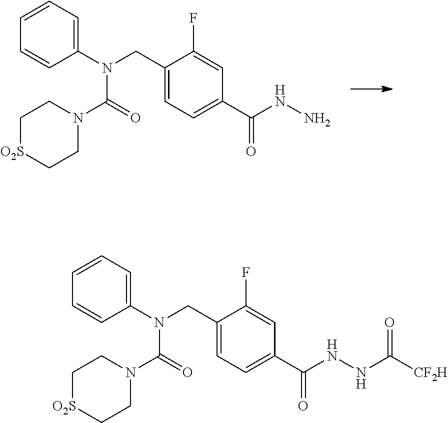 [SMILES: NNC(=O)c1ccc(CN(C(=O)N2CCS(=O)(=O)CC2)c2ccccc2)c(F)c1 ; O=C(NNC(=O)C(F)F)c1ccc(CN(C(=O)N2CCS(=O)(=O)CC2)c2ccccc2)c(F)c1]

A solution of N-(2-fluoro-4-(hydrazinecarbonyl)benzyl)-N-phenylthiomorpholine-4-carboxamide 1,1-dioxide (0.615 g, 1.463 mmol) prepared in Step 2, triethylamine (0.304 mL, 2.194 mmol) and difluoroacetic anhydride (0.164 mL, 1.316 mmol) in dichloromethane (10 mL) was stirred at the room temperature for 18 hr. Then, saturated aqueous sodium bicarbonate solution was added to the reaction mixture, followed by extraction with ethyl acetate. The organic layer was washed with brine, dried (anhydrous MgSO₄), filtered, and concentrated in vacuo. The concentrate was purified and concentrated by column chromatography (SiO₂, 24 g cartridge; methanol/dichloromethane=0% to 3%) to give the title compound N-(4-(2-(2,2-difluoroacetyl)hydrazine-1-carbonyl)-2-fluorobenzyl)-N-phenylthiomorpholine-4-carboxamide 1,1-dioxide as white solid (0.462 g, 63.4%).

### [Step 4] Synthesis of compound 40

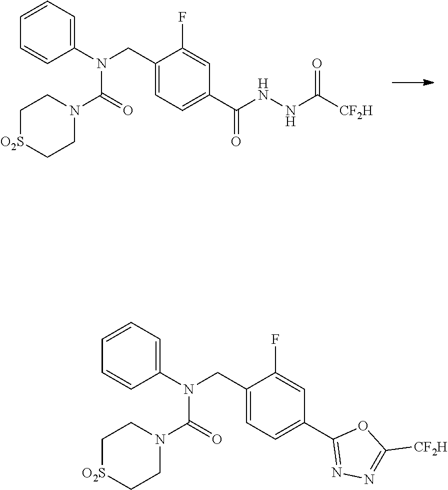 [SMILES: O=C(NNC(=O)C(F)F)c1ccc(CN(C(=O)N2CCS(=O)(=O)CC2)c2ccccc2)c(F)c1 ; O=C(N1CCS(=O)(=O)CC1)N(Cc1ccc(-c2nnc(C(F)F)o2)cc1F)c1ccccc1]

A mixture of N-(4-(2-(2,2-difluoroacetyl)hydrazine-1-carbonyl)-2-fluorobenzyl)-N-phenylthiomorpholine-4-carboxamide 1,1-dioxide (0.462 g, 0.927 mmol) prepared in Step 3 and 1-methoxy-N-triethylammoniosulfonyl-methanimidate (burgess reagent, 0.331 g, 1.390 mmol) in tetrahydrofuran (10 mL) was heated at 150° C. for 30 min under the microwaves, and cooled down to the room temperature to terminate the reaction. Then, saturated aqueous sodium bicarbonate solution was added to the reaction mixture, followed by extraction with dichloromethane. The bi-phasic mixture was passed through a plastic frit to remove solid residues and aqueous layer, and the organic layer collected was concentrated in vacuo. The concentrate was purified and concentrated by column chromatography (SiO₂, 12 g cartridge; ethyl acetate/hexane=0% to 50%) to give the title compound N-(4-(5-(difluoromethyl)-1,3,4-oxadiazol-2-yl)-2-fluorobenzyl)-N-phenylthiomorpholine-4-carboxamide 1,1-dioxide as white solid (0.337 g, 75.7%).

¹H NMR (400 MHz, CDCl₃) δ 7.87-7.85 (m, 1H), 7.75-7.72 (m, 1H), 7.67-7.64 (m, 1H), 7.38-7.34 (m, 2H), 7.25-7.20 (m, 1H), 7.13-7.10 (m, 2H), 7.03-6.77 (m, 1H), 4.92 (s, 2H), 3.71-3.67 (m, 4H), 2.77-2.74 (m, 4H); LRMS (ES) m/z 481.1 (M++1).

### Synthesis Example 3. Synthesis of the compound 239, N-(3-chlorophenyl)-N-((5-(5-(difluoromethyl)-1,3,4-oxadiazol-2-yl)pyridin-2-yl)methyl)thiomorpholine-4-carboxamide 1,1-dioxide

### [Step 1] Synthesis of N-(3-chlorophenyl)thiomorpholine-4-carboxamide 1,1-dioxide

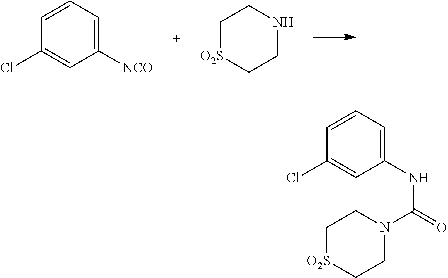

A solution of 1-chloro-3-isocyanatobenzene (1.000 g, 6.512 mmol) and thiomorpholine 1,1-dioxide (0.871 g, 6.447 mmol) in diethyl ether (20 mL) was stirred at the room temperature for 18 hr. The precipitates were filtered, washed by diethyl ether, and dried to give the title compound as white solid (1.811 g, 96.3%).

### [Step 2] Synthesis of Methyl 6-((N-(3-chlorophenyl)-1,1-dioxidothiomorpholine-4-carboxamido)methyl)nicotinate

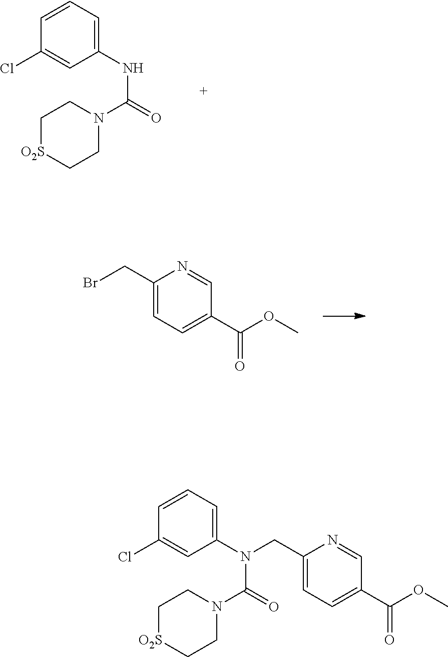 [SMILES: O=C(Nc1cccc(Cl)c1)N1CCS(=O)(=O)CC1 ; COC(=O)c1ccc(CBr)nc1 ; COC(=O)c1ccc(CN(C(=O)N2CCS(=O)(=O)CC2)c2cccc(Cl)c2)nc1]

To a solution of N-(3-chlorophenyl)thiomorpholine-4-carboxamide 1,1-dioxide (0.200 g, 0.693 mmol) prepared in Step 1 in N,N-dimethylformamide (5 mL) was added at 0° C. sodium hydride (60.00%, 0.028 g, 0.693 mmol). The reaction mixture was stirred at the same temperature for 1 hr, added at the same temperature with methyl 6-(bromomethyl)nicotinate (0.159 g, 0.693 mmol), and stirred for additional 2 hr. Then, water was added to the reaction mixture, followed by extraction with ethyl acetate. The organic layer was washed with aqueous saturated sodium chloride solution, dried (anhydrous MgSO₄), filtered, and concentrated under the reduced pressure. The residue was purified and concentrated by chromatography (SiO₂, 12 g cartridge; methanol/dichloromethane=0% to 5%) to give the title compound as brown oil (0.261 g, 86.0%).

### [Step 3] Synthesis of N-(3-chlorophenyl)-N-((5-(hydrazinecarbonyl)pyridin-2-yl)methyl)thiomorpholine-4-carboxamide 1,1-dioxide

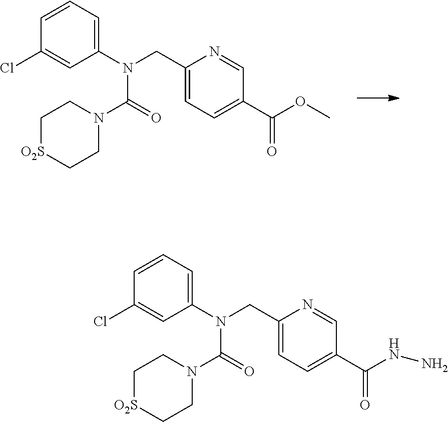 [SMILES: COC(=O)c1ccc(CN(C(=O)N2CCS(=O)(=O)CC2)c2cccc(Cl)c2)nc1 ; NNC(=O)c1ccc(CN(C(=O)N2CCS(=O)(=O)CC2)c2cccc(Cl)c2)nc1]

Methyl 6-((N-(3-chlorophenyl)-1,1-dioxidothiomorpholine-4-carboxamido)methyl)nicotinate (0.261 g, 0.596 mmol) prepared in Step 2 and hydrazine monohydrate (0.290 mL, 5.958 mmol) were mixed at the room temperature in ethanol (2 mL) and then stirred at 110° C. for 18 hr and cooled down to the room temperature to terminate the reaction. The reaction mixture was concentrated under the reduced pressure to remove the solvent. Then, water was added to the obtained concentrate, followed by extraction with dichloromethane. The biphasic mixture was passed through a plastic frit to remove the solid residues and aqueous layer, and the organic layer collected was concentrated under the reduced pressure. The residue was purified and concentrated by chromatography (SiO₂, 4 g cartridge; methanol/dichloromethane=5% to 15%) to give the title compound as brown oil (0.261 g, 100.0%).

### [Step 4] Synthesis of compound 239

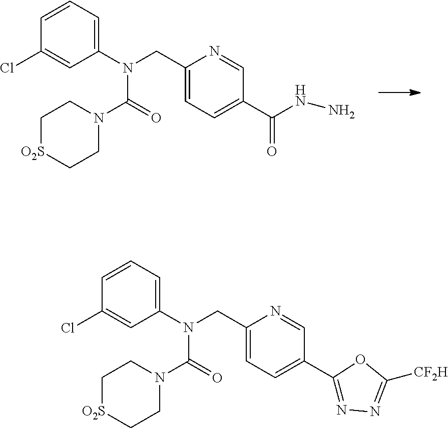 [SMILES: NNC(=O)c1ccc(CN(C(=O)N2CCS(=O)(=O)CC2)c2cccc(Cl)c2)nc1 ; O=C(N1CCS(=O)(=O)CC1)N(Cc1ccc(-c2nnc(C(F)F)o2)cn1)c1cccc(Cl)c1]

N-(3-chlorophenyl)-N-((5-(hydrazinecarbonyl)pyridin-2-yl)methyl)thiomorpholine-4-carboxamide 1,1-dioxide (0.261 g, 0.596 mmol) prepared in Step 3, triethylamine (0.415 mL, 2.980 mmol) and 2,2-difluoroacetic anhydride (0.195 mL, 1.788 mmol) were mixed at the room temperature in tetrahydrofuran (2 mL) and then the obtained solution was stirred at 80° C. for 18 hr and cooled down to the room temperature to terminate the reaction. The reaction mixture was concentrated under the reduced pressure to remove the solvent. Then, water was added to the obtained concentrate, followed by extraction with dichloromethane. The biphasic mixture was passed through a plastic frit to remove the solid residues and aqueous layer, and the organic layer collected was concentrated under the reduced pressure. The residue was purified and concentrated by chromatography (SiO₂, 4 g cartridge; methanol/dichloromethane=0% to 3%) to give the title compound as yellow foam (0.087 g, 29.3%).

¹H NMR (400 MHz, CDCl₃) δ 9.27 (dd, 1H, J=2.2, 0.8 Hz), 8.43 (dd, 1H, J=8.2, 2.2 Hz), 7.55 (dd, 1H, J=8.2, 0.9 Hz), 7.31 (t, 1H, J=8.0 Hz), 7.23 (t, 1H, J=2.1 Hz), 7.21-7.10 (m, 2H), 7.10 (t, 1H), 5.12 (s, 2H), 3.75 (t, 4H, J=5.3 Hz), 3.06-2.99 (m, 4H); LRMS (ES) m/z 498.3 (M⁺+1).

### Synthesis Example 4. Synthesis of the compound 285: N-(4-(5-(difluoromethyl)-1,3,4-oxadiazol-2-yl)-2-fluorobenzyl)-N-(4-fluorophenyl)thiomorpholine-4-carboxamide 1,1-dioxide

### [Step 1] N-(4-fluorophenyl)thiomorpholine-4-carboxamide 1,1-dioxide

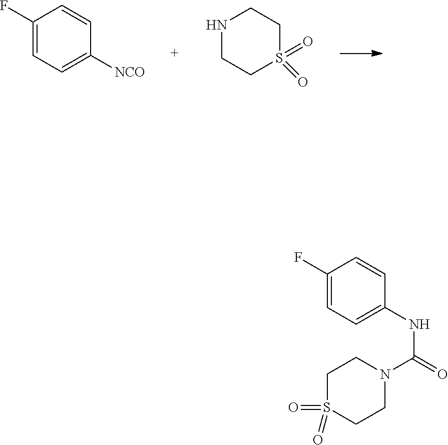

A solution of 1-fluoro-4-isocyanatobenzene (0.500 g, 3.647 mmol) in diethylether (10 mL) was mixed at 0° C. with thiomorpholine 1,1-dioxide (0.493 g, 3.647 mmol), and stirred at the same temperature for 1 hr. The reaction mixture was stirred at the room temperature for additional 4 hr. The precipitates were collected by filtration, washed by diethylether, and dried to give N-(4-fluorophenyl)thiomorpholine-4-carboxamide 1,1-dioxide as white solid (0.920 g, 92.7%).

### [Step 2] Methyl 3-fluoro-4-((N-(4-fluorophenyl)-1,1-dioxidothiomorpholine-4-carboxamido)methyl)benzoate

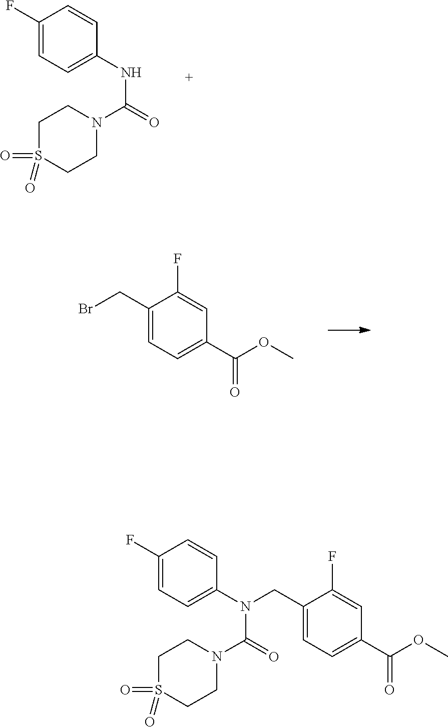 [SMILES: O=C(Nc1ccc(F)cc1)N1CCS(=O)(=O)CC1 ; COC(=O)c1ccc(CBr)c(F)c1 ; COC(=O)c1ccc(CN(C(=O)N2CCS(=O)(=O)CC2)c2ccc(F)cc2)c(F)c1]

A solution of N-(4-fluorophenyl)thiomorpholine-4-carboxamide 1,1-dioxide (0.300 g, 1.102 mmol) prepared in Step 1 and sodium hydride (60.00%, 0.048 g, 1.212 mmol) in N,N-dimethylformamide (5 mL) was stirred at 0° C. for 2 hr, and mixed with methyl 4-(bromomethyl)-3-fluorobenzoate (0.299 g, 1.212 mmol). The reaction mixture was stirred at the room temperature for additional 17 hr, quenched at the room temperature by the addition of water (2 mL, 10 min stirring). Then, water was added to the reaction mixture, followed by extraction with dichloromethane. The bi-phasic mixture was passed through a plastic frit to remove the solid residues and aqueous layer, and the organic layer collected was concentrated in vacuo. The crude product was crystallized at the room temperature using dichloromethane (3 mL). The resulting precipitates were filtered, washed by dichloromethane, and dried to give methyl 3-fluoro-4-((N-(4-fluorophenyl)-1,1-dioxidothiomorpholine-4-carboxamido)methyl)benzoate as white solid (0.212 g, 43.9%).

### [Step 3] N-(2-fluoro-4-(hydrazinecarbonyl)benzyl)-N-(4-fluorophenyl)thiomorpholine-4-carboxamide 1,1-dioxide

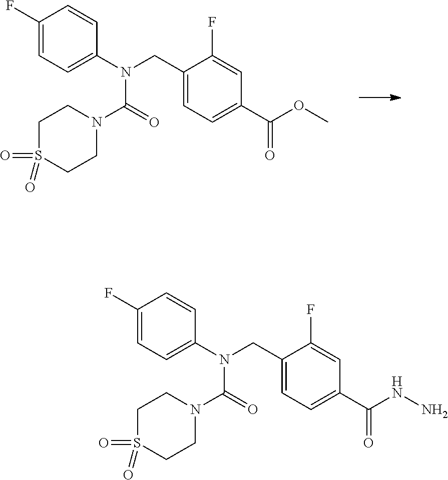 [SMILES: COC(=O)c1ccc(CN(C(=O)N2CCS(=O)(=O)CC2)c2ccc(F)cc2)c(F)c1 ; NNC(=O)c1ccc(CN(C(=O)N2CCS(=O)(=O)CC2)c2ccc(F)cc2)c(F)c1]

Methyl 3-fluoro-4-((N-(4-fluorophenyl)-1,1-dioxidothiomorpholine-4-carboxamido)methyl)benzoate (0.212 g, 0.484 mmol) prepared in Step 2 and hydrazine monohydrate (0.470 mL, 9.670 mmol) in ethanol (4 mL) was mixed at the room temperature and then heated at 120° C. under the microwaves for 1 hr and cooled down to the room temperature to terminate the reaction. The reaction mixture was concentrated under the reduced pressure to remove the solvent. Then, water was added to the reaction mixture, followed by extraction with dichloromethane. The bi-phasic mixture was passed through a plastic frit to remove the solid residues and aqueous layer, and the organic layer collected was concentrated in vacuo. The residue was diluted with diethylether (5 mL) and ethyl acetate (1 mL) and stirred at the ambient temperature. The resulting precipitates were collected by filtration, washed by hexane, and dried to give N-(2-fluoro-4-(hydrazinecarbonyl)benzyl)-N-(4-fluorophenyl)thiomorpholine-4-carboxamide 1,1-dioxide as white solid (0.179 g, 84.4%).

### [Step 4] Synthesis of compound 285

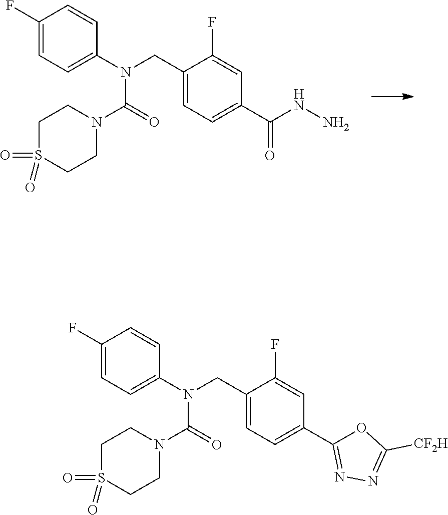 [SMILES: NNC(=O)c1ccc(CN(C(=O)N2CCS(=O)(=O)CC2)c2ccc(F)cc2)c(F)c1 ; O=C(N1CCS(=O)(=O)CC1)N(Cc1ccc(-c2nnc(C(F)F)o2)cc1F)c1ccc(F)cc1]

A solution of N-(2-fluoro-4-(hydrazinecarbonyl)benzyl)-N-(4-fluorophenyl)thiomorpholine-4-carboxamide 1,1-dioxide (0.100 g, 0.228 mmol) prepared in Step 3 and triethylamine (0.095 mL, 0.684 mmol) in dichloromethane (4 mL) was mixed at the room temperature with 2,2-difluoroacetic anhydride (0.028 mL, 0.228 mmol), and stirred at the same temperature for 17 hr. Then, saturated aqueous sodium bicarbonate solution was added to the reaction mixture, followed by extraction with dichloromethane. The bi-phasic mixture was passed through a plastic frit to remove the solid residues and aqueous layer, and the organic layer collected was concentrated in vacuo. The residue was chromatographed (SiO₂, 4 g cartridge; ethyl acetate/hexane=20% to 50%) to give N-(4-(5-(difluoromethyl)-1,3,4-oxadiazol-2-yl)-2-fluorobenzyl)-N-(4-fluorophenyl)thiomorpholine-4-carboxamide 1,1-dioxide as white solid (0.053 g, 46.6%).

¹H NMR (400 MHz, CDCl₃) δ 7.90 (dd, 1H, J=8.0, 1.6 Hz), 7.77 (dd, 1H, J=10.1, 1.6 Hz), 7.69 (t, 1H, J=7.6 Hz), 7.14-6.81 (m, 5H), 4.90 (s, 2H), 3.74-3.71 (m, 4H), 2.85-2.82 (m, 4H); LRMS (ES) m/z 499.3 (M⁺+1).

### Synthesis Example 5. Synthesis of the compound 295, N-((5-(5-(difluoromethyl)-1,3,4-oxadiazol-2-yl)pyridin-2-yl)methyl)-N-(4-fluorophenyl)thiomorpholine-4-carboxamide 1,1-dioxide

### [Step 1] Methyl 6-((N-(4-fluorophenyl)-1,1-dioxidothiomorpholine-4-carboxamido)methyl)nicotinate

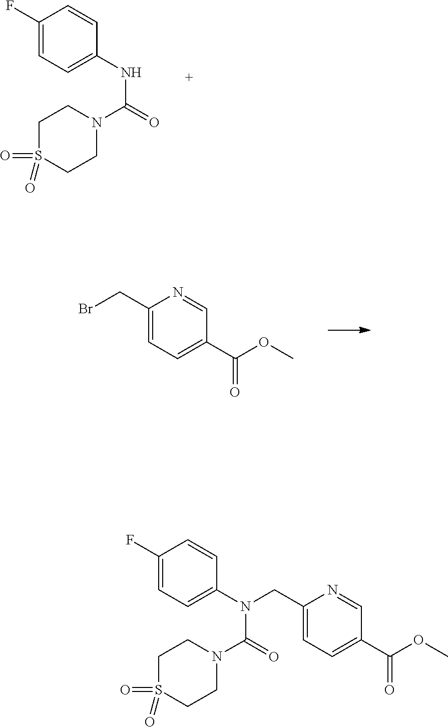 [SMILES: O=C(Nc1ccc(F)cc1)N1CCS(=O)(=O)CC1 ; COC(=O)c1ccc(CBr)nc1 ; COC(=O)c1ccc(CN(C(=O)N2CCS(=O)(=O)CC2)c2ccc(F)cc2)nc1]

A solution of N-(4-fluorophenyl)thiomorpholine-4-carboxamide 1,1-dioxide (0.500 g, 1.836 mmol) prepared in Step 1 of Synthesis Example 4 (Compound 285) d sodium hydride (60.00%, 0.081 g, 2.020 mmol) in N,N-dimethylformamide (10 mL) was stirred at 0° C. for 30 min, and mixed with methyl 6-(bromomethyl)nicotinate (0.465 g, 2.020 mmol). The reaction mixture was stirred at the room temperature for additional 5 hr, quenched at the room temperature by the addition of water (5 mL, 10 min stirring). Then, water was added to the reaction mixture, followed by extraction with ethyl acetate. The organic layer was washed with aqueous saturated sodium chloride solution, dried (anhydrous MgSO₄), filtered, and concentrated in vacuo. methyl 6-((N-(4-fluorophenyl)-1,1-dioxidothiomorpholine-4-carboxamido)methyl)nicotinate was used without further purification (0.450 g, 58.1%, brown solid).

### [Step 2] N-(4-fluorophenyl)-N-((5-(hydrazinecarbonyl)pyridin-2-yl)methyl)thiomorpholine-4-carboxamide 1,1-dioxide

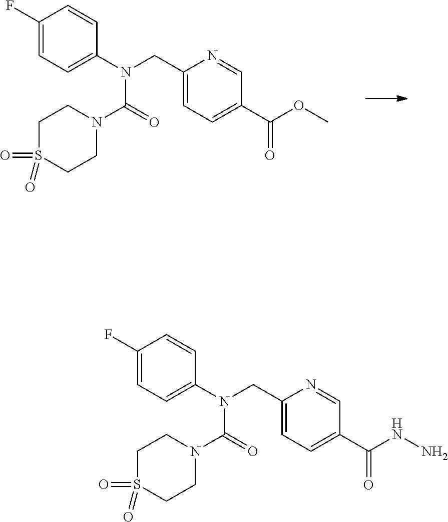 [SMILES: COC(=O)c1ccc(CN(C(=O)N2CCS(=O)(=O)CC2)c2ccc(F)cc2)nc1 ; NNC(=O)c1ccc(CN(C(=O)N2CCS(=O)(=O)CC2)c2ccc(F)cc2)nc1]

Methyl 6-((N-(4-fluorophenyl)-1,1-dioxidothiomorpholine-4-carboxamido)methyl)nicotinate (0.150 g, 0.356 mmol) prepared in Step 1 and hydrazine monohydrate (0.346 mL, 7.118 mmol) were mixed at the room temperature in ethanol (5 mL) and then stirred at 100° C. for 17 hr, cooled down to the room temperature. The precipitates were collected by filtration, washed by ethanol, and dried to give N-(4-fluorophenyl)-N-((5-(hydrazinecarbonyl)pyridin-2-yl)methyl)thiomorpholine-4-carboxamide 1,1-dioxide as pale yellow solid (0.111 g, 74.0%).

### [Step 3] N-((5-(2-(2,2-difluoroacetyl)hydrazine-1-carbonyl)pyridin-2-yl)methyl)-N-(4-fluorophenyl)thiomorpholine-4-carboxamide 1,1-dioxide

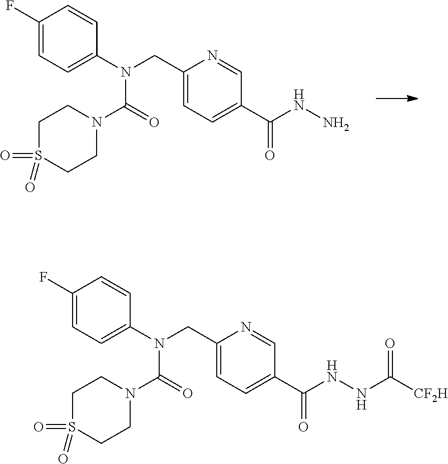 [SMILES: NNC(=O)c1ccc(CN(C(=O)N2CCS(=O)(=O)CC2)c2ccc(F)cc2)nc1 ; O=C(NNC(=O)C(F)F)c1ccc(CN(C(=O)N2CCS(=O)(=O)CC2)c2ccc(F)cc2)nc1]

A solution of N-(4-fluorophenyl)-N-((5-(hydrazinecarbonyl)pyridin-2-yl)methyl)thiomorpholine-4-carboxamide 1,1-dioxide (0.111 g, 0.263 mmol) prepared in Step 2 and triethylamine (0.110 mL, 0.790 mmol) in dichloromethane (5 mL) was mixed at the room temperature with 2,2-difluoroacetic anhydride (0.065 mL, 0.527 mmol), and stirred at the same temperature for 1 hr. Then, water was added to the reaction mixture, followed by extraction with dichloromethane. The bi-phasic mixture was passed through a plastic frit to remove the solid residues and aqueous layer, and the organic layer collected was concentrated in vacuo. The crude product was used without further purification (0.082 g, 62.3%, yellow solid).

### [Step 4] Synthesis of compound 295

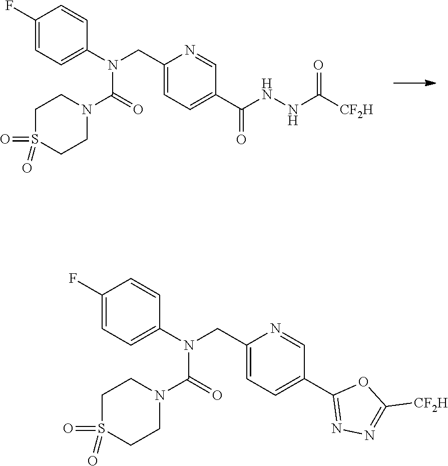 [SMILES: O=C(NNC(=O)C(F)F)c1ccc(CN(C(=O)N2CCS(=O)(=O)CC2)c2ccc(F)cc2)nc1 ; O=C(N1CCS(=O)(=O)CC1)N(Cc1ccc(-c2nnc(C(F)F)o2)cn1)c1ccc(F)cc1]

N-((5-(2-(2,2-difluoroacetyl)hydrazine-1-carbonyl)pyridin-2-yl)methyl)-N-(4-fluorophenyl)thiomorpholine-4-carboxamide 1,1-dioxide (0.082 g, 0.164 mmol) prepared in Step 3 and 1-methoxy-N-triethylammoniosulfonyl-methanimidate (Burgess reagent, 0.117 g, 0.493 mmol) were mixed at the room temperature in tetrahydrofuran (5 mL) and then stirred at 70° C. for 5 hr, cooled down to the room temperature, filtered to remove solids, and concentrated under the reduced pressure. The residue was chromatographed (SiO₂, 4 g cartridge; methanol/dichloromethane=0% to 10%) to give N-((5-(5-(difluoromethyl)-1,3,4-oxadiazol-2-yl)pyridin-2-yl)methyl)-N-(4-fluorophenyl)thiomorpholine-4-carboxamide 1,1-dioxide as white solid (0.015 g, 19.0%).

¹H NMR (400 MHz, CDCl₃) δ 9.27 (d, 1H, J=1.6 Hz), 8.43 (dd, 1H, J=8.2, 2.2 Hz), 7.58 (d, 2H, J=8.2 Hz), 7.25-7.21 (m, 2H), 7.10-6.84 (m, 3H), 5.08 (s, 2H), 3.73 (t, 4H, J=5.1 Hz), 2.98 (t, 4H, J=5.2 Hz); LRMS (ES) m/z 482.1 (M⁺+1).

### Synthesis Example 6. Synthesis of the compound 296, N-((5-(5-(difluoromethyl)-1,3,4-oxadiazol-2-yl)pyridin-2-yl)methyl)-N-(3-fluorophenyl)thiomorpholine-4-carboxamide 1,1-dioxide

### [Step 1] Methyl 6-((N-(3-fluorophenyl)-1,1-dioxidothiomorpholine-4-carboxamido)methyl)nicotinate

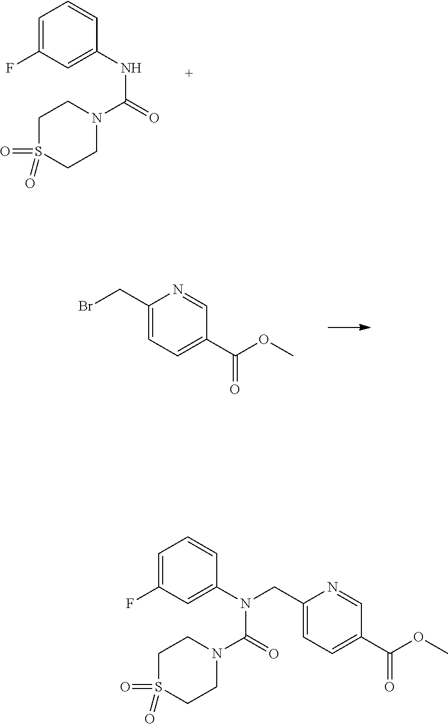 [SMILES: O=C(Nc1cccc(F)c1)N1CCS(=O)(=O)CC1 ; COC(=O)c1ccc(CBr)nc1 ; COC(=O)c1ccc(CN(C(=O)N2CCS(=O)(=O)CC2)c2cccc(F)c2)nc1]

A solution of 1-fluoro-3-isocyanatobenzene (0.500 g, 3.647 mmol) in diethylether (10 mL) was mixed at 0° C. with thiomorpholine 1,1-dioxide (0.493 g, 3.647 mmol), and stirred at the same temperature for 1 hr. The reaction mixture was stirred at the room temperature for additional 4 hr. The precipitates were collected by filtration, washed by diethylether, and dried to give N-(3-fluorophenyl)thiomorpholine-4-carboxamide 1,1-dioxide as white solid (0.870 g, 87.6%).

A solution of N-(3-fluorophenyl)thiomorpholine-4-carboxamide 1,1-dioxide (0.500 g, 1.836 mmol) prepared above and sodium hydride (60.00%, 0.081 g, 2.020 mmol) in N,N-dimethylformamide (10 mL) was stirred at 0° C. for 30 min, and mixed with methyl 6-(bromomethyl)nicotinate (0.465 g, 2.020 mmol). The reaction mixture was stirred at the room temperature for additional 5 hr, quenched at the room temperature by the addition of water (5 mL, 10 min stirring). Then, water was added to the reaction mixture, followed by extraction with ethyl acetate. The organic layer was washed with aqueous saturated sodium chloride solution, dried (anhydrous MgSO₄), filtered, and concentrated in vacuo. Methyl 6-((N-(3-fluorophenyl)-1,1-dioxidothiomorpholine-4-carboxamido)methyl)nicotinate was used without further purification (0.450 g, 58.1%, brown solid).

### [Step 2] N-(3-fluorophenyl)-N-((5-(hydrazinecarbonyl)pyridin-2-yl)methyl)thiomorpholine-4-carboxamide 1,1-dioxide

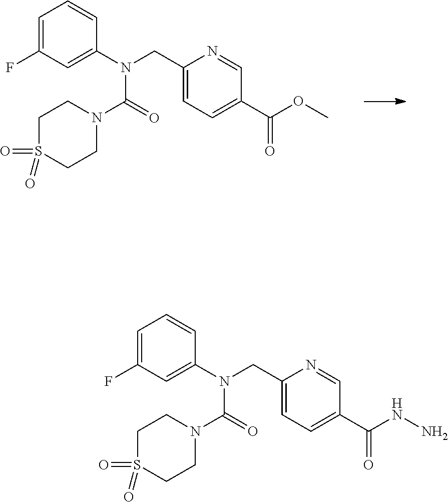 [SMILES: COC(=O)c1ccc(CN(C(=O)N2CCS(=O)(=O)CC2)c2cccc(F)c2)nc1 ; NNC(=O)c1ccc(CN(C(=O)N2CCS(=O)(=O)CC2)c2cccc(F)c2)nc1]

Methyl 6-((N-(3-fluorophenyl)-1,1-dioxidothiomorpholine-4-carboxamido)methyl)nicotinate (0.150 g, 0.356 mmol) prepared in Step 1 and hydrazine monohydrate (0.346 mL, 7.118 mmol) were mixed at the room temperature in ethanol (5 mL) and then stirred at 100° C. for 17 hr, cooled down to the room temperature. The precipitates were collected by filtration, washed by ethanol, and dried to give N-(3-fluorophenyl)-N-((5-(hydrazinecarbonyl)pyridin-2-yl)methyl)thiomorpholine-4-carboxamide 1,1-dioxide as pale yellow solid (0.113 g, 75.3%).

### [Step 3] N-((5-(2-(2,2-difluoroacetyl)hydrazine-1-carbonyl)pyridin-2-yl)methyl)-N-(3-fluorophenyl)thiomorpholine-4-carboxamide 1,1-dioxide

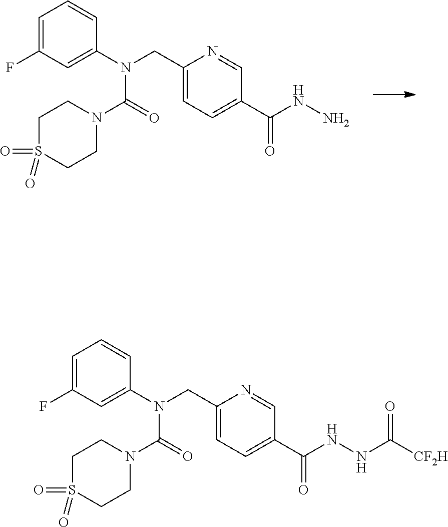 [SMILES: NNC(=O)c1ccc(CN(C(=O)N2CCS(=O)(=O)CC2)c2cccc(F)c2)nc1 ; O=C(NNC(=O)C(F)F)c1ccc(CN(C(=O)N2CCS(=O)(=O)CC2)c2cccc(F)c2)nc1]

A solution of N-(3-fluorophenyl)-N-((5-(hydrazinecarbonyl)pyridin-2-yl)methyl)thiomorpholine-4-carboxamide 1,1-dioxide (0.113 g, 0.268 mmol) prepared in Step 2 and triethylamine (0.112 mL, 0.804 mmol) in dichloromethane (5 mL) was mixed at the room temperature with 2,2-difluoroacetic anhydride (0.067 mL, 0.536 mmol), and stirred at the same temperature for 1 hr. Then, water was added to the reaction mixture, followed by extraction with dichloromethane. The bi-phasic mixture was passed through a plastic frit to remove the solid residues and aqueous layer, and the organic layer collected was concentrated in vacuo. N-((5-(2-(2,2-difluoroacetyl)hydrazine-1-carbonyl)pyridin-2-yl)methyl)-N-(3-fluorophenyl)thiomorpholine-4-carboxamide 1,1-dioxide was used without further purification (0.090 g, 67.2%, yellow solid).

### [Step 4] Synthesis of Compound 296

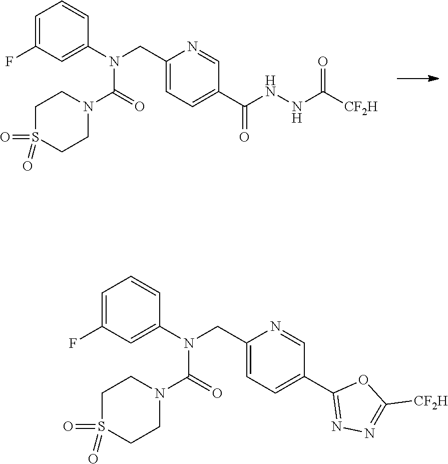 [SMILES: O=C(NNC(=O)C(F)F)c1ccc(CN(C(=O)N2CCS(=O)(=O)CC2)c2cccc(F)c2)nc1 ; O=C(N1CCS(=O)(=O)CC1)N(Cc1ccc(-c2nnc(C(F)F)o2)cn1)c1cccc(F)c1]

N-((5-(2-(2,2-difluoroacetyl)hydrazine-1-carbonyl)pyridin-2-yl)methyl)-N-(3-fluorophenyl)thiomorpholine-4-carboxamide 1,1-dioxide (0.090 g, 0.180 mmol) prepared in Step 3 and 1-methoxy-N-triethylammoniosulfonyl-methanimidate (Burgess reagent, 0.129 g, 0.541 mmol) were mixed at the room temperature in tetrahydrofuran (5 mL) and then stirred at 70° C. for 5 hr, cooled down to the room temperature, filtered to remove solids, and concentrated under the reduced pressure. The residue was chromatographed (SiO₂, 4 g cartridge; methanol/dichloromethane=0% to 10%) to give N-((5-(5-(difluoromethyl)-1,3,4-oxadiazol-2-yl)pyridin-2-yl)methyl)-N-(3-fluorophenyl)thiomorpholine-4-carboxamide 1,1-dioxide as white solid (0.044 g, 50.7%).

¹H NMR (400 MHz, CDCl₃) δ 9.28 (d, 1H, J=1.6 Hz), 8.46 (dd, 1H, J=8.2, 2.2 Hz), 7.58 (d, 1H, J=8.2 Hz), 7.37-7.32 (m, 1H), 7.10-6.92 (m, 4H), 5.14 (s, 2H), 3.76 (t, 4H, J=5.1 Hz), 3.03 (t, 4H, J=5.2 Hz); LRMS (ES) m/z 482.3 (M⁺+1).

### <Example 1> Confirmation of Effect on H9c2 Cells

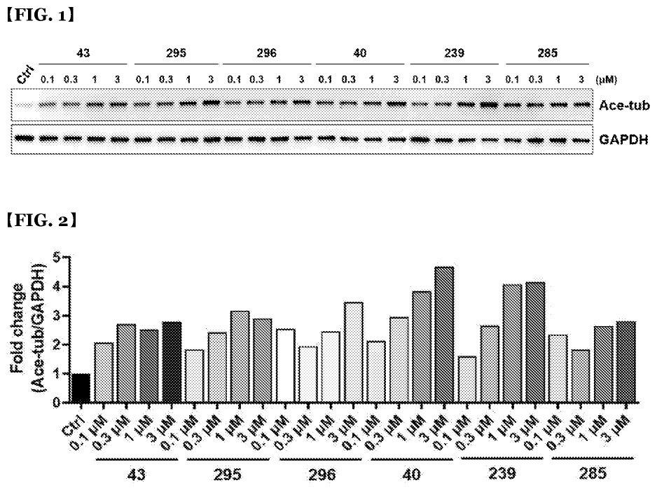

H9c2 (2.0×10⁵ cells/well, Rat, heart, myoblast) cells were seeded in a six-well plate and treated with drugs (compounds 43, 295, 296, 40, 239, and 285) for each concentration. In four hours later at 37° C., proteins were extracted with lysis buffer and quantified by Bradford method. 5 g of proteins were dissolved in sample buffer, electrophoresed on 4-12% gradient gel, transferred to a nitrocellulose membrane for seven minutes, and blocked in 3% BSA solution for one hour. Anti-acetyl tubulin (1:1,000) and GAPDH (1:2,000) were added to a 3% BSA solution, after which the membrane was immersed therein and reacted at 4° C. for 10 hours, and then washed three times with 1×TBST for 10 minutes each. IgG-HRP antibody (1:5,000) was added to 5% BSA, after which the membrane was immersed therein and reacted at room temperature for one hour, and then washed three times with 1×TBST for 10 minutes each. The expression level of the protein was confirmed using an ECL solution, and the results thereof are shown in FIGS. 1 and 2.

As confirmed in above FIGS. 1 and 2, it could be seen that the compounds 43, 295, 296, 40, 239, and 285 of the present disclosure increase the concentration of acetyl tubulin in H9c2 cells in a concentration-correlated manner.

### <Example 2> Confirmation of therapeutic effect on heart damage

The effect of the compound of formula 1 (compound 43: compound of Synthesis Example 1) on treating heart damage was confirmed by administering the compound of formula 1 (compound 43: compound of Synthesis Example 1) into an animal model.

### 1) Preparation of Test Method and Animal Models

The Langendorff test method was used to evaluate the therapeutic effect of the compound on heart damage. The Langendorff test method is known as a test method that helps to determine the direct effect of the compound on the heart in terms of efficacy and safety. In particular, the method is easy to measure actual cardiac contractility and heart rates by removing peripheral hemodynamic variables that may extracardially interfere in experimental animals, and has the advantage of being able to intensively investigate a correlation between chemical structure and pharmacological action using various doses of the compound.

In this example, an attempt was made to confirm the improvement effect of the compound of formula 1 (compound 43: compound of Synthesis Example 1) and the differentiation thereof from existing drugs in the heart damage conditions induced by tachycardia pacing.

Male rabbits having the following conditions were prepared.

| | | | | Acclimation |
|---|---|---|---|---|
| Type | Source | Gender | Weight | period |
| Rabbit | Orient Bio | Male | 1.2 to 1.5 kg | 1 week |

Tachycardia pacing-induced heart damage occurred to the above male rabbits.

Rabbits were supplied from Orient Bio and fed on a standard diet (Central Lab Animal, Inc.) and kept under the conditions of constant temperature (20±2° C.), humidity (40±10%) and lighting (12 h light-dark cycle) with a free access to drinking. All experimental procedures were approved and performed according to the Institutional Animal Care and Use Committee (IACUC) of the external testing agency (with the IACUC animal study protocol approval number: BnH2015-9E).

The rabbits were grouped into a normal group (Control or Sham), a drug-untreated group (Comparative group, vehicle), and a drug-treated group (Compound) with five rabbits in each group.

### 2) Test Method

### 2-1) Langendorff Experimental Method

The male rabbits were anesthetized by breathing, and the hearts were removed therefrom. After removing the connective tissues, the hearts were perfused with 37° C. physiological solution (modified KrebsHenseleit bicarbonate buffer; composition: 116 mM/L NaCl, 4.7 mM/L KCl, 1.1 mM/L MgSO₄, 1.17 mM/L KH₂PO₄, 24.9 mM/L NaHCO₃, 2.52 mM/L CaCl₂), 8.32 mM/L glucose) saturated with 95% O₂/5% CO₂ under constant pressure perfusion, and electrodes were connected at the atrium and ventricle so as to confirm a stabilization state by measuring an electrocardiogram for one hour.

2-2) Method for Evaluating Ventricular Functions with Langendorff Experimental Method

After confirming the stabilization state of the heart for one hour through an electrocardiogram, a heart damage stimulation with 50 Hz tachycardia pacing was applied for 30 minutes. After measuring the electrocardiogram of the atrium and ventricle, a test target compound (compound 43: the compound of Preparation Example 1) was administered to physiological solution, and the electrocardiogram was measured in one hour later. In order to check whether a drug effect is maintained even after the compound is removed from the physiological solution, the physiological solution containing the compound, in which the rabbit heart was immersed, was washed out, and the physiological solution without the compound was administered, so as to measure the electrocardiogram in one hour later.

2-3) Method for Evaluating Heart Safety with Langendorff Experimental Method

After confirming the stabilization state of the heart through the electrocardiogram for one hour, the compound was administered to the physiological solution, and the electrocardiogram was measured in 30 minutes later so as to analyze the Q-T interval.

### 3) Test Results

3-1) Validation of Efficacy of Compound in Ex Vivo Conditions

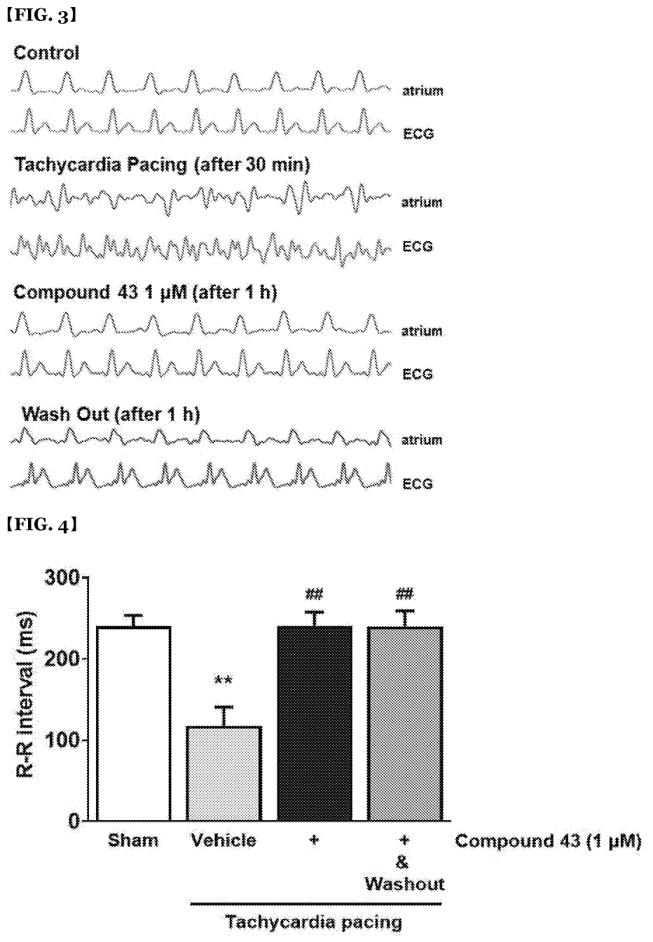

The effect of the compound on improving ventricular functions was analyzed in ex vivo heart damage mimicking conditions (Tachycardia pacing), and the results thereof are shown in FIGS. 3 and 4 and Table 1. In above FIG. 4, ** on the graph may refer to P<0.01 when compared with the normal group(Sham), and ## may mean P<0.01 when compared with Tachycardia pacing (Comparative group, vehicle).

TABLE 1
R-R interval (ms)

| | Normal group | Comparative | Compound | Wash |
|---|---|---|---|---|
| | (control) | group (vehicle) | 43 | out |
| Average (ms) | 240.3 | 117.5 | 240.8 | 239.9 |

As understood from FIG. 3, it could be confirmed that the electrocardiogram is improved to a degree of the normal group (Control) without irregular ventricular functions induced when treated with 1 μM compound 43, and it was also found that a cardioversion effect is maintained for at least one hour even after the compound is removed.

In addition, as confirmed in FIG. 4 and Table 1, an R-R interval was improved to a degree similar to that of the normal group (Sham) when treated with 1 M compound and when the compound was removed.

### 3-2) Securing of Heart Safety Compared to Existing Drugs

Using dofetilide as a control drug, the effect of the compound according to the present disclosure (compound 43: compound of Synthesis Example 1) on the QT interval of ECG was confirmed in normal rabbits.

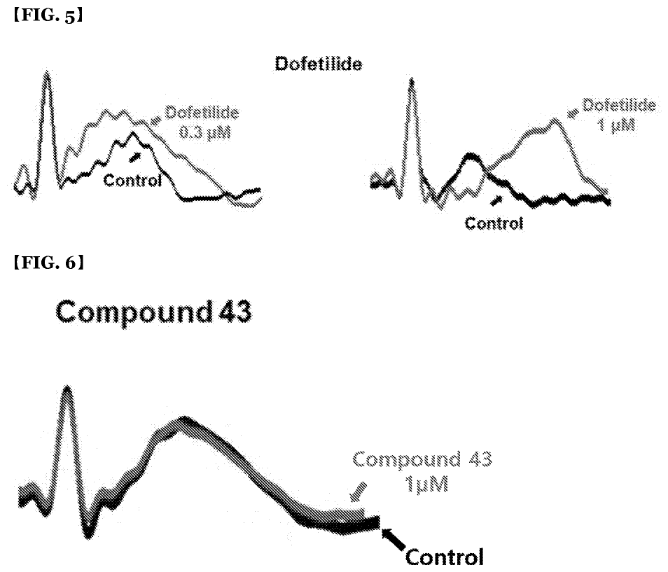

Rabbits were prepared under the same conditions as in 1) and divided into a normal group, a dofetilide-treated group, and a compound 43-treated group, after which the hearts of the rabbits were extracted according to the Langendorff experimental method in 2-2), so as to analyze the Q-T interval according to 2-3), and the results thereof are shown in FIGS. 5 and 6 and in Tables 2 and 3 below. In above FIGS. 5 and 6 and Tables 2 and 3, the Control may refer to a normal group, and all data values in Tables 2 and 3 are expressed as mean standard error.

TABLE 2
Q-T interval (ms)

| | Normal group | | Normal group | |
|---|---|---|---|---|
| | (control) | 0.3 μM | (control) | 1 μM |
| Dofetilide | 121.1 ± 15.4 | 199.2 ± 17.8 | 110.1 ± 12.7 | 205.4 ± 26.3 |
| (n = 6) | | | | |

As can be confirmed in above Table 2 and FIG. 5, the QT interval was increased at both 0.3 μM and 1 μM when treated with dofetilide compared to the QT interval of normal EGG.

TABLE 3
Q-T interval (ms)

| Compound 43 | Normal group | |
|---|---|---|
| (n = 6) | (control) | 1 μM |
| Average (ms) | 128.6 ± 1.6 | 125.4 ± 1.7 |

As confirmed in FIG. 6 and Table 3, the QT interval of the normal ECG was not affected in the normal group when treated with compound 43.

Thus, it can be seen that the compound of formula I according to the present disclosure is a safe drug, without causing any adverse effects on the heart.

### <Example 3> Confirmation of Anti-Fibrotic Activity Effect

### 1) Production of Heart Disease Model and Preparation of Experimental Animals

Beagle dogs having the following conditions were used to confirm the anti-fibrotic activity effect of the compound according to the present disclose in heart failure.

| | | | | Acclimation |
|---|---|---|---|---|
| Type | Source | Gender | Age | period |
| beagle dog | Orient Bio | Male | 11 months | 1 week |

Beagle dogs were supplied from Orient Bio and fed on a standard diet (Central Lab Animal, Inc.) and kept under the conditions of constant temperature (23±3° C.), humidity (55±15%) and lighting (12 h light-dark cycle) with a free access to drinking. All experimental procedures were approved and performed according to the Institutional Animal Care and Use Committee (IACUC) of the external testing agency (with the IACUC animal study protocol approval number: KNOTUS IACUC 18-KE-268).

For the introduction of anesthetic to the beagle dogs (male, 11 months old), pentobarbital (25 mg/kg) was intravenously administered thereto, and anesthesia was maintained through Isoflurane. A pacemaker's in vivo implantable electrode (bipolar pacing lead, Medtronic, IRE) was placed in the right atrial appendage by using C-arm (ARCADIS Varic, SIEMENS Co.) equipment and vascular contrast media, and then it was checked if the same was correctly inserted through a contrast image, and then the pacemaker's lead was fixed in the right atrial appendage. After stabilization for one week after surgery, the pacemaker was operated at 400 bpm to confirm normal induction by electrocardiography, and then tachycardia pacing was induced. Hemodynamic parameters for each group were measured.

In order to confirm a drug administration effect of the compound according to the present disclosure, beagle dogs with induced heart disease were divided into each group of two animals, and each of the groups was classified as shown in Table 4 below according to an administered substance [vehicle (Veh), compound], a route of administration [oral administration (P.O.)], and an administration interval [daily (Bid)].

TABLE 4
Constitution of test groups

| | | Tachycardia | Number |
|---|---|---|---|
| Group | Administered Dose | pacing | of animals |
| Normal group | Empty capsule | X | 2 |
| (control) | | | |
| Drug-untreated | Empty capsule | ◯ | 2 |
| group (vehicle) | | | |
| Drug-treated group | 1 mg/kg; capsule, | ◯ | 2 |
| (compound) | Bid | | |

In above Table 4, the normal group was a group without Tachycardia pacing induced, the drug-untreated group was a group with Tachycardia pacing induced, but without the drug administered, and the drug-treated group was a group with Tachycardia pacing induced and dosed with compound 43 (Synthesis Example 1).

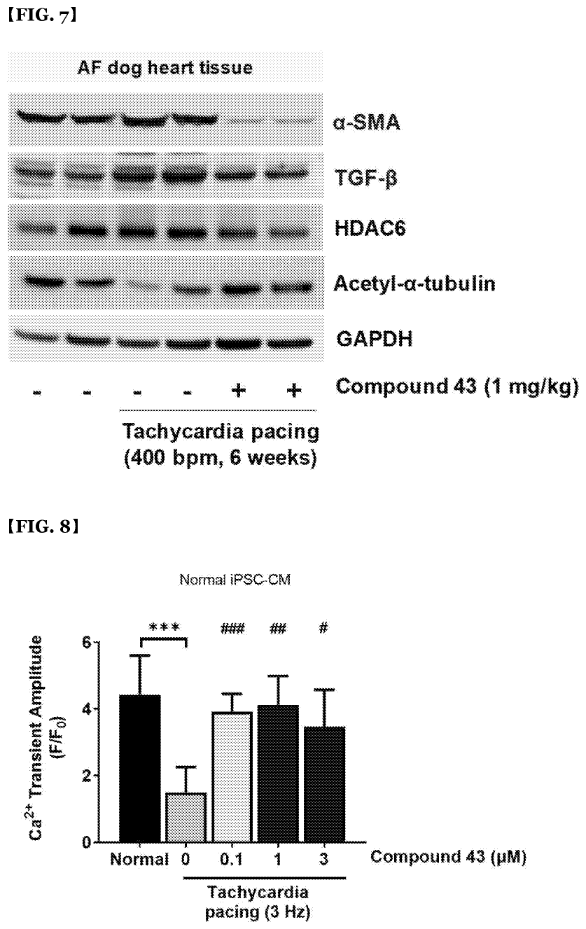

In order to confirm the effect of the compound according to the present disclosure on preventing or treating heart failure disease, the expression of α-SMA and TGF-β, which are directly related to fibrosis in cardiac tissues, was analyzed. After the hearts of each group were isolated, proteins were isolated from each heart tissue. The expression of α-SMA and TGF-β was analyzed for each protein through western blot, so as to compare the degree of cardiac fibrosis between groups, and the results thereof are shown in FIG. 7.

As a result, as confirmed in FIG. 7, it was observed that the group dosed with compound 43 according to the present disclosure exhibits a reduced expression of α-SMA and TGF-β compared to the group not dosed with the drug in heart damage mimicking conditions (Tachycardia pacing), respectively. Thus, it could be seen that the compound according to the present disclosure shows an anti-fibrotic effect, and thus is advantageously used in preventing or treating heart failure disease.

### <Example 4> Confirmation of Effect on Ca²⁺ Transient Activity in DCMP iPSC-CM Accompanied by TPM1 Mutation

In order to confirm the effect of the compound according to the present disclosure on preventing or treating heart failure disease, Ca²⁺ transients in iPSC-CM myocardial cells derived from patients with dilated cardiomyopathy (DCMP) heart failure were analyzed.

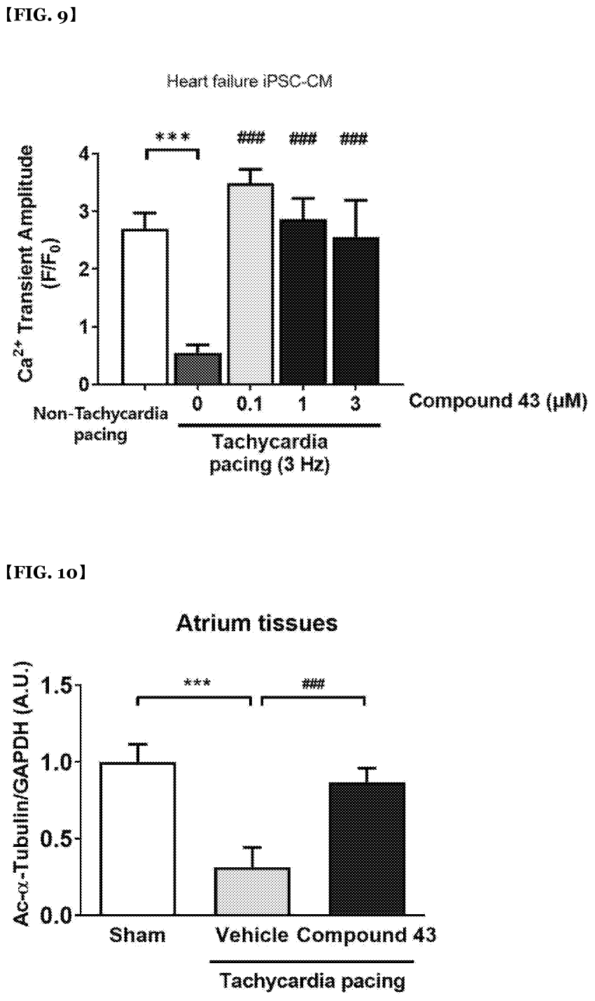

Myocardial cells derived from patients with heart failure were treated with compound 43 (Synthesis Example 1) for each concentration for 24 hours. After that, 3 Hz stimulation was given to both iPSC-CM myocardial cells derived from normal people (normal group, Normal) and iPSC-CM myocardial cells derived from patients with dilated cardiomyopathy (DCMP) heart failure for 24 hours in heart damage mimicking conditions (Tachycardia pacing). After each group was treated with Fluo-4 (AM, cell permeant-Thermo Fisher Scientific), a cell-permeable calcium dye, Ca²⁺ images were taken in real time with a confocal microscope, so as to compare and analyze Ca²⁺ transients between groups. Then, the results of the cells obtained from normal people are shown in FIG. 8, while the results of the cells obtained from patients with DCMP heart failure are shown in FIG. 9. Normal in above FIG. 8 may refer to myocardial cells not treated with Tachycardia pacing, and 0, 0.1, 1 and 3 in FIGS. 8 and 9 may represent the concentrations of compound 43 (compound of Synthesis Example 1) in cells treated with Tachycardia pacing. In addition, in FIGS. 8 and 9, *** may mean P<0.001 when compared to non-Tachycardia pacing (Normal), and #, ## and ### may represent P<0.05, less than P<0.01, and P<0.001, respectively, when compared to the Tachycardia pacing group.

As can be confirmed in FIG. 9, it was observed that Ca²⁺ transients are decreased upon stimulation with Tachycardia pacing as a result of comparative analysis of Ca²⁺ transients in myocardial cells derived from patients with DCMP heart failure. In the group dosed with compound 43 according to the present disclosure, it was observed that Ca²⁺ transients are recovered and compound 43 is effective even when administered for each concentration (0.1-3 μM). Thus, it was confirmed that the compound according to the present disclosure exhibits a Ca²⁺ transient stabilizing effect, and thus may be advantageously used in preventing or treating heart failure disease and exhibit a sufficient therapeutic effect even in severe heart failure.

### <Example 5> Confirmation of Effect on Acetylated Tubulin in Atrial and Ventricular Tissues in Heart Damage Mimicking Conditions (Tachycardia Pacing)

In order to confirm the effect of the compound according to the present disclosure on preventing or treating heart failure disease, an expression of acetylated tubulin was analyzed.

Male rabbits were prepared in the same manner as in Example 1, and the rabbits were divided into a normal group (Sham), a drug-untreated group (vehicle), and a drug-treated group (Compound).

After that, the heart was evaluated by the Langendorff experimental method as in 2-1) of 2) in Example 2. For the drug-treated group, the heart was treated with the drug by administering compound 43 (compound according to Synthesis Example 1) to physiological solution as in 2-2) of 2) in Example 2.

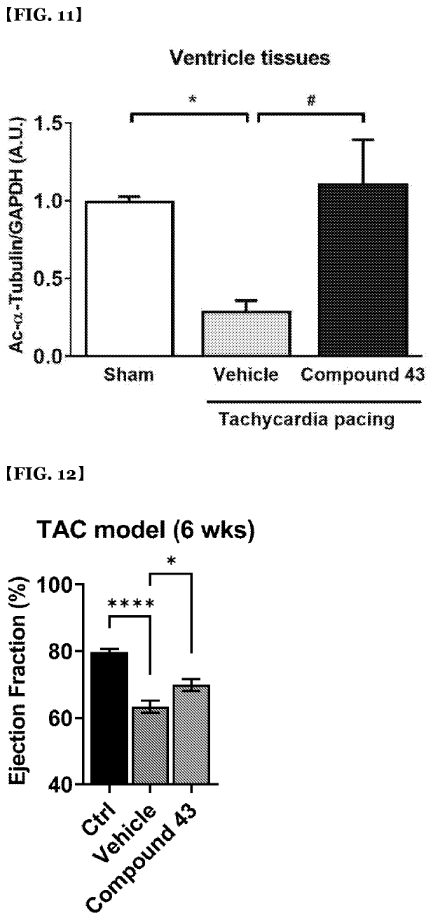

Then, the atria and the ventricles were separated, and then proteins were isolated from each tissue. The expression of acetylated tubulin was analyzed for each protein through western blot, so as to compare the expression levels between groups, and the results thereof are shown in FIGS. 10 and 11. * may mean P<0.05, ** may refer to P<0.01, and *** may represent less than P<0.001.

As can be confirmed in FIGS. 10 and 11, the group dosed with compound 43 according to the present disclosure had an expression of acetylated tubulin restored to that of the normal group (Sham) compared to the vehicle group not dosed with the drug in heart damage mimicking conditions (Tachycardia pacing).

Thus, it was confirmed that the compound according to the present disclosure shows a tubulin stabilization effect, and thus is advantageously used in preventing or treating heart failure disease.

### <Example 6> Confirmation of Effect on Heart Function Improvement in Animal Model with Heart Failure

In order to confirm the effect of the compound of the present disclosure (compound 43: compound of Synthesis Example 1) on improving heart function, compound of the present disclosure (compound 43: compound of Synthesis Example 1) was administered into TAC (transverse aortic constriction) model of the heart failure using the Sprague-Dawley rat and the results were analyzed.

### 1) Preparation of Animal Models and Classification of Group

Sprague-Dawley rat having the following conditions were prepared.

| | | | | Acclimation |
|---|---|---|---|---|
| Type | Source | Gender | Age | period |
| Sprague- | Koatech | Male | Seven weeks | 1 week |
| Dawley rat | | | | |

Rats were supplied from Koatech and fed on a standard diet (Central Lab Animal, Inc.) and kept under the conditions of constant temperature (23±3° C.), humidity (55±15%) and lighting (12 h light-dark cycle) with a free access to drinking. All experimental procedures were approved and performed according to the Institutional Animal Care and Use Committee (IACUC) of the external testing agency (with the IACUC animal study protocol approval number: KNOTUS IACUC 22-KE-0333).

Following the acclimation period, the animals were weighed and randomly divided into groups so that the average body weight of each group was distributed as uniformly as possible according to the weights ranked. As a result, the rats were divided into the normal group (control; ctrl), comparative group (vehicle), drug administration group (compound 43).

### 2) Preparation of TAC Model of the Heart Failure

After general anesthesia of the Sprague-Dawley rat, an incision was made in the second to third intercostal space on the left side while observing the electrocardiogram, and the transverse aorta in the middle of the left and right carotid arteries was isolated and constricted to 22 gauge needle using suture thread. Then, heart failure animal model was prepared by removing the needle and suturing the thoracic cavity and skin.

### 3) Drug Administration

The heart failure was induced in the Comparative group (vehicle) and the drug administration group (compound 43) according to the method of 2). The vehicle (0.5% Methylcellulose, 5 mL/kg) was administered orally into the Normal group (ctrl) and the Comparative group (vehicle) and compound 43 was administered orally into the Drug administrate group for 6 weeks from the date of induction of heart failure.

Table 5 Constitution of test groups

| | | | Number |
|---|---|---|---|
| Group | Administered dose | Usage | of animals |
| Normal group | 0.5% Methylcellulose, | Oral | Seven |
| | 5 mL/kg | bid(twice a day) | |
| Comparative | 0.5% Methylcellulose, | Oral | Seven |
| group | 5 mL/kg | bid (twice a day) | |
| Drug | Compound 43, 10 mg/Kg | Oral | Seven |
| administration | 0.5% Methylcellulose, | bid (twice a day) | |
| group | 5 mL/kg | | |

### 4) Ultrasound Measurement

On the sixth week after inducing TAC in the animal model and administering the drug, changes in heart according to ejection fraction were measured using an ultrasound device (S-Sharp ProspectTI, Taiwan). All data were presented as ±standard error, and statistical significance was analyzed using One-way ANOVA (multiple comparisons) for comparison between the vehicle-administered comparative group and the other group. * may represent P<0.05 and **** may represent P<0.0001.

### 5) Necropsy of Animal Model

On the sixth week after inducing TAC in the animal model and administering the drug, thoracotomy was performed, and heart and lung were removed and weighed. All data were presented as ±standard error, and statistical significance was analyzed using One-way ANOVA (multiple comparisons) for comparison between the vehicle-administered comparative group and the other group. * may represent P<0.05, and ** may represent P<0.01.

### 6) Confirming Pharmacological Effect by Measuring Ejection Fraction

As shown in FIG. 12, the compound according to the present disclosure increased ejection fraction in the heart failure animal model when ejection fraction decreased due to heart failure as a result of inducing TAC.

Therefore, it is confirmed that the compound according to the present disclosure is advantageously used for preventing or treating heart failure disease.

### 7) Confirming Pharmacological Effect by Analyzing the Weight of Heart and Lung

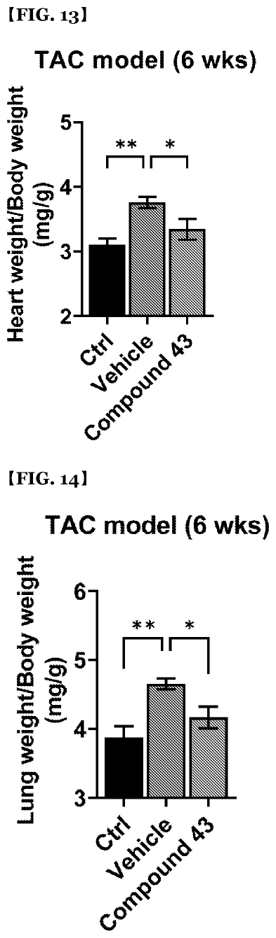

As shown in FIG. 13, the compound according to the present disclosure reduced the weight of heart in the heart failure animal model when the weight of heart increased due to heart failure as a result of inducing TAC.

Therefore, it is confirmed that the compound according to the present disclosure is advantageously used for preventing or treating heart failure disease.

As shown in FIG. 14, the compound according to the present disclosure reduced the weight of lung in the heart failure animal model when the weight of lung increased due to heart failure as a result of inducing TAC.

Therefore, it is confirmed that the compound according to the present disclosure is advantageously used for preventing or treating heart failure disease.

The present disclosure provides a pharmaceutical composition, a method, and a use as follow:

Item 1. A pharmaceutical composition for preventing or treating heart failure, comprising a compound represented by the above-mentioned formula I the above, optical isomers thereof or pharmaceutically acceptable salts thereof as an active ingredient.

Item 2. The pharmaceutical composition of item 1, wherein the compound represented by formula I is at least one selected from the group consisting of the above-mentioned compound 1 to 450 which is described in the above-mentioned Table A.

Item 3. The pharmaceutical composition of item 1 or 2, wherein the compound represented by formula I is at least one selected from the group consisting of the compound 40, the compound 43, the compound 239, the compound 285, the compound 295 and the compound 296 which is described in the above-mentioned Table B.

Item 4. A method for preventing or treating heart failure, including administering a compound represented by above formula I, optical isomers thereof or pharmaceutically acceptable salts thereof described in item 1 to 3 into an individual.

Item 5. A use of the compound represented by the above formula I, optical isomers thereof or pharmaceutically acceptable salts thereof described in items 1 to 3 for preventing or treating heart failure.

Item 6. A use of the compound represented by above formula I, optical isomers thereof or pharmaceutically acceptable salts thereof described in items 1 to 3 in preparing a medicament for preventing or treating heart failure.

Item 7. The pharmaceutical composition according to any one of items 1 to 3, the method according to item 4, or the use according to item 5 or 6, wherein the heart failure is at least one selected from the group consisting of Heart Failure with Preserved Ejection Fraction (HFpEF), Heart Failure with Midrange Ejection Fraction (HFmrEF) and Heart Failure with Reduced Ejection Fraction (HFrEF).

Item 8. The pharmaceutical composition according to any one of items 1 to 3, the method according to item 4, or the use according to item 5 or 6, wherein the heart failure includes Cardiomyopathy.

Item 9. The pharmaceutical composition according to any one of items 1 to 3, the method according to item 4, or the use according to item 5 or 6, wherein the Cardiomyopathy is at least one selected from the group consisting of Hypertrophic Cardiomyopathy (HCMP), Restrictive Cardiomyopathy and Dilated Cardiomyopathy (DCMP).

Item 10. The pharmaceutical composition according to any one of items 1 to 3, 7, 8 and 9, wherein the pharmaceutical composition is orally administered.

Item 11. The method according to any one of items 4, 7, 8 and 9, or the use according to any one of items 5 to 9, wherein the compound represented by above formula I, optical isomers thereof or pharmaceutically acceptable salts thereof described in item 1 to 3 is orally administered.

While specific portions of the present invention have been described in detail above, it is apparent to those skilled in the art that such detailed descriptions are set forth to illustrate exemplary embodiments only, but are not construed to limit the scope of the present invention. Thus, it should be understood that the substantial scope of the present invention is defined by the accompanying claims and equivalents thereto.

1. A pharmaceutical composition for preventing or treating heart failure, comprising a compound represented by formula I below, optical isomers thereof or pharmaceutically acceptable salts thereof as an active ingredient: 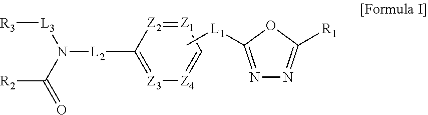
in formula I,
wherein L₁, L₂ or L₃ are each independently a bond or —(C₁-C₂ alkylene)-;
R₁ is —CX₂H or —CX₃;
R₂ is —NR^{A}R^{B}—OR^{C}, 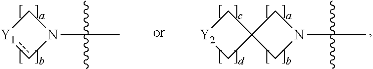
wherein, at least one H of 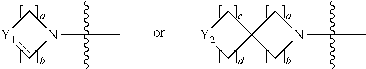
may be substituted with —X, —OH, —O(C₁-C₄ alkyl), —NR^{D}R^{E}, —(C₁-C₄ alkyl), —CF₃, —CF₂H, —CN, -aryl, -heteroaryl, —(C₁-C₄ alkyl)-aryl or —(C₁-C₄ alkyl)-heteroaryl, [wherein at least one H of the -aryl, -heteroaryl, —(C₁-C₄ alkyl)-aryl or —(C₁-C₄ alkyl)-heteroaryl may be substituted with —X, —OH, —CF₃ or —CF₂H];
R₃ is —H, —(C₁-C₄ alkyl), —(C₁-C₄ alkyl)-O(C₁-C₄ alkyl), —(C₁-C₄ alkyl)-C(═O)—O(C₁-C₄ alkyl), —(C₃-C₇ cycloalkyl), —(C₂-C₆ cycloheteroalkyl), -aryl, -heteroaryl, -adamantyl, 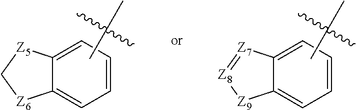
wherein, at least one H of —(C₁-C₄ alkyl) may be substituted with —X or —OH,
at least one H of -aryl or -heteroaryl each independently may be substituted with —X, —OH, —O(C₁-C₄ alkyl), —OCF₃, —O-aryl, —NR^{D}R^{E}, —(C₁-C₄ alkyl), —CF₃, —CF₂H, —C(═O)—(C₁-C₄ alkyl), —C(═O)—O(C₁-C₄ alkyl), —C(═O)—NR^{D}R^{E}, —S(═O)₂—(C₁-C₄ alkyl), aryl, heteroaryl, 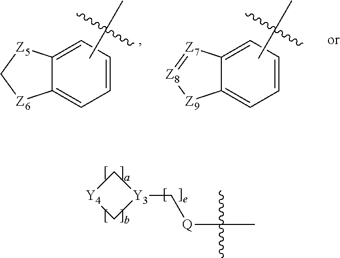
wherein, at least one H of 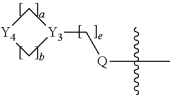
may be substituted with —X, —(C₁-C₄ alkyl), —NR^{D}R^{E}, —CF₃ or —CF₂H,
at least one H of —(C₃-C₇ cycloalkyl), —(C₂-C₆ cycloheteroalkyl), adamantyl, 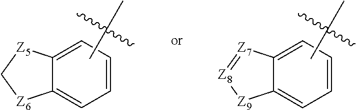
may be each independently substituted with —X, —OH or —(C₁-C₄ alkyl);
Y₁, Y₂ and Y₄ are each independently —CH₂—, —NR^{F}—, —O—, —C(═O)— or —S(═O)₂—;
Y₃ is —CH— or —N—;
Z₁ to Z₄ are each independently N or CR^{Z}, wherein at least three of Z₁ to Z₄ may not be simultaneously N, and R^{Z} is —H, —X or —O(C₁-C₄ alkyl);
Z₅ and Z₆ are each independently —CH₂— or —O—;
Z₇ and Z₈ are each independently ═CH— or ═N—;
Z₉ is —NR^{G}— or —S—;
R^{A} and R^{B} are each independently —H, —(C₁-C₄ alkyl), —(C₁-C₄ alkyl)-OH, —(C₁-C₄ alkyl)-NR^{D}R^{E}, -aryl, —(C₁-C₄ alkyl)-aryl, -heteroaryl, —(C₁-C₄ aryl)-heteroaryl, —(C₃-C₇ cycloalkyl), —(C₂-C₆ heterocycloalkyl) or 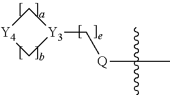
wherein, at least one H of the —(C₁-C₄ alkyl), —(C₁-C₄ alkyl)-OH or —(C₁-C₄ alkyl)-NR^{D}R^{E} may be substituted with —X,
at least one H of the -aryl, —(C₁-C₄ alkyl)-aryl, -heteroaryl, —(C₁-C₄ alkyl)-heteroaryl, —(C₃-C₇ cycloalkyl) or —(C₂-C₆ heterocycloalkyl) may be substituted with —X, —OH, —O(C₁-C₄ alkyl), —(C₁-C₄ alkyl), —CF₃, —CF₂H or —CN,
at least one H of 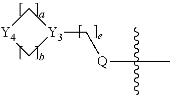
may be substituted with n-X, —OH, —O(C₁-C₄ alkyl), —(C₁-C₄ alkyl), —CF₃, —CF₂H, —CN, —(C₂-C₆ heterocycloalkyl), -aryl, —(C₁-C₄ alkyl)-aryl, -heteroaryl or -heteroaryl-(C₁-C₄ alkyl);
R^{C} is —(C₁-C₄ alkyl), -aryl, —(C₁-C₄ alkyl)-aryl, -heteroaryl or —(C₁-C₄ alkyl)-heteroaryl, wherein, at least one H of —(C₁-C₄ alkyl) may be substituted with —X or —OH, at least one H of -aryl, —(C₁-C₄ alkyl)-aryl, -heteroaryl or —(C₁-C₄ alkyl)-heteroaryl may be substituted with —X, —OH, —CF₃ or —CF₂H;
R^{D} and R^{E} are each independently —H, —(C₁-C₄ alkyl), -aryl or —(C₁-C₄ alkyl)-aryl, wherein, at least one H of —(C₁-C₄ alkyl) may be substituted with —X or —OH, at least one H of -aryl or —(C₁-C₄ alkyl)-aryl may be substituted with —X, —OH, —CF₃ or —CF₂H;
R^{F} is —H, —(C₁-C₆ alkyl), —(C₁-C₄ alkyl)-OH, —(C₁-C₄ alkyl)-O—(C₁-C₄ alkyl), —C(═O)—(C₁-C₄ alkyl), —C(═O)—O(C₁-C₄ alkyl), —(C₁-C₄ alkyl)-C(═O)—O(C₁-C₄ alkyl), —(C₁-C₄ alkyl)-NR^{D}R^{E}, —S(═O)₂—(C₁-C₄ alkyl), -aryl, —(C₁-C₄ alkyl)-aryl, —(C₂-C₄ alkenyl)-aryl, -heteroaryl, —(C₁-C₄ alkyl)-heteroaryl, —C(═O)—(C₃-C₇ cycloalkyl), —(C₂-C₆ heterocycloalkyl) or —(C₁-C₄ alkyl)-C(═O)—(C₂-C₆ heterocycloalkyl)
wherein at least one H of —(C₁-C₄ alkyl), —(C₁-C₄ alkyl)-OH, —(C₁-C₄ alkyl)-O—(C₁-C₄ alkyl), —C(═O)—(C₁-C₄ alkyl), —C(═O)—O(C₁-C₄ alkyl), —(C₁-C₄ alkyl)-C(═O)—O(C₁-C₄ alkyl), —(C₁-C₄ alkyl)-NR^{D}R^{E} or —S(═O)₂—(C₁-C₄ alkyl) may be substituted with —X,
at least one H of -aryl, —(C₁-C₄alkyl)-aryl, —(C₂-C₄ alkenyl)-aryl, -heteroaryl, —(C₁-C₄ alkyl)-heteroaryl, —C(═O)—(C₃-C₇ cycloalkyl), —C₂-C₆ heterocycloalkyl or —(C₁-C₄alkyl)-C(═O)—(C₂-C₆heterocycloalkyl) may be substituted with —X, —OH, —CF₃ or —CF₂H;
R^{G} is —H or —(C₁-C₄ alkyl);
Q is —O— or a bond; [symbol]
is a single bond or double bond, provided that, [symbol] is a double bond, Y₁ is ═CH—;
a to e are each independently an integer of 0, 1, 2, 3 or 4 provided that, a and b may not be simultaneously 0, and c and d may not be simultaneously 0;
X is each independently F, Cl, Br or I.

2. The pharmaceutical composition of claim 1, wherein in the compound represented by formula I,
L₁, L₂ or L₃ are each independently a bond or —(C₁-C₂alkylene)-;
R₁ is —CX₂H or —CX₃;
R₂ is —NR^{A}R^{B}, —OR^{C}, 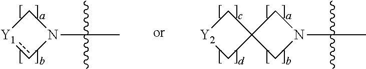
wherein at least one of H of 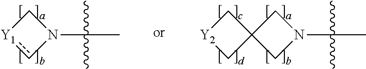
may be substituted with —X, —OH, —NR^{D}R^{E}—(C₁-C₄ alkyl);
R₃ is —(C₁-C₄ alkyl), —(C₃-C₇ cycloalkyl), -aryl, -heteroaryl, -adamantyl, 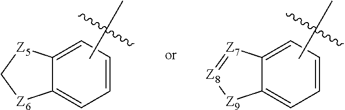
wherein at least one H of -aryl or -heteroaryl may be each independently substituted with —X, —O(C₁-C₄alkyl), —OCF₃, —O-aryl, —NR^{D}R^{E}, —(C₁-C₄ alkyl), —CF₃, —S(═O)₂—(C₁-C₄alkyl), -aryl, -heteroaryl 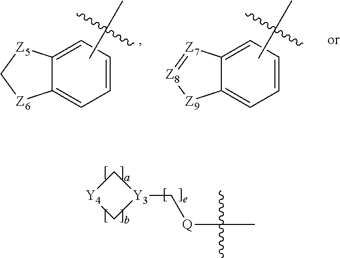
wherein, at least one H of 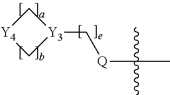
may be substituted with —NR^{D}R^{E} or —(C₁-C₄ alkyl),
at least one H of 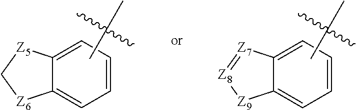
maybe each independently substituted with —(C₁-C₄ alkyl);
Y₁, Y₂ and Y₄ are each independently —CH₂—, —NR^{F}—, —O—, —C(═O)— or —S(═O)₂—;
Y₃ is —CH— or —N—;
Z₁ to Z₄ is each independently N or CR^{Z} wherein at least three of Z₁ to Z₄ may not be simultaneously N, and R^{Z} is —H, —X or —O(C₁-C₄ alkyl);
Z₅ and Z₆ are each independently —CH₂— or —O—;
Z₇ and Z₈ are each independently ═CH— or ═N—;
Z₉ is —NR^{G}— or —S—;
R^{A} and R^{B} are each independently —H, —(C₁-C₄ alkyl), —(C₁-C₄ alkyl)-OH, —(C₁-C₄ alkyl)-NR^{D}R^{E}, -aryl, —(C₁-C₄alkyl)-aryl, —(C₃-C₇ cycloalkyl) or 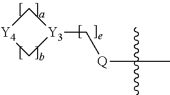
wherein, at least one H of 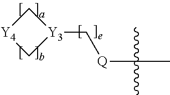
may be substituted with —X, —(C₁-C₄alkyl), —CF₃, —(C₂-C₆ heterocycloalkyl), —(C₁-C₄ alkyl)-aryl, -heteroaryl or heteroaryl-(C₁-C₄ alkyl);
R^{C} is —(C₁-C₄ alkyl) or -aryl;
R^{D} and R^{E} are each independently —H, —(C₁-C₄alkyl) or —(C₁-C₄ alkyl)-aryl;
R^{F} is —H, —(C₁-C₆alkyl), —(C₁-C₄alkyl)-OH, —(C₁-C₄ alkyl)-O—(C₁-C₄ alkyl), —C(═O)—(C₁-C₄alkyl), —C(═O)—O(C₁-C₄ alkyl), —(C₁-C₄ alkyl)-C(═O)—O(C₁-C₄alkyl), —(C₁-C₄alkyl)-NR^{D}R^{E}, —S(═O)₂—(C₁-C₄ alkyl), -aryl, —(C₁-C₄ alkyl)-aryl, —(C₂-C₄ alkenyl)-aryl, -heteroaryl, —(C₁-C₄ alkyl)-heteroaryl, —C(═O)—(C₃-C₇ cycloalkyl), —(C₂-C₆ heterocycloalkyl) or —(C₁-C₄ alkyl)-C(═O)—(C₂-C₆ heterocycloalkyl)
wherein at least one H of —(C₁-C₄alkyl) or —C(═O)—O(C₁-C₄alkyl) may be substituted with —X,
at least one H of -aryl may be substituted with —X;
R^{G} is —(C₁-C₄ alkyl);
Q is —O— or a bond; [symbol]
is a single bond or a double bond provided that [symbol] is a double bond, Y₁ is —CH—;
a to e are each independently an integer of 0, 1, 2, 3 or 4 provided that a and b may not be simultaneously 0, and c and d may not be simultaneously 0;
X is each independently F, Cl, Br or I.

3. The pharmaceutical composition of claim 1, wherein the compound represented by formula I is the compound represented by formula Ia: 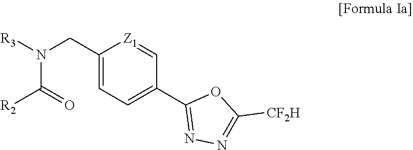
in formula Ia, wherein,
R₂ is 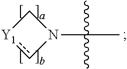
R₃ is -aryl wherein, at least one H of -aryl may be each independently substituted with —X;
Y₁ is —O— or —S(═O)₂—;
Z₁ is N or CR^{Z} wherein, R^{Z} is —X;
a and b are each independently an integer of 0, 1, 2, 3 or 4 wherein, a and b may not be simultaneously 0;
X is each independently F, Cl, Br or I.

4. The pharmaceutical composition of claim 3, wherein in the compound represented by formula Ia,
R₂ is 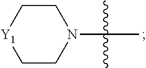
R₃ is -phenyl wherein, at least one H of -phenyl each independently is substituted with —F or —Cl;
Y₁ is —O— or —S(═O)₂—;
Z₁ is N or CF.

5. A pharmaceutical composition for preventing or treating heart failure, comprising a compound, optical isomers thereof or pharmaceutically acceptable salts thereof as an active ingredient, wherein the compound has the following structure:

| Compound | Structure |
|---|---|
| 1 | 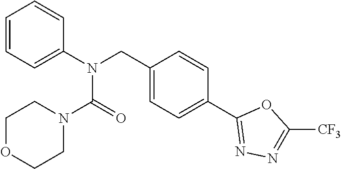 [SMILES: O=C(N1CCOCC1)N(Cc1ccc(-c2nnc(C(F)(F)F)o2)cc1)c1ccccc1] |
| 2 | 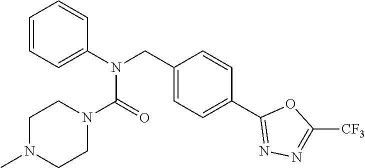 [SMILES: CN1CCN(C(=O)N(Cc2ccc(-c3nnc(C(F)(F)F)o3)cc2)c2ccccc2)CC1] |
| 3 | 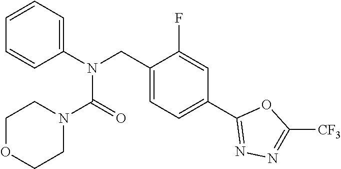 [SMILES: O=C(N1CCOCC1)N(Cc1ccc(-c2nnc(C(F)(F)F)o2)cc1F)c1ccccc1] |
| 4 | 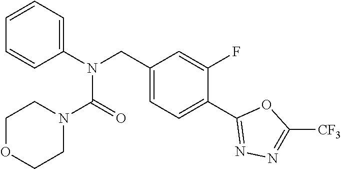 [SMILES: O=C(N1CCOCC1)N(Cc1ccc(-c2nnc(C(F)(F)F)o2)c(F)c1)c1ccccc1] |
| 5 | 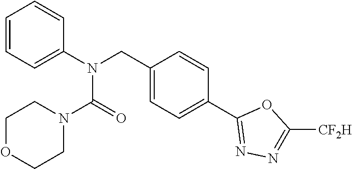 [SMILES: O=C(N1CCOCC1)N(Cc1ccc(-c2nnc(C(F)F)o2)cc1)c1ccccc1] |
| 6 | 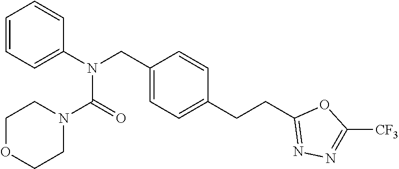 [SMILES: O=C(N1CCOCC1)N(Cc1ccc(CCc2nnc(C(F)(F)F)o2)cc1)c1ccccc1] |
| 7 | 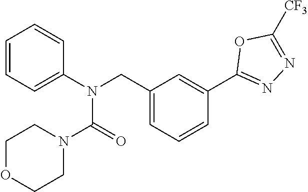 [SMILES: O=C(N1CCOCC1)N(Cc1cccc(-c2nnc(C(F)(F)F)o2)c1)c1ccccc1] |
| 8 | 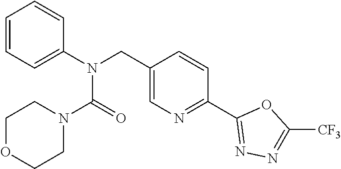 [SMILES: O=C(N1CCOCC1)N(Cc1ccc(-c2nnc(C(F)(F)F)o2)nc1)c1ccccc1] |
| 9 | 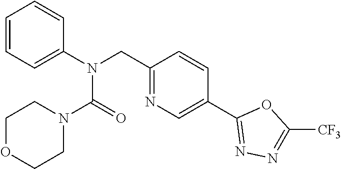 [SMILES: O=C(N1CCOCC1)N(Cc1ccc(-c2nnc(C(F)(F)F)o2)cn1)c1ccccc1] |
| 10 | 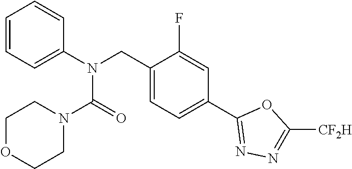 [SMILES: O=C(N1CCOCC1)N(Cc1ccc(-c2nnc(C(F)F)o2)cc1F)c1ccccc1] |
| 11 | 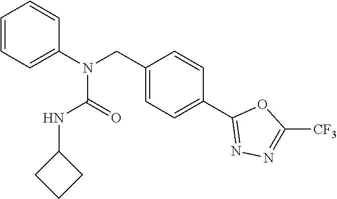 [SMILES: O=C(NC1CCC1)N(Cc1ccc(-c2nnc(C(F)(F)F)o2)cc1)c1ccccc1] |
| 12 | 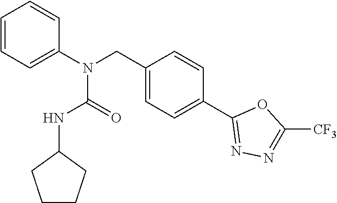 [SMILES: O=C(NC1CCCC1)N(Cc1ccc(-c2nnc(C(F)(F)F)o2)cc1)c1ccccc1] |
| 13 | 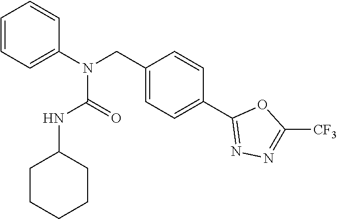 [SMILES: O=C(NC1CCCCC1)N(Cc1ccc(-c2nnc(C(F)(F)F)o2)cc1)c1ccccc1] |
| 14 | 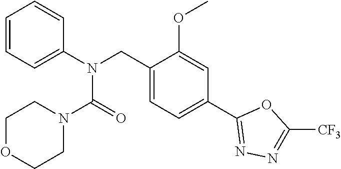 [SMILES: COc1cc(-c2nnc(C(F)(F)F)o2)ccc1CN(C(=O)N1CCOCC1)c1ccccc1] |
| 15 | 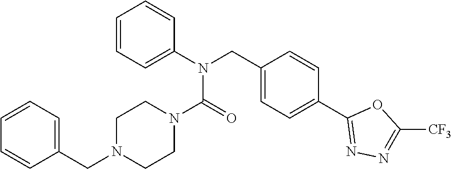 [SMILES: O=C(N1CCN(Cc2ccccc2)CC1)N(Cc1ccc(-c2nnc(C(F)(F)F)o2)cc1)c1ccccc1] |
| 16 | 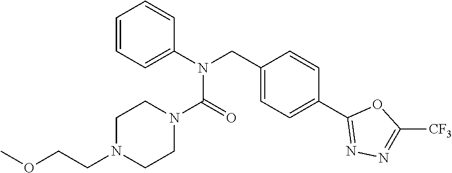 [SMILES: COCCN1CCN(C(=O)N(Cc2ccc(-c3nnc(C(F)(F)F)o3)cc2)c2ccccc2)CC1] |
| 17 | 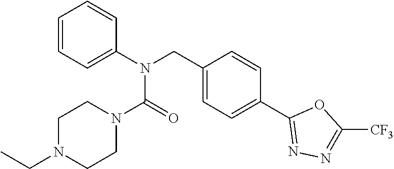 [SMILES: CCN1CCN(C(=O)N(Cc2ccc(-c3nnc(C(F)(F)F)o3)cc2)c2ccccc2)CC1] |
| 18 | 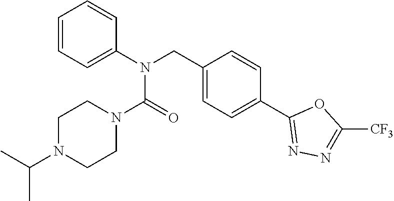 [SMILES: CC(C)N1CCN(C(=O)N(Cc2ccc(-c3nnc(C(F)(F)F)o3)cc2)c2ccccc2)CC1] |
| 19 | 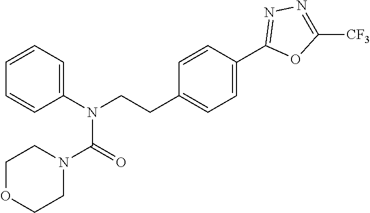 [SMILES: O=C(N1CCOCC1)N(CCc1ccc(-c2nnc(C(F)(F)F)o2)cc1)c1ccccc1] |
| 20 | 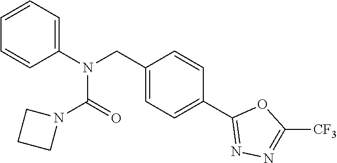 [SMILES: O=C(N1CCC1)N(Cc1ccc(-c2nnc(C(F)(F)F)o2)cc1)c1ccccc1] |
| 21 | 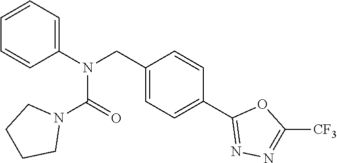 [SMILES: O=C(N1CCCC1)N(Cc1ccc(-c2nnc(C(F)(F)F)o2)cc1)c1ccccc1] |
| 22 | 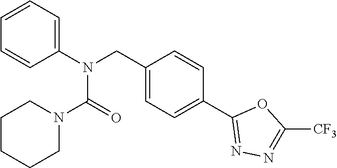 [SMILES: O=C(N1CCCCC1)N(Cc1ccc(-c2nnc(C(F)(F)F)o2)cc1)c1ccccc1] |
| 23 | 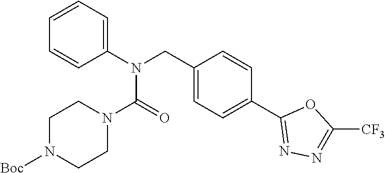 [SMILES: CC(C)(C)OC(=O)N1CCN(C(=O)N(Cc2ccc(-c3nnc(C(F)(F)F)o3)cc2)c2ccccc2)CC1] |
| 24 | 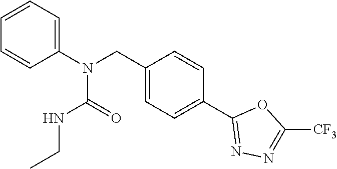 [SMILES: CCNC(=O)N(Cc1ccc(-c2nnc(C(F)(F)F)o2)cc1)c1ccccc1] |
| 25 | 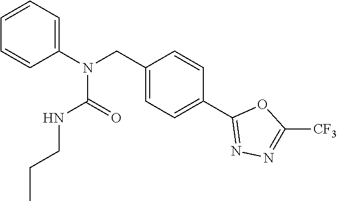 [SMILES: CCCNC(=O)N(Cc1ccc(-c2nnc(C(F)(F)F)o2)cc1)c1ccccc1] |
| 26 | 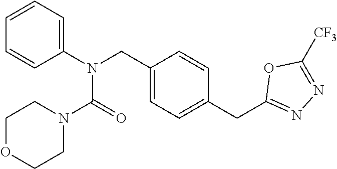 [SMILES: O=C(N1CCOCC1)N(Cc1ccc(Cc2nnc(C(F)(F)F)o2)cc1)c1ccccc1] |
| 27 | 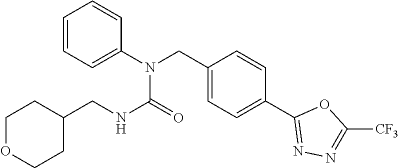 [SMILES: O=C(NCC1CCOCC1)N(Cc1ccc(-c2nnc(C(F)(F)F)o2)cc1)c1ccccc1] |
| 28 | 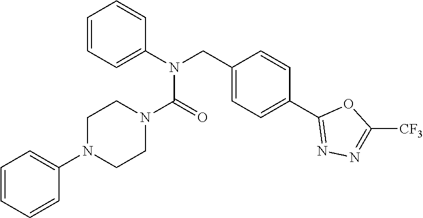 [SMILES: O=C(N1CCN(c2ccccc2)CC1)N(Cc1ccc(-c2nnc(C(F)(F)F)o2)cc1)c1ccccc1] |
| 29 | 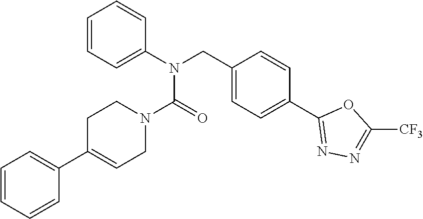 [SMILES: O=C(N1CC=C(c2ccccc2)CC1)N(Cc1ccc(-c2nnc(C(F)(F)F)o2)cc1)c1ccccc1] |
| 30 | 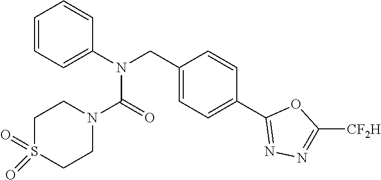 [SMILES: O=C(N1CCS(=O)(=O)CC1)N(Cc1ccc(-c2nnc(C(F)F)o2)cc1)c1ccccc1] |
| 31 | 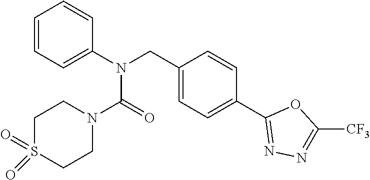 [SMILES: O=C(N1CCS(=O)(=O)CC1)N(Cc1ccc(-c2nnc(C(F)(F)F)o2)cc1)c1ccccc1] |
| 32 | 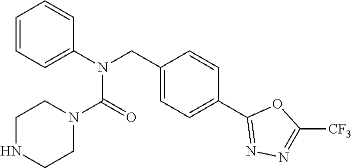 [SMILES: O=C(N1CCNCC1)N(Cc1ccc(-c2nnc(C(F)(F)F)o2)cc1)c1ccccc1] |
| 33 | 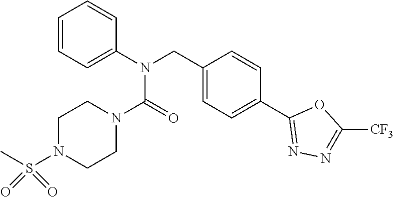 [SMILES: CS(=O)(=O)N1CCN(C(=O)N(Cc2ccc(-c3nnc(C(F)(F)F)o3)cc2)c2ccccc2)CC1] |
| 34 | 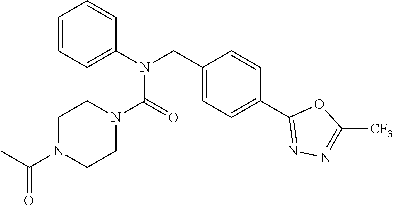 [SMILES: CC(=O)N1CCN(C(=O)N(Cc2ccc(-c3nnc(C(F)(F)F)o3)cc2)c2ccccc2)CC1] |
| 35 | 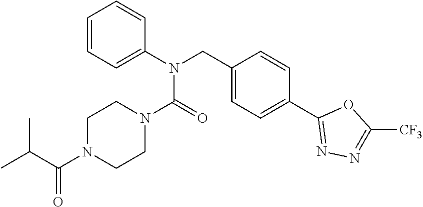 [SMILES: CC(C)C(=O)N1CCN(C(=O)N(Cc2ccc(-c3nnc(C(F)(F)F)o3)cc2)c2ccccc2)CC1] |
| 36 | 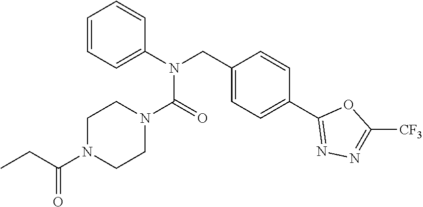 [SMILES: CCC(=O)N1CCN(C(=O)N(Cc2ccc(-c3nnc(C(F)(F)F)o3)cc2)c2ccccc2)CC1] |
| 37 | 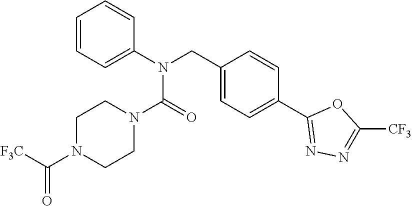 [SMILES: O=C(N1CCN(C(=O)C(F)(F)F)CC1)N(Cc1ccc(-c2nnc(C(F)(F)F)o2)cc1)c1ccccc1] |
| 38 | 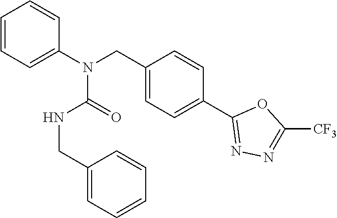 [SMILES: O=C(NCc1ccccc1)N(Cc1ccc(-c2nnc(C(F)(F)F)o2)cc1)c1ccccc1] |
| 39 | 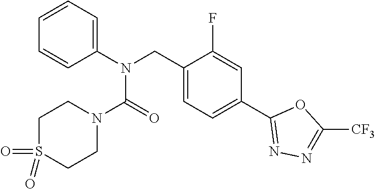 [SMILES: O=C(N1CCS(=O)(=O)CC1)N(Cc1ccc(-c2nnc(C(F)(F)F)o2)cc1F)c1ccccc1] |
| 40 | 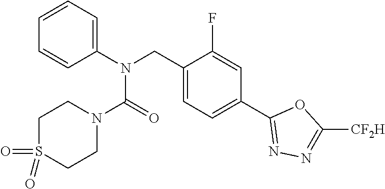 [SMILES: O=C(N1CCS(=O)(=O)CC1)N(Cc1ccc(-c2nnc(C(F)F)o2)cc1F)c1ccccc1] |
| 41 | 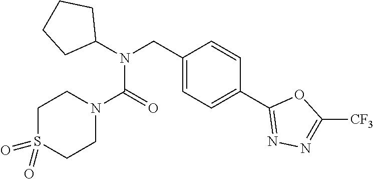 [SMILES: O=C(N1CCS(=O)(=O)CC1)N(Cc1ccc(-c2nnc(C(F)(F)F)o2)cc1)C1CCCC1] |
| 42 | 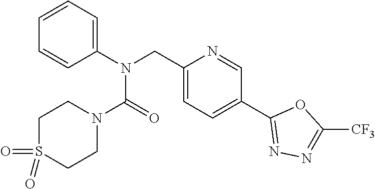 [SMILES: O=C(N1CCS(=O)(=O)CC1)N(Cc1ccc(-c2nnc(C(F)(F)F)o2)cn1)c1ccccc1] |
| 43 | 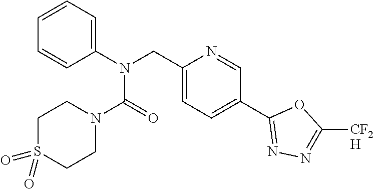 [SMILES: O=C(N1CCS(=O)(=O)CC1)N(Cc1ccc(-c2nnc(C(F)F)o2)cn1)c1ccccc1] |
| 44 | 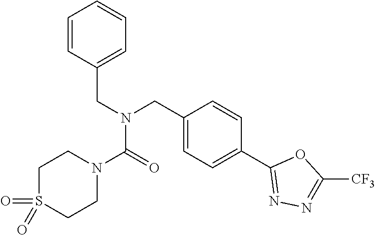 [SMILES: O=C(N1CCS(=O)(=O)CC1)N(Cc1ccccc1)Cc1ccc(-c2nnc(C(F)(F)F)o2)cc1] |
| 45 | 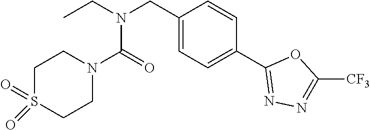 [SMILES: CCN(Cc1ccc(-c2nnc(C(F)(F)F)o2)cc1)C(=O)N1CCS(=O)(=O)CC1] |
| 46 | 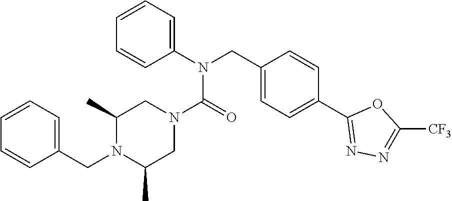 [SMILES: C[C@@H]1CN(C(=O)N(Cc2ccc(-c3nnc(C(F)(F)F)o3)cc2)c2ccccc2)C[C@H](C)N1Cc1ccccc1] |
| 47 | 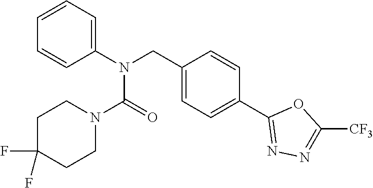 [SMILES: O=C(N1CCC(F)(F)CC1)N(Cc1ccc(-c2nnc(C(F)(F)F)o2)cc1)c1ccccc1] |
| 48 | 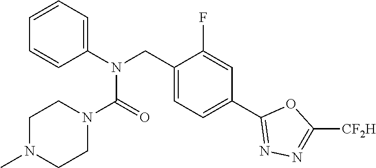 [SMILES: CN1CCN(C(=O)N(Cc2ccc(-c3nnc(C(F)F)o3)cc2F)c2ccccc2)CC1] |
| 49 | 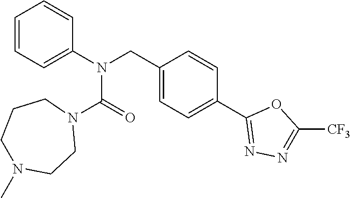 [SMILES: CN1CCCN(C(=O)N(Cc2ccc(-c3nnc(C(F)(F)F)o3)cc2)c2ccccc2)CC1] |
| 50 | 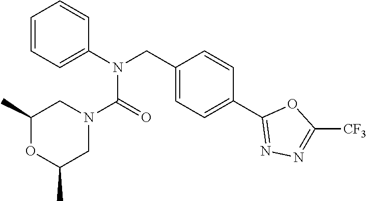 [SMILES: C[C@@H]1CN(C(=O)N(Cc2ccc(-c3nnc(C(F)(F)F)o3)cc2)c2ccccc2)C[C@H](C)O1] |
| 51 | 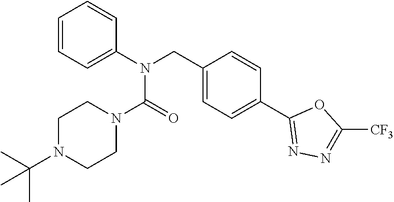 [SMILES: CC(C)(C)N1CCN(C(=O)N(Cc2ccc(-c3nnc(C(F)(F)F)o3)cc2)c2ccccc2)CC1] |
| 52 | 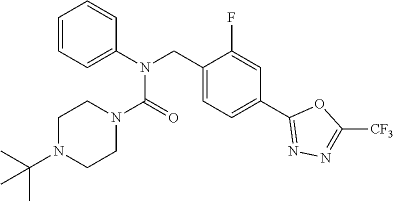 [SMILES: CC(C)(C)N1CCN(C(=O)N(Cc2ccc(-c3nnc(C(F)(F)F)o3)cc2F)c2ccccc2)CC1] |
| 53 | 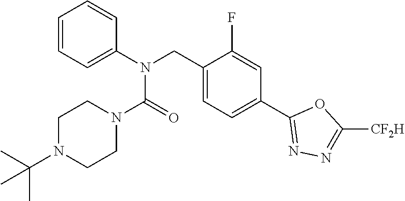 [SMILES: CC(C)(C)N1CCN(C(=O)N(Cc2ccc(-c3nnc(C(F)F)o3)cc2F)c2ccccc2)CC1] |
| 54 | 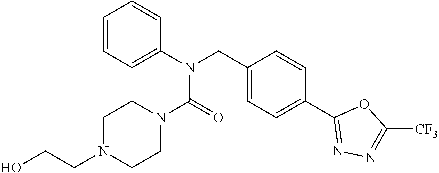 [SMILES: O=C(N1CCN(CCO)CC1)N(Cc1ccc(-c2nnc(C(F)(F)F)o2)cc1)c1ccccc1] |
| 55 | 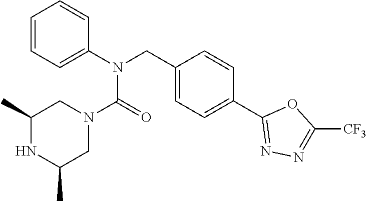 [SMILES: C[C@@H]1CN(C(=O)N(Cc2ccc(-c3nnc(C(F)(F)F)o3)cc2)c2ccccc2)C[C@H](C)N1] |
| 56 | 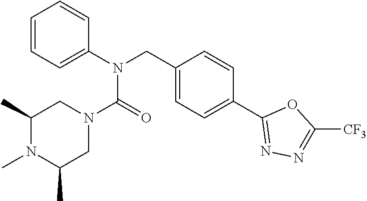 [SMILES: C[C@@H]1CN(C(=O)N(Cc2ccc(-c3nnc(C(F)(F)F)o3)cc2)c2ccccc2)C[C@H](C)N1C] |
| 57 | 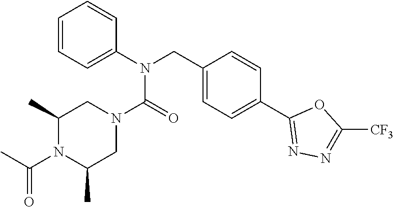 [SMILES: CC(=O)N1[C@H](C)CN(C(=O)N(Cc2ccc(-c3nnc(C(F)(F)F)o3)cc2)c2ccccc2)C[C@@H]1C] |
| 58 | 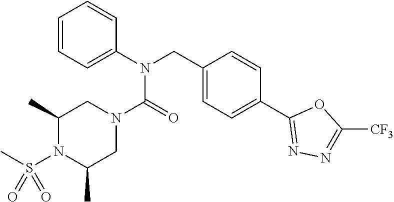 [SMILES: C[C@@H]1CN(C(=O)N(Cc2ccc(-c3nnc(C(F)(F)F)o3)cc2)c2ccccc2)C[C@H](C)N1S(C)(=O)=O] |
| 59 | 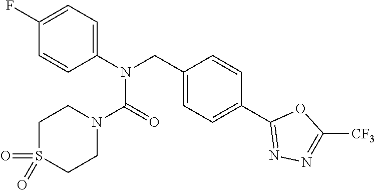 [SMILES: O=C(N1CCS(=O)(=O)CC1)N(Cc1ccc(-c2nnc(C(F)(F)F)o2)cc1)c1ccc(F)cc1] |
| 60 | 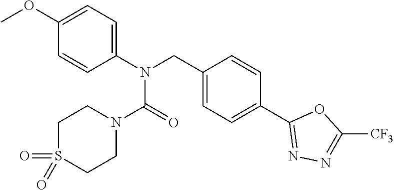 [SMILES: COc1ccc(N(Cc2ccc(-c3nnc(C(F)(F)F)o3)cc2)C(=O)N2CCS(=O)(=O)CC2)cc1] |
| 61 | 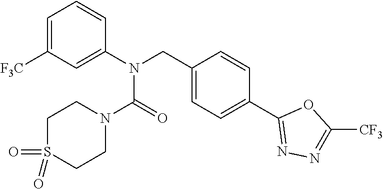 [SMILES: O=C(N1CCS(=O)(=O)CC1)N(Cc1ccc(-c2nnc(C(F)(F)F)o2)cc1)c1cccc(C(F)(F)F)c1] |
| 62 | 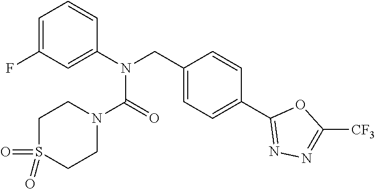 [SMILES: O=C(N1CCS(=O)(=O)CC1)N(Cc1ccc(-c2nnc(C(F)(F)F)o2)cc1)c1cccc(F)c1] |
| 63 | 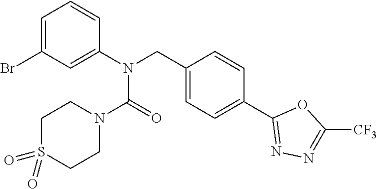 [SMILES: O=C(N1CCS(=O)(=O)CC1)N(Cc1ccc(-c2nnc(C(F)(F)F)o2)cc1)c1cccc(Br)c1] |
| 64 | 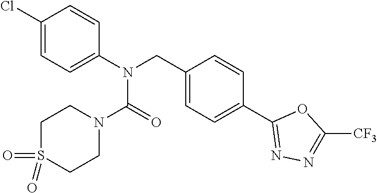 [SMILES: O=C(N1CCS(=O)(=O)CC1)N(Cc1ccc(-c2nnc(C(F)(F)F)o2)cc1)c1ccc(Cl)cc1] |
| 65 | 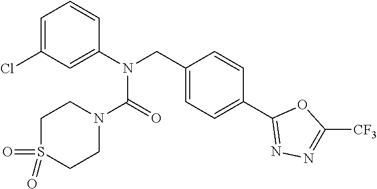 [SMILES: O=C(N1CCS(=O)(=O)CC1)N(Cc1ccc(-c2nnc(C(F)(F)F)o2)cc1)c1cccc(Cl)c1] |
| 66 | 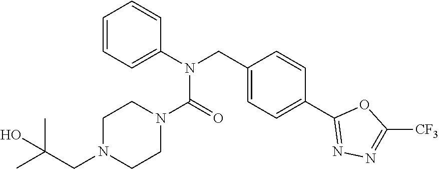 [SMILES: CC(C)(O)CN1CCN(C(=O)N(Cc2ccc(-c3nnc(C(F)(F)F)o3)cc2)c2ccccc2)CC1] |
| 67 | 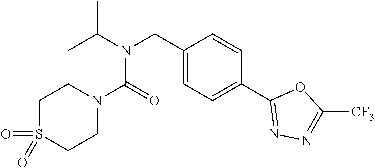 [SMILES: CC(C)N(Cc1ccc(-c2nnc(C(F)(F)F)o2)cc1)C(=O)N1CCS(=O)(=O)CC1] |
| 68 | 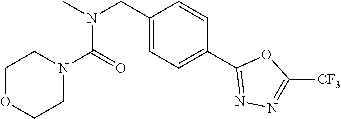 [SMILES: CN(Cc1ccc(-c2nnc(C(F)(F)F)o2)cc1)C(=O)N1CCOCC1] |
| 69 | 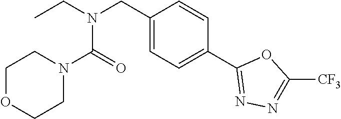 [SMILES: CCN(Cc1ccc(-c2nnc(C(F)(F)F)o2)cc1)C(=O)N1CCOCC1] |
| 70 | 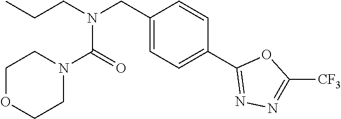 [SMILES: CCCN(Cc1ccc(-c2nnc(C(F)(F)F)o2)cc1)C(=O)N1CCOCC1] |
| 71 | 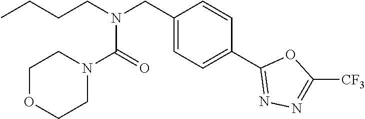 [SMILES: CCCCN(Cc1ccc(-c2nnc(C(F)(F)F)o2)cc1)C(=O)N1CCOCC1] |
| 72 | 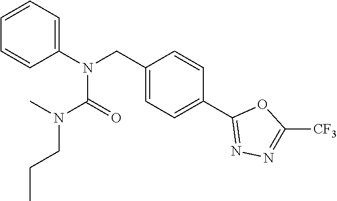 [SMILES: CCCN(C)C(=O)N(Cc1ccc(-c2nnc(C(F)(F)F)o2)cc1)c1ccccc1] |
| 73 | 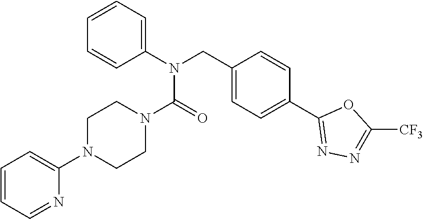 [SMILES: O=C(N1CCN(c2ccccn2)CC1)N(Cc1ccc(-c2nnc(C(F)(F)F)o2)cc1)c1ccccc1] |
| 74 | 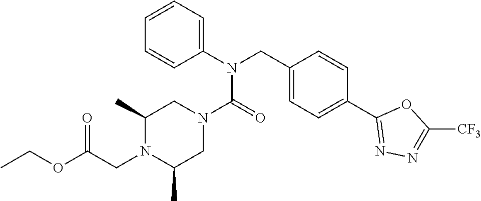 [SMILES: CCOC(=O)CN1[C@H](C)CN(C(=O)N(Cc2ccc(-c3nnc(C(F)(F)F)o3)cc2)c2ccccc2)C[C@@H]1C] |
| 75 | 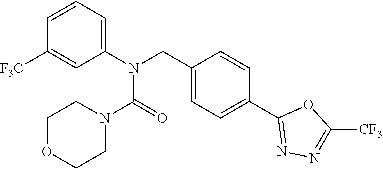 [SMILES: O=C(N1CCOCC1)N(Cc1ccc(-c2nnc(C(F)(F)F)o2)cc1)c1cccc(C(F)(F)F)c1] |
| 76 | 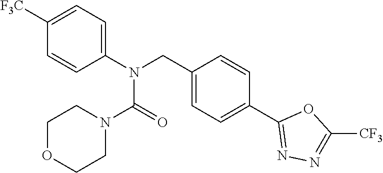 [SMILES: O=C(N1CCOCC1)N(Cc1ccc(-c2nnc(C(F)(F)F)o2)cc1)c1ccc(C(F)(F)F)cc1] |
| 77 | 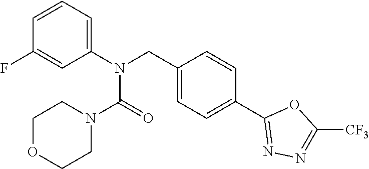 [SMILES: O=C(N1CCOCC1)N(Cc1ccc(-c2nnc(C(F)(F)F)o2)cc1)c1cccc(F)c1] |
| 78 | 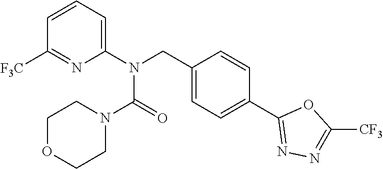 [SMILES: O=C(N1CCOCC1)N(Cc1ccc(-c2nnc(C(F)(F)F)o2)cc1)c1cccc(C(F)(F)F)n1] |
| 79 | 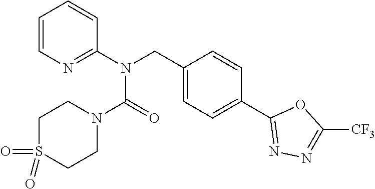 [SMILES: O=C(N1CCS(=O)(=O)CC1)N(Cc1ccc(-c2nnc(C(F)(F)F)o2)cc1)c1ccccn1] |
| 80 | 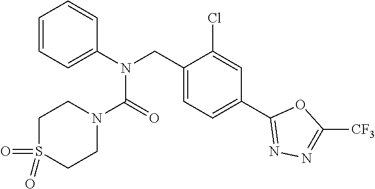 [SMILES: O=C(N1CCS(=O)(=O)CC1)N(Cc1ccc(-c2nnc(C(F)(F)F)o2)cc1Cl)c1ccccc1] |
| 81 | 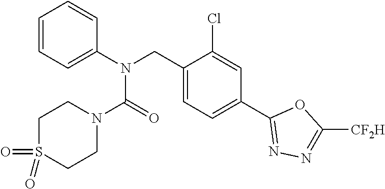 [SMILES: O=C(N1CCS(=O)(=O)CC1)N(Cc1ccc(-c2nnc(C(F)F)o2)cc1Cl)c1ccccc1] |
| 82 | 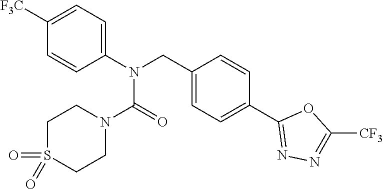 [SMILES: O=C(N1CCS(=O)(=O)CC1)N(Cc1ccc(-c2nnc(C(F)(F)F)o2)cc1)c1ccc(C(F)(F)F)cc1] |
| 83 | 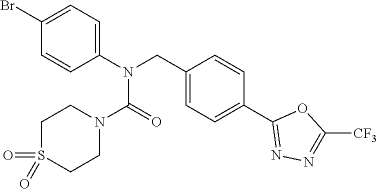 [SMILES: O=C(N1CCS(=O)(=O)CC1)N(Cc1ccc(-c2nnc(C(F)(F)F)o2)cc1)c1ccc(Br)cc1] |
| 84 | 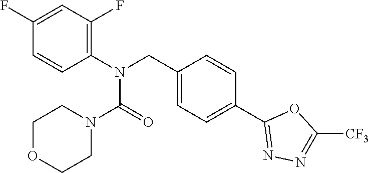 [SMILES: O=C(N1CCOCC1)N(Cc1ccc(-c2nnc(C(F)(F)F)o2)cc1)c1ccc(F)cc1F] |
| 85 | 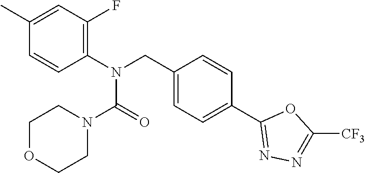 [SMILES: Cc1ccc(N(Cc2ccc(-c3nnc(C(F)(F)F)o3)cc2)C(=O)N2CCOCC2)c(F)c1] |
| 86 | 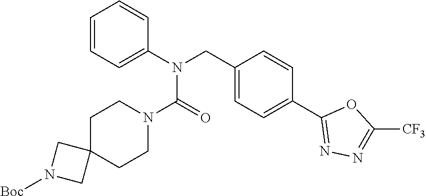 [SMILES: CC(C)(C)OC(=O)N1CC2(CCN(C(=O)N(Cc3ccc(-c4nnc(C(F)(F)F)o4)cc3)c3ccccc3)CC2)C1] |
| 87 | 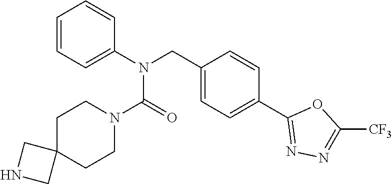 [SMILES: O=C(N1CCC2(CC1)CNC2)N(Cc1ccc(-c2nnc(C(F)(F)F)o2)cc1)c1ccccc1] |
| 88 | 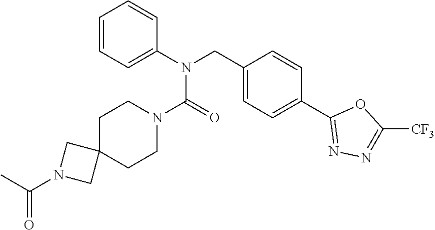 [SMILES: CC(=O)N1CC2(CCN(C(=O)N(Cc3ccc(-c4nnc(C(F)(F)F)o4)cc3)c3ccccc3)CC2)C1] |
| 89 | 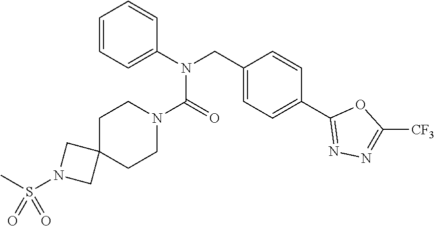 [SMILES: CS(=O)(=O)N1CC2(CCN(C(=O)N(Cc3ccc(-c4nnc(C(F)(F)F)o4)cc3)c3ccccc3)CC2)C1] |
| 90 | 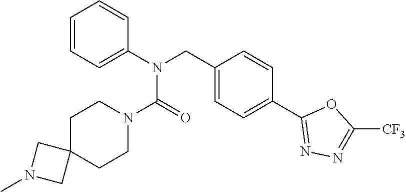 [SMILES: CN1CC2(CCN(C(=O)N(Cc3ccc(-c4nnc(C(F)(F)F)o4)cc3)c3ccccc3)CC2)C1] |
| 91 | 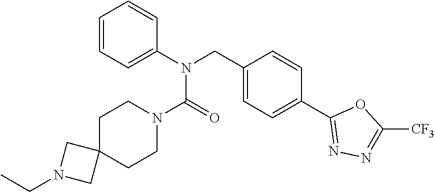 [SMILES: CCN1CC2(CCN(C(=O)N(Cc3ccc(-c4nnc(C(F)(F)F)o4)cc3)c3ccccc3)CC2)C1] |
| 92 | 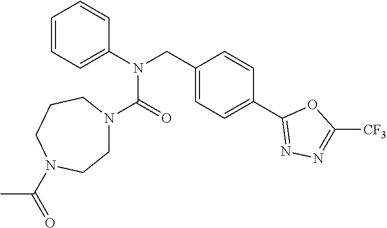 [SMILES: CC(=O)N1CCCN(C(=O)N(Cc2ccc(-c3nnc(C(F)(F)F)o3)cc2)c2ccccc2)CC1] |
| 93 | 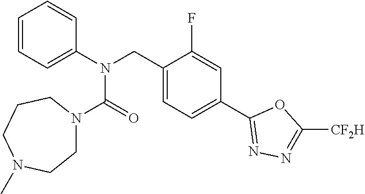 [SMILES: CN1CCCN(C(=O)N(Cc2ccc(-c3nnc(C(F)F)o3)cc2F)c2ccccc2)CC1] |
| 94 | 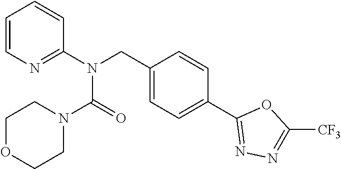 [SMILES: O=C(N1CCOCC1)N(Cc1ccc(-c2nnc(C(F)(F)F)o2)cc1)c1ccccn1] |
| 95 | 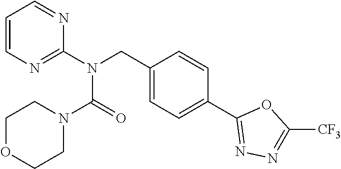 [SMILES: O=C(N1CCOCC1)N(Cc1ccc(-c2nnc(C(F)(F)F)o2)cc1)c1ncccn1] |
| 96 | 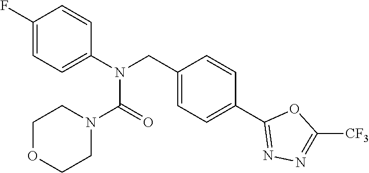 [SMILES: O=C(N1CCOCC1)N(Cc1ccc(-c2nnc(C(F)(F)F)o2)cc1)c1ccc(F)cc1] |
| 97 | 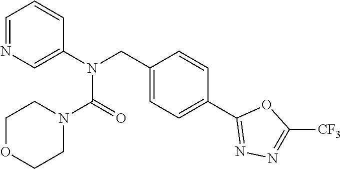 [SMILES: O=C(N1CCOCC1)N(Cc1ccc(-c2nnc(C(F)(F)F)o2)cc1)c1cccnc1] |
| 98 | 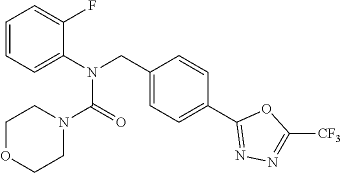 [SMILES: O=C(N1CCOCC1)N(Cc1ccc(-c2nnc(C(F)(F)F)o2)cc1)c1ccccc1F] |
| 99 | 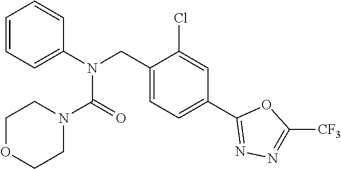 [SMILES: O=C(N1CCOCC1)N(Cc1ccc(-c2nnc(C(F)(F)F)o2)cc1Cl)c1ccccc1] |
| 100 | 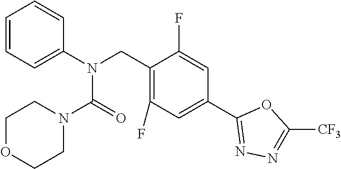 [SMILES: O=C(N1CCOCC1)N(Cc1c(F)cc(-c2nnc(C(F)(F)F)o2)cc1F)c1ccccc1] |
| 101 | 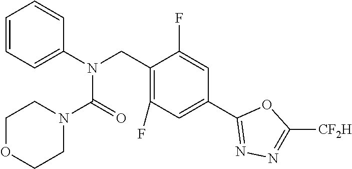 [SMILES: O=C(N1CCOCC1)N(Cc1c(F)cc(-c2nnc(C(F)F)o2)cc1F)c1ccccc1] |
| 102 | 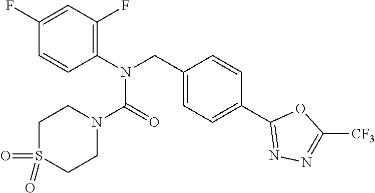 [SMILES: O=C(N1CCS(=O)(=O)CC1)N(Cc1ccc(-c2nnc(C(F)(F)F)o2)cc1)c1ccc(F)cc1F] |
| 103 | 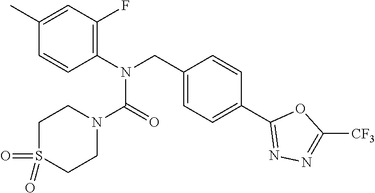 [SMILES: Cc1ccc(N(Cc2ccc(-c3nnc(C(F)(F)F)o3)cc2)C(=O)N2CCS(=O)(=O)CC2)c(F)c1] |
| 104 | 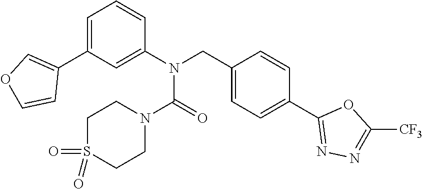 [SMILES: O=C(N1CCS(=O)(=O)CC1)N(Cc1ccc(-c2nnc(C(F)(F)F)o2)cc1)c1cccc(-c2ccoc2)c1] |
| 105 | 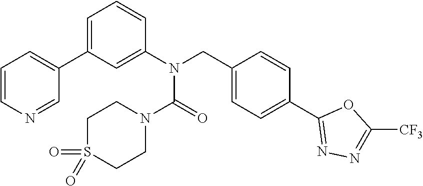 [SMILES: O=C(N1CCS(=O)(=O)CC1)N(Cc1ccc(-c2nnc(C(F)(F)F)o2)cc1)c1cccc(-c2cccnc2)c1] |
| 106 | 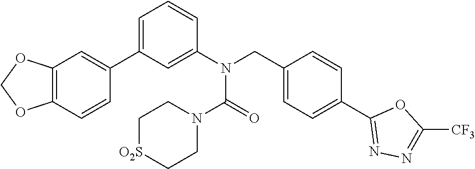 [SMILES: O=C(N1CCS(=O)(=O)CC1)N(Cc1ccc(-c2nnc(C(F)(F)F)o2)cc1)c1cccc(-c2ccc3c(c2)OCO3)c1] |
| 107 | 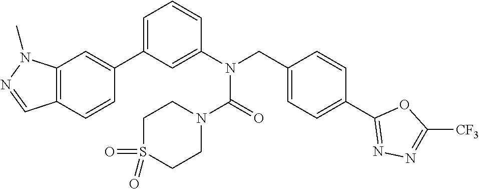 [SMILES: Cn1ncc2ccc(-c3cccc(N(Cc4ccc(-c5nnc(C(F)(F)F)o5)cc4)C(=O)N4CCS(=O)(=O)CC4)c3)cc21] |
| 108 | 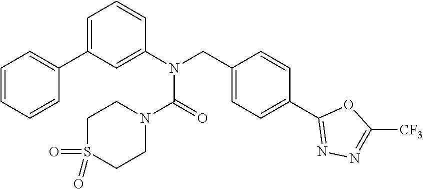 [SMILES: O=C(N1CCS(=O)(=O)CC1)N(Cc1ccc(-c2nnc(C(F)(F)F)o2)cc1)c1cccc(-c2ccccc2)c1] |
| 109 | 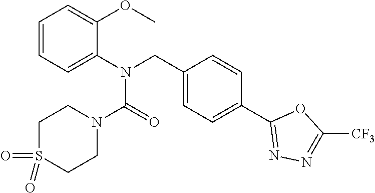 [SMILES: COc1ccccc1N(Cc1ccc(-c2nnc(C(F)(F)F)o2)cc1)C(=O)N1CCS(=O)(=O)CC1] |
| 110 | 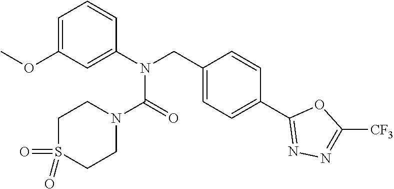 [SMILES: COc1cccc(N(Cc2ccc(-c3nnc(C(F)(F)F)o3)cc2)C(=O)N2CCS(=O)(=O)CC2)c1] |
| 111 | 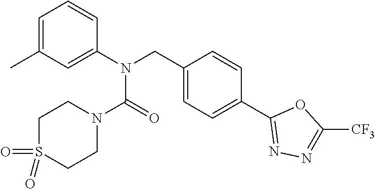 [SMILES: Cc1cccc(N(Cc2ccc(-c3nnc(C(F)(F)F)o3)cc2)C(=O)N2CCS(=O)(=O)CC2)c1] |
| 112 | 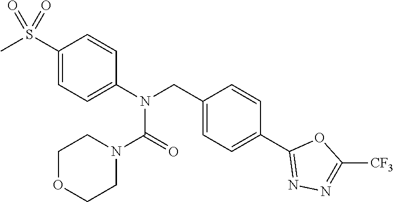 [SMILES: CS(=O)(=O)c1ccc(N(Cc2ccc(-c3nnc(C(F)(F)F)o3)cc2)C(=O)N2CCOCC2)cc1] |
| 113 | 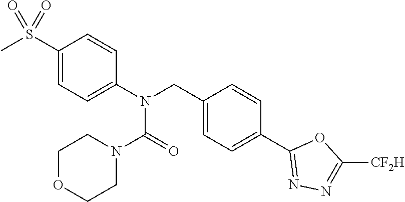 [SMILES: CS(=O)(=O)c1ccc(N(Cc2ccc(-c3nnc(C(F)F)o3)cc2)C(=O)N2CCOCC2)cc1] |
| 114 | 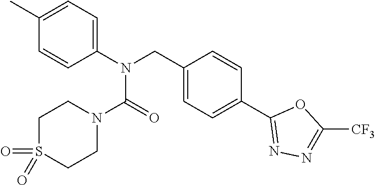 [SMILES: Cc1ccc(N(Cc2ccc(-c3nnc(C(F)(F)F)o3)cc2)C(=O)N2CCS(=O)(=O)CC2)cc1] |
| 115 | 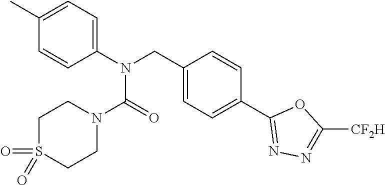 [SMILES: Cc1ccc(N(Cc2ccc(-c3nnc(C(F)F)o3)cc2)C(=O)N2CCS(=O)(=O)CC2)cc1] |
| 116 | 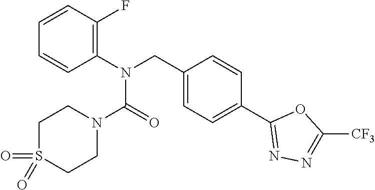 [SMILES: O=C(N1CCS(=O)(=O)CC1)N(Cc1ccc(-c2nnc(C(F)(F)F)o2)cc1)c1ccccc1F] |
| 117 | 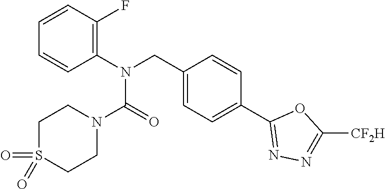 [SMILES: O=C(N1CCS(=O)(=O)CC1)N(Cc1ccc(-c2nnc(C(F)F)o2)cc1)c1ccccc1F] |
| 118 | 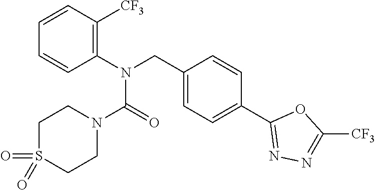 [SMILES: O=C(N1CCS(=O)(=O)CC1)N(Cc1ccc(-c2nnc(C(F)(F)F)o2)cc1)c1ccccc1C(F)(F)F] |
| 119 | 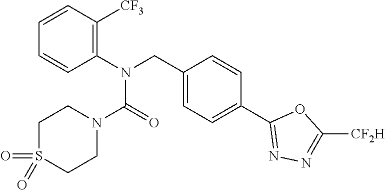 [SMILES: O=C(N1CCS(=O)(=O)CC1)N(Cc1ccc(-c2nnc(C(F)F)o2)cc1)c1ccccc1C(F)(F)F] |
| 120 | 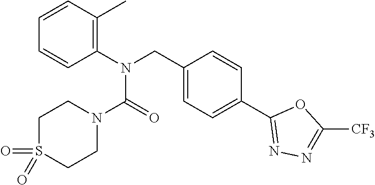 [SMILES: Cc1ccccc1N(Cc1ccc(-c2nnc(C(F)(F)F)o2)cc1)C(=O)N1CCS(=O)(=O)CC1] |
| 121 | 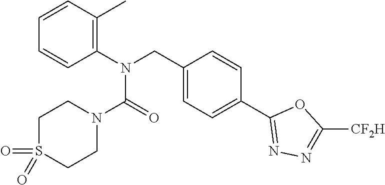 [SMILES: Cc1ccccc1N(Cc1ccc(-c2nnc(C(F)F)o2)cc1)C(=O)N1CCS(=O)(=O)CC1] |
| 122 | 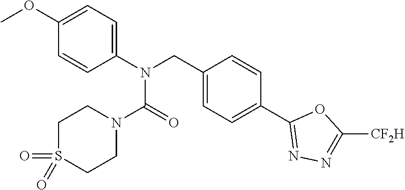 [SMILES: COc1ccc(N(Cc2ccc(-c3nnc(C(F)F)o3)cc2)C(=O)N2CCS(=O)(=O)CC2)cc1] |
| 123 | 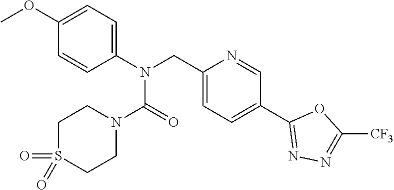 [SMILES: COc1ccc(N(Cc2ccc(-c3nnc(C(F)(F)F)o3)cn2)C(=O)N2CCS(=O)(=O)CC2)cc1] |
| 124 | 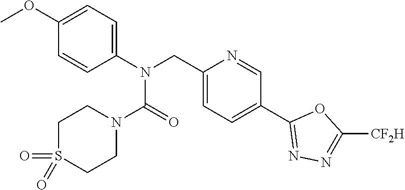 [SMILES: COc1ccc(N(Cc2ccc(-c3nnc(C(F)F)o3)cn2)C(=O)N2CCS(=O)(=O)CC2)cc1] |
| 125 | 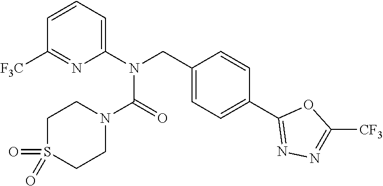 [SMILES: O=C(N1CCS(=O)(=O)CC1)N(Cc1ccc(-c2nnc(C(F)(F)F)o2)cc1)c1cccc(C(F)(F)F)n1] |
| 126 | 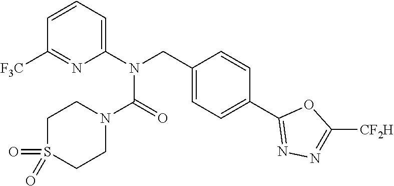 [SMILES: O=C(N1CCS(=O)(=O)CC1)N(Cc1ccc(-c2nnc(C(F)F)o2)cc1)c1cccc(C(F)(F)F)n1] |
| 127 | 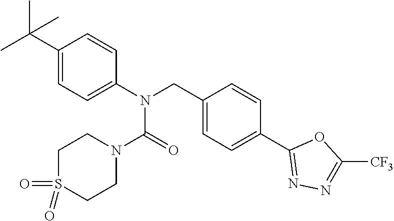 [SMILES: CC(C)(C)c1ccc(N(Cc2ccc(-c3nnc(C(F)(F)F)o3)cc2)C(=O)N2CCS(=O)(=O)CC2)cc1] |
| 128 | 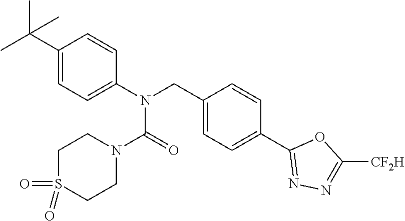 [SMILES: CC(C)(C)c1ccc(N(Cc2ccc(-c3nnc(C(F)F)o3)cc2)C(=O)N2CCS(=O)(=O)CC2)cc1] |
| 129 | 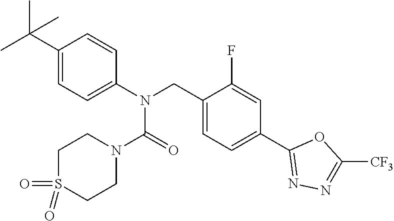 [SMILES: CC(C)(C)c1ccc(N(Cc2ccc(-c3nnc(C(F)(F)F)o3)cc2F)C(=O)N2CCS(=O)(=O)CC2)cc1] |
| 130 | 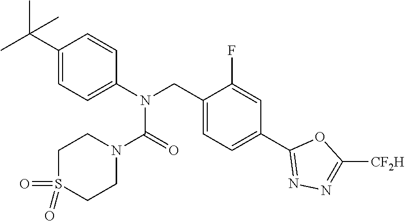 [SMILES: CC(C)(C)c1ccc(N(Cc2ccc(-c3nnc(C(F)F)o3)cc2F)C(=O)N2CCS(=O)(=O)CC2)cc1] |
| 131 | 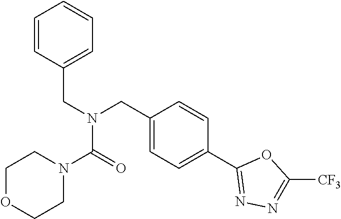 [SMILES: O=C(N1CCOCC1)N(Cc1ccccc1)Cc1ccc(-c2nnc(C(F)(F)F)o2)cc1] |
| 132 | 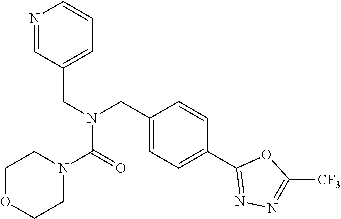 [SMILES: O=C(N1CCOCC1)N(Cc1ccc(-c2nnc(C(F)(F)F)o2)cc1)Cc1cccnc1] |
| 133 | 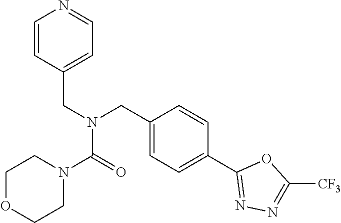 [SMILES: O=C(N1CCOCC1)N(Cc1ccncc1)Cc1ccc(-c2nnc(C(F)(F)F)o2)cc1] |
| 134 | 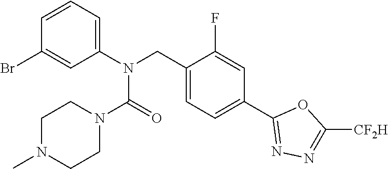 [SMILES: CN1CCN(C(=O)N(Cc2ccc(-c3nnc(C(F)F)o3)cc2F)c2cccc(Br)c2)CC1] |
| 135 | 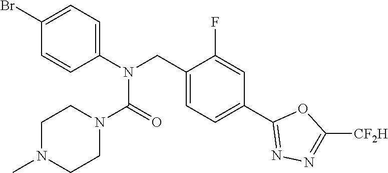 [SMILES: CN1CCN(C(=O)N(Cc2ccc(-c3nnc(C(F)F)o3)cc2F)c2ccc(Br)cc2)CC1] |
| 136 | 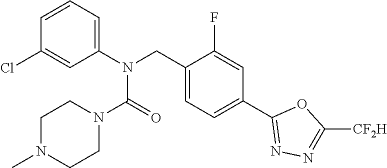 [SMILES: CN1CCN(C(=O)N(Cc2ccc(-c3nnc(C(F)F)o3)cc2F)c2cccc(Cl)c2)CC1] |
| 137 | 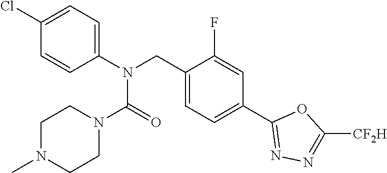 [SMILES: CN1CCN(C(=O)N(Cc2ccc(-c3nnc(C(F)F)o3)cc2F)c2ccc(Cl)cc2)CC1] |
| 138 | 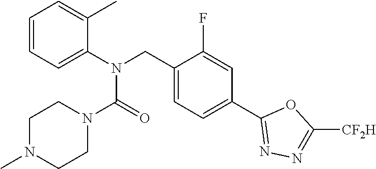 [SMILES: Cc1ccccc1N(Cc1ccc(-c2nnc(C(F)F)o2)cc1F)C(=O)N1CCN(C)CC1] |
| 139 | 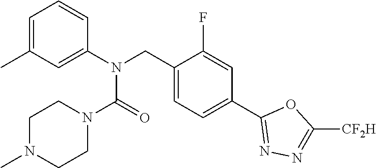 [SMILES: Cc1cccc(N(Cc2ccc(-c3nnc(C(F)F)o3)cc2F)C(=O)N2CCN(C)CC2)c1] |
| 140 | 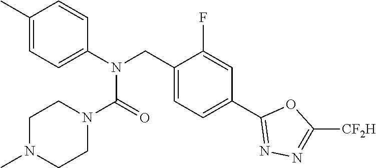 [SMILES: Cc1ccc(N(Cc2ccc(-c3nnc(C(F)F)o3)cc2F)C(=O)N2CCN(C)CC2)cc1] |
| 141 | 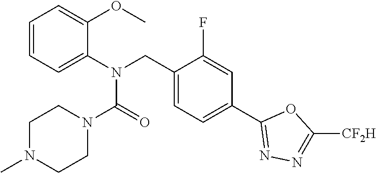 [SMILES: COc1ccccc1N(Cc1ccc(-c2nnc(C(F)F)o2)cc1F)C(=O)N1CCN(C)CC1] |
| 142 | 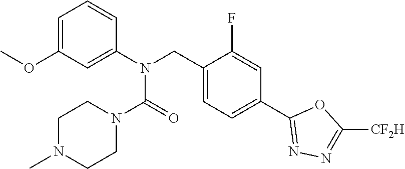 [SMILES: COc1cccc(N(Cc2ccc(-c3nnc(C(F)F)o3)cc2F)C(=O)N2CCN(C)CC2)c1] |
| 143 | 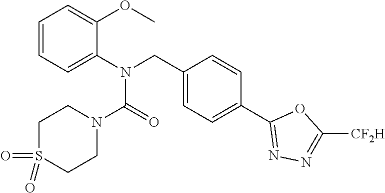 [SMILES: COc1ccccc1N(Cc1ccc(-c2nnc(C(F)F)o2)cc1)C(=O)N1CCS(=O)(=O)CC1] |
| 144 | 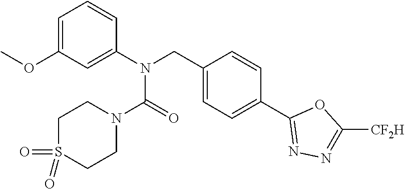 [SMILES: COc1cccc(N(Cc2ccc(-c3nnc(C(F)F)o3)cc2)C(=O)N2CCS(=O)(=O)CC2)c1] |
| 145 | 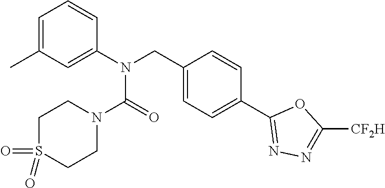 [SMILES: Cc1cccc(N(Cc2ccc(-c3nnc(C(F)F)o3)cc2)C(=O)N2CCS(=O)(=O)CC2)c1] |
| 146 | 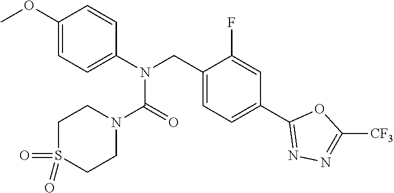 [SMILES: COc1ccc(N(Cc2ccc(-c3nnc(C(F)(F)F)o3)cc2F)C(=O)N2CCS(=O)(=O)CC2)cc1] |
| 147 | 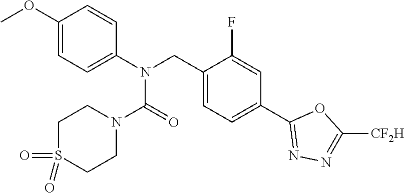 [SMILES: COc1ccc(N(Cc2ccc(-c3nnc(C(F)F)o3)cc2F)C(=O)N2CCS(=O)(=O)CC2)cc1] |
| 148 | 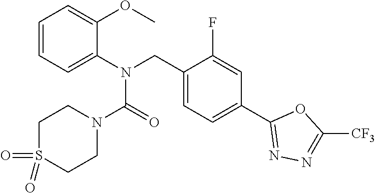 [SMILES: COc1ccccc1N(Cc1ccc(-c2nnc(C(F)(F)F)o2)cc1F)C(=O)N1CCS(=O)(=O)CC1] |
| 149 | 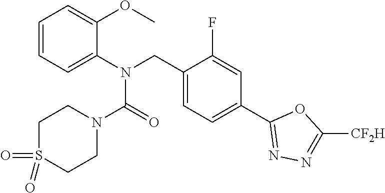 [SMILES: COc1ccccc1N(Cc1ccc(-c2nnc(C(F)F)o2)cc1F)C(=O)N1CCS(=O)(=O)CC1] |
| 150 | 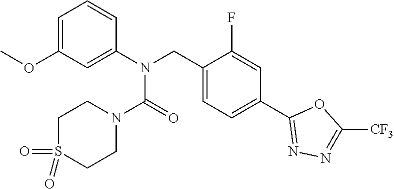 [SMILES: COc1cccc(N(Cc2ccc(-c3nnc(C(F)(F)F)o3)cc2F)C(=O)N2CCS(=O)(=O)CC2)c1] |
| 151 | 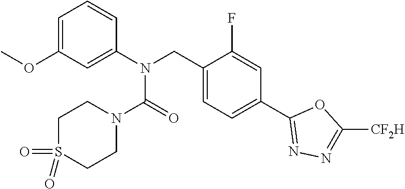 [SMILES: COc1cccc(N(Cc2ccc(-c3nnc(C(F)F)o3)cc2F)C(=O)N2CCS(=O)(=O)CC2)c1] |
| 152 | 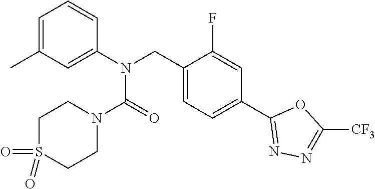 [SMILES: Cc1cccc(N(Cc2ccc(-c3nnc(C(F)(F)F)o3)cc2F)C(=O)N2CCS(=O)(=O)CC2)c1] |
| 153 | 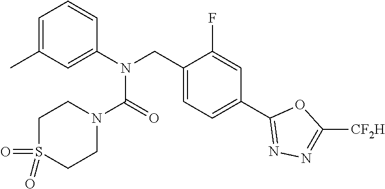 [SMILES: Cc1cccc(N(Cc2ccc(-c3nnc(C(F)F)o3)cc2F)C(=O)N2CCS(=O)(=O)CC2)c1] |
| 154 | 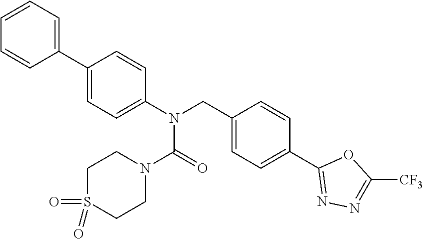 [SMILES: O=C(N1CCS(=O)(=O)CC1)N(Cc1ccc(-c2nnc(C(F)(F)F)o2)cc1)c1ccc(-c2ccccc2)cc1] |
| 155 | 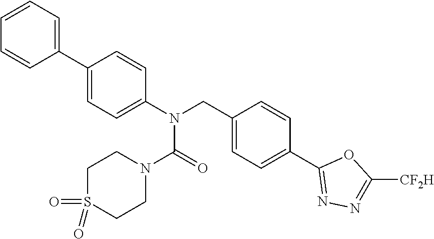 [SMILES: O=C(N1CCS(=O)(=O)CC1)N(Cc1ccc(-c2nnc(C(F)F)o2)cc1)c1ccc(-c2ccccc2)cc1] |
| 156 | 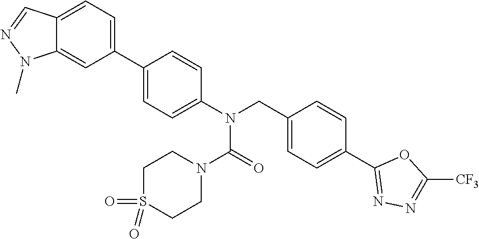 [SMILES: Cn1ncc2ccc(-c3ccc(N(Cc4ccc(-c5nnc(C(F)(F)F)o5)cc4)C(=O)N4CCS(=O)(=O)CC4)cc3)cc21] |
| 157 | 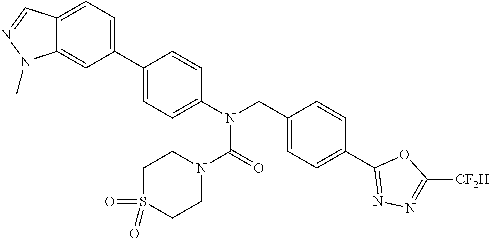 [SMILES: Cn1ncc2ccc(-c3ccc(N(Cc4ccc(-c5nnc(C(F)F)o5)cc4)C(=O)N4CCS(=O)(=O)CC4)cc3)cc21] |
| 158 | 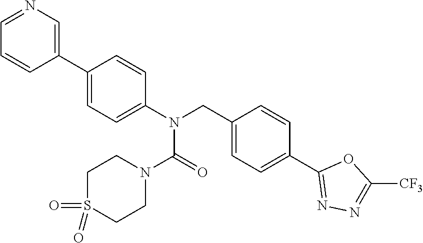 [SMILES: O=C(N1CCS(=O)(=O)CC1)N(Cc1ccc(-c2nnc(C(F)(F)F)o2)cc1)c1ccc(-c2cccnc2)cc1] |
| 159 | 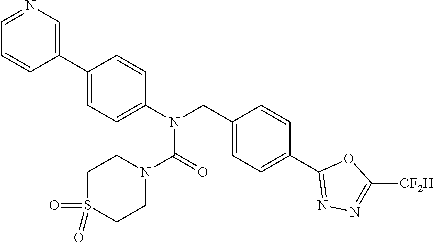 [SMILES: O=C(N1CCS(=O)(=O)CC1)N(Cc1ccc(-c2nnc(C(F)F)o2)cc1)c1ccc(-c2cccnc2)cc1] |
| 160 | 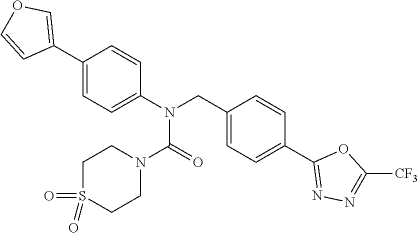 [SMILES: O=C(N1CCS(=O)(=O)CC1)N(Cc1ccc(-c2nnc(C(F)(F)F)o2)cc1)c1ccc(-c2ccoc2)cc1] |
| 161 | 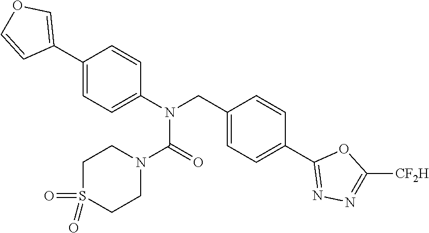 [SMILES: O=C(N1CCS(=O)(=O)CC1)N(Cc1ccc(-c2nnc(C(F)F)o2)cc1)c1ccc(-c2ccoc2)cc1] |
| 162 | 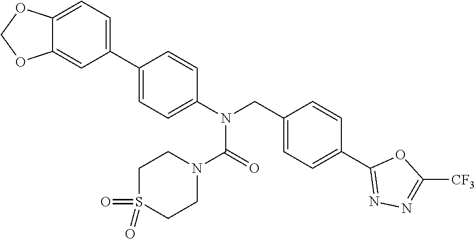 [SMILES: O=C(N1CCS(=O)(=O)CC1)N(Cc1ccc(-c2nnc(C(F)(F)F)o2)cc1)c1ccc(-c2ccc3c(c2)OCO3)cc1] |
| 163 | 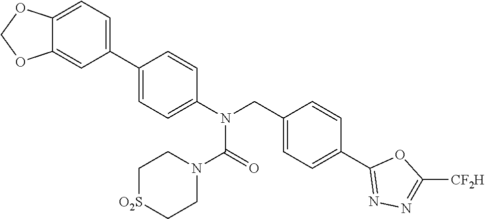 [SMILES: O=C(N1CCS(=O)(=O)CC1)N(Cc1ccc(-c2nnc(C(F)F)o2)cc1)c1ccc(-c2ccc3c(c2)OCO3)cc1] |
| 164 | 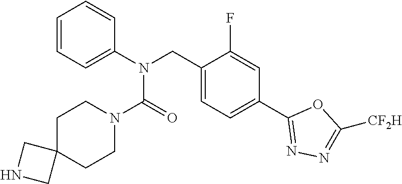 [SMILES: O=C(N1CCC2(CC1)CNC2)N(Cc1ccc(-c2nnc(C(F)F)o2)cc1F)c1ccccc1] |
| 165 | 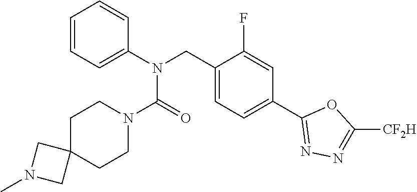 [SMILES: CN1CC2(CCN(C(=O)N(Cc3ccc(-c4nnc(C(F)F)o4)cc3F)c3ccccc3)CC2)C1] |
| 166 | 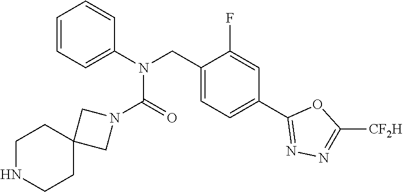 [SMILES: O=C(N1CC2(CCNCC2)C1)N(Cc1ccc(-c2nnc(C(F)F)o2)cc1F)c1ccccc1] |
| 167 | 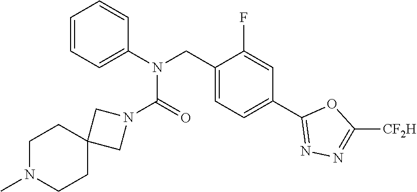 [SMILES: CN1CCC2(CC1)CN(C(=O)N(Cc1ccc(-c3nnc(C(F)F)o3)cc1F)c1ccccc1)C2] |
| 168 | 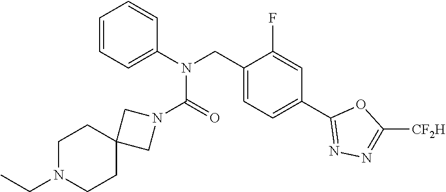 [SMILES: CCN1CCC2(CC1)CN(C(=O)N(Cc1ccc(-c3nnc(C(F)F)o3)cc1F)c1ccccc1)C2] |
| 169 | 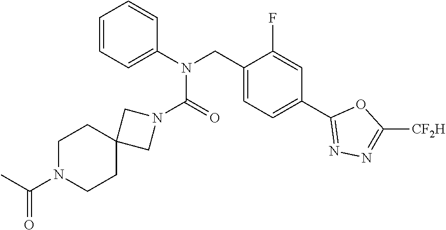 [SMILES: CC(=O)N1CCC2(CC1)CN(C(=O)N(Cc1ccc(-c3nnc(C(F)F)o3)cc1F)c1ccccc1)C2] |
| 170 | 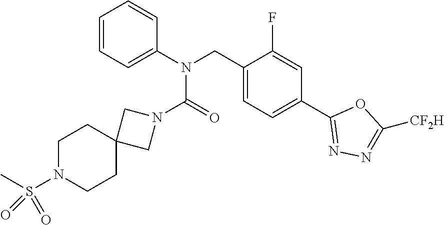 [SMILES: CS(=O)(=O)N1CCC2(CC1)CN(C(=O)N(Cc1ccc(-c3nnc(C(F)F)o3)cc1F)c1ccccc1)C2] |
| 171 | 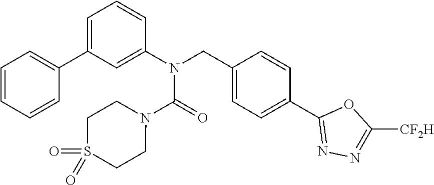 [SMILES: O=C(N1CCS(=O)(=O)CC1)N(Cc1ccc(-c2nnc(C(F)F)o2)cc1)c1cccc(-c2ccccc2)c1] |
| 172 | 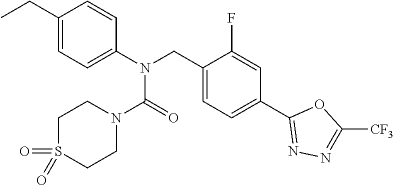 [SMILES: CCc1ccc(N(Cc2ccc(-c3nnc(C(F)(F)F)o3)cc2F)C(=O)N2CCS(=O)(=O)CC2)cc1] |
| 173 | 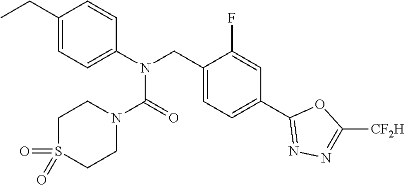 [SMILES: CCc1ccc(N(Cc2ccc(-c3nnc(C(F)F)o3)cc2F)C(=O)N2CCS(=O)(=O)CC2)cc1] |
| 174 | 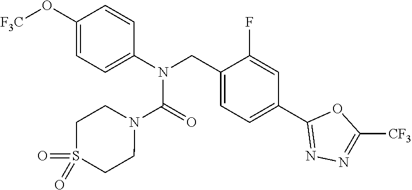 [SMILES: O=C(N1CCS(=O)(=O)CC1)N(Cc1ccc(-c2nnc(C(F)(F)F)o2)cc1F)c1ccc(OC(F)(F)F)cc1] |
| 175 | 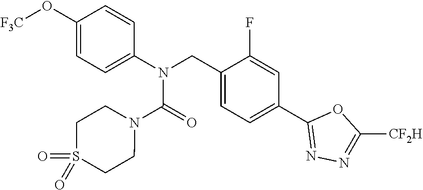 [SMILES: O=C(N1CCS(=O)(=O)CC1)N(Cc1ccc(-c2nnc(C(F)F)o2)cc1F)c1ccc(OC(F)(F)F)cc1] |
| 176 | 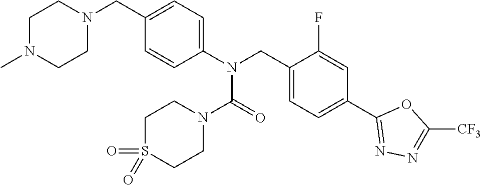 [SMILES: CN1CCN(Cc2ccc(N(Cc3ccc(-c4nnc(C(F)(F)F)o4)cc3F)C(=O)N3CCS(=O)(=O)CC3)cc2)CC1] |
| 177 | 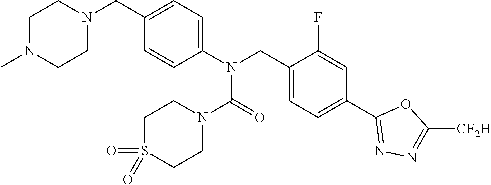 [SMILES: CN1CCN(Cc2ccc(N(Cc3ccc(-c4nnc(C(F)F)o4)cc3F)C(=O)N3CCS(=O)(=O)CC3)cc2)CC1] |
| 178 | 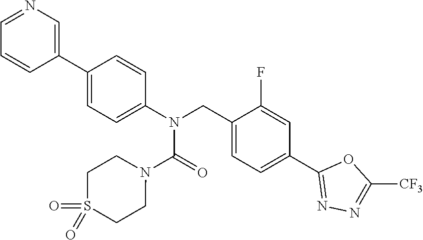 [SMILES: O=C(N1CCS(=O)(=O)CC1)N(Cc1ccc(-c2nnc(C(F)(F)F)o2)cc1F)c1ccc(-c2cccnc2)cc1] |
| 179 | 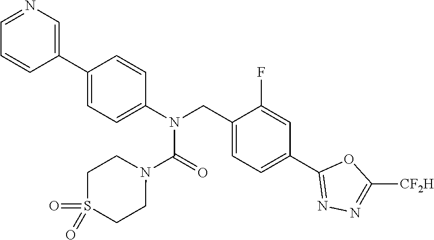 [SMILES: O=C(N1CCS(=O)(=O)CC1)N(Cc1ccc(-c2nnc(C(F)F)o2)cc1F)c1ccc(-c2cccnc2)cc1] |
| 180 | 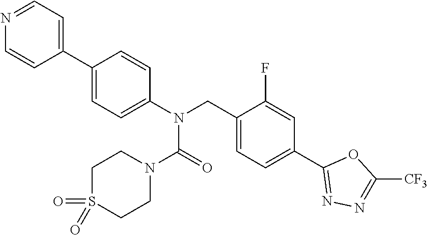 [SMILES: O=C(N1CCS(=O)(=O)CC1)N(Cc1ccc(-c2nnc(C(F)(F)F)o2)cc1F)c1ccc(-c2ccncc2)cc1] |
| 181 | 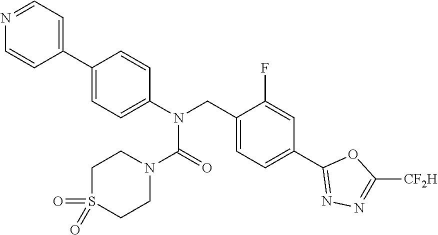 [SMILES: O=C(N1CCS(=O)(=O)CC1)N(Cc1ccc(-c2nnc(C(F)F)o2)cc1F)c1ccc(-c2ccncc2)cc1] |
| 182 | 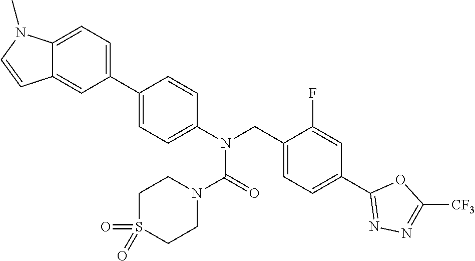 [SMILES: Cn1ccc2cc(-c3ccc(N(Cc4ccc(-c5nnc(C(F)(F)F)o5)cc4F)C(=O)N4CCS(=O)(=O)CC4)cc3)ccc21] |
| 183 | 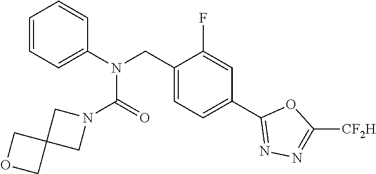 [SMILES: O=C(N1CC2(COC2)C1)N(Cc1ccc(-c2nnc(C(F)F)o2)cc1F)c1ccccc1] |
| 184 | 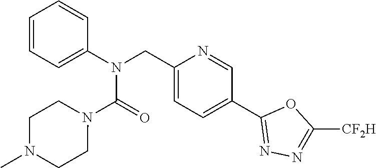 [SMILES: CN1CCN(C(=O)N(Cc2ccc(-c3nnc(C(F)F)o3)cn2)c2ccccc2)CC1] |
| 185 | 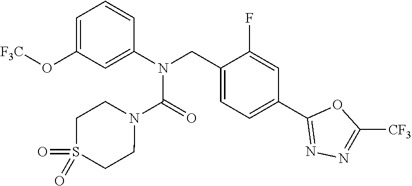 [SMILES: O=C(N1CCS(=O)(=O)CC1)N(Cc1ccc(-c2nnc(C(F)(F)F)o2)cc1F)c1cccc(OC(F)(F)F)c1] |
| 186 | 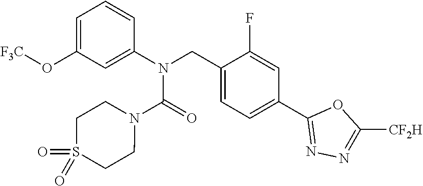 [SMILES: O=C(N1CCS(=O)(=O)CC1)N(Cc1ccc(-c2nnc(C(F)F)o2)cc1F)c1cccc(OC(F)(F)F)c1] |
| 187 | 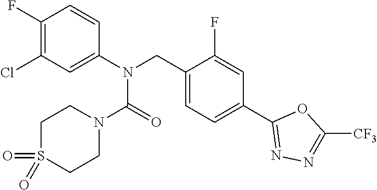 [SMILES: O=C(N1CCS(=O)(=O)CC1)N(Cc1ccc(-c2nnc(C(F)(F)F)o2)cc1F)c1ccc(F)c(Cl)c1] |
| 188 | 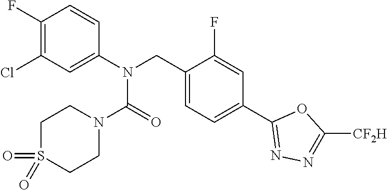 [SMILES: O=C(N1CCS(=O)(=O)CC1)N(Cc1ccc(-c2nnc(C(F)F)o2)cc1F)c1ccc(F)c(Cl)c1] |
| 189 | 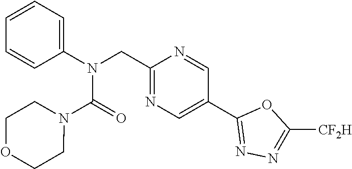 [SMILES: O=C(N1CCOCC1)N(Cc1ncc(-c2nnc(C(F)F)o2)cn1)c1ccccc1] |
| 190 | 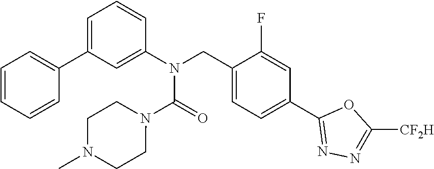 [SMILES: CN1CCN(C(=O)N(Cc2ccc(-c3nnc(C(F)F)o3)cc2F)c2cccc(-c3ccccc3)c2)CC1] |
| 191 | 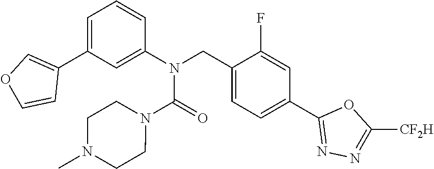 [SMILES: CN1CCN(C(=O)N(Cc2ccc(-c3nnc(C(F)F)o3)cc2F)c2cccc(-c3ccoc3)c2)CC1] |
| 192 | 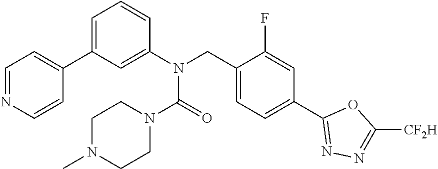 [SMILES: CN1CCN(C(=O)N(Cc2ccc(-c3nnc(C(F)F)o3)cc2F)c2cccc(-c3ccncc3)c2)CC1] |
| 193 | 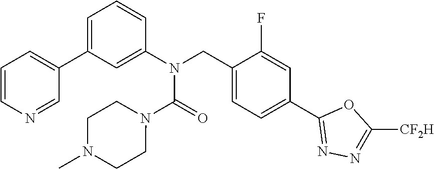 [SMILES: CN1CCN(C(=O)N(Cc2ccc(-c3nnc(C(F)F)o3)cc2F)c2cccc(-c3cccnc3)c2)CC1] |
| 194 | 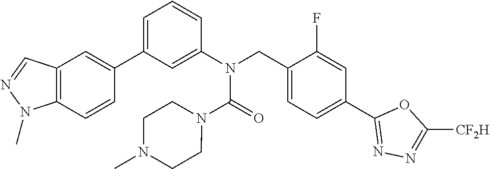 [SMILES: CN1CCN(C(=O)N(Cc2ccc(-c3nnc(C(F)F)o3)cc2F)c2cccc(-c3ccc4c(cnn4C)c3)c2)CC1] |
| 195 | 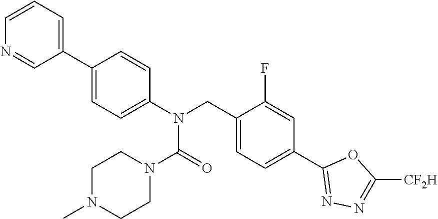 [SMILES: CN1CCN(C(=O)N(Cc2ccc(-c3nnc(C(F)F)o3)cc2F)c2ccc(-c3cccnc3)cc2)CC1] |
| 196 | 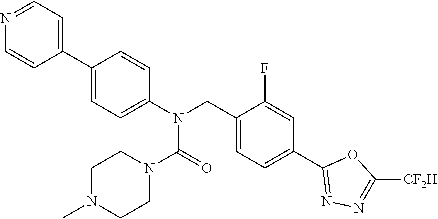 [SMILES: CN1CCN(C(=O)N(Cc2ccc(-c3nnc(C(F)F)o3)cc2F)c2ccc(-c3ccncc3)cc2)CC1] |
| 197 | 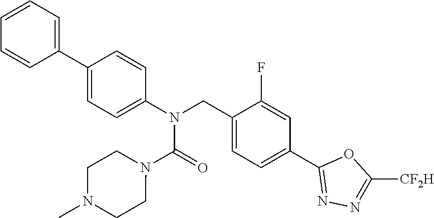 [SMILES: CN1CCN(C(=O)N(Cc2ccc(-c3nnc(C(F)F)o3)cc2F)c2ccc(-c3ccccc3)cc2)CC1] |
| 198 | 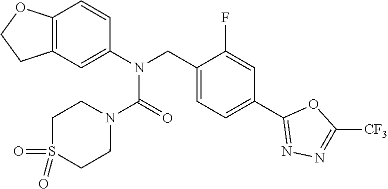 [SMILES: O=C(N1CCS(=O)(=O)CC1)N(Cc1ccc(-c2nnc(C(F)(F)F)o2)cc1F)c1ccc2c(c1)CCO2] |
| 199 | 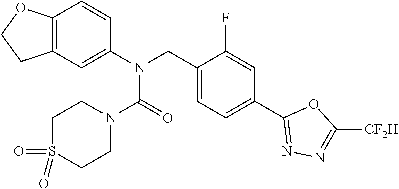 [SMILES: O=C(N1CCS(=O)(=O)CC1)N(Cc1ccc(-c2nnc(C(F)F)o2)cc1F)c1ccc2c(c1)CCO2] |
| 200 | 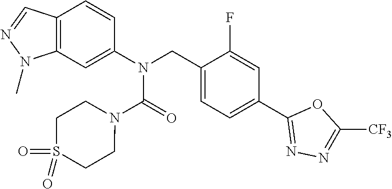 [SMILES: Cn1ncc2ccc(N(Cc3ccc(-c4nnc(C(F)(F)F)o4)cc3F)C(=O)N3CCS(=O)(=O)CC3)cc21] |
| 201 | 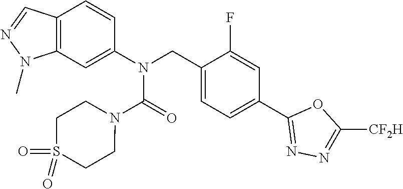 [SMILES: Cn1ncc2ccc(N(Cc3ccc(-c4nnc(C(F)F)o4)cc3F)C(=O)N3CCS(=O)(=O)CC3)cc21] |
| 202 | 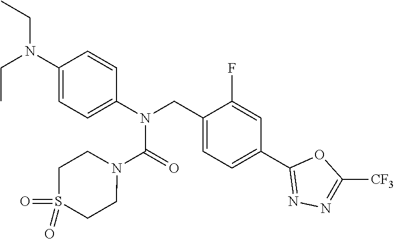 [SMILES: CCN(CC)c1ccc(N(Cc2ccc(-c3nnc(C(F)(F)F)o3)cc2F)C(=O)N2CCS(=O)(=O)CC2)cc1] |
| 203 | 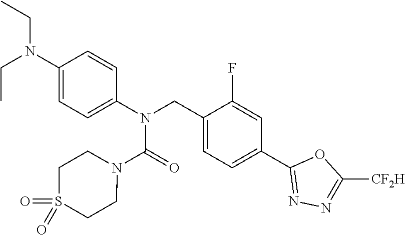 [SMILES: CCN(CC)c1ccc(N(Cc2ccc(-c3nnc(C(F)F)o3)cc2F)C(=O)N2CCS(=O)(=O)CC2)cc1] |
| 204 | 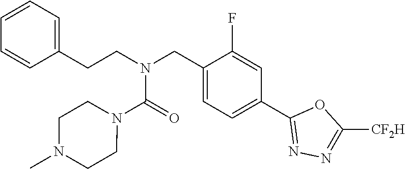 [SMILES: CN1CCN(C(=O)N(CCc2ccccc2)Cc2ccc(-c3nnc(C(F)F)o3)cc2F)CC1] |
| 205 | 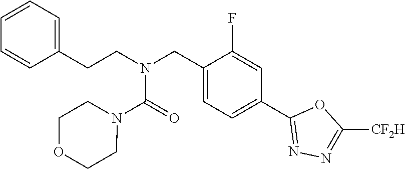 [SMILES: O=C(N1CCOCC1)N(CCc1ccccc1)Cc1ccc(-c2nnc(C(F)F)o2)cc1F] |
| 206 | 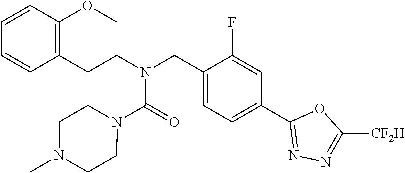 [SMILES: COc1ccccc1CCN(Cc1ccc(-c2nnc(C(F)F)o2)cc1F)C(=O)N1CCN(C)CC1] |
| 207 | 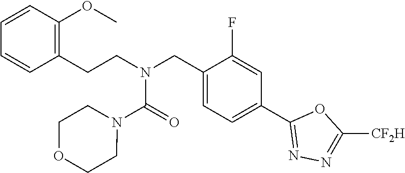 [SMILES: COc1ccccc1CCN(Cc1ccc(-c2nnc(C(F)F)o2)cc1F)C(=O)N1CCOCC1] |
| 208 | 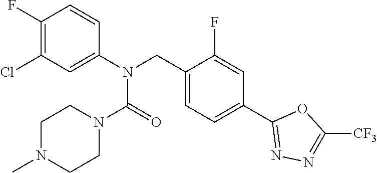 [SMILES: CN1CCN(C(=O)N(Cc2ccc(-c3nnc(C(F)(F)F)o3)cc2F)c2ccc(F)c(Cl)c2)CC1] |
| 209 | 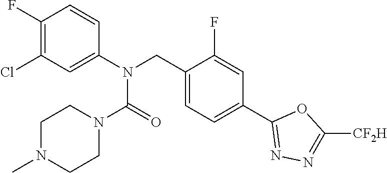 [SMILES: CN1CCN(C(=O)N(Cc2ccc(-c3nnc(C(F)F)o3)cc2F)c2ccc(F)c(Cl)c2)CC1] |
| 210 | 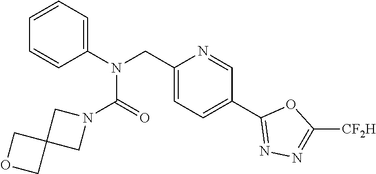 [SMILES: O=C(N1CC2(COC2)C1)N(Cc1ccc(-c2nnc(C(F)F)o2)cn1)c1ccccc1] |
| 211 | 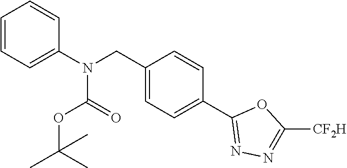 [SMILES: CC(C)(C)OC(=O)N(Cc1ccc(-c2nnc(C(F)F)o2)cc1)c1ccccc1] |
| 212 | 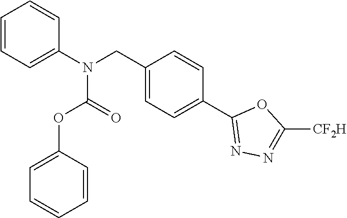 [SMILES: O=C(Oc1ccccc1)N(Cc1ccc(-c2nnc(C(F)F)o2)cc1)c1ccccc1] |
| 213 | 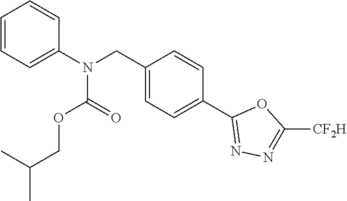 [SMILES: CC(C)COC(=O)N(Cc1ccc(-c2nnc(C(F)F)o2)cc1)c1ccccc1] |
| 214 | 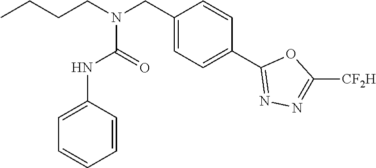 [SMILES: CCCCN(Cc1ccc(-c2nnc(C(F)F)o2)cc1)C(=O)Nc1ccccc1] |
| 215 | 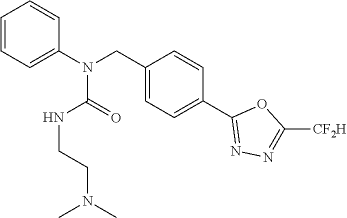 [SMILES: CN(C)CCNC(=O)N(Cc1ccc(-c2nnc(C(F)F)o2)cc1)c1ccccc1] |
| 216 | 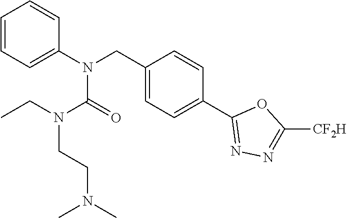 [SMILES: CCN(CCN(C)C)C(=O)N(Cc1ccc(-c2nnc(C(F)F)o2)cc1)c1ccccc1] |
| 217 | 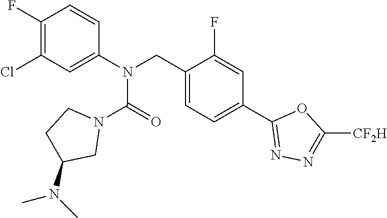 [SMILES: CN(C)[C@H]1CCN(C(=O)N(Cc2ccc(-c3nnc(C(F)F)o3)cc2F)c2ccc(F)c(Cl)c2)C1] |
| 218 | 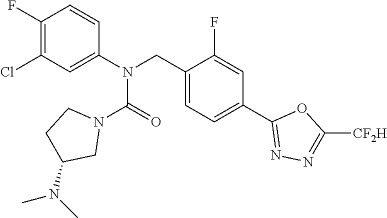 [SMILES: CN(C)[C@@H]1CCN(C(=O)N(Cc2ccc(-c3nnc(C(F)F)o3)cc2F)c2ccc(F)c(Cl)c2)C1] |
| 219 | 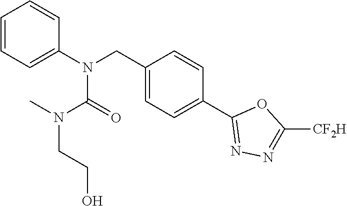 [SMILES: CN(CCO)C(=O)N(Cc1ccc(-c2nnc(C(F)F)o2)cc1)c1ccccc1] |
| 220 | 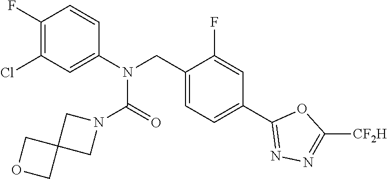 [SMILES: O=C(N1CC2(COC2)C1)N(Cc1ccc(-c2nnc(C(F)F)o2)cc1F)c1ccc(F)c(Cl)c1] |
| 221 | 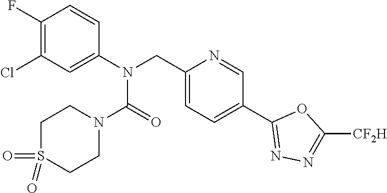 [SMILES: O=C(N1CCS(=O)(=O)CC1)N(Cc1ccc(-c2nnc(C(F)F)o2)cn1)c1ccc(F)c(Cl)c1] |
| 222 | 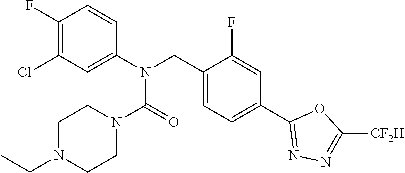 [SMILES: CCN1CCN(C(=O)N(Cc2ccc(-c3nnc(C(F)F)o3)cc2F)c2ccc(F)c(Cl)c2)CC1] |
| 223 | 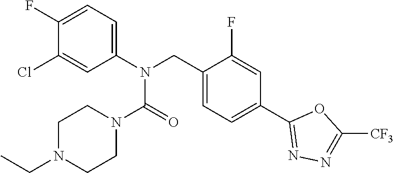 [SMILES: CCN1CCN(C(=O)N(Cc2ccc(-c3nnc(C(F)(F)F)o3)cc2F)c2ccc(F)c(Cl)c2)CC1] |
| 224 | 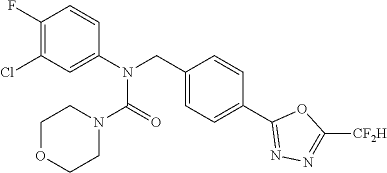 [SMILES: O=C(N1CCOCC1)N(Cc1ccc(-c2nnc(C(F)F)o2)cc1)c1ccc(F)c(Cl)c1] |
| 225 | 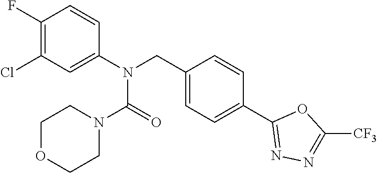 [SMILES: O=C(N1CCOCC1)N(Cc1ccc(-c2nnc(C(F)(F)F)o2)cc1)c1ccc(F)c(Cl)c1] |
| 226 | 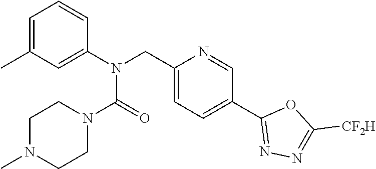 [SMILES: Cc1cccc(N(Cc2ccc(-c3nnc(C(F)F)o3)cn2)C(=O)N2CCN(C)CC2)c1] |
| 227 | 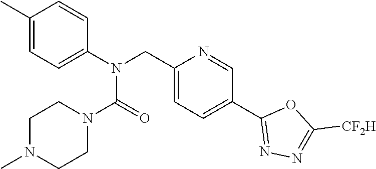 [SMILES: Cc1ccc(N(Cc2ccc(-c3nnc(C(F)F)o3)cn2)C(=O)N2CCN(C)CC2)cc1] |
| 228 | 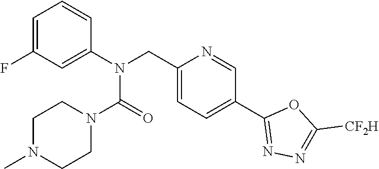 [SMILES: CN1CCN(C(=O)N(Cc2ccc(-c3nnc(C(F)F)o3)cn2)c2cccc(F)c2)CC1] |
| 229 | 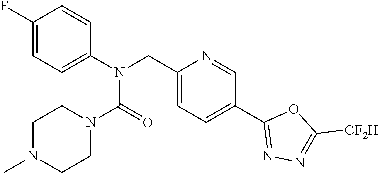 [SMILES: CN1CCN(C(=O)N(Cc2ccc(-c3nnc(C(F)F)o3)cn2)c2ccc(F)cc2)CC1] |
| 230 | 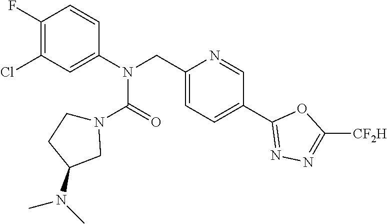 [SMILES: CN(C)[C@H]1CCN(C(=O)N(Cc2ccc(-c3nnc(C(F)F)o3)cn2)c2ccc(F)c(Cl)c2)C1] |
| 231 | 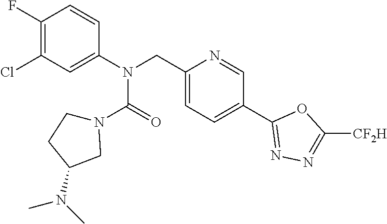 [SMILES: CN(C)[C@@H]1CCN(C(=O)N(Cc2ccc(-c3nnc(C(F)F)o3)cn2)c2ccc(F)c(Cl)c2)C1] |
| 232 | 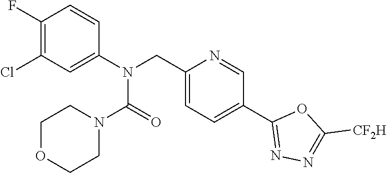 [SMILES: O=C(N1CCOCC1)N(Cc1ccc(-c2nnc(C(F)F)o2)cn1)c1ccc(F)c(Cl)c1] |
| 233 | 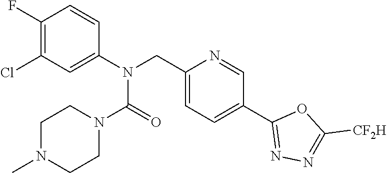 [SMILES: CN1CCN(C(=O)N(Cc2ccc(-c3nnc(C(F)F)o3)cn2)c2ccc(F)c(Cl)c2)CC1] |
| 234 | 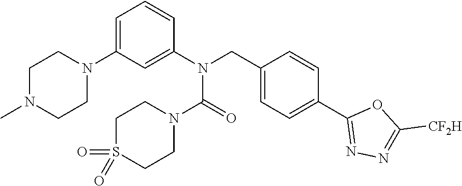 [SMILES: CN1CCN(c2cccc(N(Cc3ccc(-c4nnc(C(F)F)o4)cc3)C(=O)N3CCS(=O)(=O)CC3)c2)CC1] |
| 235 | 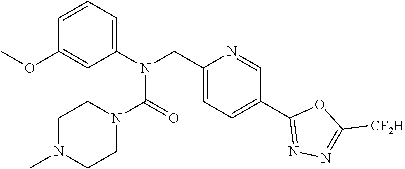 [SMILES: COc1cccc(N(Cc2ccc(-c3nnc(C(F)F)o3)cn2)C(=O)N2CCN(C)CC2)c1] |
| 236 | 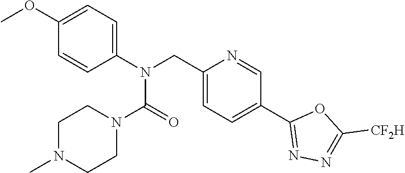 [SMILES: COc1ccc(N(Cc2ccc(-c3nnc(C(F)F)o3)cn2)C(=O)N2CCN(C)CC2)cc1] |
| 237 | 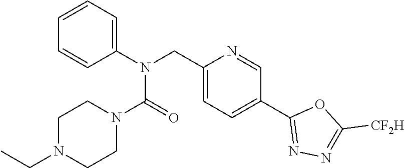 [SMILES: CCN1CCN(C(=O)N(Cc2ccc(-c3nnc(C(F)F)o3)cn2)c2ccccc2)CC1] |
| 238 | 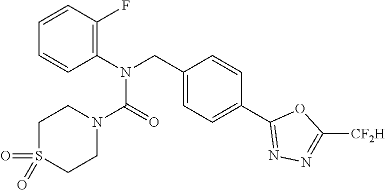 [SMILES: O=C(N1CCS(=O)(=O)CC1)N(Cc1ccc(-c2nnc(C(F)F)o2)cc1)c1ccccc1F] |
| 239 | 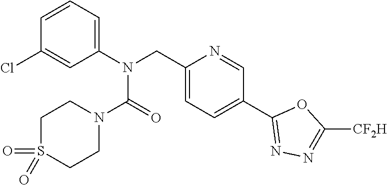 [SMILES: O=C(N1CCS(=O)(=O)CC1)N(Cc1ccc(-c2nnc(C(F)F)o2)cn1)c1cccc(Cl)c1] |
| 240 | 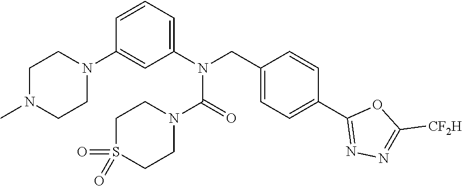 [SMILES: CN1CCN(c2cccc(N(Cc3ccc(-c4nnc(C(F)F)o4)cc3)C(=O)N3CCS(=O)(=O)CC3)c2)CC1] |
| 241 | 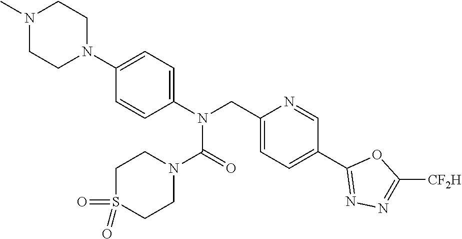 [SMILES: CN1CCN(c2ccc(N(Cc3ccc(-c4nnc(C(F)F)o4)cn3)C(=O)N3CCS(=O)(=O)CC3)cc2)CC1] |
| 242 | 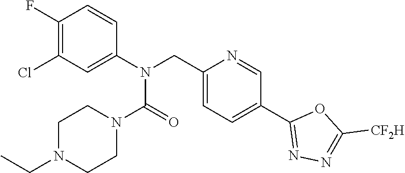 [SMILES: CCN1CCN(C(=O)N(Cc2ccc(-c3nnc(C(F)F)o3)cn2)c2ccc(F)c(Cl)c2)CC1] |
| 243 | 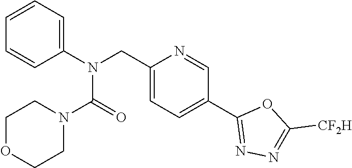 [SMILES: O=C(N1CCOCC1)N(Cc1ccc(-c2nnc(C(F)F)o2)cn1)c1ccccc1] |
| 244 | 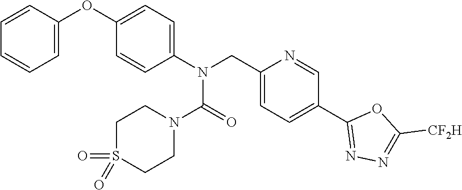 [SMILES: O=C(N1CCS(=O)(=O)CC1)N(Cc1ccc(-c2nnc(C(F)F)o2)cn1)c1ccc(Oc2ccccc2)cc1] |
| 245 | 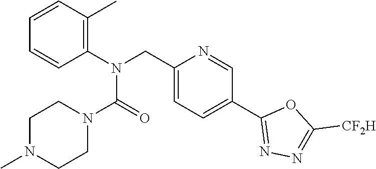 [SMILES: Cc1ccccc1N(Cc1ccc(-c2nnc(C(F)F)o2)cn1)C(=O)N1CCN(C)CC1] |
| 246 | 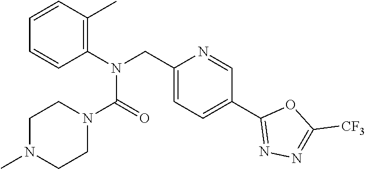 [SMILES: Cc1ccccc1N(Cc1ccc(-c2nnc(C(F)(F)F)o2)cn1)C(=O)N1CCN(C)CC1] |
| 247 | 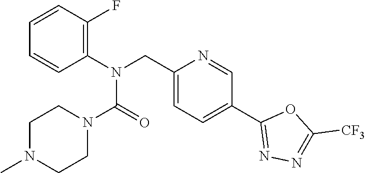 [SMILES: CN1CCN(C(=O)N(Cc2ccc(-c3nnc(C(F)(F)F)o3)cn2)c2ccccc2F)CC1] |
| 248 | 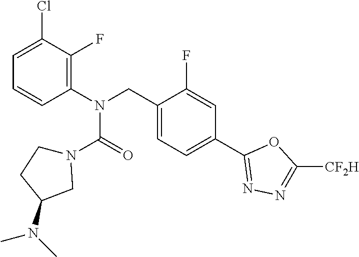 [SMILES: CN(C)[C@H]1CCN(C(=O)N(Cc2ccc(-c3nnc(C(F)F)o3)cc2F)c2cccc(Cl)c2F)C1] |
| 249 | 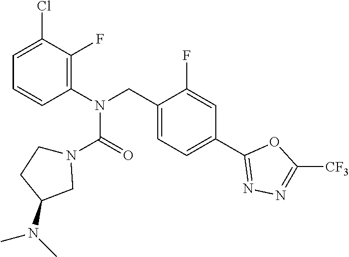 [SMILES: CN(C)[C@H]1CCN(C(=O)N(Cc2ccc(-c3nnc(C(F)(F)F)o3)cc2F)c2cccc(Cl)c2F)C1] |
| 250 | 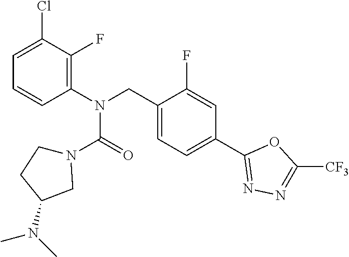 [SMILES: CN(C)[C@@H]1CCN(C(=O)N(Cc2ccc(-c3nnc(C(F)(F)F)o3)cc2F)c2cccc(Cl)c2F)C1] |
| 251 | 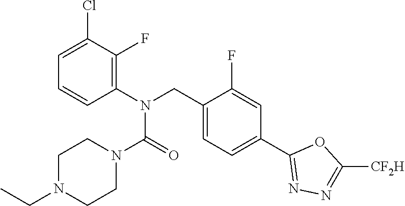 [SMILES: CCN1CCN(C(=O)N(Cc2ccc(-c3nnc(C(F)F)o3)cc2F)c2cccc(Cl)c2F)CC1] |
| 252 | 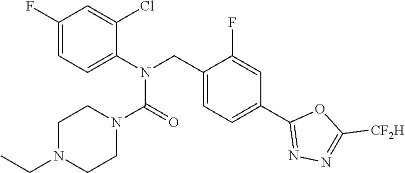 [SMILES: CCN1CCN(C(=O)N(Cc2ccc(-c3nnc(C(F)F)o3)cc2F)c2ccc(F)cc2Cl)CC1] |
| 253 | 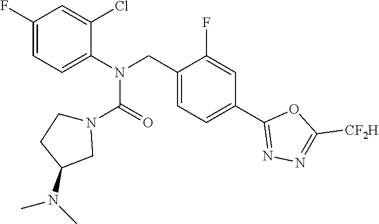 [SMILES: CN(C)[C@H]1CCN(C(=O)N(Cc2ccc(-c3nnc(C(F)F)o3)cc2F)c2ccc(F)cc2Cl)C1] |
| 254 | 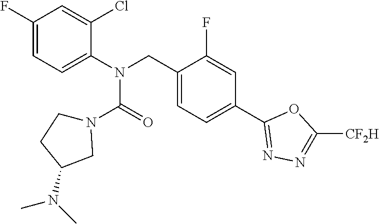 [SMILES: CN(C)[C@@H]1CCN(C(=O)N(Cc2ccc(-c3nnc(C(F)F)o3)cc2F)c2ccc(F)cc2Cl)C1] |
| 255 | 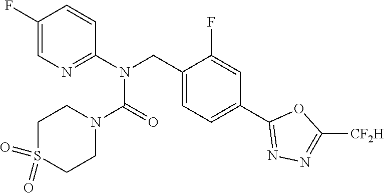 [SMILES: O=C(N1CCS(=O)(=O)CC1)N(Cc1ccc(-c2nnc(C(F)F)o2)cc1F)c1ccc(F)cn1] |
| 256 | 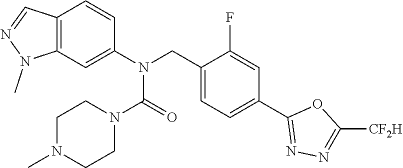 [SMILES: CN1CCN(C(=O)N(Cc2ccc(-c3nnc(C(F)F)o3)cc2F)c2ccc3cnn(C)c3c2)CC1] |
| 257 | 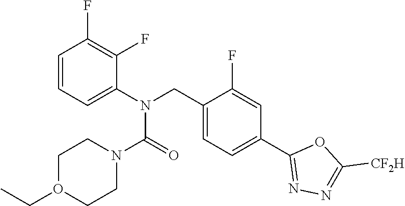 |
| 258 | 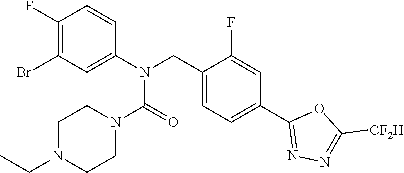 [SMILES: CCN1CCN(C(=O)N(Cc2ccc(-c3nnc(C(F)F)o3)cc2F)c2ccc(F)c(Br)c2)CC1] |
| 259 | 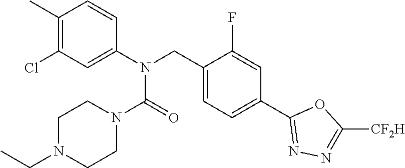 [SMILES: CCN1CCN(C(=O)N(Cc2ccc(-c3nnc(C(F)F)o3)cc2F)c2ccc(C)c(Cl)c2)CC1] |
| 260 | 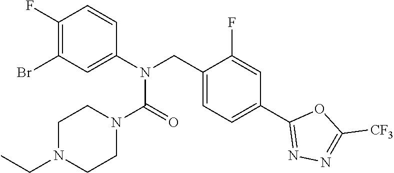 [SMILES: CCN1CCN(C(=O)N(Cc2ccc(-c3nnc(C(F)(F)F)o3)cc2F)c2ccc(F)c(Br)c2)CC1] |
| 261 | 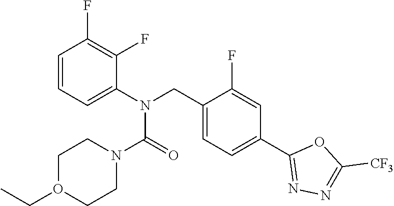 |
| 262 | 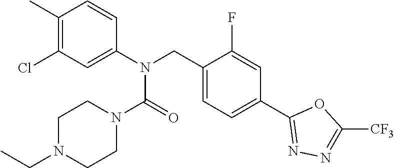 [SMILES: CCN1CCN(C(=O)N(Cc2ccc(-c3nnc(C(F)(F)F)o3)cc2F)c2ccc(C)c(Cl)c2)CC1] |
| 263 | 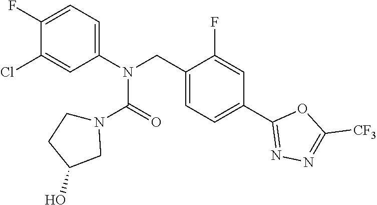 [SMILES: O=C(N1CC[C@@H](O)C1)N(Cc1ccc(-c2nnc(C(F)(F)F)o2)cc1F)c1ccc(F)c(Cl)c1] |
| 264 | 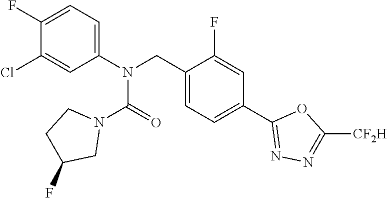 [SMILES: O=C(N1CC[C@H](F)C1)N(Cc1ccc(-c2nnc(C(F)F)o2)cc1F)c1ccc(F)c(Cl)c1] |
| 265 | 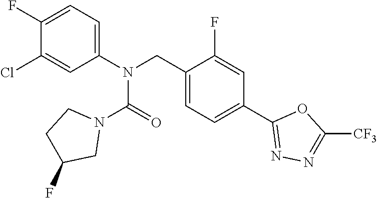 [SMILES: O=C(N1CC[C@H](F)C1)N(Cc1ccc(-c2nnc(C(F)(F)F)o2)cc1F)c1ccc(F)c(Cl)c1] |
| 266 | 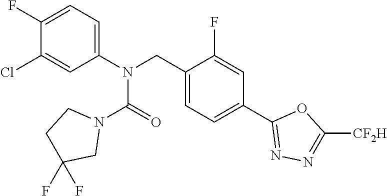 [SMILES: O=C(N1CCC(F)(F)C1)N(Cc1ccc(-c2nnc(C(F)F)o2)cc1F)c1ccc(F)c(Cl)c1] |
| 267 | 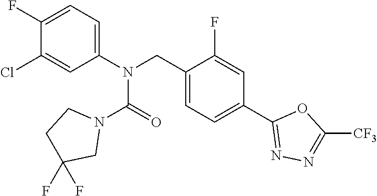 [SMILES: O=C(N1CCC(F)(F)C1)N(Cc1ccc(-c2nnc(C(F)(F)F)o2)cc1F)c1ccc(F)c(Cl)c1] |
| 268 | 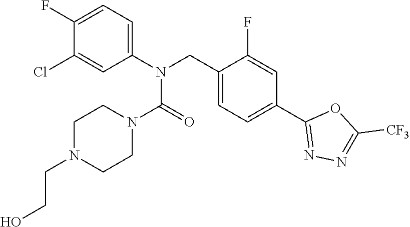 [SMILES: O=C(N1CCN(CCO)CC1)N(Cc1ccc(-c2nnc(C(F)(F)F)o2)cc1F)c1ccc(F)c(Cl)c1] |
| 269 | 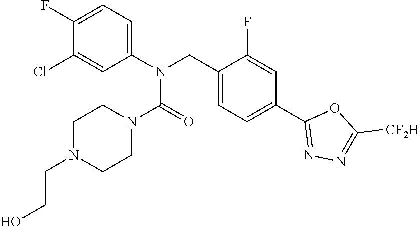 [SMILES: O=C(N1CCN(CCO)CC1)N(Cc1ccc(-c2nnc(C(F)F)o2)cc1F)c1ccc(F)c(Cl)c1] |
| 270 | 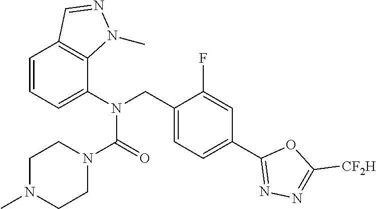 [SMILES: CN1CCN(C(=O)N(Cc2ccc(-c3nnc(C(F)F)o3)cc2F)c2cccc3cnn(C)c23)CC1] |
| 271 | 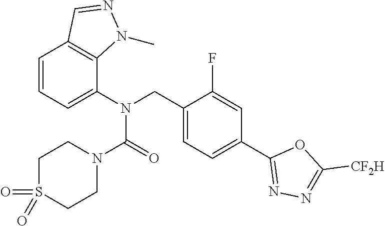 [SMILES: Cn1ncc2cccc(N(Cc3ccc(-c4nnc(C(F)F)o4)cc3F)C(=O)N3CCS(=O)(=O)CC3)c21] |
| 272 | 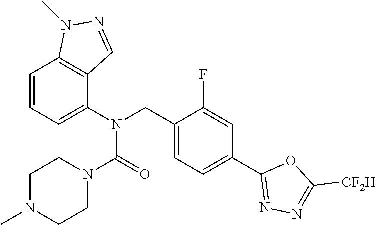 [SMILES: CN1CCN(C(=O)N(Cc2ccc(-c3nnc(C(F)F)o3)cc2F)c2cccc3c2cnn3C)CC1] |
| 273 | 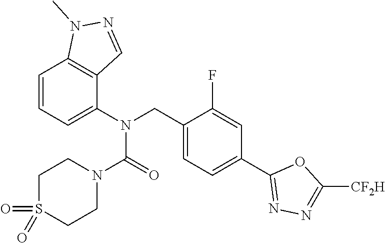 [SMILES: Cn1ncc2c(N(Cc3ccc(-c4nnc(C(F)F)o4)cc3F)C(=O)N3CCS(=O)(=O)CC3)cccc21] |
| 274 | 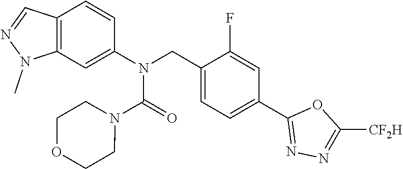 [SMILES: Cn1ncc2ccc(N(Cc3ccc(-c4nnc(C(F)F)o4)cc3F)C(=O)N3CCOCC3)cc21] |
| 275 | 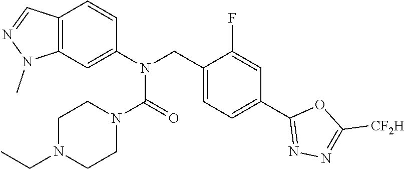 [SMILES: CCN1CCN(C(=O)N(Cc2ccc(-c3nnc(C(F)F)o3)cc2F)c2ccc3cnn(C)c3c2)CC1] |
| 276 | 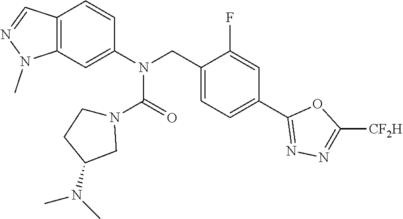 [SMILES: CN(C)[C@@H]1CCN(C(=O)N(Cc2ccc(-c3nnc(C(F)F)o3)cc2F)c2ccc3cnn(C)c3c2)C1] |
| 277 | 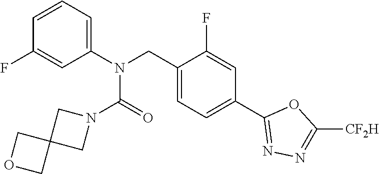 [SMILES: O=C(N1CC2(COC2)C1)N(Cc1ccc(-c2nnc(C(F)F)o2)cc1F)c1cccc(F)c1] |
| 278 | 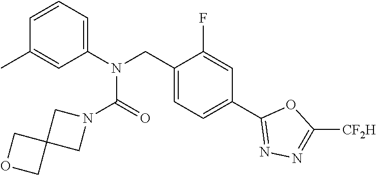 [SMILES: Cc1cccc(N(Cc2ccc(-c3nnc(C(F)F)o3)cc2F)C(=O)N2CC3(COC3)C2)c1] |
| 279 | 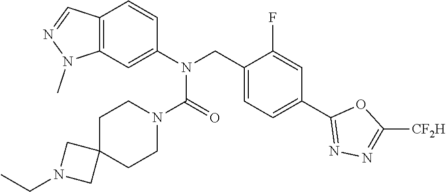 [SMILES: CCN1CC2(CCN(C(=O)N(Cc3ccc(-c4nnc(C(F)F)o4)cc3F)c3ccc4cnn(C)c4c3)CC2)C1] |
| 280 | 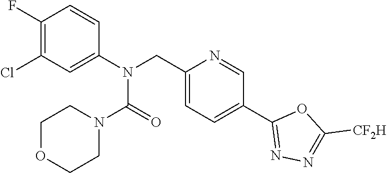 [SMILES: O=C(N1CCOCC1)N(Cc1ccc(-c2nnc(C(F)F)o2)cn1)c1ccc(F)c(Cl)c1] |
| 281 | 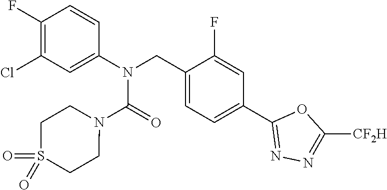 [SMILES: O=C(N1CCS(=O)(=O)CC1)N(Cc1ccc(-c2nnc(C(F)F)o2)cc1F)c1ccc(F)c(Cl)c1] |
| 282 | 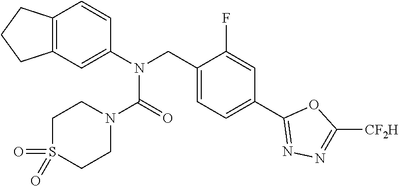 [SMILES: O=C(N1CCS(=O)(=O)CC1)N(Cc1ccc(-c2nnc(C(F)F)o2)cc1F)c1ccc2c(c1)CCC2] |
| 283 | 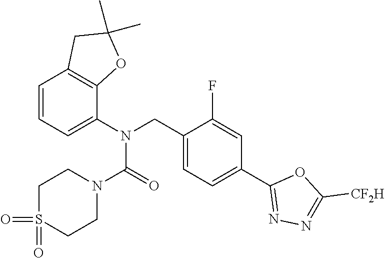 [SMILES: CC1(C)Cc2cccc(N(Cc3ccc(-c4nnc(C(F)F)o4)cc3F)C(=O)N3CCS(=O)(=O)CC3)c2O1] |
| 284 | 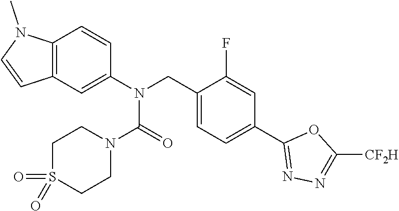 [SMILES: Cn1ccc2cc(N(Cc3ccc(-c4nnc(C(F)F)o4)cc3F)C(=O)N3CCS(=O)(=O)CC3)ccc21] |
| 285 | 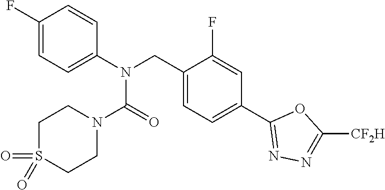 [SMILES: O=C(N1CCS(=O)(=O)CC1)N(Cc1ccc(-c2nnc(C(F)F)o2)cc1F)c1ccc(F)cc1] |
| 286 | 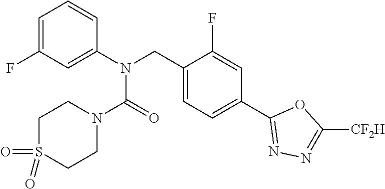 [SMILES: O=C(N1CCS(=O)(=O)CC1)N(Cc1ccc(-c2nnc(C(F)F)o2)cc1F)c1cccc(F)c1] |
| 287 | 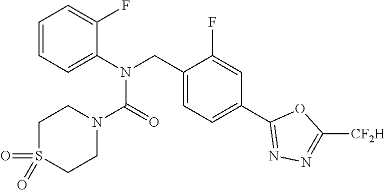 [SMILES: O=C(N1CCS(=O)(=O)CC1)N(Cc1ccc(-c2nnc(C(F)F)o2)cc1F)c1ccccc1F] |
| 288 | 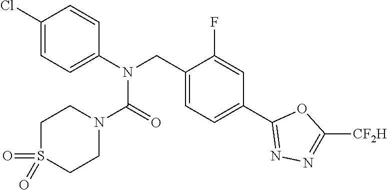 [SMILES: O=C(N1CCS(=O)(=O)CC1)N(Cc1ccc(-c2nnc(C(F)F)o2)cc1F)c1ccc(Cl)cc1] |
| 289 | 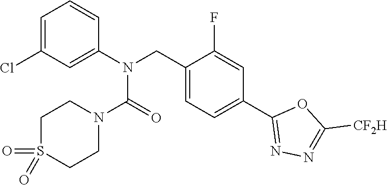 [SMILES: O=C(N1CCS(=O)(=O)CC1)N(Cc1ccc(-c2nnc(C(F)F)o2)cc1F)c1cccc(Cl)c1] |
| 290 | 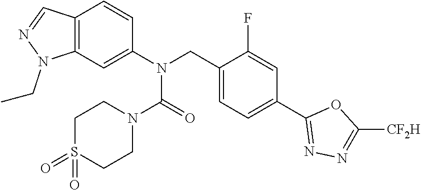 [SMILES: CCn1ncc2ccc(N(Cc3ccc(-c4nnc(C(F)F)o4)cc3F)C(=O)N3CCS(=O)(=O)CC3)cc21] |
| 291 | 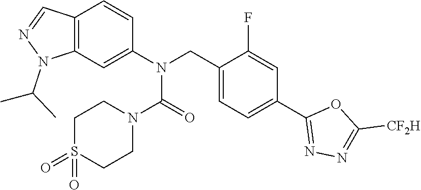 [SMILES: CC(C)n1ncc2ccc(N(Cc3ccc(-c4nnc(C(F)F)o4)cc3F)C(=O)N3CCS(=O)(=O)CC3)cc21] |
| 292 | 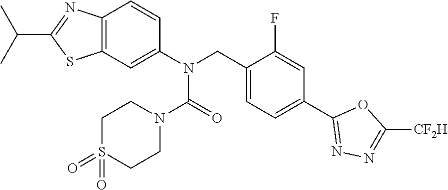 [SMILES: CC(C)c1nc2ccc(N(Cc3ccc(-c4nnc(C(F)F)o4)cc3F)C(=O)N3CCS(=O)(=O)CC3)cc2s1] |
| 293 | 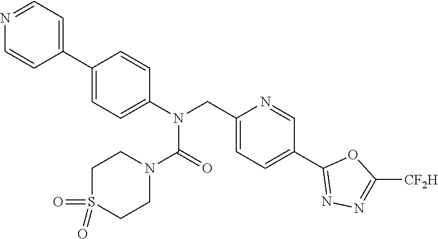 [SMILES: O=C(N1CCS(=O)(=O)CC1)N(Cc1ccc(-c2nnc(C(F)F)o2)cn1)c1ccc(-c2ccncc2)cc1] |
| 294 | 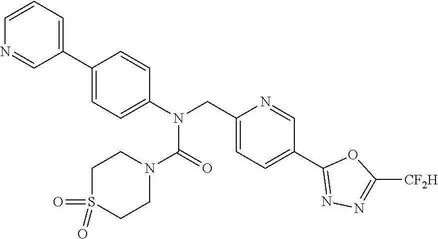 [SMILES: O=C(N1CCS(=O)(=O)CC1)N(Cc1ccc(-c2nnc(C(F)F)o2)cn1)c1ccc(-c2cccnc2)cc1] |
| 295 | 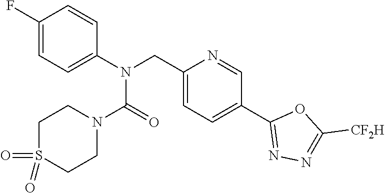 [SMILES: O=C(N1CCS(=O)(=O)CC1)N(Cc1ccc(-c2nnc(C(F)F)o2)cn1)c1ccc(F)cc1] |
| 296 | 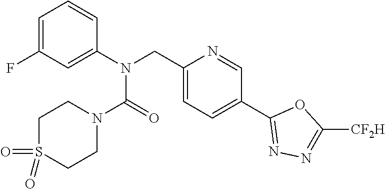 [SMILES: O=C(N1CCS(=O)(=O)CC1)N(Cc1ccc(-c2nnc(C(F)F)o2)cn1)c1cccc(F)c1] |
| 297 | 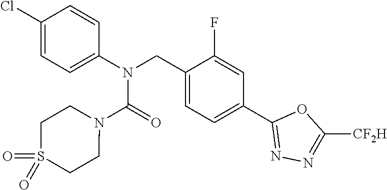 [SMILES: O=C(N1CCS(=O)(=O)CC1)N(Cc1ccc(-c2nnc(C(F)F)o2)cc1F)c1ccc(Cl)cc1] |
| 298 | 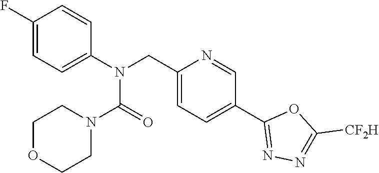 [SMILES: O=C(N1CCOCC1)N(Cc1ccc(-c2nnc(C(F)F)o2)cn1)c1ccc(F)cc1] |
| 299 | 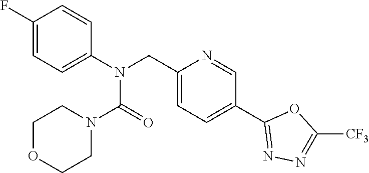 [SMILES: O=C(N1CCOCC1)N(Cc1ccc(-c2nnc(C(F)(F)F)o2)cn1)c1ccc(F)cc1] |
| 300 | 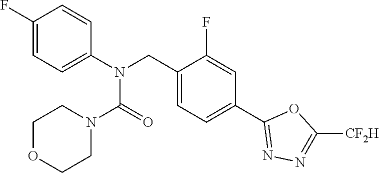 [SMILES: O=C(N1CCOCC1)N(Cc1ccc(-c2nnc(C(F)F)o2)cc1F)c1ccc(F)cc1] |
| 301 | 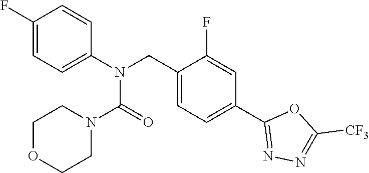 [SMILES: O=C(N1CCOCC1)N(Cc1ccc(-c2nnc(C(F)(F)F)o2)cc1F)c1ccc(F)cc1] |
| 302 | 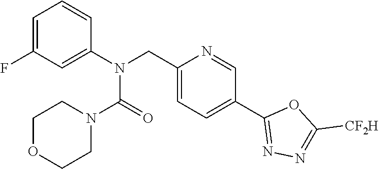 [SMILES: O=C(N1CCOCC1)N(Cc1ccc(-c2nnc(C(F)F)o2)cn1)c1cccc(F)c1] |
| 303 | 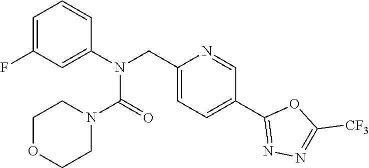 [SMILES: O=C(N1CCOCC1)N(Cc1ccc(-c2nnc(C(F)(F)F)o2)cn1)c1cccc(F)c1] |
| 304 | 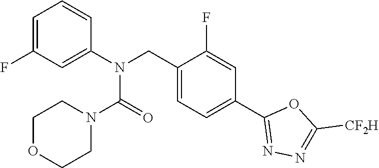 [SMILES: O=C(N1CCOCC1)N(Cc1ccc(-c2nnc(C(F)F)o2)cc1F)c1cccc(F)c1] |
| 305 | 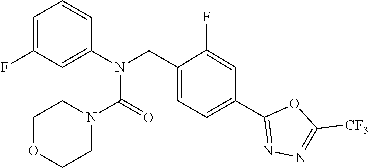 [SMILES: O=C(N1CCOCC1)N(Cc1ccc(-c2nnc(C(F)(F)F)o2)cc1F)c1cccc(F)c1] |
| 306 | 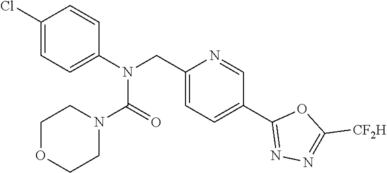 [SMILES: O=C(N1CCOCC1)N(Cc1ccc(-c2nnc(C(F)F)o2)cn1)c1ccc(Cl)cc1] |
| 307 | 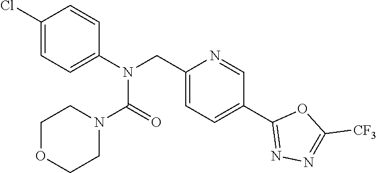 [SMILES: O=C(N1CCOCC1)N(Cc1ccc(-c2nnc(C(F)(F)F)o2)cn1)c1ccc(Cl)cc1] |
| 308 | 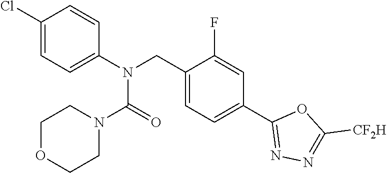 [SMILES: O=C(N1CCOCC1)N(Cc1ccc(-c2nnc(C(F)F)o2)cc1F)c1ccc(Cl)cc1] |
| 309 | 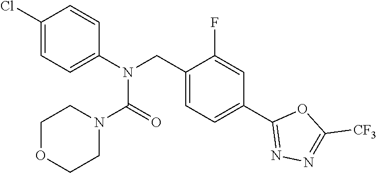 [SMILES: O=C(N1CCOCC1)N(Cc1ccc(-c2nnc(C(F)(F)F)o2)cc1F)c1ccc(Cl)cc1] |
| 310 | 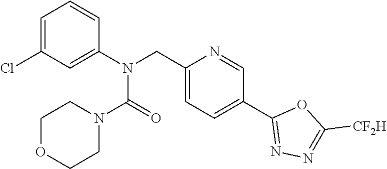 [SMILES: O=C(N1CCOCC1)N(Cc1ccc(-c2nnc(C(F)F)o2)cn1)c1cccc(Cl)c1] |
| 311 | 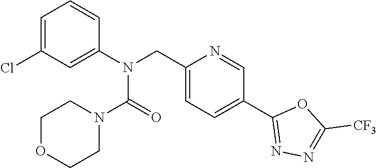 [SMILES: O=C(N1CCOCC1)N(Cc1ccc(-c2nnc(C(F)(F)F)o2)cn1)c1cccc(Cl)c1] |
| 312 | 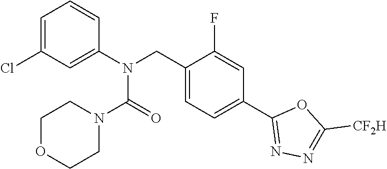 [SMILES: O=C(N1CCOCC1)N(Cc1ccc(-c2nnc(C(F)F)o2)cc1F)c1cccc(Cl)c1] |
| 313 | 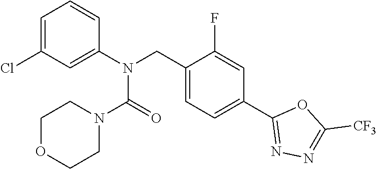 [SMILES: O=C(N1CCOCC1)N(Cc1ccc(-c2nnc(C(F)(F)F)o2)cc1F)c1cccc(Cl)c1] |
| 314 | 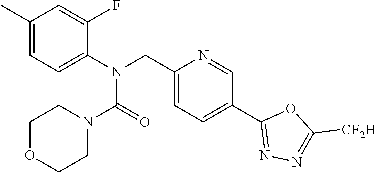 [SMILES: Cc1ccc(N(Cc2ccc(-c3nnc(C(F)F)o3)cn2)C(=O)N2CCOCC2)c(F)c1] |
| 315 | 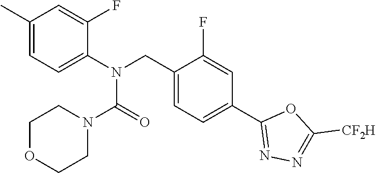 [SMILES: Cc1ccc(N(Cc2ccc(-c3nnc(C(F)F)o3)cc2F)C(=O)N2CCOCC2)c(F)c1] |
| 316 | 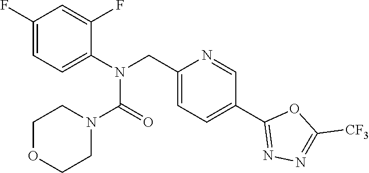 [SMILES: O=C(N1CCOCC1)N(Cc1ccc(-c2nnc(C(F)(F)F)o2)cn1)c1ccc(F)cc1F] |
| 317 | 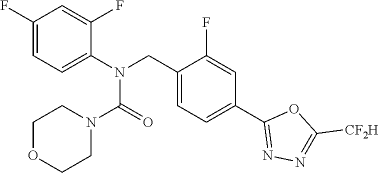 [SMILES: O=C(N1CCOCC1)N(Cc1ccc(-c2nnc(C(F)F)o2)cc1F)c1ccc(F)cc1F] |
| 318 | 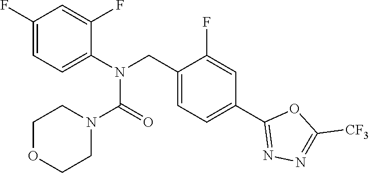 [SMILES: O=C(N1CCOCC1)N(Cc1ccc(-c2nnc(C(F)(F)F)o2)cc1F)c1ccc(F)cc1F] |
| 319 | 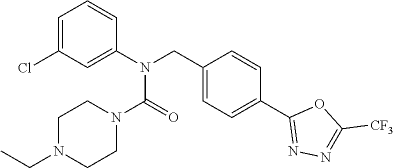 [SMILES: CCN1CCN(C(=O)N(Cc2ccc(-c3nnc(C(F)(F)F)o3)cc2)c2cccc(Cl)c2)CC1] |
| 320 | 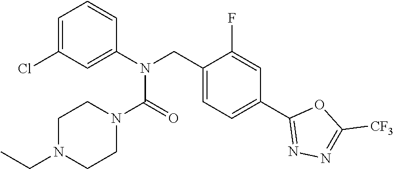 [SMILES: CCN1CCN(C(=O)N(Cc2ccc(-c3nnc(C(F)(F)F)o3)cc2F)c2cccc(Cl)c2)CC1] |
| 321 | 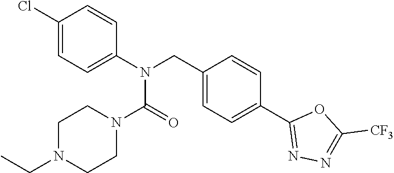 [SMILES: CCN1CCN(C(=O)N(Cc2ccc(-c3nnc(C(F)(F)F)o3)cc2)c2ccc(Cl)cc2)CC1] |
| 322 | 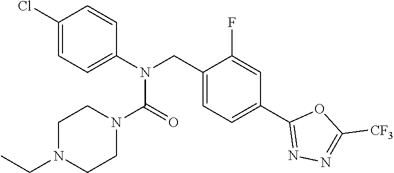 [SMILES: CCN1CCN(C(=O)N(Cc2ccc(-c3nnc(C(F)(F)F)o3)cc2F)c2ccc(Cl)cc2)CC1] |
| 323 | 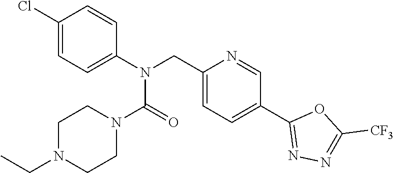 [SMILES: CCN1CCN(C(=O)N(Cc2ccc(-c3nnc(C(F)(F)F)o3)cn2)c2ccc(Cl)cc2)CC1] |
| 324 | 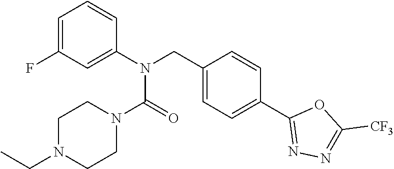 [SMILES: CCN1CCN(C(=O)N(Cc2ccc(-c3nnc(C(F)(F)F)o3)cc2)c2cccc(F)c2)CC1] |
| 325 | 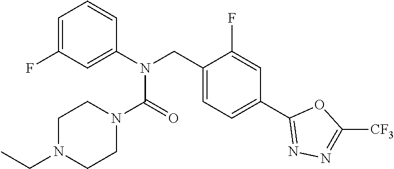 [SMILES: CCN1CCN(C(=O)N(Cc2ccc(-c3nnc(C(F)(F)F)o3)cc2F)c2cccc(F)c2)CC1] |
| 326 | 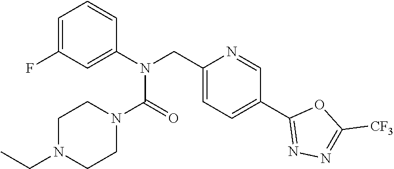 [SMILES: CCN1CCN(C(=O)N(Cc2ccc(-c3nnc(C(F)(F)F)o3)cn2)c2cccc(F)c2)CC1] |
| 327 | 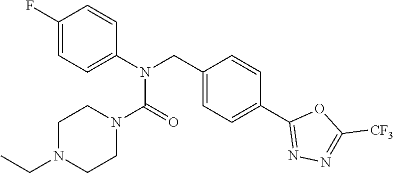 [SMILES: CCN1CCN(C(=O)N(Cc2ccc(-c3nnc(C(F)(F)F)o3)cc2)c2ccc(F)cc2)CC1] |
| 328 | 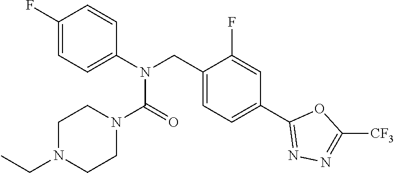 [SMILES: CCN1CCN(C(=O)N(Cc2ccc(-c3nnc(C(F)(F)F)o3)cc2F)c2ccc(F)cc2)CC1] |
| 329 | 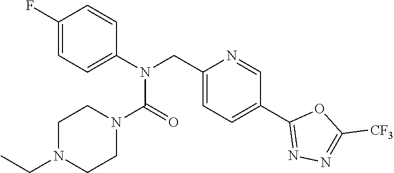 [SMILES: CCN1CCN(C(=O)N(Cc2ccc(-c3nnc(C(F)(F)F)o3)cn2)c2ccc(F)cc2)CC1] |
| 330 | 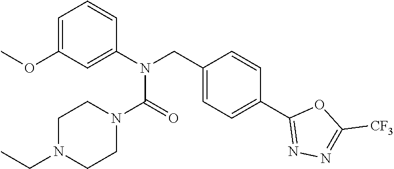 [SMILES: CCN1CCN(C(=O)N(Cc2ccc(-c3nnc(C(F)(F)F)o3)cc2)c2cccc(OC)c2)CC1] |
| 331 | 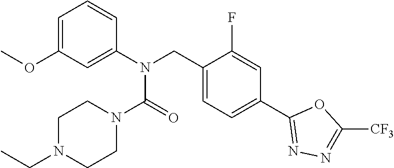 [SMILES: CCN1CCN(C(=O)N(Cc2ccc(-c3nnc(C(F)(F)F)o3)cc2F)c2cccc(OC)c2)CC1] |
| 332 | 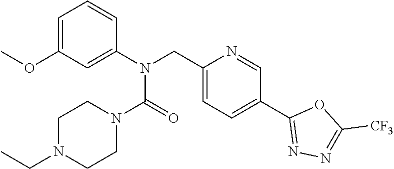 [SMILES: CCN1CCN(C(=O)N(Cc2ccc(-c3nnc(C(F)(F)F)o3)cn2)c2cccc(OC)c2)CC1] |
| 333 | 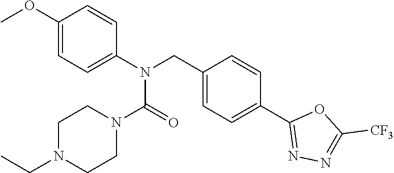 [SMILES: CCN1CCN(C(=O)N(Cc2ccc(-c3nnc(C(F)(F)F)o3)cc2)c2ccc(OC)cc2)CC1] |
| 334 | 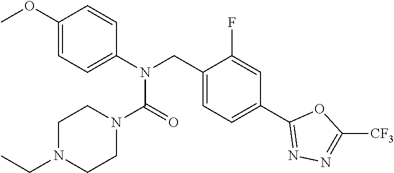 [SMILES: CCN1CCN(C(=O)N(Cc2ccc(-c3nnc(C(F)(F)F)o3)cc2F)c2ccc(OC)cc2)CC1] |
| 335 | 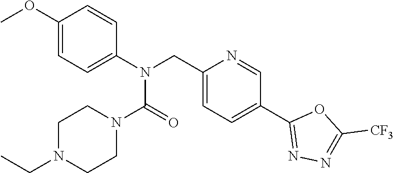 [SMILES: CCN1CCN(C(=O)N(Cc2ccc(-c3nnc(C(F)(F)F)o3)cn2)c2ccc(OC)cc2)CC1] |
| 336 | 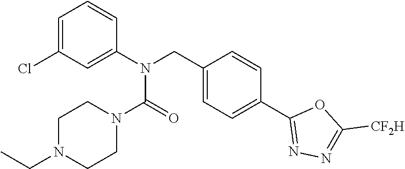 [SMILES: CCN1CCN(C(=O)N(Cc2ccc(-c3nnc(C(F)F)o3)cc2)c2cccc(Cl)c2)CC1] |
| 337 | 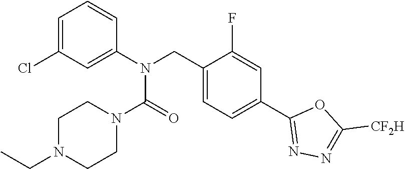 [SMILES: CCN1CCN(C(=O)N(Cc2ccc(-c3nnc(C(F)F)o3)cc2F)c2cccc(Cl)c2)CC1] |
| 338 | 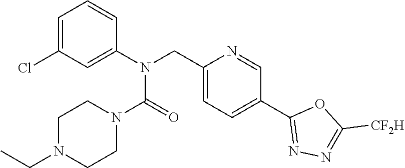 [SMILES: CCN1CCN(C(=O)N(Cc2ccc(-c3nnc(C(F)F)o3)cn2)c2cccc(Cl)c2)CC1] |
| 339 | 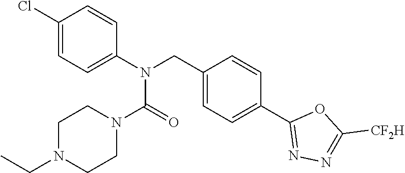 [SMILES: CCN1CCN(C(=O)N(Cc2ccc(-c3nnc(C(F)F)o3)cc2)c2ccc(Cl)cc2)CC1] |
| 340 | 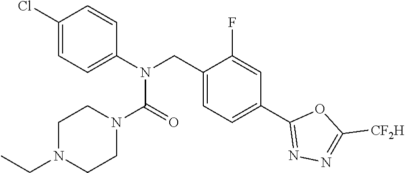 [SMILES: CCN1CCN(C(=O)N(Cc2ccc(-c3nnc(C(F)F)o3)cc2F)c2ccc(Cl)cc2)CC1] |
| 341 | 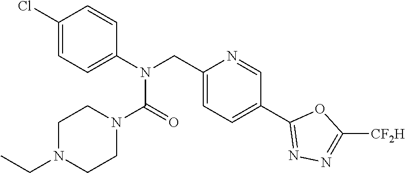 [SMILES: CCN1CCN(C(=O)N(Cc2ccc(-c3nnc(C(F)F)o3)cn2)c2ccc(Cl)cc2)CC1] |
| 342 | 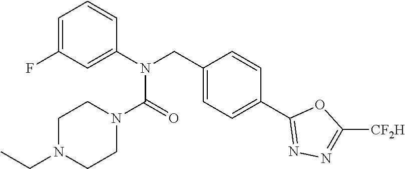 [SMILES: CCN1CCN(C(=O)N(Cc2ccc(-c3nnc(C(F)F)o3)cc2)c2cccc(F)c2)CC1] |
| 343 | 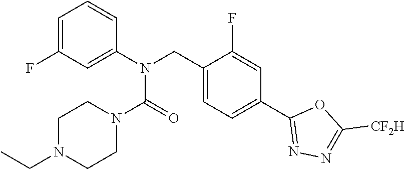 [SMILES: CCN1CCN(C(=O)N(Cc2ccc(-c3nnc(C(F)F)o3)cc2F)c2cccc(F)c2)CC1] |
| 344 | 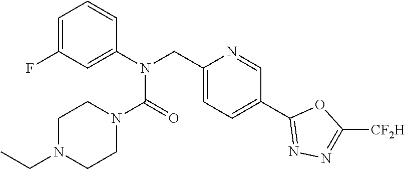 [SMILES: CCN1CCN(C(=O)N(Cc2ccc(-c3nnc(C(F)F)o3)cn2)c2cccc(F)c2)CC1] |
| 345 | 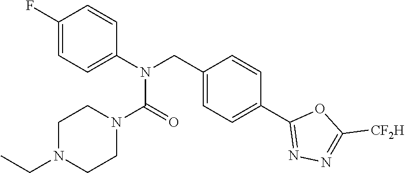 [SMILES: CCN1CCN(C(=O)N(Cc2ccc(-c3nnc(C(F)F)o3)cc2)c2ccc(F)cc2)CC1] |
| 346 | 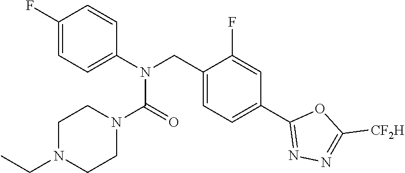 [SMILES: CCN1CCN(C(=O)N(Cc2ccc(-c3nnc(C(F)F)o3)cc2F)c2ccc(F)cc2)CC1] |
| 347 | 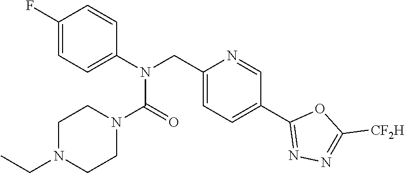 [SMILES: CCN1CCN(C(=O)N(Cc2ccc(-c3nnc(C(F)F)o3)cn2)c2ccc(F)cc2)CC1] |
| 348 | 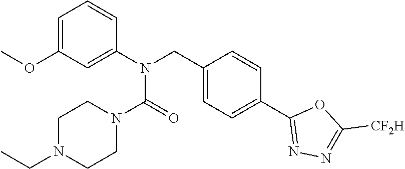 [SMILES: CCN1CCN(C(=O)N(Cc2ccc(-c3nnc(C(F)F)o3)cc2)c2cccc(OC)c2)CC1] |
| 349 | 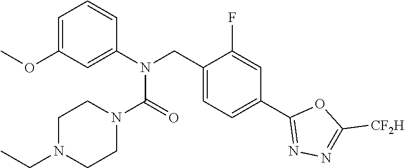 [SMILES: CCN1CCN(C(=O)N(Cc2ccc(-c3nnc(C(F)F)o3)cc2F)c2cccc(OC)c2)CC1] |
| 350 | 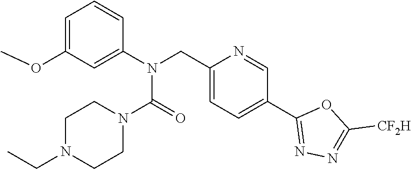 [SMILES: CCN1CCN(C(=O)N(Cc2ccc(-c3nnc(C(F)F)o3)cn2)c2cccc(OC)c2)CC1] |
| 351 | 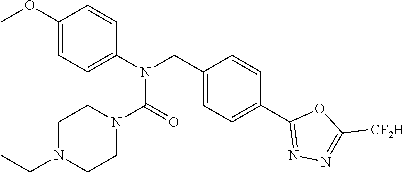 [SMILES: CCN1CCN(C(=O)N(Cc2ccc(-c3nnc(C(F)F)o3)cc2)c2ccc(OC)cc2)CC1] |
| 352 | 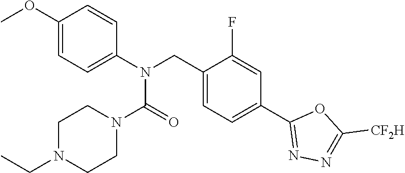 [SMILES: CCN1CCN(C(=O)N(Cc2ccc(-c3nnc(C(F)F)o3)cc2F)c2ccc(OC)cc2)CC1] |
| 353 | 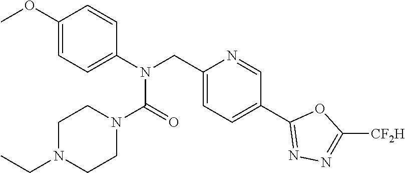 [SMILES: CCN1CCN(C(=O)N(Cc2ccc(-c3nnc(C(F)F)o3)cn2)c2ccc(OC)cc2)CC1] |
| 354 | 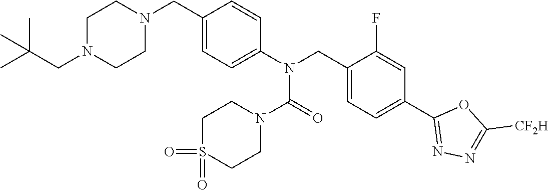 [SMILES: CC(C)(C)CN1CCN(Cc2ccc(N(Cc3ccc(-c4nnc(C(F)F)o4)cc3F)C(=O)N3CCS(=O)(=O)CC3)cc2)CC1] |
| 355 | 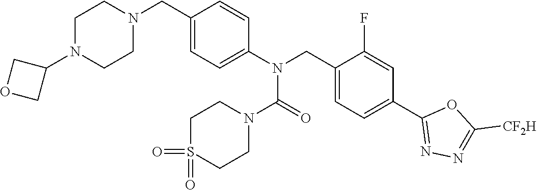 [SMILES: O=C(N1CCS(=O)(=O)CC1)N(Cc1ccc(-c2nnc(C(F)F)o2)cc1F)c1ccc(CN2CCN(C3COC3)CC2)cc1] |
| 356 | 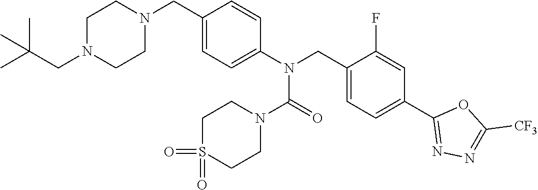 [SMILES: CC(C)(C)CN1CCN(Cc2ccc(N(Cc3ccc(-c4nnc(C(F)(F)F)o4)cc3F)C(=O)N3CCS(=O)(=O)CC3)cc2)CC1] |
| 357 | 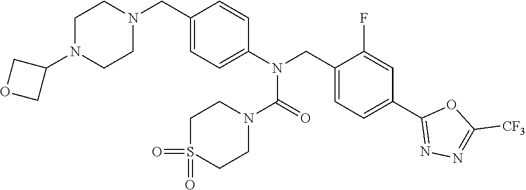 [SMILES: O=C(N1CCS(=O)(=O)CC1)N(Cc1ccc(-c2nnc(C(F)(F)F)o2)cc1F)c1ccc(CN2CCN(C3COC3)CC2)cc1] |
| 358 | 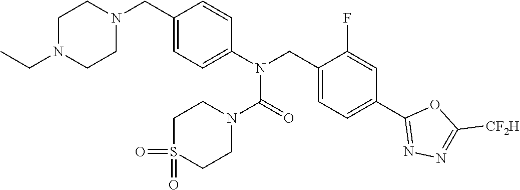 [SMILES: CCN1CCN(Cc2ccc(N(Cc3ccc(-c4nnc(C(F)F)o4)cc3F)C(=O)N3CCS(=O)(=O)CC3)cc2)CC1] |
| 359 | 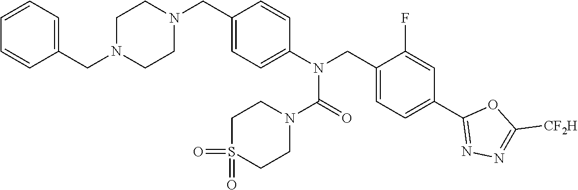 [SMILES: O=C(N1CCS(=O)(=O)CC1)N(Cc1ccc(-c2nnc(C(F)F)o2)cc1F)c1ccc(CN2CCN(Cc3ccccc3)CC2)cc1] |
| 360 | 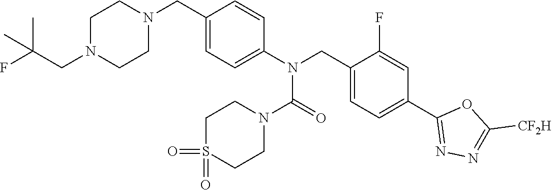 [SMILES: CC(C)(F)CN1CCN(Cc2ccc(N(Cc3ccc(-c4nnc(C(F)F)o4)cc3F)C(=O)N3CCS(=O)(=O)CC3)cc2)CC1] |
| 361 | 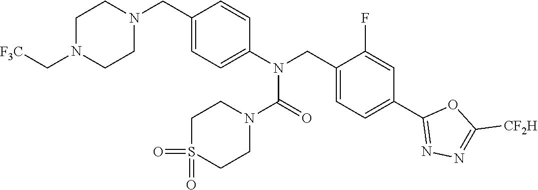 [SMILES: O=C(N1CCS(=O)(=O)CC1)N(Cc1ccc(-c2nnc(C(F)F)o2)cc1F)c1ccc(CN2CCN(CC(F)(F)F)CC2)cc1] |
| 362 | 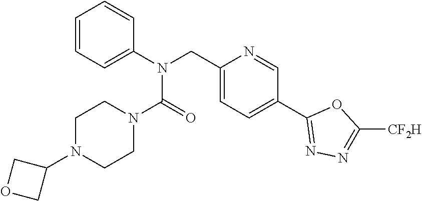 [SMILES: O=C(N1CCN(C2COC2)CC1)N(Cc1ccc(-c2nnc(C(F)F)o2)cn1)c1ccccc1] |
| 363 | 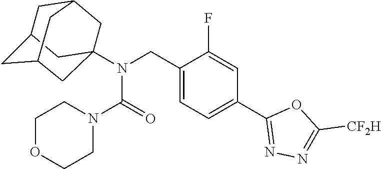 [SMILES: O=C(N1CCOCC1)N(Cc1ccc(-c2nnc(C(F)F)o2)cc1F)C12CC3CC(CC(C3)C1)C2] |
| 364 | 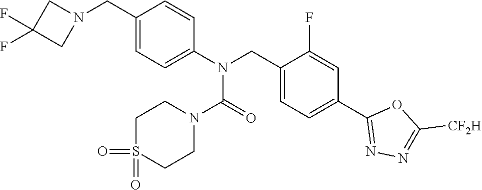 [SMILES: O=C(N1CCS(=O)(=O)CC1)N(Cc1ccc(-c2nnc(C(F)F)o2)cc1F)c1ccc(CN2CC(F)(F)C2)cc1] |
| 365 | 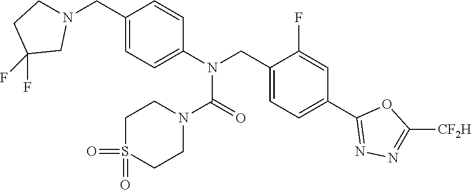 [SMILES: O=C(N1CCS(=O)(=O)CC1)N(Cc1ccc(-c2nnc(C(F)F)o2)cc1F)c1ccc(CN2CCC(F)(F)C2)cc1] |
| 366 | 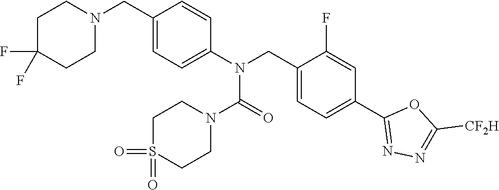 [SMILES: O=C(N1CCS(=O)(=O)CC1)N(Cc1ccc(-c2nnc(C(F)F)o2)cc1F)c1ccc(CN2CCC(F)(F)CC2)cc1] |
| 367 | 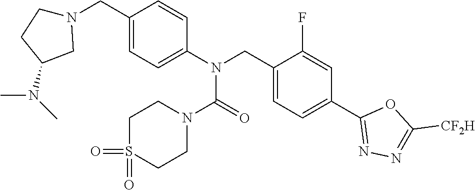 [SMILES: CN(C)[C@@H]1CCN(Cc2ccc(N(Cc3ccc(-c4nnc(C(F)F)o4)cc3F)C(=O)N3CCS(=O)(=O)CC3)cc2)C1] |
| 368 | 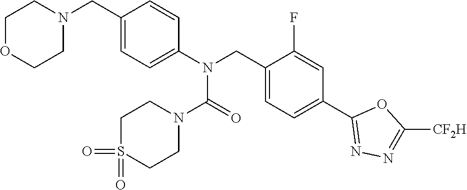 [SMILES: O=C(N1CCS(=O)(=O)CC1)N(Cc1ccc(-c2nnc(C(F)F)o2)cc1F)c1ccc(CN2CCOCC2)cc1] |
| 369 | 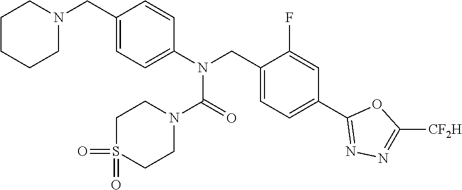 [SMILES: O=C(N1CCS(=O)(=O)CC1)N(Cc1ccc(-c2nnc(C(F)F)o2)cc1F)c1ccc(CN2CCCCC2)cc1] |
| 370 | 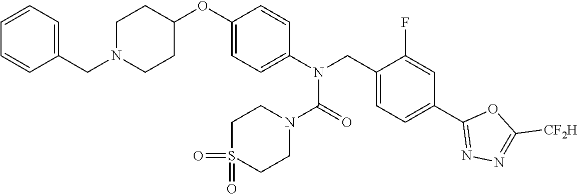 [SMILES: O=C(N1CCS(=O)(=O)CC1)N(Cc1ccc(-c2nnc(C(F)F)o2)cc1F)c1ccc(OC2CCN(Cc3ccccc3)CC2)cc1] |
| 371 | 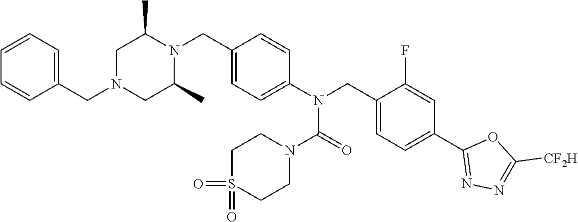 [SMILES: C[C@@H]1CN(Cc2ccccc2)C[C@H](C)N1Cc1ccc(N(Cc2ccc(-c3nnc(C(F)F)o3)cc2F)C(=O)N2CCS(=O)(=O)CC2)cc1] |
| 372 | 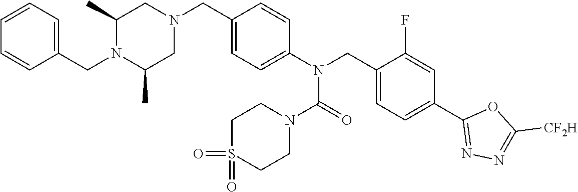 [SMILES: C[C@@H]1CN(Cc2ccc(N(Cc3ccc(-c4nnc(C(F)F)o4)cc3F)C(=O)N3CCS(=O)(=O)CC3)cc2)C[C@H](C)N1Cc1ccccc1] |
| 373 | 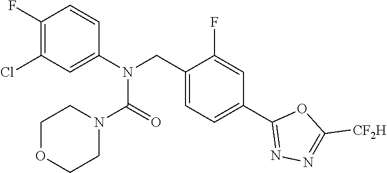 [SMILES: O=C(N1CCOCC1)N(Cc1ccc(-c2nnc(C(F)F)o2)cc1F)c1ccc(F)c(Cl)c1] |
| 374 | 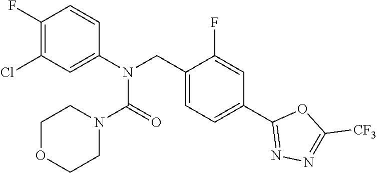 [SMILES: O=C(N1CCOCC1)N(Cc1ccc(-c2nnc(C(F)(F)F)o2)cc1F)c1ccc(F)c(Cl)c1] |
| 375 | 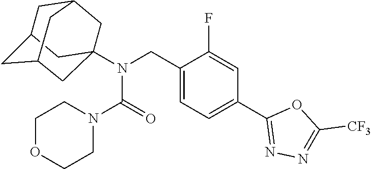 [SMILES: O=C(N1CCOCC1)N(Cc1ccc(-c2nnc(C(F)(F)F)o2)cc1F)C12CC3CC(CC(C3)C1)C2] |
| 376 | 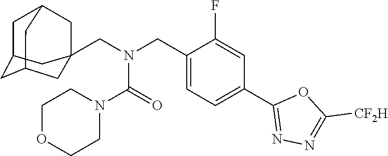 [SMILES: O=C(N1CCOCC1)N(Cc1ccc(-c2nnc(C(F)F)o2)cc1F)CC12CC3CC(CC(C3)C1)C2] |
| 377 | 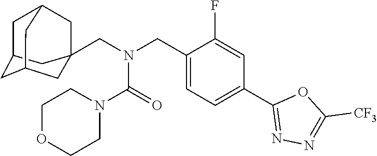 [SMILES: O=C(N1CCOCC1)N(Cc1ccc(-c2nnc(C(F)(F)F)o2)cc1F)CC12CC3CC(CC(C3)C1)C2] |
| 378 | 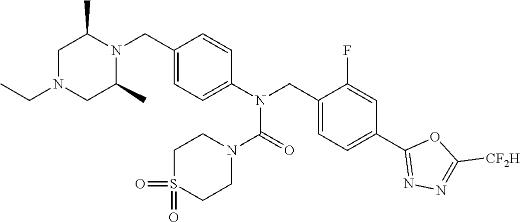 [SMILES: CCN1C[C@@H](C)N(Cc2ccc(N(Cc3ccc(-c4nnc(C(F)F)o4)cc3F)C(=O)N3CCS(=O)(=O)CC3)cc2)[C@@H](C)C1] |
| 379 | 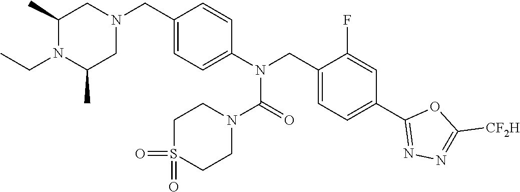 [SMILES: CCN1[C@H](C)CN(Cc2ccc(N(Cc3ccc(-c4nnc(C(F)F)o4)cc3F)C(=O)N3CCS(=O)(=O)CC3)cc2)C[C@@H]1C] |
| 380 | 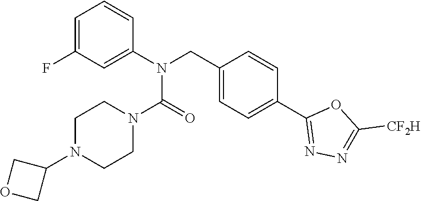 [SMILES: O=C(N1CCN(C2COC2)CC1)N(Cc1ccc(-c2nnc(C(F)F)o2)cc1)c1cccc(F)c1] |
| 381 | 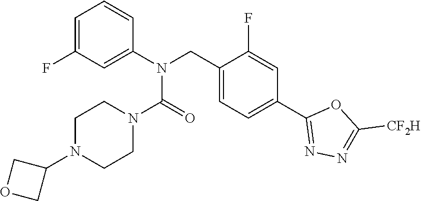 [SMILES: O=C(N1CCN(C2COC2)CC1)N(Cc1ccc(-c2nnc(C(F)F)o2)cc1F)c1cccc(F)c1] |
| 382 | 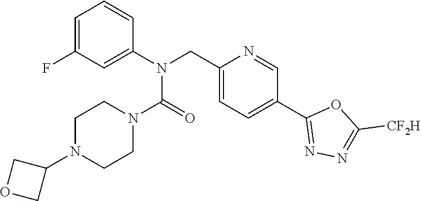 [SMILES: O=C(N1CCN(C2COC2)CC1)N(Cc1ccc(-c2nnc(C(F)F)o2)cn1)c1cccc(F)c1] |
| 383 | 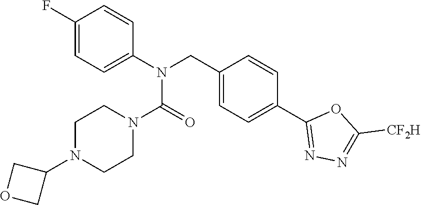 [SMILES: O=C(N1CCN(C2COC2)CC1)N(Cc1ccc(-c2nnc(C(F)F)o2)cc1)c1ccc(F)cc1] |
| 384 | 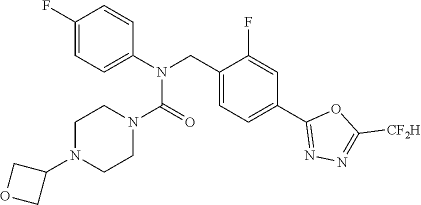 [SMILES: O=C(N1CCN(C2COC2)CC1)N(Cc1ccc(-c2nnc(C(F)F)o2)cc1F)c1ccc(F)cc1] |
| 385 | 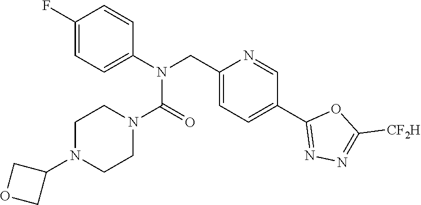 [SMILES: O=C(N1CCN(C2COC2)CC1)N(Cc1ccc(-c2nnc(C(F)F)o2)cn1)c1ccc(F)cc1] |
| 386 | 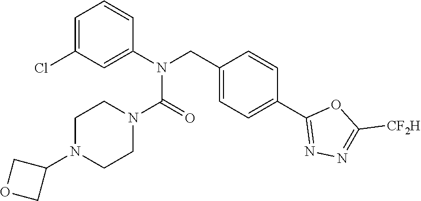 [SMILES: O=C(N1CCN(C2COC2)CC1)N(Cc1ccc(-c2nnc(C(F)F)o2)cc1)c1cccc(Cl)c1] |
| 387 | 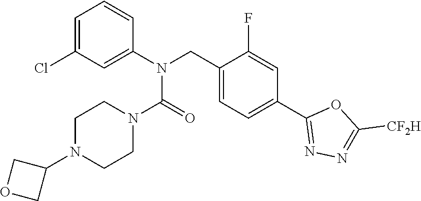 [SMILES: O=C(N1CCN(C2COC2)CC1)N(Cc1ccc(-c2nnc(C(F)F)o2)cc1F)c1cccc(Cl)c1] |
| 388 | 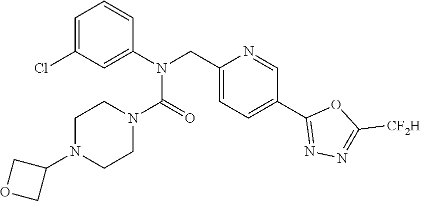 [SMILES: O=C(N1CCN(C2COC2)CC1)N(Cc1ccc(-c2nnc(C(F)F)o2)cn1)c1cccc(Cl)c1] |
| 389 | 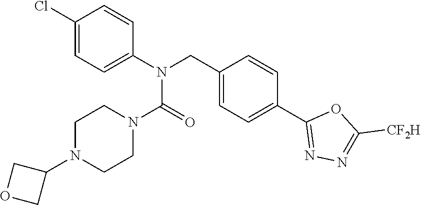 [SMILES: O=C(N1CCN(C2COC2)CC1)N(Cc1ccc(-c2nnc(C(F)F)o2)cc1)c1ccc(Cl)cc1] |
| 390 | 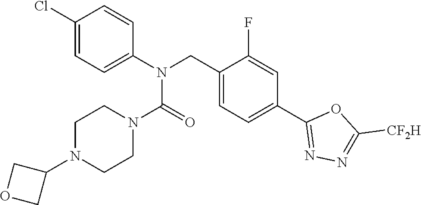 [SMILES: O=C(N1CCN(C2COC2)CC1)N(Cc1ccc(-c2nnc(C(F)F)o2)cc1F)c1ccc(Cl)cc1] |
| 391 | 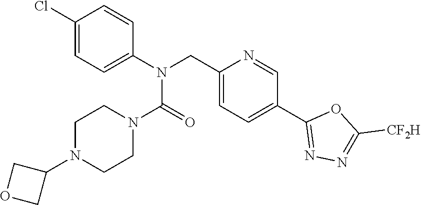 [SMILES: O=C(N1CCN(C2COC2)CC1)N(Cc1ccc(-c2nnc(C(F)F)o2)cn1)c1ccc(Cl)cc1] |
| 392 | 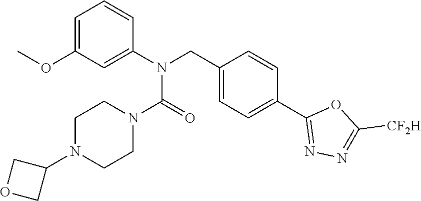 [SMILES: COc1cccc(N(Cc2ccc(-c3nnc(C(F)F)o3)cc2)C(=O)N2CCN(C3COC3)CC2)c1] |
| 393 | 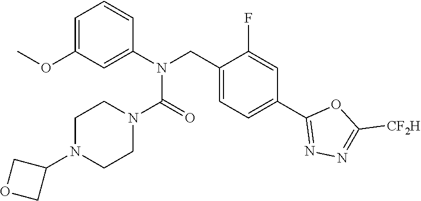 [SMILES: COc1cccc(N(Cc2ccc(-c3nnc(C(F)F)o3)cc2F)C(=O)N2CCN(C3COC3)CC2)c1] |
| 394 | 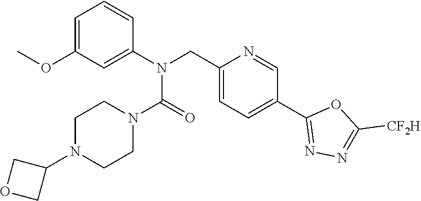 [SMILES: COc1cccc(N(Cc2ccc(-c3nnc(C(F)F)o3)cn2)C(=O)N2CCN(C3COC3)CC2)c1] |
| 395 | 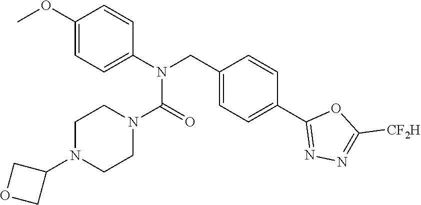 [SMILES: COc1ccc(N(Cc2ccc(-c3nnc(C(F)F)o3)cc2)C(=O)N2CCN(C3COC3)CC2)cc1] |
| 396 | 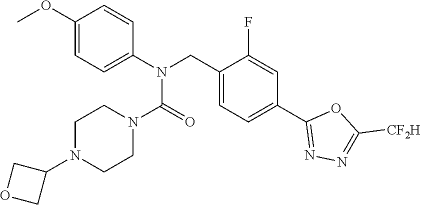 [SMILES: COc1ccc(N(Cc2ccc(-c3nnc(C(F)F)o3)cc2F)C(=O)N2CCN(C3COC3)CC2)cc1] |
| 397 | 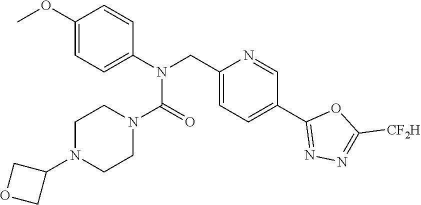 [SMILES: COc1ccc(N(Cc2ccc(-c3nnc(C(F)F)o3)cn2)C(=O)N2CCN(C3COC3)CC2)cc1] |
| 398 | 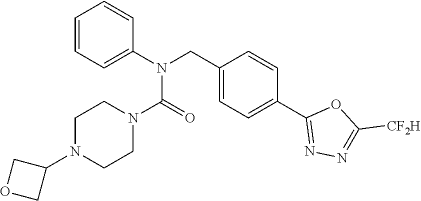 [SMILES: O=C(N1CCN(C2COC2)CC1)N(Cc1ccc(-c2nnc(C(F)F)o2)cc1)c1ccccc1] |
| 399 | 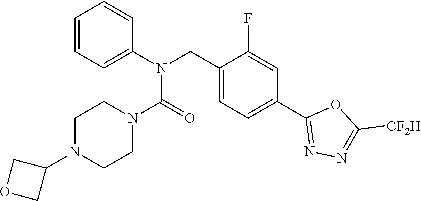 [SMILES: O=C(N1CCN(C2COC2)CC1)N(Cc1ccc(-c2nnc(C(F)F)o2)cc1F)c1ccccc1] |
| 400 | 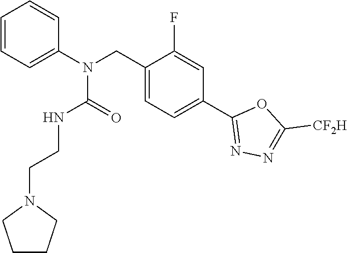 [SMILES: O=C(NCCN1CCCC1)N(Cc1ccc(-c2nnc(C(F)F)o2)cc1F)c1ccccc1] |
| 401 | 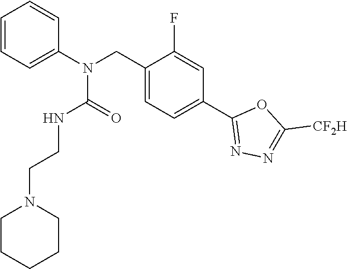 [SMILES: O=C(NCCN1CCCCC1)N(Cc1ccc(-c2nnc(C(F)F)o2)cc1F)c1ccccc1] |
| 402 | 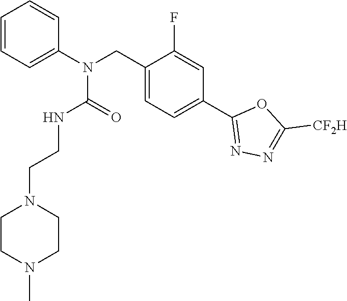 [SMILES: CN1CCN(CCNC(=O)N(Cc2ccc(-c3nnc(C(F)F)o3)cc2F)c2ccccc2)CC1] |
| 403 | 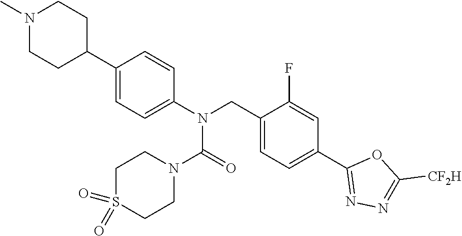 [SMILES: CN1CCC(c2ccc(N(Cc3ccc(-c4nnc(C(F)F)o4)cc3F)C(=O)N3CCS(=O)(=O)CC3)cc2)CC1] |
| 404 | 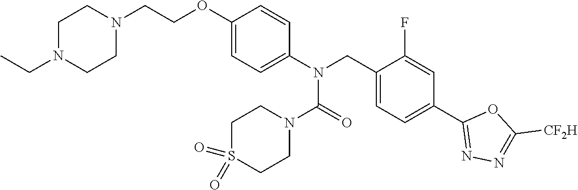 [SMILES: CCN1CCN(CCOc2ccc(N(Cc3ccc(-c4nnc(C(F)F)o4)cc3F)C(=O)N3CCS(=O)(=O)CC3)cc2)CC1] |
| 405 | 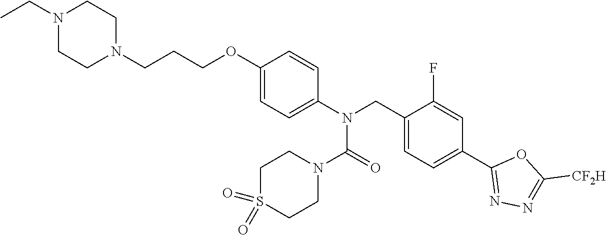 [SMILES: CCN1CCN(CCCOc2ccc(N(Cc3ccc(-c4nnc(C(F)F)o4)cc3F)C(=O)N3CCS(=O)(=O)CC3)cc2)CC1] |
| 406 | 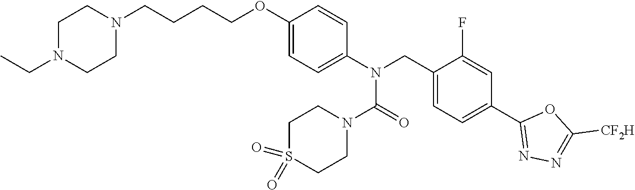 [SMILES: CCN1CCN(CCCCOc2ccc(N(Cc3ccc(-c4nnc(C(F)F)o4)cc3F)C(=O)N3CCS(=O)(=O)CC3)cc2)CC1] |
| 407 | 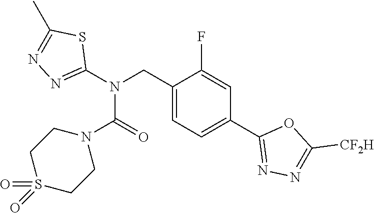 [SMILES: Cc1nnc(N(Cc2ccc(-c3nnc(C(F)F)o3)cc2F)C(=O)N2CCS(=O)(=O)CC2)s1] |
| 408 | 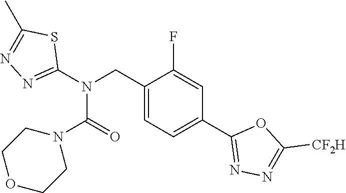 [SMILES: Cc1nnc(N(Cc2ccc(-c3nnc(C(F)F)o3)cc2F)C(=O)N2CCOCC2)s1] |
| 409 | 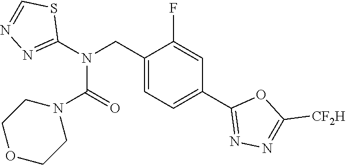 [SMILES: O=C(N1CCOCC1)N(Cc1ccc(-c2nnc(C(F)F)o2)cc1F)c1nncs1] |
| 410 | 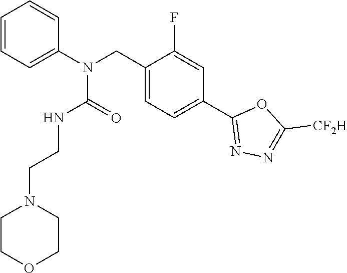 [SMILES: O=C(NCCN1CCOCC1)N(Cc1ccc(-c2nnc(C(F)F)o2)cc1F)c1ccccc1] |
| 411 | 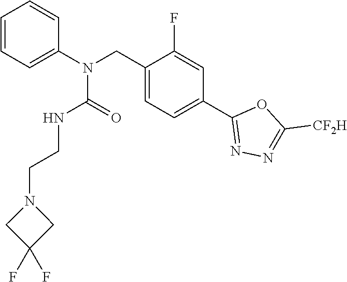 [SMILES: O=C(NCCN1CC(F)(F)C1)N(Cc1ccc(-c2nnc(C(F)F)o2)cc1F)c1ccccc1] |
| 412 | 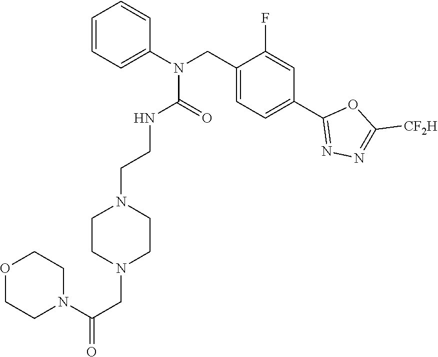 [SMILES: O=C(CN1CCN(CCNC(=O)N(Cc2ccc(-c3nnc(C(F)F)o3)cc2F)c2ccccc2)CC1)N1CCOCC1] |
| 413 | 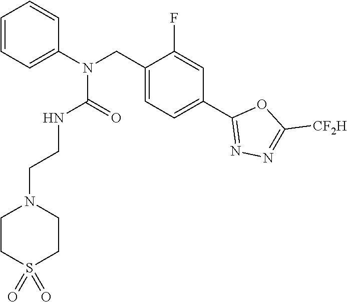 [SMILES: O=C(NCCN1CCS(=O)(=O)CC1)N(Cc1ccc(-c2nnc(C(F)F)o2)cc1F)c1ccccc1] |
| 414 | 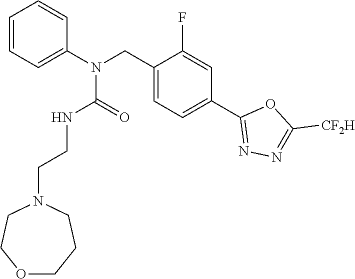 [SMILES: O=C(NCCN1CCCOCC1)N(Cc1ccc(-c2nnc(C(F)F)o2)cc1F)c1ccccc1] |
| 415 | 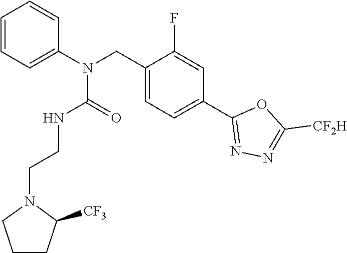 [SMILES: O=C(NCCN1CCC[C@@H]1C(F)(F)F)N(Cc1ccc(-c2nnc(C(F)F)o2)cc1F)c1ccccc1] |
| 416 | 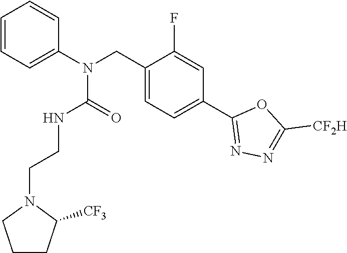 [SMILES: O=C(NCCN1CCC[C@H]1C(F)(F)F)N(Cc1ccc(-c2nnc(C(F)F)o2)cc1F)c1ccccc1] |
| 417 | 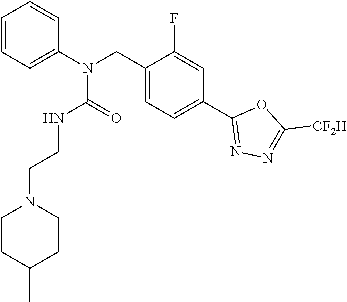 [SMILES: CC1CCN(CCNC(=O)N(Cc2ccc(-c3nnc(C(F)F)o3)cc2F)c2ccccc2)CC1] |
| 418 | 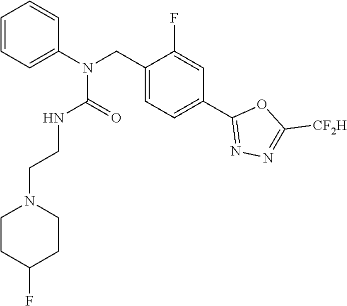 [SMILES: O=C(NCCN1CCC(F)CC1)N(Cc1ccc(-c2nnc(C(F)F)o2)cc1F)c1ccccc1] |
| 419 | 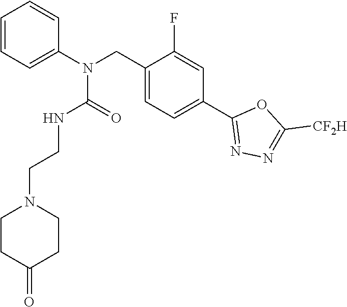 [SMILES: O=C1CCN(CCNC(=O)N(Cc2ccc(-c3nnc(C(F)F)o3)cc2F)c2ccccc2)CC1] |
| 420 | 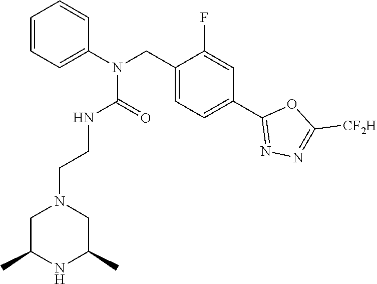 [SMILES: C[C@@H]1CN(CCNC(=O)N(Cc2ccc(-c3nnc(C(F)F)o3)cc2F)c2ccccc2)C[C@H](C)N1] |
| 421 | 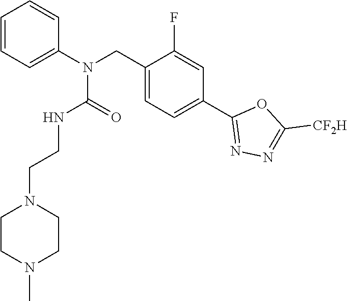 [SMILES: CN1CCN(CCNC(=O)N(Cc2ccc(-c3nnc(C(F)F)o3)cc2F)c2ccccc2)CC1] |
| 422 | 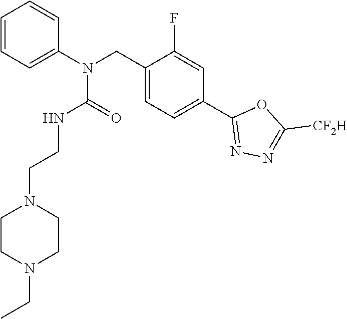 [SMILES: CCN1CCN(CCNC(=O)N(Cc2ccc(-c3nnc(C(F)F)o3)cc2F)c2ccccc2)CC1] |
| 423 | 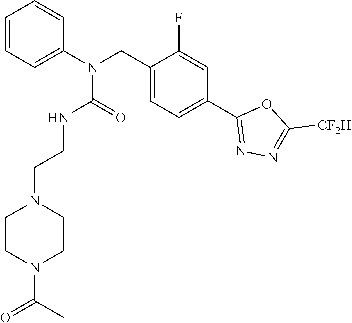 [SMILES: CC(=O)N1CCN(CCNC(=O)N(Cc2ccc(-c3nnc(C(F)F)o3)cc2F)c2ccccc2)CC1] |
| 424 | 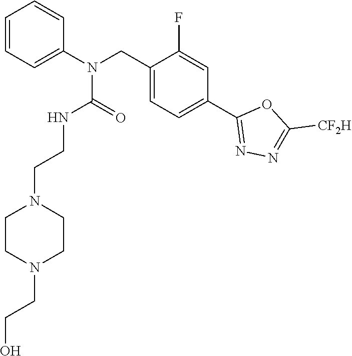 [SMILES: O=C(NCCN1CCN(CCO)CC1)N(Cc1ccc(-c2nnc(C(F)F)o2)cc1F)c1ccccc1] |
| 425 | 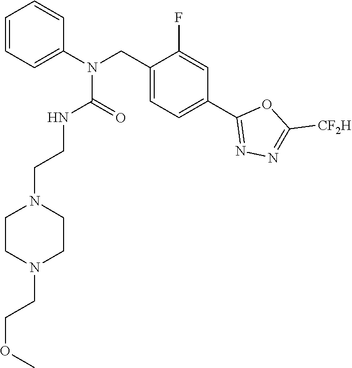 [SMILES: COCCN1CCN(CCNC(=O)N(Cc2ccc(-c3nnc(C(F)F)o3)cc2F)c2ccccc2)CC1] |
| 426 | 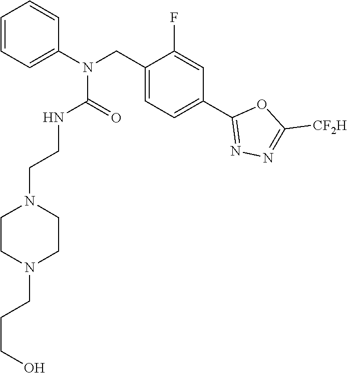 [SMILES: O=C(NCCN1CCN(CCCO)CC1)N(Cc1ccc(-c2nnc(C(F)F)o2)cc1F)c1ccccc1] |
| 427 | 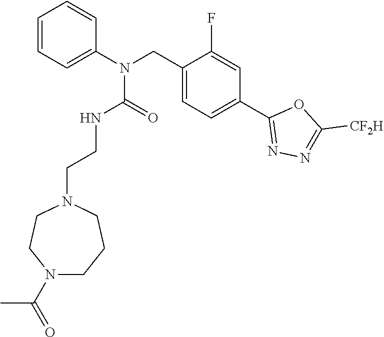 [SMILES: CC(=O)N1CCCN(CCNC(=O)N(Cc2ccc(-c3nnc(C(F)F)o3)cc2F)c2ccccc2)CC1] |
| 428 | 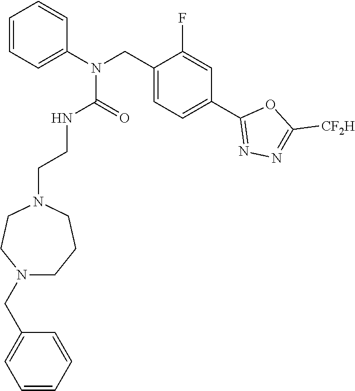 [SMILES: O=C(NCCN1CCCN(Cc2ccccc2)CC1)N(Cc1ccc(-c2nnc(C(F)F)o2)cc1F)c1ccccc1] |
| 429 | 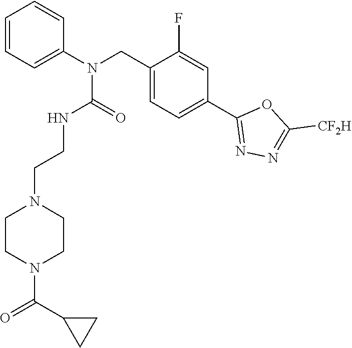 [SMILES: O=C(C1CC1)N1CCN(CCNC(=O)N(Cc2ccc(-c3nnc(C(F)F)o3)cc2F)c2ccccc2)CC1] |
| 430 | 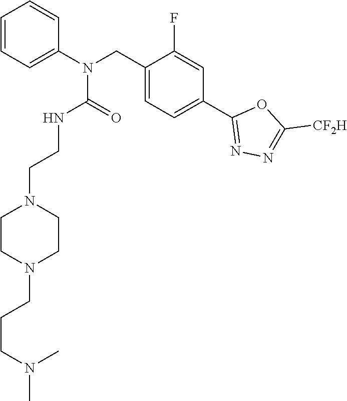 [SMILES: CN(C)CCCN1CCN(CCNC(=O)N(Cc2ccc(-c3nnc(C(F)F)o3)cc2F)c2ccccc2)CC1] |
| 431 | 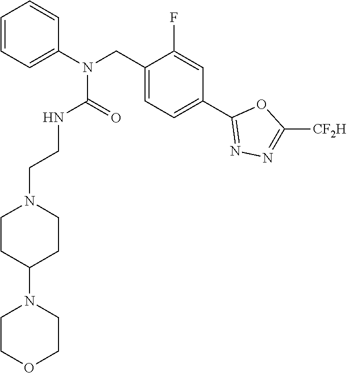 [SMILES: O=C(NCCN1CCC(N2CCOCC2)CC1)N(Cc1ccc(-c2nnc(C(F)F)o2)cc1F)c1ccccc1] |
| 432 | 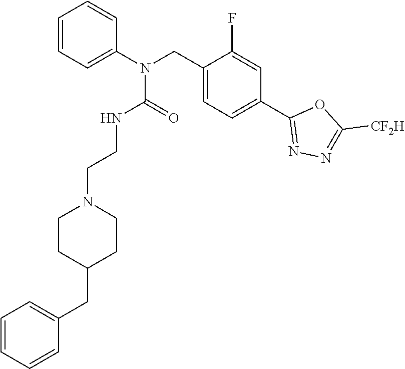 [SMILES: O=C(NCCN1CCC(Cc2ccccc2)CC1)N(Cc1ccc(-c2nnc(C(F)F)o2)cc1F)c1ccccc1] |
| 433 | 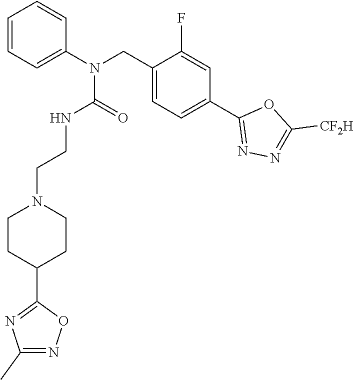 [SMILES: Cc1noc(C2CCN(CCNC(=O)N(Cc3ccc(-c4nnc(C(F)F)o4)cc3F)c3ccccc3)CC2)n1] |
| 434 | 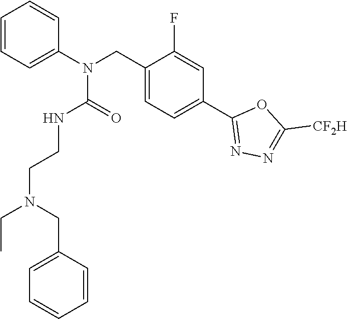 [SMILES: CCN(CCNC(=O)N(Cc1ccc(-c2nnc(C(F)F)o2)cc1F)c1ccccc1)Cc1ccccc1] |
| 435 | 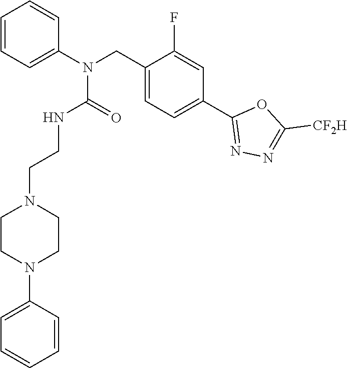 [SMILES: O=C(NCCN1CCN(c2ccccc2)CC1)N(Cc1ccc(-c2nnc(C(F)F)o2)cc1F)c1ccccc1] |
| 436 | 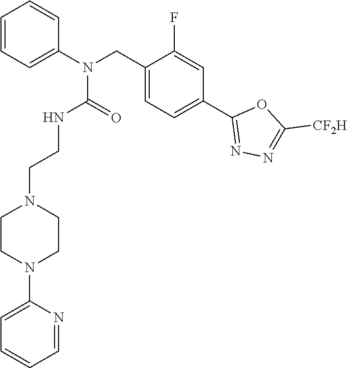 [SMILES: O=C(NCCN1CCN(c2ccccn2)CC1)N(Cc1ccc(-c2nnc(C(F)F)o2)cc1F)c1ccccc1] |
| 437 | 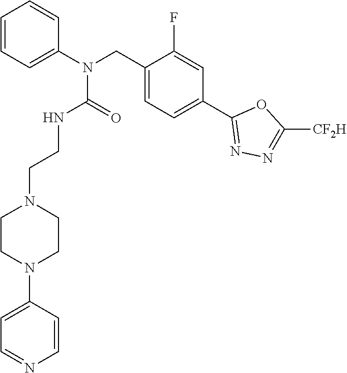 [SMILES: O=C(NCCN1CCN(c2ccncc2)CC1)N(Cc1ccc(-c2nnc(C(F)F)o2)cc1F)c1ccccc1] |
| 438 | 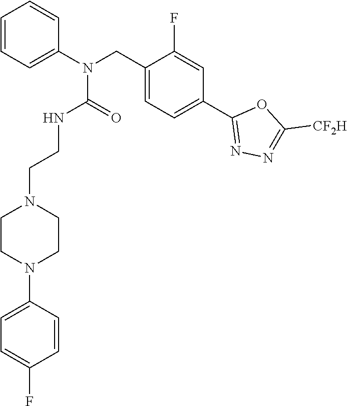 [SMILES: O=C(NCCN1CCN(c2ccc(F)cc2)CC1)N(Cc1ccc(-c2nnc(C(F)F)o2)cc1F)c1ccccc1] |
| 439 | 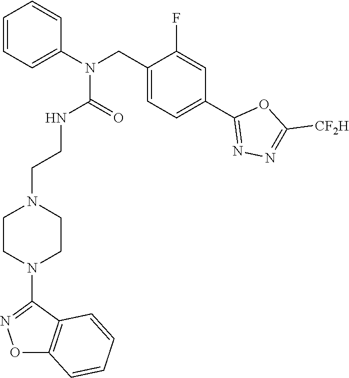 [SMILES: O=C(NCCN1CCN(c2noc3ccccc23)CC1)N(Cc1ccc(-c2nnc(C(F)F)o2)cc1F)c1ccccc1] |
| 440 | 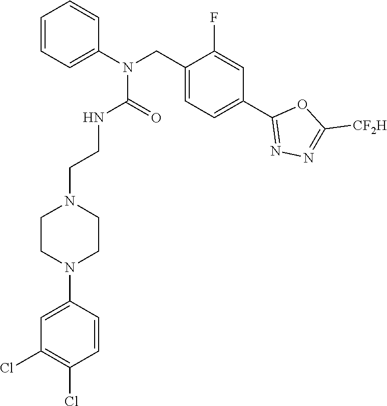 [SMILES: O=C(NCCN1CCN(c2ccc(Cl)c(Cl)c2)CC1)N(Cc1ccc(-c2nnc(C(F)F)o2)cc1F)c1ccccc1] |
| 441 | 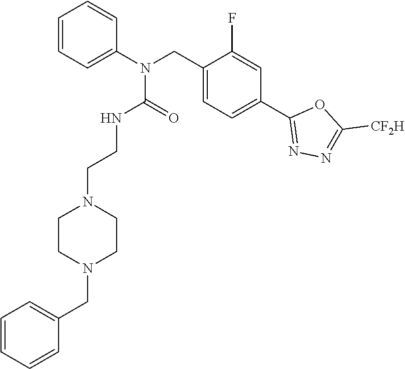 [SMILES: O=C(NCCN1CCN(Cc2ccccc2)CC1)N(Cc1ccc(-c2nnc(C(F)F)o2)cc1F)c1ccccc1] |
| 442 | 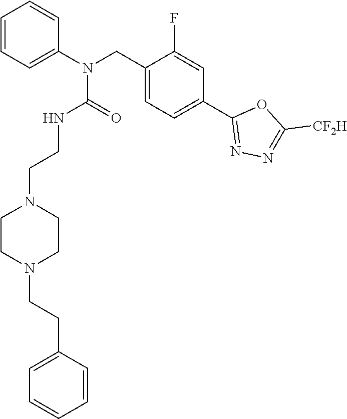 [SMILES: O=C(NCCN1CCN(CCc2ccccc2)CC1)N(Cc1ccc(-c2nnc(C(F)F)o2)cc1F)c1ccccc1] |
| 443 | 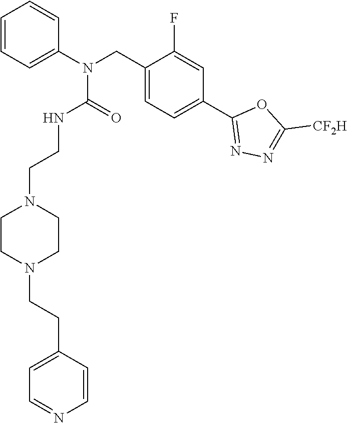 [SMILES: O=C(NCCN1CCN(CCc2ccncc2)CC1)N(Cc1ccc(-c2nnc(C(F)F)o2)cc1F)c1ccccc1] |
| 444 | 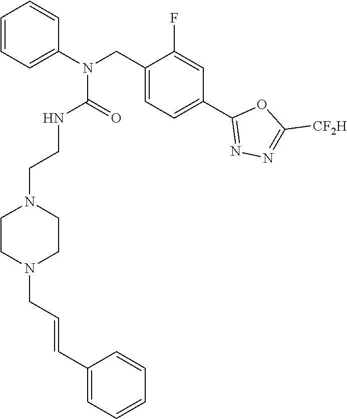 [SMILES: O=C(NCCN1CCN(C/C=C/c2ccccc2)CC1)N(Cc1ccc(-c2nnc(C(F)F)o2)cc1F)c1ccccc1] |
| 445 | 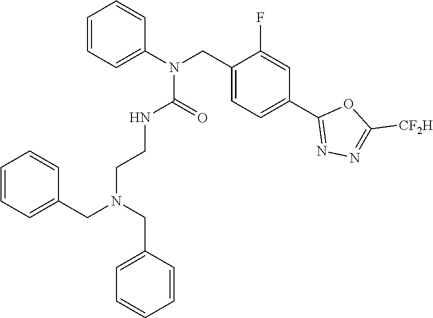 [SMILES: O=C(NCCN(Cc1ccccc1)Cc1ccccc1)N(Cc1ccc(-c2nnc(C(F)F)o2)cc1F)c1ccccc1] |
| 446 | 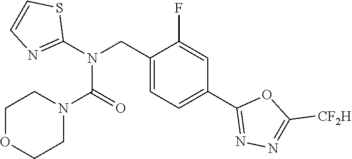 [SMILES: O=C(N1CCOCC1)N(Cc1ccc(-c2nnc(C(F)F)o2)cc1F)c1nccs1] |
| 447 | 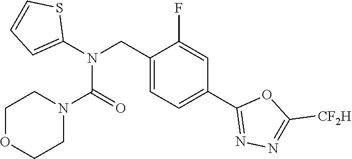 [SMILES: O=C(N1CCOCC1)N(Cc1ccc(-c2nnc(C(F)F)o2)cc1F)c1cccs1] |
| 448 | 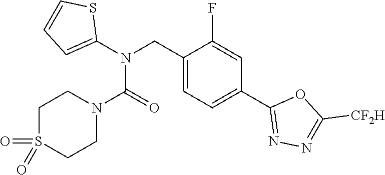 [SMILES: O=C(N1CCS(=O)(=O)CC1)N(Cc1ccc(-c2nnc(C(F)F)o2)cc1F)c1cccs1] |
| 449 | 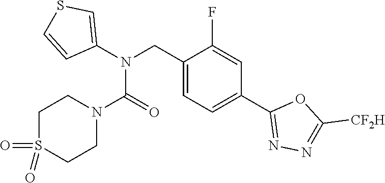 [SMILES: O=C(N1CCS(=O)(=O)CC1)N(Cc1ccc(-c2nnc(C(F)F)o2)cc1F)c1ccsc1] |
| 450 | 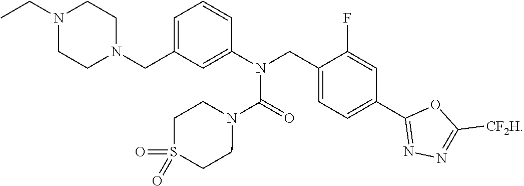 [SMILES: CCN1CCN(Cc2cccc(N(Cc3ccc(-c4nnc(C(F)F)o4)cc3F)C(=O)N3CCS(=O)(=O)CC3)c2)CC1] |

6. A pharmaceutical composition for preventing or treating heart failure, comprising a compound, optical isomers thereof or pharmaceutically acceptable salts thereof as an active ingredient, wherein the compound has the following structure:

| Com- | |
|---|---|
| pound | Structure |
| 40 | 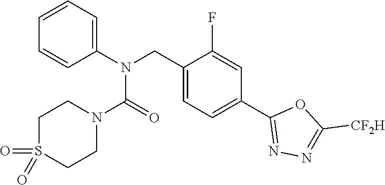 [SMILES: O=C(N1CCS(=O)(=O)CC1)N(Cc1ccc(-c2nnc(C(F)F)o2)cc1F)c1ccccc1] |
| 43 | 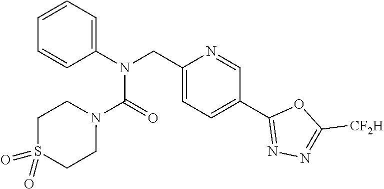 [SMILES: O=C(N1CCS(=O)(=O)CC1)N(Cc1ccc(-c2nnc(C(F)F)o2)cn1)c1ccccc1] |
| 239 | 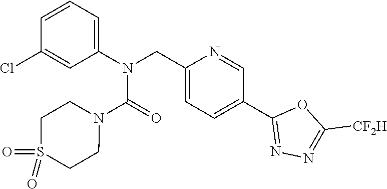 [SMILES: O=C(N1CCS(=O)(=O)CC1)N(Cc1ccc(-c2nnc(C(F)F)o2)cn1)c1cccc(Cl)c1] |
| 285 | 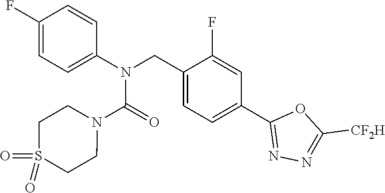 [SMILES: O=C(N1CCS(=O)(=O)CC1)N(Cc1ccc(-c2nnc(C(F)F)o2)cc1F)c1ccc(F)cc1] |
| 295 | 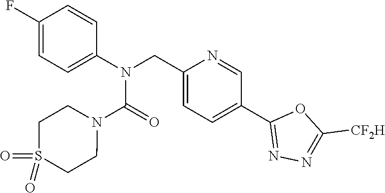 [SMILES: O=C(N1CCS(=O)(=O)CC1)N(Cc1ccc(-c2nnc(C(F)F)o2)cn1)c1ccc(F)cc1] |
| 296 | 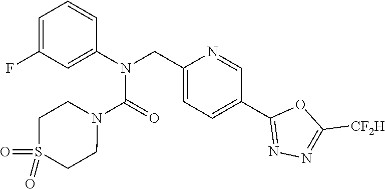 [SMILES: O=C(N1CCS(=O)(=O)CC1)N(Cc1ccc(-c2nnc(C(F)F)o2)cn1)c1cccc(F)c1] |

7. The pharmaceutical composition of claim 1, wherein the heart failure is at least one selected from the group consisting of Heart Failure with Preserved Ejection Fraction (HFpEF), Heart Failure with Midrange Ejection Fraction (HFmrEF) and Heart Failure with Reduced Ejection Fraction (HFrEF).

8. The pharmaceutical composition of claim 1, wherein the heart failure includes Cardiomyopathy.

9. The pharmaceutical composition of claim 8, wherein the Cardiomyopathy is at least one selected from the group consisting of Hypertrophic Cardiomyopathy (HCMP), Restrictive Cardiomyopathy and Dilated Cardiomyopathy (DCMP).

10. The pharmaceutical composition of claim 1, wherein the pharmaceutical composition is orally administered.

11. A method for preventing or treating heart failure, including administering a compound represented by formula I, optical isomers thereof or pharmaceutically acceptable salts thereof into an individual, wherein the formula I is the same as in claim 1.

12. A method for preventing or treating heart failure, including administering a compound, optical isomers thereof or pharmaceutically acceptable salts thereof into an individual, wherein the compound has the following structure:

| Com- | |
|---|---|
| pound | Structure |
| 40 | 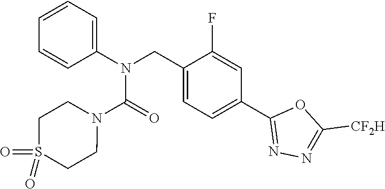 [SMILES: O=C(N1CCS(=O)(=O)CC1)N(Cc1ccc(-c2nnc(C(F)F)o2)cc1F)c1ccccc1] |
| 43 | 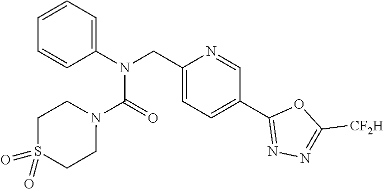 [SMILES: O=C(N1CCS(=O)(=O)CC1)N(Cc1ccc(-c2nnc(C(F)F)o2)cn1)c1ccccc1] |
| 239 | 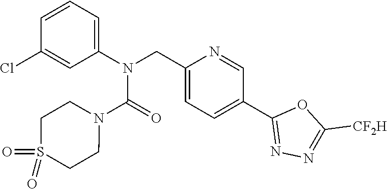 [SMILES: O=C(N1CCS(=O)(=O)CC1)N(Cc1ccc(-c2nnc(C(F)F)o2)cn1)c1cccc(Cl)c1] |
| 285 | 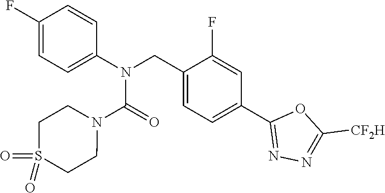 [SMILES: O=C(N1CCS(=O)(=O)CC1)N(Cc1ccc(-c2nnc(C(F)F)o2)cc1F)c1ccc(F)cc1] |
| 295 | 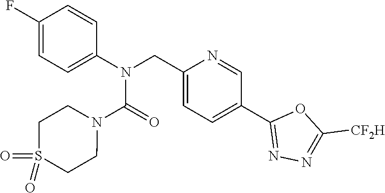 [SMILES: O=C(N1CCS(=O)(=O)CC1)N(Cc1ccc(-c2nnc(C(F)F)o2)cn1)c1ccc(F)cc1] |
| 296 | 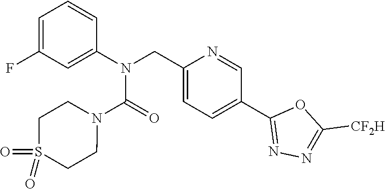 [SMILES: O=C(N1CCS(=O)(=O)CC1)N(Cc1ccc(-c2nnc(C(F)F)o2)cn1)c1cccc(F)c1] |

13. A use of a compound represented by the above formula I, optical isomers thereof or pharmaceutically acceptable salts thereof for preventing or treating heart failure, wherein the formula I is the same as in claim 1.

14. A use of a compound, optical isomers thereof or pharmaceutically acceptable salts thereof for preventing or treating heart failure, wherein the compound has the following structure:

| Com- | |
|---|---|
| pound | Structure |
| 40 | 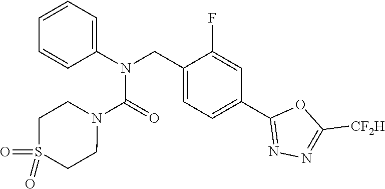 [SMILES: O=C(N1CCS(=O)(=O)CC1)N(Cc1ccc(-c2nnc(C(F)F)o2)cc1F)c1ccccc1] |
| 43 | 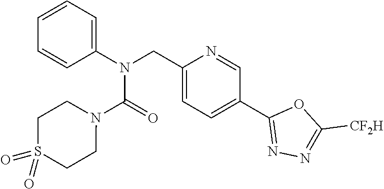 [SMILES: O=C(N1CCS(=O)(=O)CC1)N(Cc1ccc(-c2nnc(C(F)F)o2)cn1)c1ccccc1] |
| 239 | 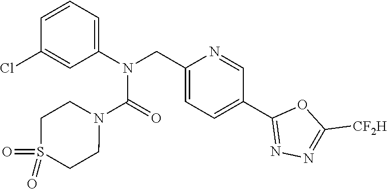 [SMILES: O=C(N1CCS(=O)(=O)CC1)N(Cc1ccc(-c2nnc(C(F)F)o2)cn1)c1cccc(Cl)c1] |
| 285 | 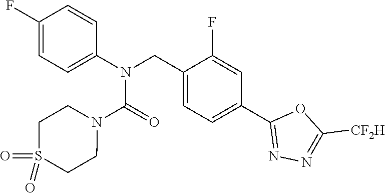 [SMILES: O=C(N1CCS(=O)(=O)CC1)N(Cc1ccc(-c2nnc(C(F)F)o2)cc1F)c1ccc(F)cc1] |
| 295 | 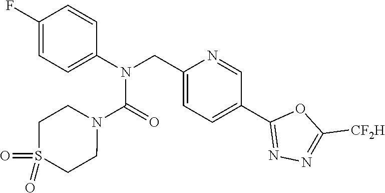 [SMILES: O=C(N1CCS(=O)(=O)CC1)N(Cc1ccc(-c2nnc(C(F)F)o2)cn1)c1ccc(F)cc1] |
| 296 | 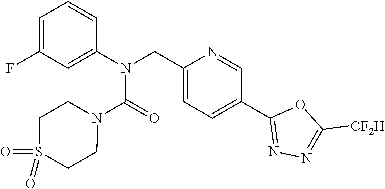 [SMILES: O=C(N1CCS(=O)(=O)CC1)N(Cc1ccc(-c2nnc(C(F)F)o2)cn1)c1cccc(F)c1] |

15. A use of the compound represented by above formula I, optical isomers thereof or pharmaceutically acceptable salts thereof in preparing a medicament for preventing or treating heart failure, wherein the formula I is the same as in claim 1.

16. A use of a compound, optical isomers thereof or pharmaceutically acceptable salts thereof in preparing a medicament for preventing or treating heart failure, wherein the compound has the following structure:

| Com- | |
|---|---|
| pound | Structure |
| 40 | 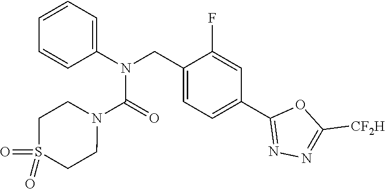 [SMILES: O=C(N1CCS(=O)(=O)CC1)N(Cc1ccc(-c2nnc(C(F)F)o2)cc1F)c1ccccc1] |
| 43 | 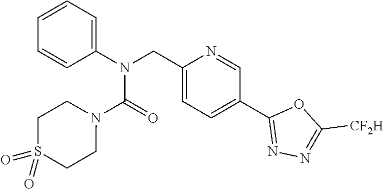 [SMILES: O=C(N1CCS(=O)(=O)CC1)N(Cc1ccc(-c2nnc(C(F)F)o2)cn1)c1ccccc1] |
| 239 | 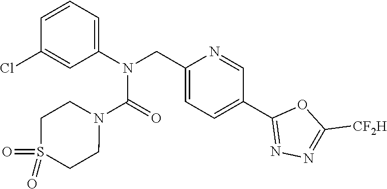 [SMILES: O=C(N1CCS(=O)(=O)CC1)N(Cc1ccc(-c2nnc(C(F)F)o2)cn1)c1cccc(Cl)c1] |
| 285 | 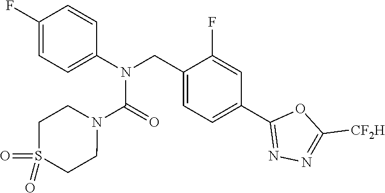 [SMILES: O=C(N1CCS(=O)(=O)CC1)N(Cc1ccc(-c2nnc(C(F)F)o2)cc1F)c1ccc(F)cc1] |
| 295 | 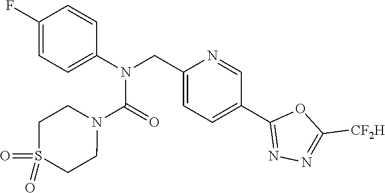 [SMILES: O=C(N1CCS(=O)(=O)CC1)N(Cc1ccc(-c2nnc(C(F)F)o2)cn1)c1ccc(F)cc1] |
| 296 | 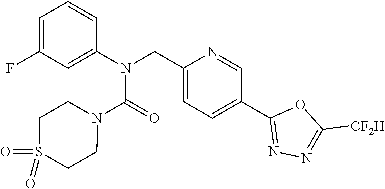 [SMILES: O=C(N1CCS(=O)(=O)CC1)N(Cc1ccc(-c2nnc(C(F)F)o2)cn1)c1cccc(F)c1] |
